(19) United States
(12) Patent Application Publication
(10) Pub. No.: US 2012/0095217 A1
(43) Pub. Date: Apr. 19, 2012

(54) FLUORINE CONTAINING COMPOUNDS AND METHODS OF USE THEREOF

(76) Inventors: Tobias Ritter, Cambridge, MA (US); Laura Brass, Newton, MA (US); Curtis Keith, Boston, MA (US); Alan Watson, Lexington, MA (US); David J. Greenblatt, Boston, MA (US)

(21) Appl. No.: 13/143,705

(22) PCT Filed: Jan. 8, 2010
(86) PCT No.: PCT/US2010/020544

§ 371 Date: Dec. 27, 2011

Related U.S. Application Data
(60) Provisional application No. 61/143,661, filed on Jan. 9, 2009.
(60) Provisional application No. 61/143,643, filed on Jan. 9, 2009.
(60) Provisional application No. 61/143,689, filed on Jan. 9, 2009.
(60) Provisional application No. 61/143,686, filed on Jan. 9, 2009.
(60) Provisional application No. 61/143,665, filed on Jan. 9, 2009.
(60) Provisional application No. 61/143,681, filed on Jan. 9, 2009.
(60) Provisional application No. 61/143,663, filed on Jan. 9, 2009.
(60) Provisional application No. 61/143,682, filed on Jan. 9, 2009.
(60) Provisional application No. 61/143,588, filed on Jan. 9, 2009.
(60) Provisional application No. 61/143,690, filed on Jan. 9, 2009.
(60) Provisional application No. 61/143,587, filed on Jan. 9, 2009.

(51) Int. Cl. C07D 413/04; C07D 471/08; C07C 217/62; C07C 211/42; C07D 471/04; C07C 211/29; C07D 223/28; C07D 403/06; C07C 211/32; C07C 211/31; C07D 313/12; C07D 333/20; C07D 471/14

(52) U.S. Cl. 540/551; 549/75; 544/343; 564/347; 564/308; 544/362; 540/578; 540/592; 544/366; 564/442; 564/316; 549/354; 564/336

(57) ABSTRACT

Fluorinated compounds and methods of making fluorinated compounds are described herein.

### RELATED APPLICATIONS

The present application claims priority under 35 U.S.C. §119(e) to U.S. provisional applications: U.S. Ser. No. 61/143,587, filed Jan. 9, 2009; U.S. Ser. No. 61/143,681, filed Jan. 9, 2009; U.S. Ser. No. 61/143,682, filed Jan. 9, 2009; U.S. Ser. No. 61/143,588, filed Jan. 9, 2009; U.S. Ser. No. 61/143,643, filed Jan. 9, 2009; U.S. Ser. No. 61/143,661, filed Jan. 9, 2009; U.S. Ser. No. 61/143,663, filed Jan. 9, 2009; U.S. Ser. No. 61/143,665, filed Jan. 9, 2009; U.S. Ser. No. 61/143,686, filed Jan. 9, 2009; U.S. Ser. No. 61/143,689, filed Jan. 9, 2009; and U.S. Ser. No. 61/143,690, filed Jan. 9, 2009; each of which is incorporated herein by reference.

### BACKGROUND OF INVENTION

Functionalized fluorine containing compounds (e.g. aryl fluorides) are often used as pharmaceutical agents. In some embodiments, these products have favorable pharmacological properties such as desirable metabolic stability.

### SUMMARY OF INVENTION

Described herein are methods of making fluorine containing compounds. Also described herein are fluorinated derivatives of compounds (e.g., pharmaceutical agents). Exemplary pharmaceutical agents include a compound described herein or a fluorinated derivative thereof, such as a pharmaceutical agent described herein.

In one aspect, the invention features a method of making a fluorinated compound, such as a compound described herein, using a method described herein.

In one aspect, the invention features a fluorinated atazanavir, for example, a derivative of atazanavir wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated atazanavir has the following formula:

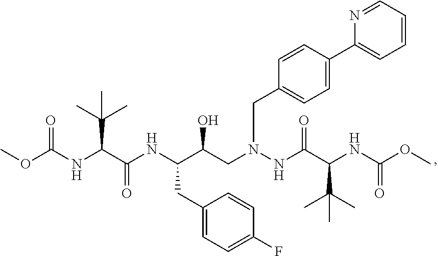

or a pharmaceutically acceptable salt thereof.

In one aspect, the invention features a method of making a fluorinated atazanavir, for example, the fluorinated atazanavir shown above, using a method described herein.

In one aspect, the invention features a fluorinated lopinavir, for example, a derivative of lopinavir wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated lopinavir is selected from one of the following:

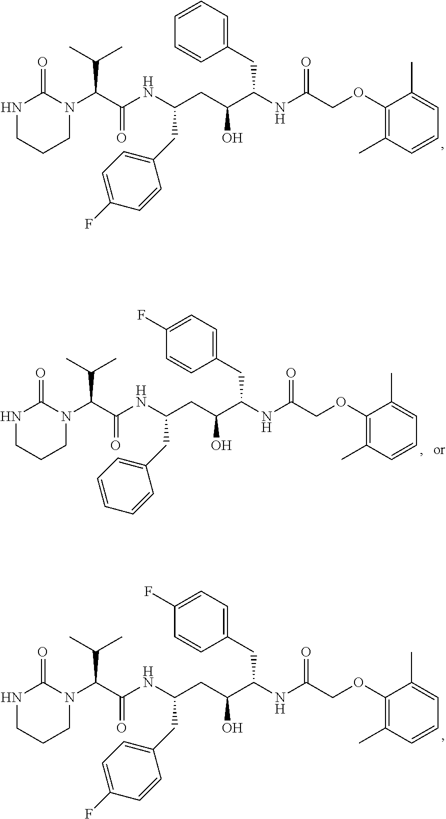

or a pharmaceutically acceptable salt thereof.

In one aspect, the invention features a method of making a fluorinated lopinavir, for example, a fluorinated lopinavir shown above, using a method described herein.

In one aspect, the invention features a fluorinated ritonavir, for example, a derivative of ritonavir wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated ritonavir is selected from one of the following:

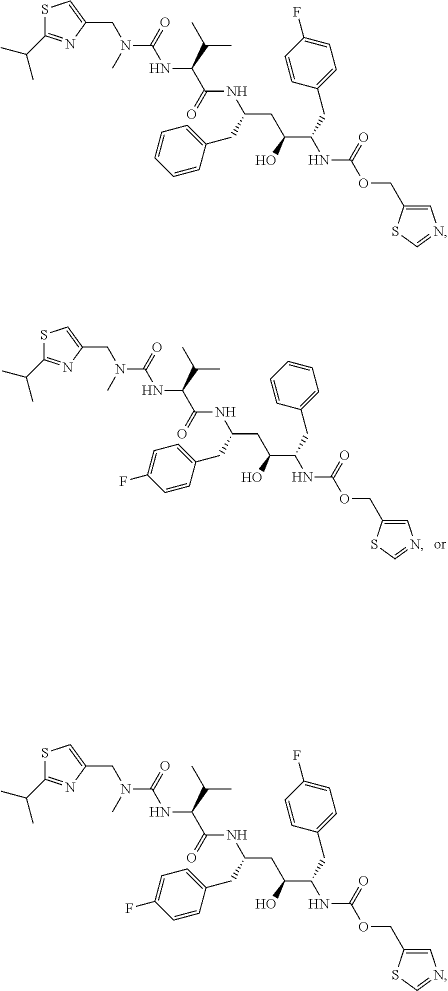

or a pharmaceutically acceptable salt thereof.

In one aspect, the invention features a method of making a fluorinated ritonavir, for example, a fluorinated ritonavir shown above, using a method described herein.

In one aspect, the invention features a fluorinated minocycline, for example, a derivative of minocycline wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated minocycline has the following formula:

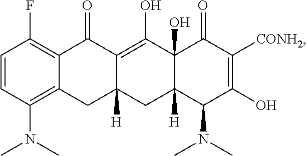

or a pharmaceutically acceptable salt thereof.

In one aspect, the invention features a method of making a fluorinated minocycline, for example, the fluorinated minocycline shown above, using a method described herein.

In one aspect, the invention features a fluorinated amoxicillin, for example, a derivative of amoxicillin wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated amoxicillin has the following formula:

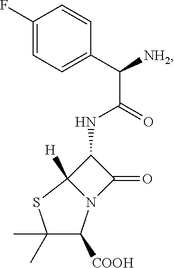

or a pharmaceutically acceptable salt thereof.

In one aspect, the invention features a method of making a fluorinated amoxicillin, for example, the fluorinated amoxicillin shown above, using a method described herein.

In one aspect, the invention features a fluorinated cephalexin, for example, a derivative of cephalexin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated cephalexin has the following formula:

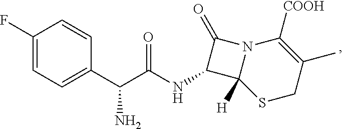

or a pharmaceutically acceptable salt thereof.

In one aspect, the invention features a method of making a fluorinated cephalexin, for example, the fluorinated cephalexin shown above, using a method described herein.

In one aspect, the invention features a fluorinated vancomycin, for example, a derivative of vancomycin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated vancomycin has the following formula:

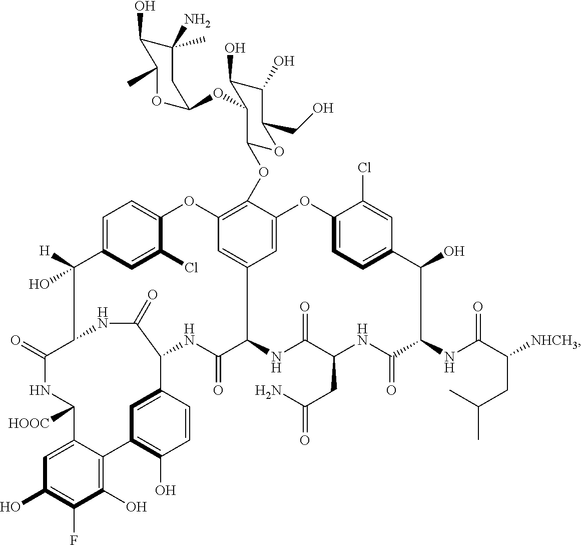

or a pharmaceutically acceptable salt thereof.

In one aspect, the invention features a method of making a fluorinated vancomycin, for example, the fluorinated vancomycin shown above, using a method described herein.

In one aspect, the invention features a fluorinated trimethoprim, for example, a derivative of trimethoprim wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated trimethoprim has the following formula:

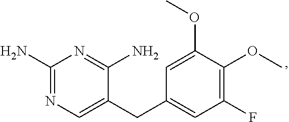

or a pharmaceutically acceptable salt thereof.

In one aspect, the invention features a method of making a fluorinated trimethoprim, for example, the fluorinated trimethoprim shown above, using a method described herein.

In one aspect, the invention features a fluorinated cefadroxil, for example, a derivative of cefadroxil wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated cefadroxil has the following formula:

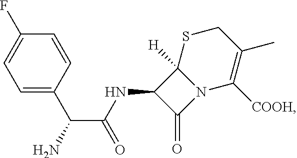

or a pharmaceutically acceptable salt thereof.

In one aspect, the invention features a method of making a fluorinated cefadroxil, for example, the fluorinated cefadroxil shown above, using a method described herein.

In one aspect, the invention features a fluorinated terconazole, for example, a derivative of terconazole wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated terconazole is selected from one of the following:

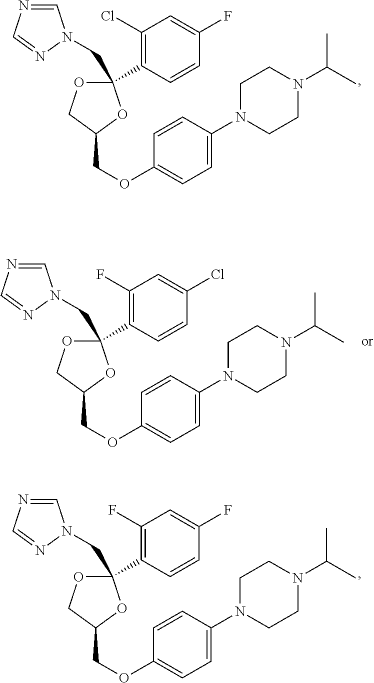

or a pharmaceutically acceptable salt thereof.

In one aspect, the invention features a method of making a fluorinated terconazole, for example, the fluorinated terconazole shown above, using a method described herein.

In one aspect, the invention features a fluorinated ampicillin, for example, a derivative of ampicillin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated ampicillin has the following formula:

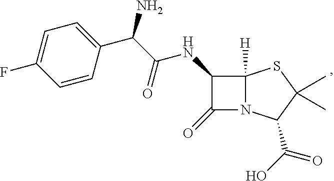

or a pharmaceutically acceptable salt thereof.

In one aspect, the invention features a method of making a fluorinated ampicillin, for example, the fluorinated ampicillin shown above, using a method described herein.

In one aspect, the invention features a fluorinated carbenicillin, for example, a derivative of carbenicillin wherein an aryl group has been substituted with one or more fluorine atoms, e.g., wherein a hydrogen of an aryl group has been replaced with a fluorine. In some embodiments, the fluorine substituent is F. In some embodiments, the fluorine substituent is ¹⁸F. In one embodiment, the fluorinated carbenicillin has the following formula:

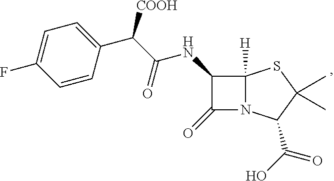

or a pharmaceutically acceptable salt thereof.

In one aspect, the invention features a method of making a fluorinated carbenicillin, for example, the fluorinated carbenicillin shown above, using a method described herein.

In one aspect, the invention features a fluorinated cefaclor, for example, a derivative of cefaclor wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated cefaclor has the following formula:

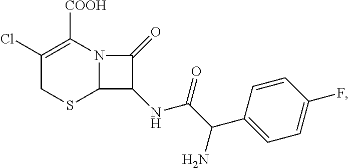

or a pharmaceutically acceptable salt thereof.

In one aspect, the invention features a method of making a fluorinated cefaclor, for example, the fluorinated cefaclor shown above, using a method described herein.

In one aspect, the invention features a fluorinated cefamandole, for example, a derivative of cefamandole wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated cefamandole has the following formula:

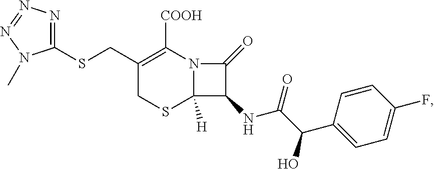

or a pharmaceutically acceptable salt thereof.

In one aspect, the invention features a method of making a fluorinated cefamandole, for example, the fluorinated cefamandole shown above, using a method described herein.

In one aspect, the invention features a fluorinated cefixime, for example, a derivative of cefixime wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated cefixime has the following formula:

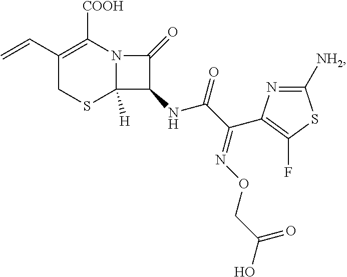

or a pharmaceutically acceptable salt thereof.

In one aspect, the invention features a method of making a fluorinated cefixime, for example, the fluorinated cefixime shown above, using a method described herein.

In one aspect, the invention features a fluorinated cefonicid, for example, a derivative of cefonicid wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated cefonicid has the following formula:

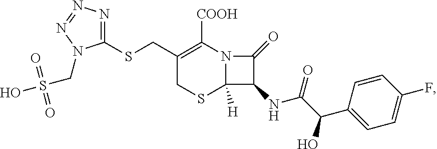

or a pharmaceutically acceptable salt thereof.

In one aspect, the invention features a method of making a fluorinated cefonicid, for example, the fluorinated cefonicid shown above, using a method described herein.

In one aspect, the invention features a fluorinated cefoperazone, for example, a derivative of cefoperazone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated cefoperazone has the following formula:

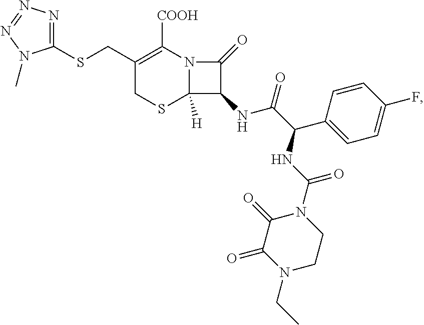

or a pharmaceutically acceptable salt thereof.

In one aspect, the invention features a method of making a fluorinated cefoperazone, for example, the fluorinated cefoperazone shown above, using a method described herein.

In one aspect, the invention features a fluorinated cefotaxime, for example, a derivative of cefotaxime wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated cefotaxime has the following formula:

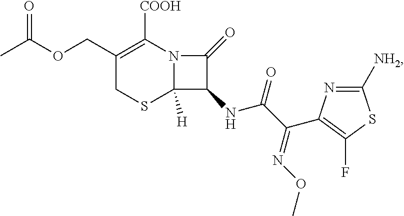

or a pharmaceutically acceptable salt thereof.

In one aspect, the invention features a method of making a fluorinated cefotaxime, for example, the fluorinated cefotaxime shown above, using a method described herein.

In one aspect, the invention features a fluorinated cefoxitin, for example, a derivative of cefoxitin wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated cefoxitin has the following formula:

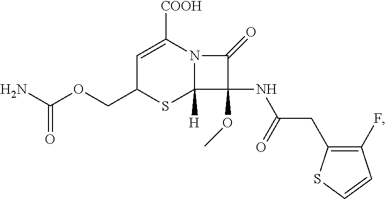

or a pharmaceutically acceptable salt thereof.

In one aspect, the invention features a method of making a fluorinated cefoxitin, for example, the fluorinated cefoxitin shown above, using a method described herein.

In one aspect, the invention features a fluorinated ceftazidime, for example, a derivative of ceftazidime wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated ceftazidime has the following formula:

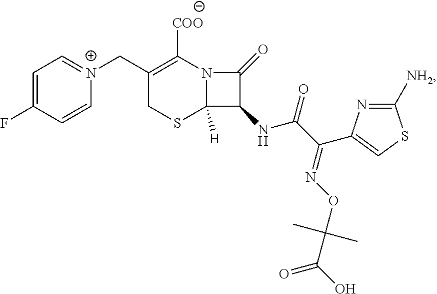

or a pharmaceutically acceptable salt thereof.

In one aspect, the invention features a method of making a fluorinated ceftazidime, for example, the fluorinated ceftazidime shown above, using a method described herein.

In one aspect, the invention features a fluorinated ceftriaxone, for example, a derivative of ceftriaxone wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated ceftriaxone has the following formula:

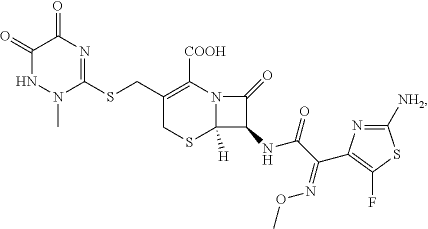

or a pharmaceutically acceptable salt thereof.

In one aspect, the invention features a method of making a fluorinated ceftriaxone, for example, the fluorinated ceftriaxone shown above, using a method described herein.

In one aspect, the invention features a fluorinated cephalothin, for example, a derivative of cephalothin wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated cephalothin has the following formula:

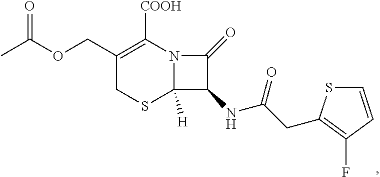

or a pharmaceutically acceptable salt thereof.

In one aspect, the invention features a method of making a fluorinated cephalothin, for example, the fluorinated cephalothin shown above, using a method described herein.

In one aspect, the invention features a fluorinated methicillin, for example, a derivative of methicillin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated methicillin has the following formula:

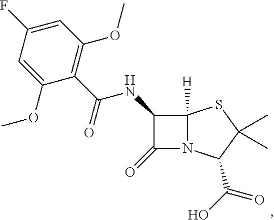

or a pharmaceutically acceptable salt thereof.

In one aspect, the invention features a method of making a fluorinated methicillin, for example, the fluorinated methicillin shown above, using a method described herein.

In one aspect, the invention features a fluorinated nafcillin, for example, a derivative of nafcillin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated nafcillin has the following formula:

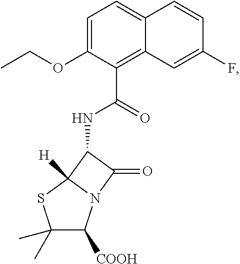

or a pharmaceutically acceptable salt thereof.

In one aspect, the invention features a method of making a fluorinated nafcillin, for example, the fluorinated nafcillin shown above, using a method described herein.

In one aspect, the invention features a fluorinated nalidixic acid, for example, a derivative of nalidixic acid wherein an aryl group has been substituted with one or more fluorine atoms, e.g., wherein a hydrogen or methyl substituent of an aryl group has been replaced with a fluorine. In some embodiments, the fluorine substituent is ¹⁹F. In some embodiments, the fluorine substituent is ¹⁸F. In one embodiment, the fluorinated nalidixic acid is selected from one of the following:

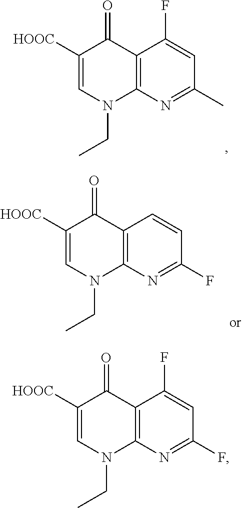

or a pharmaceutically acceptable salt thereof.

In one aspect, the invention features a method of making a fluorinated nalidixic acid, for example, a fluorinated nalidixic acid shown above, using a method described herein.

In one aspect, the invention features a fluorinated oxacillin, for example, a derivative of oxacillin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated oxacillin has the following formula:

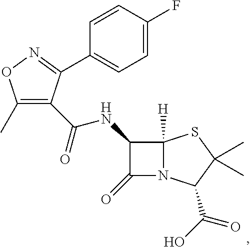

or a pharmaceutically acceptable salt thereof.

In one aspect, the invention features a method of making a fluorinated oxacillin, for example, the fluorinated oxacillin shown above, using a method described herein.

In one aspect, the invention features a fluorinated piperacillin, for example, a derivative of piperacillin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine subsitiuent is ¹⁸F. In some embodiment, the fluorinated piperacillin has the following formula:

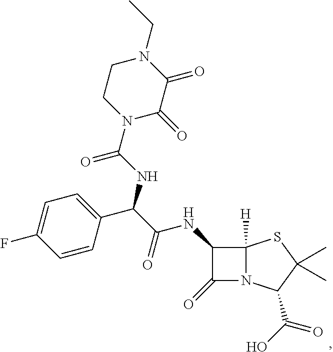

or a pharmaceutically acceptable salt thereof.

In one aspect, the invention features a method of making a fluorinated piperacillin, for example, the fluorinated piperacillin shown above, using a method described herein.

In one aspect, the invention features a fluorinated rifampin, for example, a derivative of rifampin wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated rifampin has the following formula:

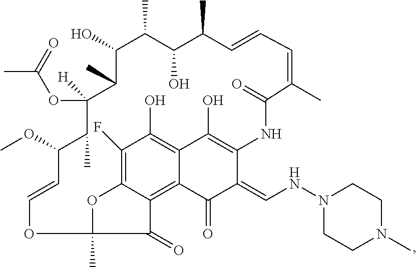

or a pharmaceutically acceptable salt thereof.

In one aspect, the invention features a method of making a fluorinated rifampin, for example, the fluorinated rifampin shown above, using a method described herein.

In one aspect, the invention features a fluorinated sulfisoxazole, for example, a derivative of sulfisoxazole wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated sulfisoxazole has the following formula:

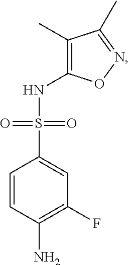

or a pharmaceutically acceptable salt thereof.

In one aspect, the invention features a method of making a fluorinated sulfisoxazole, for example, the fluorinated sulfisoxazole shown above, using a method described herein.

In one aspect, the invention features a fluorinated ticarcillin, for example, a derivative of ticarcillin wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated ticarcillin has the following formula:

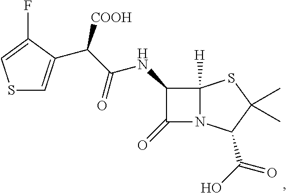

or a pharmaceutically acceptable salt thereof.

In one aspect, the invention features a method of making a fluorinated ticarcillin, for example, the fluorinated ticarcillin shown above, using a method described herein.

In one aspect, the invention features an ¹⁸F-substituted terbinafine, for example, a derivative of terbinafine wherein an aryl group has been substituted with one or more ¹⁸F atoms, e.g., wherein a hydrogen of an aryl group has been replaced with ¹⁸F. In one embodiment, the ¹⁸F-substituted terbinafine has the following formula:

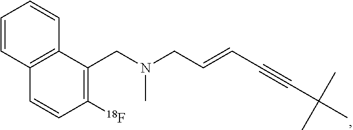

or a pharmaceutically acceptable salt thereof.

In one aspect, the invention features a method of making a fluorinated terbinafine, for example, a fluorinated terbinafine with the following formula, using a method described herein:

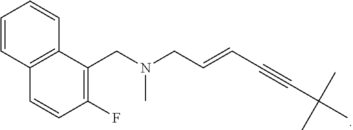

In one aspect, the invention features an ¹⁸F-substituted benzoyl peroxide, for example, a derivative of benzoyl peroxide wherein an aryl group has been substituted with one or more ¹⁸F atoms, e.g., wherein a hydrogen of an aryl group has been replaced with ¹⁸F. In one embodiment, the ¹⁸F-substituted benzoyl peroxide has the following formula:

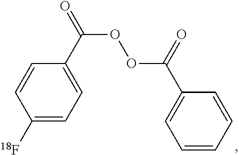

or a pharmaceutically acceptable salt thereof.

In one aspect, the invention features a method of making a fluorinated benzoyl peroxide, for example, a fluorinated benzoyl peroxide with the following formula, using a method described herein:

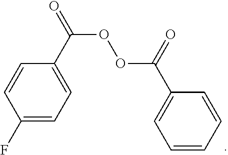

In one aspect, the invention features an ¹⁸F-substituted efavirenz, for example, a derivative of efavirenz wherein an aryl group has been substituted with one or more ¹⁸F atoms, e.g., wherein a hydrogen or halogen substituent of an aryl group has been replaced with ¹⁸F. In one embodiment, the ¹⁸F-substituted efavirenz has the following formula:

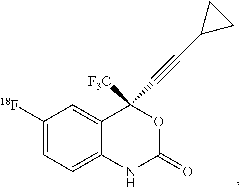

or a pharmaceutically acceptable salt thereof.

In one aspect, the invention features a method of making a fluorinated efavirenz, for example, a fluorinated efavirenz with the following formula, using a method described herein:

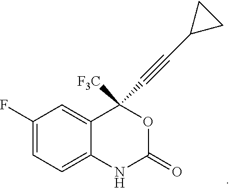

In one aspect, the invention features a fluorinated doxycycline, for example, a derivative of doxycycline wherein an aryl group has been substituted with one or more fluorine atoms, e.g., wherein a hydrogen or hydroxy substituent of an aryl group has been replaced with a fluorine. In some embodiments, the fluorinated doxycycline does not have the following formula:

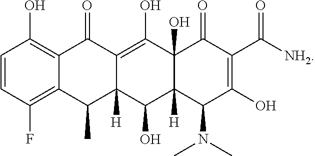

In some embodiments, the fluorine substituent is ¹⁹F. In some embodiments, the fluorine substituent is ¹⁸F. In some embodiments, the fluorinated doxycycline is selected from one of the following:

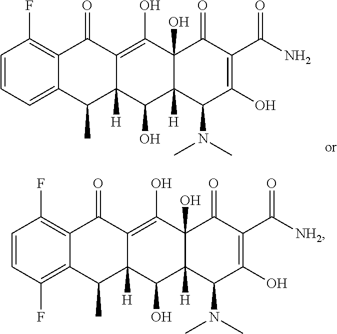

or a pharmaceutically acceptable salt thereof.

In one aspect, the invention features a method of making a fluorinated doxycycline, including any of the three fluorinated doxycycline structures shown above, using a method described herein.

In one aspect, the invention features an ¹⁸F-substituted doxycycline, for example, a derivative of doxycycline wherein an aryl group has been substituted with one or more ¹⁸F atoms, e.g., wherein a hydrogen or hydroxy substituent of an aryl group has been replaced with ¹⁸F. In one embodiment, the ¹⁸F-substituted doxycycline has the following formula:

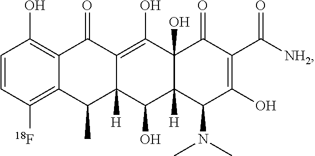

or a pharmaceutically acceptable salt thereof.

In one aspect, the invention features an ¹⁸F-substituted clotrimazole, for example, a derivative of clotrimazole wherein an aryl group has been substituted with one or more ¹⁸F atoms, e.g., wherein a hydrogen or halogen substituent of an aryl group has been replaced with ¹⁸F. In one embodiment, the ¹⁸F-substituted clotrimazole has the following formula:

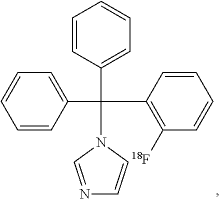

or a pharmaceutically acceptable salt thereof.

In one aspect, the invention features a method of making a fluorinated clotrimazole, for example, a fluorinated clotrimazole with the following formula, using a method described herein:

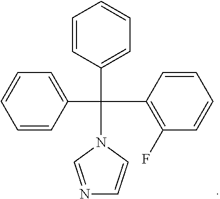

In one aspect, the invention features a fluorinated ketoconazole, for example, a derivative of ketoconazole wherein an aryl group has been substituted with one or more fluorine atoms, e.g., wherein a hydrogen or halogen substituent of an aryl group has been replaced with a fluorine. In some embodiments, the fluorinated ketoconazole does not have the following formula:

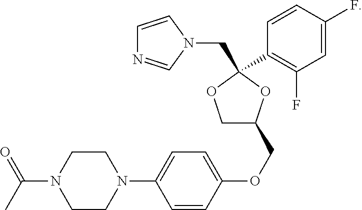

In some embodiments, the fluorine substituent is ¹⁹F. In some embodiments, the fluorine substituent is ¹⁸F. In some embodiments, the fluorinated ketoconazole is selected from one of the following:

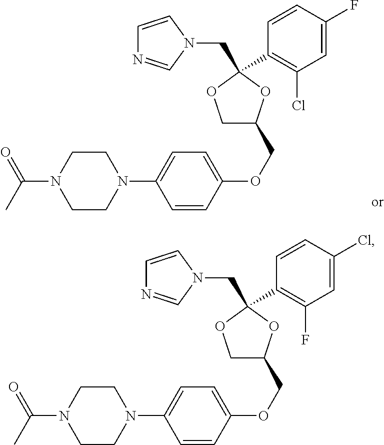

or a pharmaceutically acceptable salt thereof.

In one aspect, the invention features a method of making a fluorinated ketoconazole, including any of the three fluorinated ketoconazole structures shown above, using a method described herein.

In one aspect, the invention features an ¹⁸F-substituted ketoconazole, for example, a derivative of ketoconazole wherein an aryl group has been substituted with one or more ¹⁸F atoms, e.g., wherein a hydrogen or halogen substituent of an aryl group has been replaced with ¹⁸F. In one embodiment, the ¹⁸F-substituted ketoconazole has the following formula:

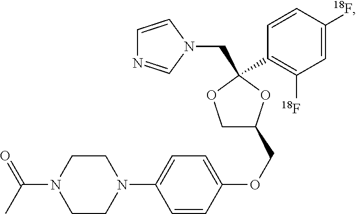

or a pharmaceutically acceptable salt thereof.

In one aspect, the invention features an ¹⁸F-substituted hydroxychloroquine, for example, a derivative of hydroxychloroquine wherein an aryl group has been substituted with one or more ¹⁸F atoms, e.g., wherein a hydrogen or halogen substituent of an aryl group has been replaced with ¹⁸F. In one embodiment, the ¹⁸F-substituted hydroxychloroquine has the following formula:

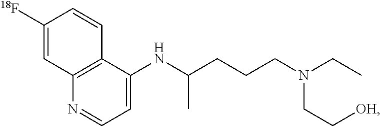

or a pharmaceutically acceptable salt thereof.

In one aspect, the invention features a method of making a fluorinated hydroxychloroquine, for example, a fluorinated hydroxychloroquine with the following formula, using a method described herein:

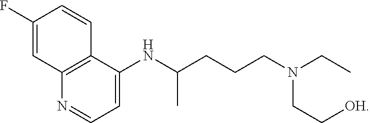

In one aspect, the invention features a fluorinated itraconazole, for example, a derivative of itraconazole wherein an aryl group has been substituted with one or more fluorine atoms, e.g., wherein a hydrogen or halogen substituent of an aryl group has been replaced with a fluorine. In some embodiments, the fluorinated itraconazole does not have the following formula:

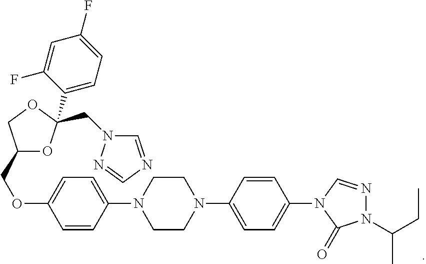

In some embodiments, the fluorine substituent is ¹⁹F. In some embodiments, the fluorine substituent is ¹⁸F. In some embodiments, the fluorinated itraconazole is selected from one of the following:

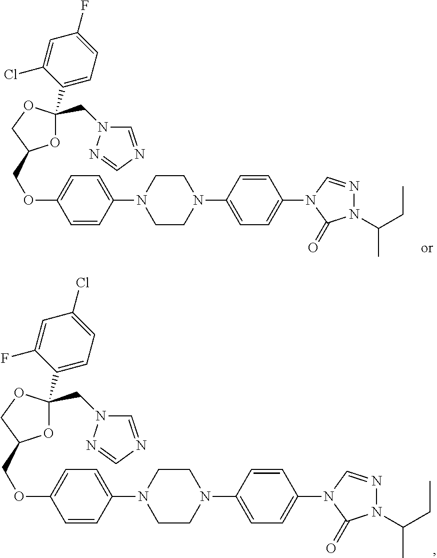

or a pharmaceutically acceptable salt thereof.

In one aspect, the invention features a method of making a fluorinated itraconazole, including any of the three fluorinated itraconazole structures shown above, using a method described herein.

In one aspect, the invention features an ¹⁸F-substituted itraconazole, for example, a derivative of itraconazole wherein an aryl group has been substituted with one or more ¹⁸F atoms, e.g., wherein a hydrogen or halogen substituent of an aryl group has been replaced with ¹⁸F. In one embodiment, the ¹⁸F-substituted itraconazole has the following formula:

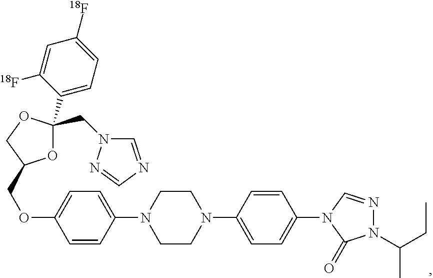

or a pharmaceutically acceptable salt thereof.

In one aspect, the invention features an ¹⁸F-substituted cephalosporin, for example, a derivative of cephalosporin wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted cephalosporin has the following formula:

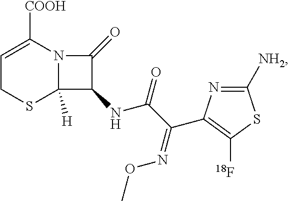

or a pharmaceutically acceptable salt thereof.

In one aspect, the invention features a method of making a fluorinated cephalosporin, for example, a fluorinated cephalosporin with the following formula, using a method described herein:

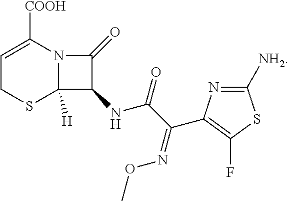

In one aspect, the invention features a fluorinated tetracycline, for example, a derivative of tetracycline wherein an aryl group has been substituted with one or more fluorine atoms, e.g., wherein a hydrogen or hydroxy substituent of an aryl group has been replaced with a fluorine. In some embodiments, the fluorinated tetracycline does not have the following formula:

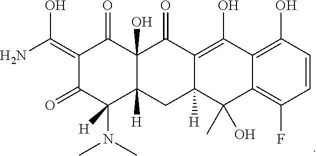

In some embodiments, the fluorine substituent is ¹⁹F. In some embodiments, the fluorine substituent is ¹⁸F. In some embodiments, the fluorinated tetracycline is selected from one of the following:

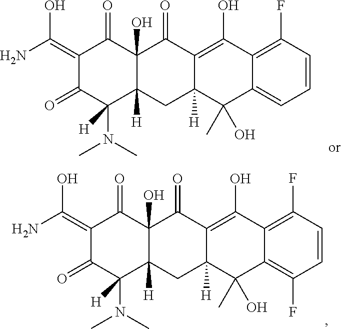

or a pharmaceutically acceptable salt thereof.

In one aspect, the invention features a method of making a fluorinated tetracycline, including any of the three fluorinated tetracycline structures shown above, using a method described herein.

In one aspect, the invention features an ¹⁸F-substituted tetracycline, for example, a derivative of tetracycline wherein an aryl group has been substituted with one or more ¹⁸F atoms, e.g., wherein a hydrogen or hydroxy substituent of an aryl group has been replaced with ¹⁸F. In one embodiment, the ¹⁸F-substituted tetracycline has the following formula:

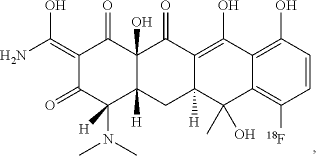

or a pharmaceutically acceptable salt thereof.

In one aspect, the invention features a fluorinated ramipril, for example, a derivative of ramipril wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated ramipril has the following formula:

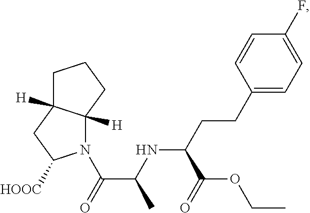

or a pharmaceutically acceptable salt thereof.

In one aspect, the invention features a method of making a fluorinated ramipril, for example, the fluorinated ramipril shown above, using a method described herein.

In one aspect, the invention features a fluorinated losartan, for example, a derivative of losartan wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated losartan has the following formula:

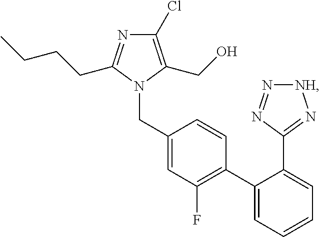

or a pharmaceutically acceptable salt thereof.

In one aspect, the invention features a method of making a fluorinated losartan, for example, the fluorinated losartan shown above, using a method described herein.

In one aspect, the invention features a fluorinated olmesartan, for example, a derivative of olmesartan wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated olmesartan has the following formula:

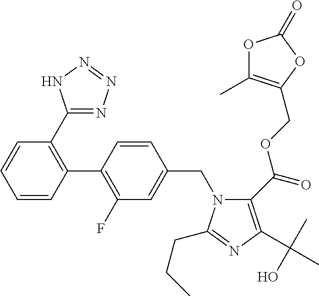

or a pharmaceutically acceptable salt thereof.

In one aspect, the invention features a method of making a fluorinated olmesartan, for example, the fluorinated olmesartan shown above, using a method described herein.

In one aspect, the invention features a fluorinated candesartan, for example, a derivative of candesartan wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated candesartan is selected from one of the following:

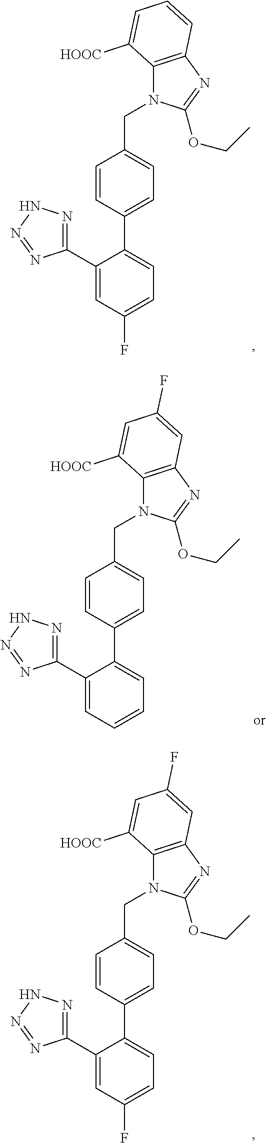

or a pharmaceutically acceptable salt thereof.

In one aspect, the invention features a method of making a fluorinated candesartan, for example, a fluorinated candesartan shown above, using a method described herein.

In one aspect, the invention features a fluorinated felodipine, for example, a derivative of felodipine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated felodipine is selected from one of the following:

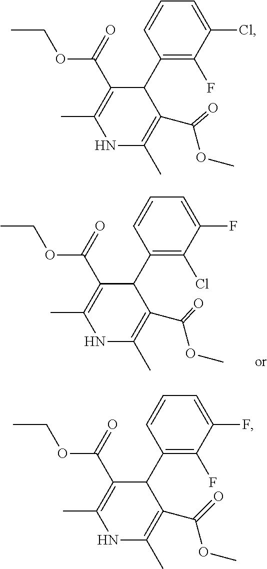

or a pharmaceutically acceptable salt thereof.

In one aspect, the invention features a method of making a fluorinated felodipine, for example, a fluorinated felodipine shown above, using a method described herein.

In one aspect, the invention features a fluorinated propranolol, for example, a derivative of propranolol wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated propranolol has the following formula:

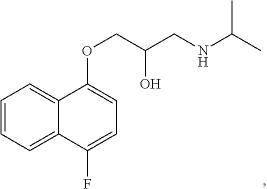

or a pharmaceutically acceptable salt thereof.

In one aspect, the invention features a method of making a fluorinated propranolol, for example, the fluorinated propranolol shown above, using a method described herein.

In one aspect, the invention features a fluorinated benazepril, for example, a derivative of benazepril wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated benazepril is selected from one of the following:

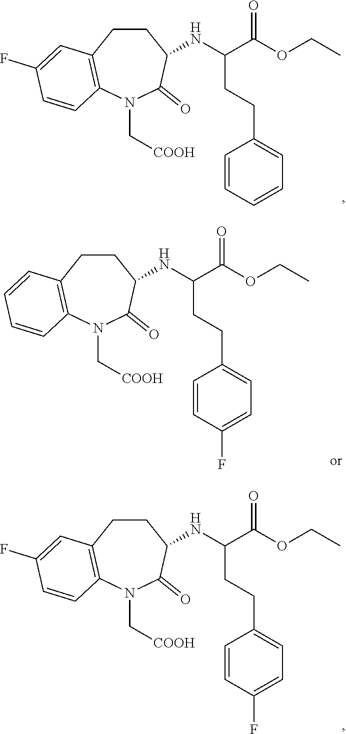

or a pharmaceutically acceptable salt thereof.

In one aspect, the invention features a method of making a fluorinated benazepril, for example, a fluorinated benazepril shown above, using a method described herein.

In one aspect, the invention features a fluorinated fosinopril, for example, a derivative of fosinopril wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated fosinopril has the following formula:

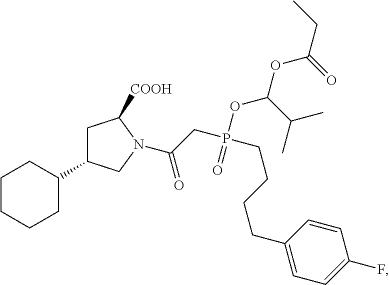

or a pharmaceutically acceptable salt thereof.

In one aspect, the invention features a method of making a fluorinated fosinopril, for example, the fluorinated fosinopril shown above, using a method described herein.

In one aspect, the invention features a fluorinated doxazosin, for example, a derivative of doxazosin wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated doxazosin is selected from one of the following:

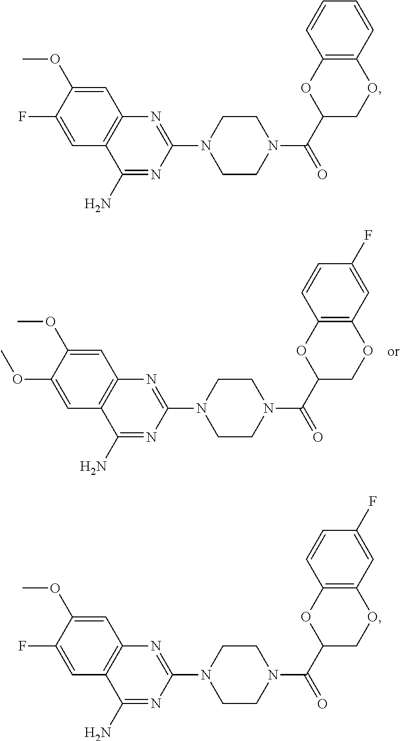

or a pharmaceutically acceptable salt thereof.

In one aspect, the invention features a method of making a fluorinated doxazosin, for example, a fluorinated doxazosin shown above, using a method described herein.

In one aspect, the invention features a fluorinated midodrine, for example, a derivative of midodrine wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated midodrine has the following formula:

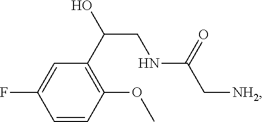

or a pharmaceutically acceptable salt thereof.

In one aspect, the invention features a method of making a fluorinated midodrine, for example, the fluorinated midodrine shown above, using a method described herein.

In one aspect, the invention features an ¹⁸F-substituted hydrochlorothiazide, for example, a derivative of hydrochlorothiazide wherein an aryl group has been substituted with one or more ¹⁸F atoms, e.g., wherein a hydrogen of an aryl group has been replaced with ¹⁸F. In one embodiment, the ¹⁸F-substituted hydrochlorothiazide has the following formula:

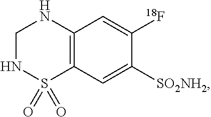

or a pharmaceutically acceptable salt thereof.

In one aspect, the invention features a method of making a fluorinated hydrochlorothiazide, for example, a fluorinated hydrochlorothiazide with the following formula, using a method described herein:

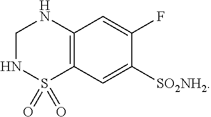

In one aspect, the invention features an ¹⁸F-substituted sildenafil, for example, a derivative of sildenafil wherein an aryl or heteroaryl group has been substituted with one or more ¹⁸F atoms, e.g., wherein a hydrogen or alkoxy substituent of an aryl or heteroaryl group has been replaced with ¹⁸F. In one embodiment, the ¹⁸F-substituted sildenafil has the following formula:

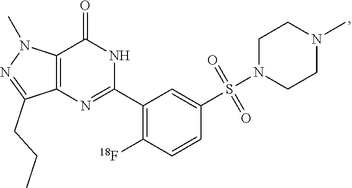

or a pharmaceutically acceptable salt thereof.

In one aspect, the invention features a method of making a fluorinated sildenafil, for example, a fluorinated sildenafil with the following formula, using a method described herein:

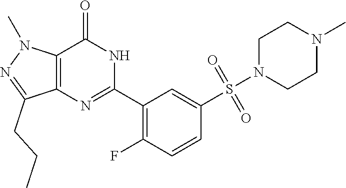

In one aspect, the invention features an ¹⁸F-substituted amlodipine, for example, a derivative of amlodipine wherein an aryl group has been substituted with one or more ¹⁸F atoms, e.g., wherein a hydrogen or halogen substituent of an aryl group has been replaced with ¹⁸F. In one embodiment, the ¹⁸F-substituted amlodipine has the following formula:

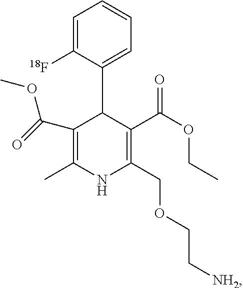

or a pharmaceutically acceptable salt thereof.

In one aspect, the invention features a method of making a fluorinated amlodipine, for example, a fluorinated amlodipine with the following formula, using a method described herein:

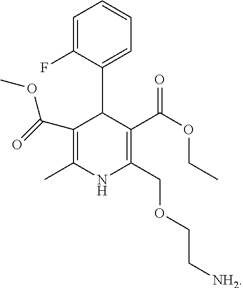

In one aspect, the invention features an ¹⁸F-substituted tadalafil, for example, a derivative of tadalafil wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted tadalafil has the following formula:

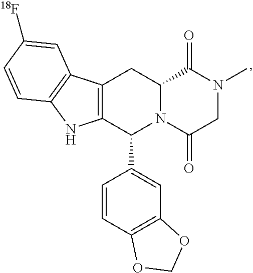

or a pharmaceutically acceptable salt thereof.

In one aspect, the invention features a method of making a fluorinated tadalafil, for example, a fluorinated tadalafil with the following formula, using a method described herein:

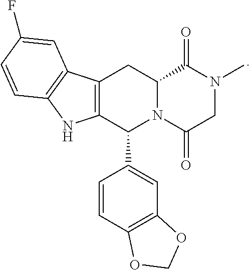

In one aspect, the invention features an ¹⁸F-substituted lisinopril, for example, a derivative of lisinopril wherein an aryl group has been substituted with one or more ¹⁸F atoms, e.g., wherein a hydrogen of an aryl group has been replaced with ¹⁸F. In one embodiment, the ¹⁸F-substituted lisinopril has the following formula:

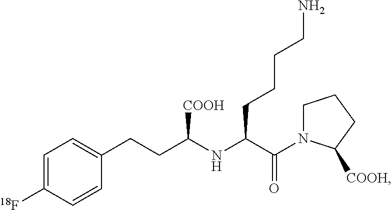

or a pharmaceutically acceptable salt thereof.

In one aspect, the invention features a method of making a fluorinated lisinopril, for example, a fluorinated lisinopril with the following formula, using a method described herein:

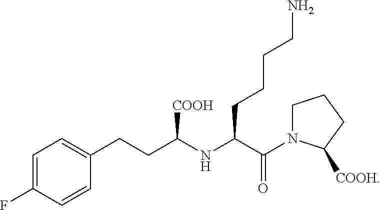

In one aspect, the invention features an ¹⁸F-substituted nifedipine, for example, a derivative of nifedipine wherein an aryl group has been substituted with one or more ¹⁸F atoms, e.g., wherein a hydrogen or nitro substituent of an aryl group has been replaced with ¹⁸F. In one embodiment, the ¹⁸F-substituted nifedipine has the following formula:

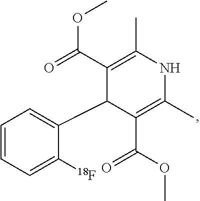

or a pharmaceutically acceptable salt thereof.

In one aspect, the invention features a method of making a fluorinated nifedipine, for example, a fluorinated nifedipine with the following formula, using a method described herein:

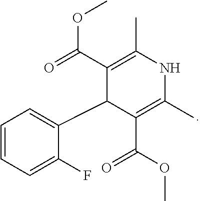

In one aspect, the invention features a fluorinated diltiazem, for example, a derivative of diltiazem wherein an aryl group has been substituted with one or more fluorine atoms, e.g., wherein a hydrogen or alkoxy substituent of an aryl group has been replaced with a fluorine. In some embodiments, the fluorinated diltiazem does not have the following formula:

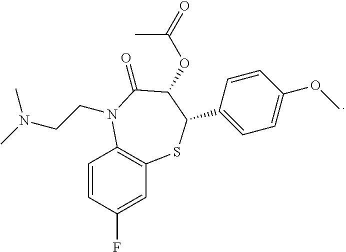

In some embodiments, the fluorine substituent is ¹⁹F. In some embodiments, the fluorine substituent is ¹⁸F. In some embodiments, the fluorinated diltiazem is selected from one of the following:

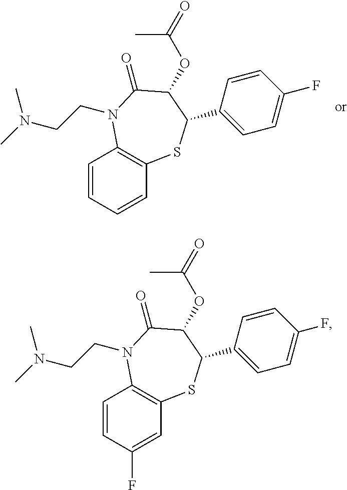

or a pharmaceutically acceptable salt thereof.

In one aspect, the invention features a method of making a fluorinated diltiazem, including any of the three fluorinated diltiazem structures shown above, using a method described herein.

In one aspect, the invention features an ¹⁸F-substituted diltiazem, for example, a derivative of diltiazem wherein an aryl group has been substituted with one or more ¹⁸F atoms, e.g., wherein a hydrogen or alkoxy substituent of an aryl group has been replaced with ¹⁸F. In one embodiment, the ¹⁸F-substituted diltiazem has the following formula:

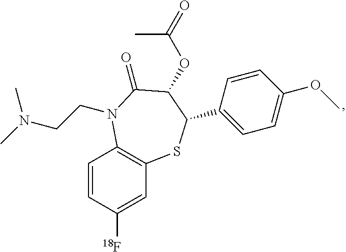

or a pharmaceutically acceptable salt thereof.

In one aspect, the invention features a fluorinated quinapril, for example, a derivative of quinapril wherein an aryl group has been substituted with one or more fluorine atoms, e.g., wherein a hydrogen or halogen substituent of an aryl group has been replaced with a fluorine. In some embodiments, the fluorinated quinapril does not have the following formula:

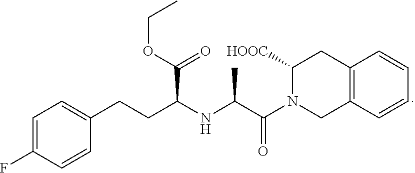

In some embodiments, the fluorine substituent is ¹⁹F. In some embodiments, the fluorine substituent is ¹⁸F. In some embodiments, the fluorinated quinapril is selected from one of the following:

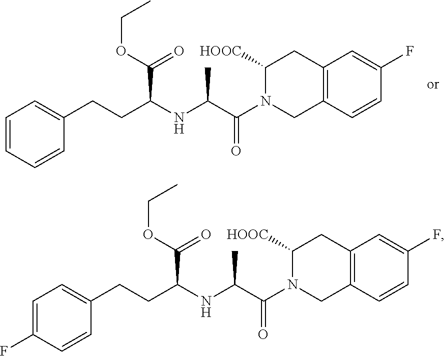

or a pharmaceutically acceptable salt thereof.

In one aspect, the invention features a method of making a fluorinated quinapril, including any of the three fluorinated quinapril structures shown above, using a method described herein.

In one aspect, the invention features an ¹⁸F-substituted quinapril, for example, a derivative of quinapril wherein an aryl group has been substituted with one or more ¹⁸F atoms, e.g., wherein a hydrogen of an aryl group has been replaced with ¹⁸F. In one embodiment, the ¹⁸F-substituted quinapril has the following formula:

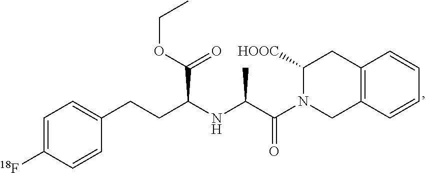

or a pharmaceutically acceptable salt thereof.

In one aspect, the invention features an ¹⁸F-substituted enalapril, for example, a derivative of enalapril wherein an aryl group has been substituted with one or more ¹⁸F atoms, e.g., wherein a hydrogen of an aryl group has been replaced with ¹⁸F. In one embodiment, the ¹⁸F-substituted enalapril has the following formula:

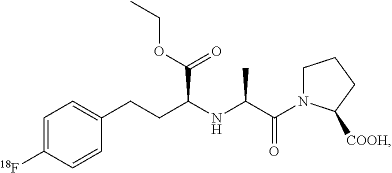

or a pharmaceutically acceptable salt thereof.

In one aspect, the invention features a method of making a fluorinated enalapril, for example, a fluorinated enalapril with the following formula, using a method described herein:

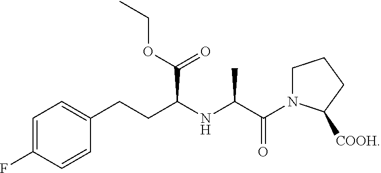

In one aspect, the invention features a fluorinated labetalol, for example, a derivative of labetalol wherein an aryl group has been substituted with one or more fluorine atoms, e.g., wherein a hydrogen or hydroxy substituent of an aryl group has been replaced with a fluorine. In some embodiments, the fluorinated labetalol does not have the following formula:

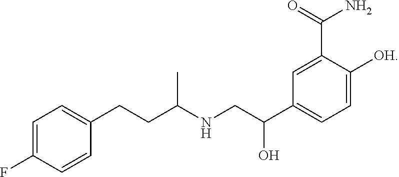

In some embodiments, the fluorine substituent is ¹⁹F. In some embodiments, the fluorine substituent is ¹⁸F. In some embodiments, the fluorinated labetalol is selected from one of the following:

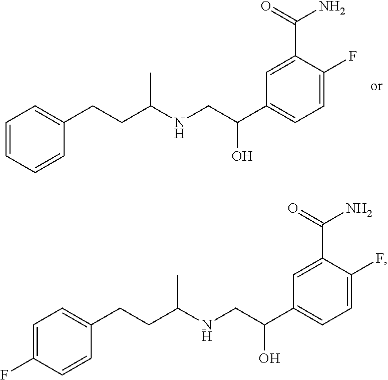

or a pharmaceutically acceptable salt thereof.

In one aspect, the invention features a method of making a fluorinated labetalol, including any of the three fluorinated labetalol structures shown above, using a method described herein.

In one aspect, the invention features an ¹⁸F-substituted labetalol, for example, a derivative of labetalol wherein an aryl group has been substituted with one or more ¹⁸F atoms, e.g., wherein a hydrogen or hydroxy substituent of an aryl group has been replaced with ¹⁸F.

In one embodiment, the ¹⁸F-substituted labetalol has the following formula:

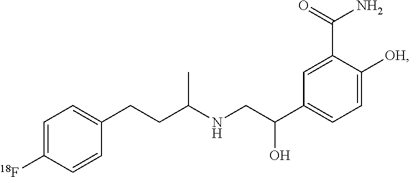

or a pharmaceutically acceptable salt thereof.

In one aspect, the invention features a fluorinated tiotropium, for example, a derivative of tiotropium wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In some embodiments, the fluorinated tiotropium has the following formula:

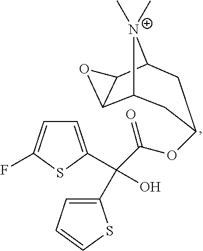

or a pharmaceutically acceptable salt thereof, e.g., a bromide salt.

In one aspect, the invention features a method of making a fluorinated tiotropium, for example, the fluorinated tiotropium shown above, using a method described herein.

In one aspect, the invention features a fluorinated salbutamol, for example, a derivative of salbutamol wherein an aryl group has been substituted with one or more fluorine atoms, e.g., wherein a hydrogen or hydroxyl substituent of an aryl group has been replaced with a fluorine. In some embodiments, the fluorine substituent is ¹⁹F. In some embodiments, the fluorine substituent is ¹⁸F. In some embodiments, the fluorinated salbutamol has the following formula:

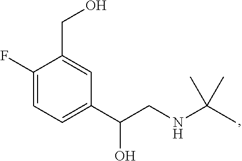

or a pharmaceutically acceptable salt thereof.

In one aspect, the invention features a method of making a fluorinated salbutamol, for example, the fluorinated salbutamol shown above, using a method described herein.

In one aspect, the invention features a fluorinated fexofenadine, for example, a derivative of fexofenadine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated fexofenadine is selected from one of the following:

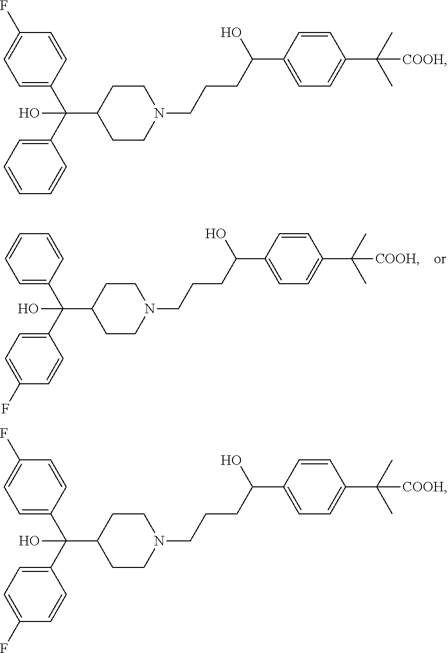

or a pharmaceutically acceptable salt thereof.

In one aspect, the invention features a method of making a fluorinated fexofenadine, for example, a fluorinated fexofenadine shown above, using a method described herein.

In one aspect, the invention features a fluorinated eletriptan, for example, a derivative of eletriptan wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated eletriptan has the following formula.

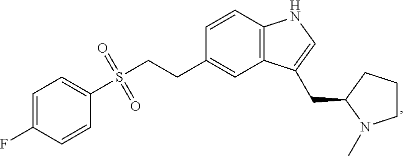

or a pharmaceutically acceptable salt thereof.

In one aspect, the invention features a method of making a fluorinated eletriptan, for example, the fluorinated eletriptan shown above, using a method described herein.

In one aspect, the invention features a fluorinated nabumetone, for example, a derivative of nabumetone wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated nabumetone has the following formula:

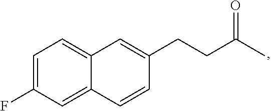

or a pharmaceutically acceptable salt thereof.

In one aspect, the invention features a method of making a fluorinated nabumetone, for example, the fluorinated nabumetone shown above, using a method described herein.

In one aspect, the invention features a fluorinated hydroxyzine, for example, a derivative of hydroxyzine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated hydroxyzine has the following formula:

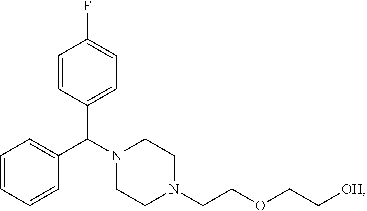

or a pharmaceutically acceptable salt thereof.

In one aspect, the invention features a method of making a fluorinated hydroxyzine, for example, the fluorinated hydroxyzine shown above, using a method described herein.

In one aspect, the invention features a fluorinated promethazine, for example, a derivative of promethazine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated promethazine is selected from one of the following:

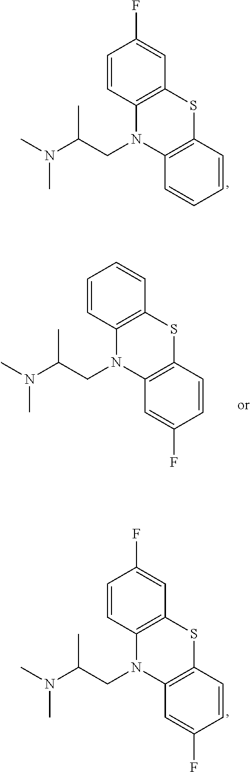

or a pharmaceutically acceptable salt thereof.

In one aspect, the invention features a method of making a fluorinated promethazine, for example, a fluorinated promethazine shown above, using a method described herein.

In one aspect, the invention features a fluorinated etodolac, for example, a derivative of etodolac wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated etodolac has the following formula:

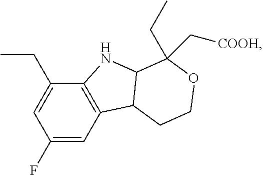

or a pharmaceutically acceptable salt thereof.

In one aspect, the invention features a method of making a fluorinated etodolac, for example, the fluorinated etodolac shown above, using a method described herein.

In one aspect, the invention features a fluorinated albuterol, for example, a derivative of albuterol wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine subsittuent is ¹⁸F. In one embodiment, the fluorinated albuterol has the following formula:

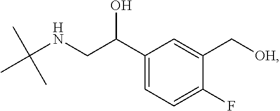

or a pharmaceutically acceptable salt thereof.

In one aspect, the invention features a method of making a fluorinated albuterol, for example, the fluorinated albuterol shown above, using a method described herein.

In one aspect, the invention features a fluorinated ipratropium, for example, a derivative of ipratropium wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated ipratropium has the following formula:

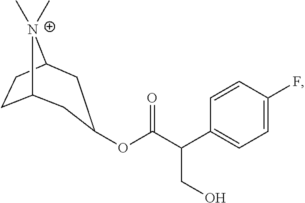

or a pharmaceutically acceptable salt thereof, e.g., a bromide salt.

In one aspect, the invention features a method of making a fluorinated ipratropium, for example, the fluorinated ipratropium shown above, using a method described herein.

In one aspect, the invention features a fluorinated meclozine, for example, a derivative of meclozine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated meclozine is selected from one of the following:

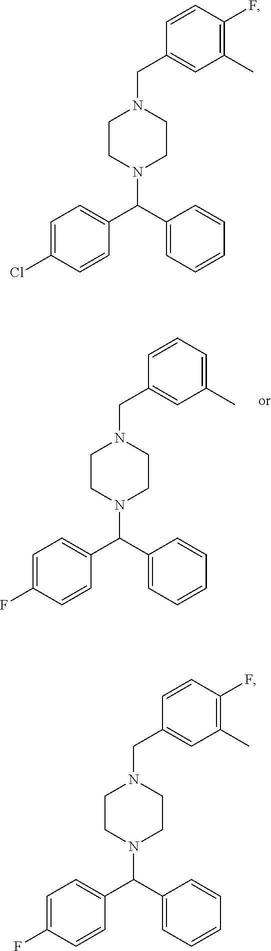

or a pharmaceutically acceptable salt thereof.

In one aspect, the invention features a method of making a fluorinated meclozine, for example, a fluorinated meclozine shown above, using a method described herein.

In one aspect, the invention features a fluorinated tolfenamic acid, for example, a derivative of tolfenamic acid wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated tolfenamic acid is selected from one of the following:

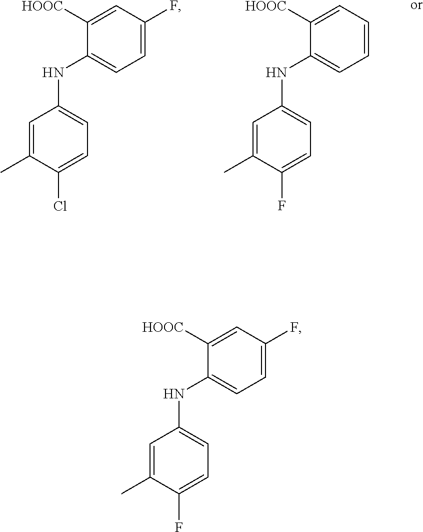

or a pharmaceutically acceptable salt thereof.

In one aspect, the invention features a method of making a fluorinated tolfenamic acid, for example, a fluorinated tolfenamic acid shown above, using a method described herein.

In one aspect, the invention features a fluorinated almotriptan, for example, a derivative of almotriptan wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated almotriptan has the following formula:

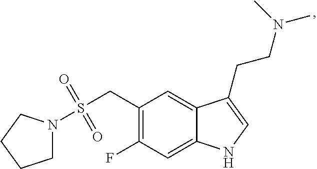

or a pharmaceutically acceptable salt thereof.

In one aspect, the invention features a method of making a fluorinated almotriptan, for example, the fluorinated almotriptan shown above, using a method described herein.

In one aspect, the invention features a fluorinated zolmitriptan, for example, a derivative of zolmitriptan wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated zolmitriptan has the following formula:

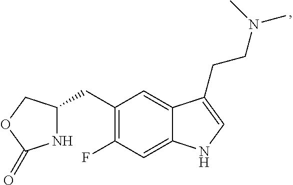

or a pharmaceutically acceptable salt thereof.

In one aspect, the invention features a method of making a fluorinated zolmitriptan, for example, the fluorinated zolmitriptan shown above, using a method described herein.

In one aspect, the invention features a fluorinated pizotifen, for example, a derivative of pizotifen wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated pizotifen has the following formula:

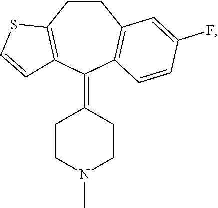

or a pharmaceutically acceptable salt thereof.

In one aspect, the invention features a method of making a fluorinated pizotifen, for example, the fluorinated pizotifen shown above, using a method described herein.

In one aspect, the invention features a fluorinated methysergide, for example, a derivative of methysergide wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated methysergide has the following formula:

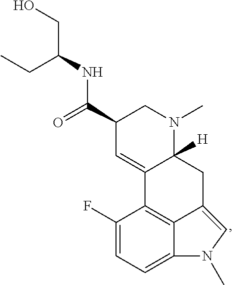

or a pharmaceutically acceptable salt thereof.

In one aspect, the invention features a method of making a fluorinated methysergide, for example, the fluorinated methysergide shown above, using a method described herein.

In one aspect, the invention features a fluorinated montelukast, for example, a derivative of montelukast wherein an aryl or heteroaryl group has been substituted with one or more fluorine atoms, e.g., wherein a hydrogen or halogen substituent of an aryl or heteroaryl ring has been replaced with a fluorine. In some embodiments, the fluorinated montelukast does not have the following formula:

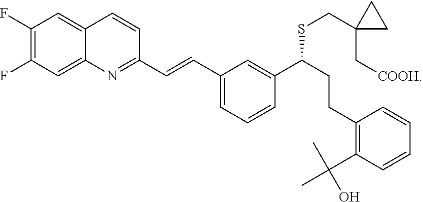

In some embodiments, the fluorine substituent is ¹⁹F. In some embodiments, the fluorine substituent is ¹⁸F. In some embodiments, the fluorinated montelukast is selected from one of the following:

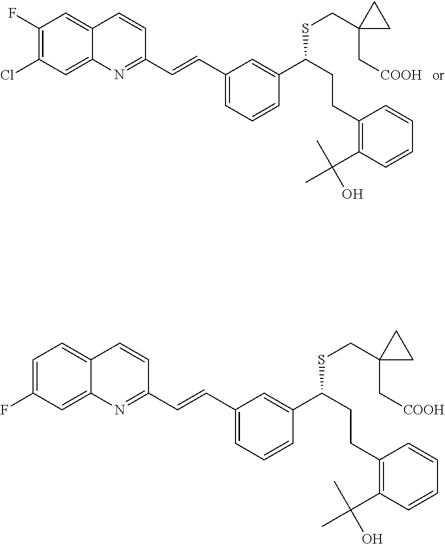

or a pharmaceutically acceptable salt thereof.

In one aspect, the invention features a method of making a fluorinated montelukast, including any of the three fluorinated montelukast structures shown above, using a method described herein.

In one aspect, the invention features an ¹⁸F-substituted montelukast, for example, a derivative of montelukast wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted montelukast has the following formula:

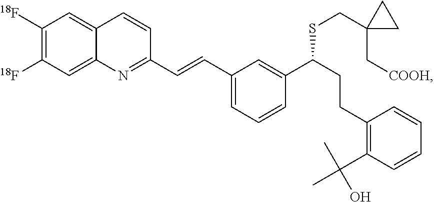

or a pharmaceutically acceptable salt thereof.

In one aspect, the invention features a method of making a fluorinated celecoxib, for example, a fluorinated celecoxib with the following formula, using a method described herein:

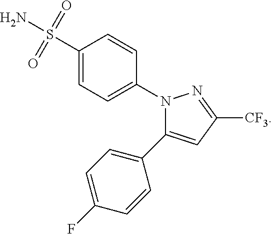

In one aspect, the invention features an ¹⁸F-substituted cetirizine, for example, a derivative of cetirizine wherein an aryl group has been substituted with one or more ¹⁸F atoms, e.g., wherein a hydrogen or halogen substituent of an aryl group has been replaced with ¹⁸F. In one embodiment, the ¹⁸F-substituted cetirizine has the following formula:

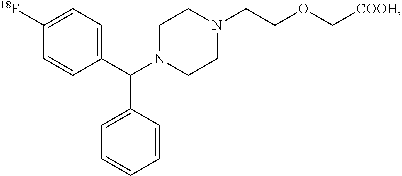

or a pharmaceutically acceptable salt thereof.

In one aspect, the invention features a method of making a fluorinated cetirizine, for example, a fluorinated cetirizine with the following formula, using a method described herein:

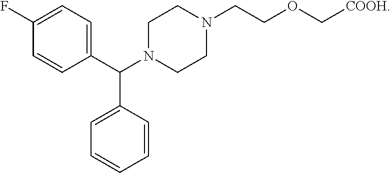

In one aspect, the invention features an ¹⁸F-substituted mesalazine, for example, a derivative of mesalazine wherein an aryl group has been substituted with one or more ¹⁸F atoms, e.g., wherein a hydrogen or hydroxy substituent of an aryl group has been replaced with ¹⁸F. In one embodiment, the ¹⁸F-substituted mesalazine has the following formula:

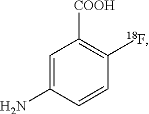

or a pharmaceutically acceptable salt thereof.

In one aspect, the invention features a method of making a fluorinated mesalazine, for example, a fluorinated mesalazine with the following formula, using a method described herein:

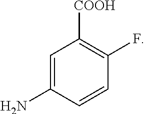

In one aspect, the invention features an ¹⁸F-substituted desloratadine, for example, a derivative of desloratadine wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted desloratadine has the following formula:

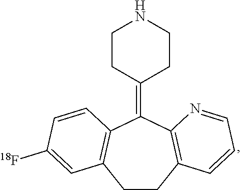

or a pharmaceutically acceptable salt thereof.

In one aspect, the invention features a method of making a fluorinated desloratadine, for example, a fluorinated desloratadine with the following formula, using a method described herein:

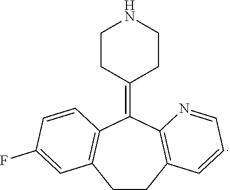

In one aspect, the invention features an ¹⁸F-substituted azelastine, for example, a derivative of azelastine wherein an aryl group has been substituted with one or more ¹⁸F atoms, e.g., wherein a hydrogen or halogen substituent of an aryl group has been replaced with ¹⁸F. In one embodiment, the ¹⁸F-substituted azelastine has the following formula:

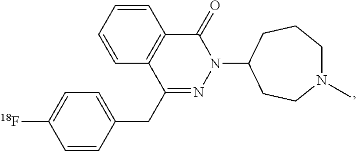

or a pharmaceutically acceptable salt thereof.

In one aspect, the invention features a method of making a fluorinated azelastine, for example, a fluorinated azelastine with the following formula, using a method described herein:

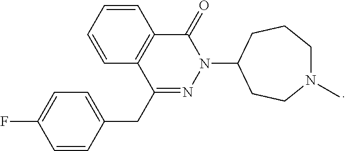

In one aspect, the invention features an ¹⁸F-substituted aspirin, for example, a derivative of aspirin wherein an aryl group has been substituted with one or more ¹⁸F atoms, e.g., wherein a hydrogen of an aryl group has been replaced with ¹⁸F. In one embodiment, the ¹⁸F-substituted aspirin has the following formula:

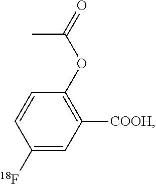

or a pharmaceutically acceptable salt thereof.

In one aspect, the invention features a method of making a fluorinated aspirin, for example, a fluorinated aspirin with the following formula, using a method described herein:

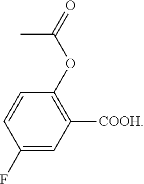

In one aspect, the invention features an ¹⁸F-substituted rizatriptan, for example, a derivative of rizatriptan wherein an aryl or heteroaryl group has been substituted with one or more ¹⁸F atoms, e.g., wherein a hydrogen or a triazole substituent of an aryl or heteroaryl group has been replaced with ¹⁸F. In one embodiment, the ¹⁸F-substituted rizatriptan has the following formula:

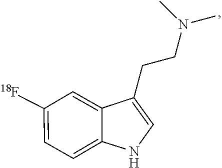

or a pharmaceutically acceptable salt thereof.

In one aspect, the invention features a method of making a fluorinated rizatriptan, for example, a fluorinated rizatriptan with the following formula, using a method described herein:

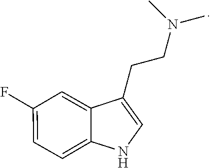

In one aspect, the invention features an ¹⁸F-substituted meloxicam, for example, a derivative of meloxicam wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted meloxicam has the following formula:

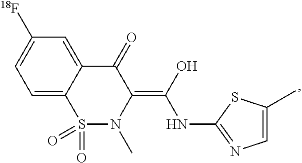

or a pharmaceutically acceptable salt thereof.

In one aspect, the invention features a method of making a fluorinated meloxicam, for example, a fluorinated meloxicam with the following formula, using a method described herein:

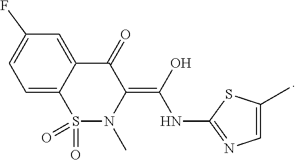

In one aspect, the invention features an ¹⁸F-substituted naproxen, for example, a derivative of naproxen wherein an aryl group has been substituted with one or more ¹⁸F atoms, e.g., wherein a hydrogen of an aryl group has been replaced with ¹⁸F. In one embodiment, the ¹⁸F-substituted naproxen has the following formula:

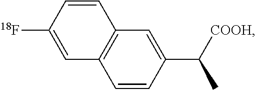

or a pharmaceutically acceptable salt thereof.

In one aspect, the invention features a method of making a fluorinated naproxen, for example, a fluorinated naproxen with the following formula, using a method described herein:

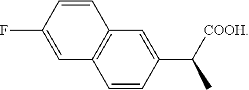

In one aspect, the invention features an ¹⁸F-substituted diclofenac, for example, a derivative of diclofenac wherein an aryl group has been substituted with one or more ¹⁸F atoms, e.g., wherein a hydrogen or a halogen substituent of an aryl group has been replaced with ¹⁸F. In one embodiment, the ¹⁸F-substituted diclofenac is selected from one of the following:

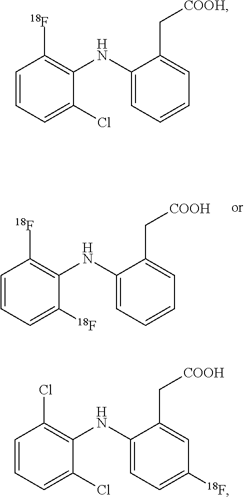

or a pharmaceutically acceptable salt thereof.

In one aspect, the invention features a method of making a fluorinated diclofenac, for example, a fluorinated diclofenac with any of the following formulae, using a method described herein:

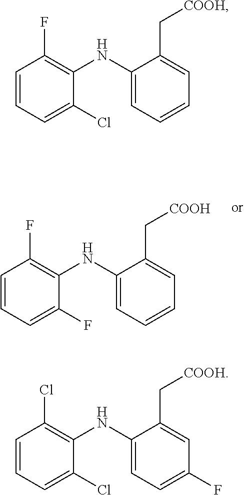

In one aspect, the invention features an ¹⁸F-substituted indomethacin, for example, a derivative of indomethacin wherein an aryl group has been substituted with one or more ¹⁸F atoms, e.g., wherein a hydrogen or a halogen or alkoxy substituent of an aryl group has been replaced with ¹⁸F. In one embodiment, the ¹⁸F-substituted indomethacin is selected from one of the following:

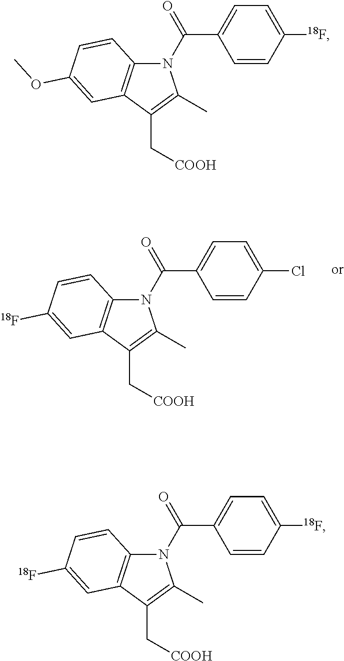

or a pharmaceutically acceptable salt thereof.

In one aspect, the invention features a method of making a fluorinated indomethacin, for example, a fluorinated indomethacin with one of the following formulae, using a method described herein:

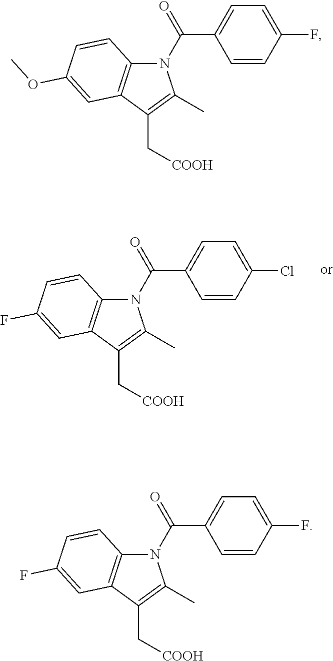

In one aspect, the invention features an ¹⁸F-substituted cinnarizine, for example, a derivative of cinnarizine wherein an aryl group has been substituted with one or more ¹⁸F atoms, e.g., wherein a hydrogen of an aryl group has been replaced with ¹⁸F. In one embodiment, the ¹⁸F-substituted cinnarizine is selected from one of the following:

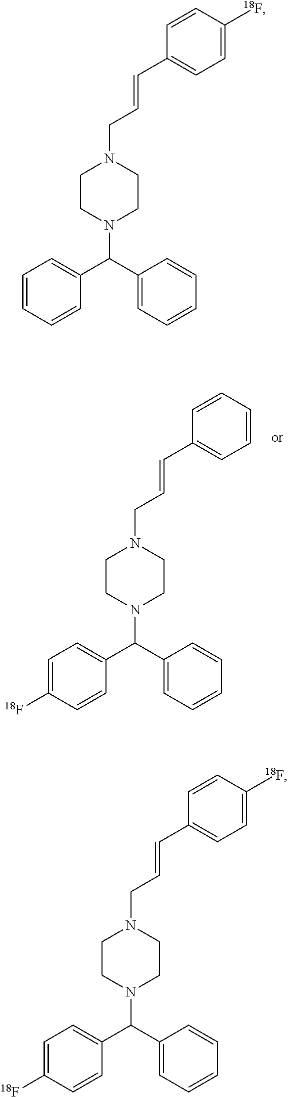

or a pharmaceutically acceptable salt thereof.

In one aspect, the invention features a method of making a fluorinated cinnarizine, for example, a fluorinated cinnarizine with one of the following formulae, using a method described herein:

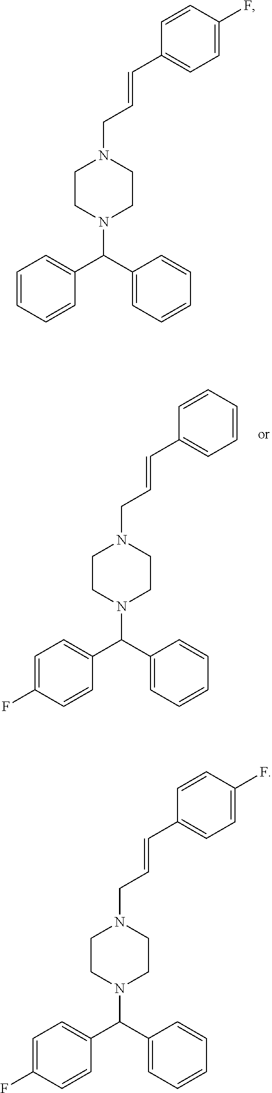

In one aspect, the invention features an ¹⁸F-substituted cyclizine, for example, a derivative of cyclizine wherein an aryl group has been substituted with one or more ¹⁸F atoms, e.g., wherein a hydrogen of an aryl group has been replaced with ¹⁸F. In one embodiment, the ¹⁸F-substituted cyclizine has the following formula:

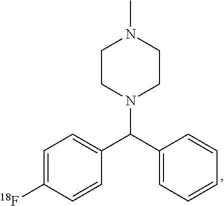

or a pharmaceutically acceptable salt thereof.

In one aspect, the invention features a method of making a fluorinated cyclizine, for example, a fluorinated cyclizine with the following formula, using a method described herein:

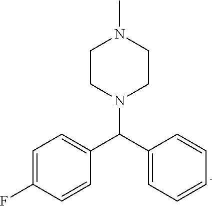

In one aspect, the invention features a fluorinated ergotamine, for example, a derivative of ergotamine wherein an aryl or heteroaryl group has been substituted with one or more fluorine atoms, e.g., wherein a hydrogen of an aryl or heteroaryl group has been replaced with a fluorine. In some embodiments, the fluorinated ergotamine does not have the following formula:

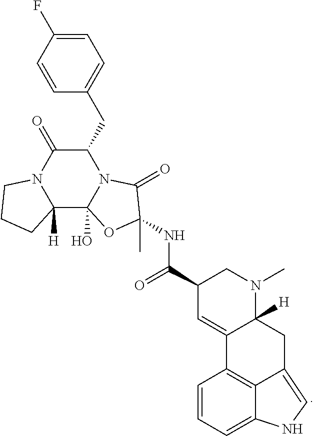

In some embodiments, the fluorine substituent is ¹⁹F. In some embodiments, the fluorine substituent is ¹⁸F. In some embodiments, the fluorinated ergotamine is selected from one of the following:

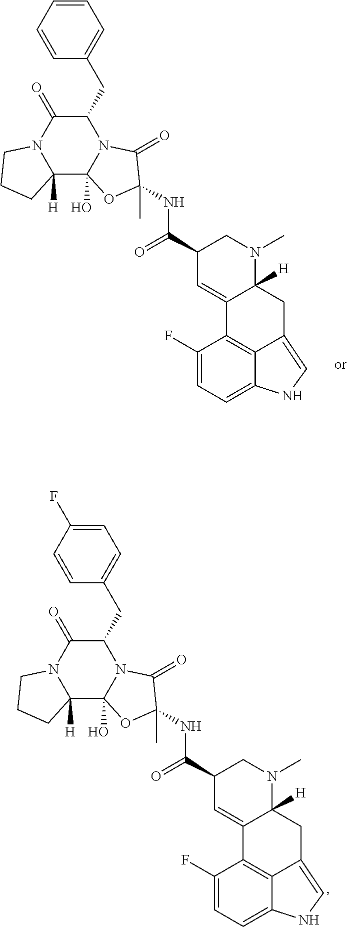

or a pharmaceutically acceptable salt thereof.

In one aspect, the invention features a method of making a fluorinated ergotamine, including any of the three fluorinated ergotamine structures shown above, using a method described herein.

In one aspect, the invention features an ¹⁸F-substituted ergotamine, for example, a derivative of ergotamine wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted ergotamine has the following formula:

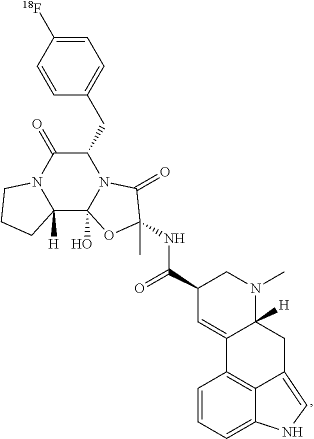

or a pharmaceutically acceptable salt thereof.

In one aspect, the invention features a fluorinated carvedilol, for example, a derivative of carvedilol wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated carvedilol has the following formula:

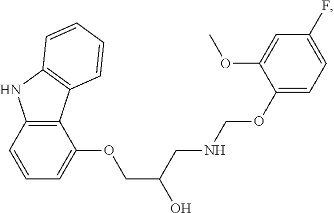

or a pharmaceutically acceptable salt thereof.

In one aspect, the invention features a method of making a fluorinated carvedilol, for example, the fluorinated carvedilol shown above, using a method described herein.

In one aspect, the invention features a fluorinated metoprolol, for example, a derivative of metoprolol wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated metoprolol has the following formula:

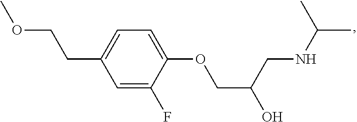

or a pharmaceutically acceptable salt thereof.

In one aspect, the invention features a method of making a fluorinated metoprolol, for example, the fluorinated metoprolol shown above, using a method described herein.

In one aspect, the invention features a fluorinated atenolol, for example, a derivative of atenolol wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated atenolol has the following formula:

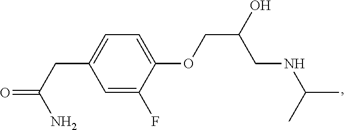

or a pharmaceutically acceptable salt thereof.

In one aspect, the invention features a method of making a fluorinated atenolol, for example, the fluorinated atenolol shown above, using a method described herein.

In one aspect, the invention features a fluorinated verapamil, for example, a derivative of verapamil wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated verapamil is selected from one of the following:

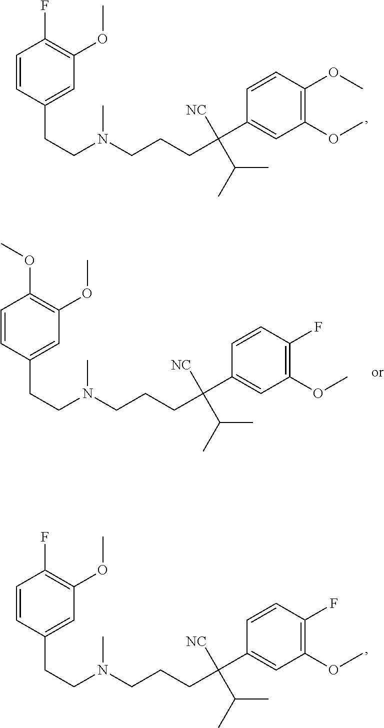

or a pharmaceutically acceptable salt thereof.

In one aspect, the invention features a method of making a fluorinated verapamil, for example, a fluorinated verapamil shown above, using a method described herein.

In one aspect, the invention features a fluorinated bisoprolol, for example, a derivative of bisoprolol wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated bisoprolol has the following formula:

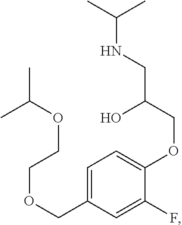

or a pharmaceutically acceptable salt thereof.

In one aspect, the invention features a method of making a fluorinated bisoprolol, for example, the fluorinated bisoprolol shown above, using a method described herein.

In one aspect, the invention features a fluorinated sotalol, for example, a derivative of sotalol wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated sotalol has the following formula:

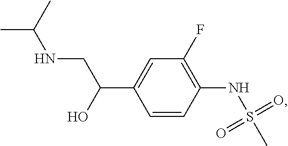

or a pharmaceutically acceptable salt thereof.

In one aspect, the invention features a method of making a fluorinated sotalol, for example, the fluorinated sotalol shown above, using a method described herein.

In one aspect, the invention features an ¹⁸F-substituted clopidogrel, for example, a derivative of clopidogrel wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted clopidogrel has the following formula:

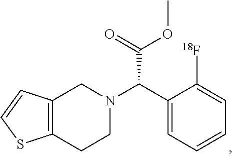

or a pharmaceutically acceptable salt thereof.

In one aspect, the invention features a method of making a fluorinated clopidogrel, for example, a fluorinated clopidogrel with the following formula, using a method described herein:

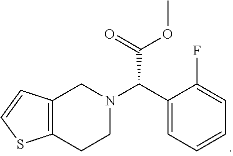

In one aspect, the invention features a fluorinated warfarin, for example, a derivative of warfarin wherein an aryl or heteroaryl group has been substituted with one or more fluorine atoms, e.g., wherein a hydrogen of an aryl or heteroaryl group has been replaced with a fluorine. In some embodiments, the fluorinated warfarin does not have either of the following formulae:

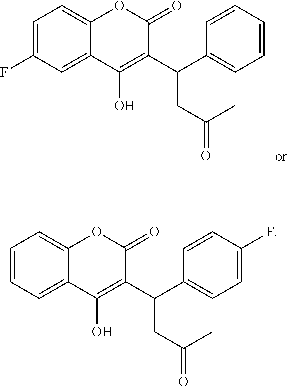

In some embodiments, the fluorine substituent is ¹⁹F. In some embodiments, the fluorine substituent is ¹⁸F. In some embodiments, the fluorinated warfarin has the following formula:

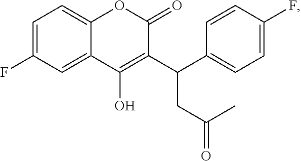

or a pharmaceutically acceptable salt thereof.

In one aspect, the invention features a method of making a fluorinated warfarin, including any of the three fluorinated warfarin structures shown above, using a method described herein.

In one aspect, the invention features an ¹⁸F-substituted warfarin, for example, a derivative of warfarin wherein an aryl group has been substituted with one or more ¹⁸F atoms, e.g., wherein a hydrogen of an aryl group has been replaced with ¹⁸F. In one embodiment, the ¹⁸F-substituted warfarin has the following formula:

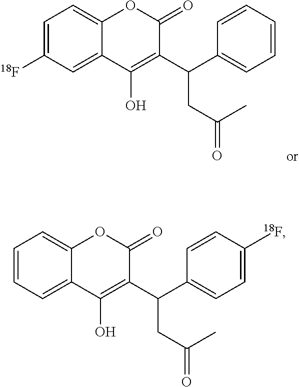

or a pharmaceutically acceptable salt thereof.

In one aspect, the invention features a fluorinated venlafaxine, for example, a derivative of venlafaxine wherein an aryl group has been substituted with one or more fluorine atoms, e.g., wherein a hydrogen or alkoxy substituent of an aryl group has been replaced with a fluorine. In some embodiments, the fluorine substituent is ¹⁹F. In some embodiments, the fluorine substituent is ¹⁸F. In some embodiments, the fluorinated venlafaxine has the following formula:

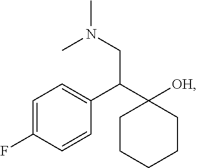

or a pharmaceutically acceptable salt thereof.

In one aspect, the invention features a method of making a fluorinated venlafaxine, for example, the fluorinated venlafaxine shown above, using a method described herein.

In one aspect, the invention features a fluorinated duloxetine, for example, a derivative of duloxetine wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated duloxetine has the following formula:

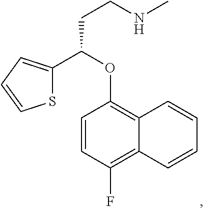

or a pharmaceutically acceptable salt thereof.

In one aspect, the invention features a method of making a fluorinated duloxetine, for example, the fluorinated duloxetine shown above, using a method described herein.

In one aspect, the invention features a fluorinated varenicline, for example, a derivative of varenicline wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated varenicline has the following formula:

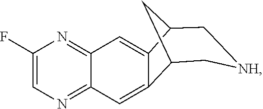

or a pharmaceutically acceptable salt thereof.

In one aspect, the invention features a method of making a fluorinated varenicline, for example, the fluorinated varenicline shown above, using a method described herein.

In one aspect, the invention features a fluorinated atomoxetine, for example, a derivative of atomoxetine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated atomoxetine has the following formula:

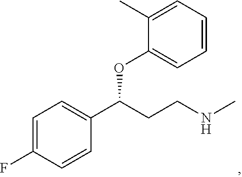

or a pharmaceutically acceptable salt thereof.

In one aspect, the invention features a method of making a fluorinated atomoxetine, for example, the fluorinated atomoxetine shown above, using a method described herein.

In one aspect, the invention features a fluorinated sertraline, for example, a derivative of sertraline wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated sertraline is selected from one of the following:

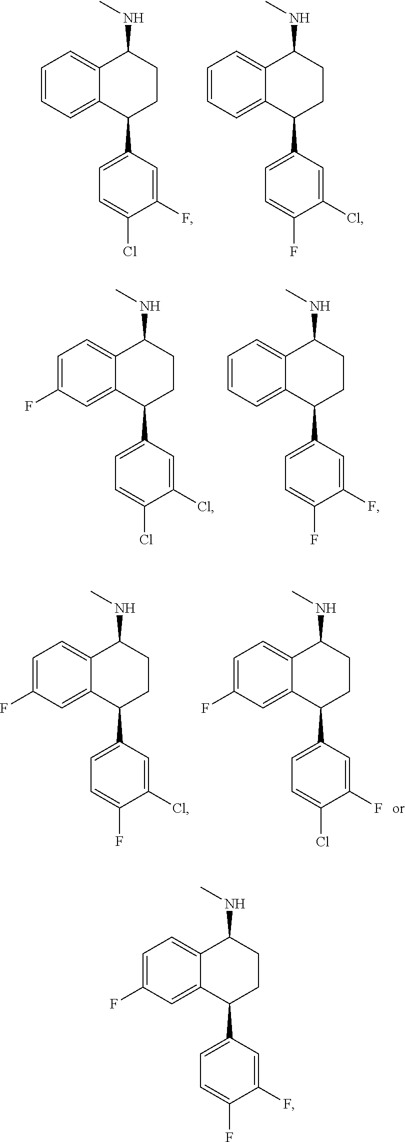

or a pharmaceutically acceptable salt thereof.

In one aspect, the invention features a method of making a fluorinated sertraline, for example, a fluorinated sertraline shown above, using a method described herein.

In one aspect, the invention features a fluorinated trazodone, for example, a derivative of trazodone wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated trazodone has the following formula:

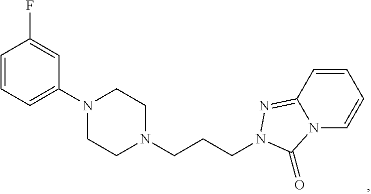

or a pharmaceutically acceptable salt thereof.

In one aspect, the invention features a method of making a fluorinated trazodone, for example, the fluorinated trazodone shown above, using a method described herein.

In one aspect, the invention features a fluorinated mirtazapine, for example, a derivative of mirtazapine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated mirtazapine has the following formula:

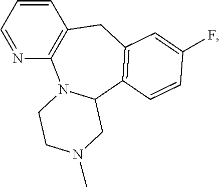

or a pharmaceutically acceptable salt thereof.

In one aspect, the invention features a method of making a fluorinated mirtazapine, for example, the fluorinated mirtazapine shown above, using a method described herein.

In one aspect, the invention features a fluorinated amitriptyline, for example, a derivative of amitriptyline wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated amitriptyline has the following formula:

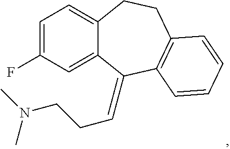

or a pharmaceutically acceptable salt thereof.

In one aspect, the invention features a method of making a fluorinated amitriptyline, for example, the fluorinated amitriptyline shown above, using a method described herein.

In one aspect, the invention features a fluorinated amoxapine, for example, a derivative of amoxapine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated amoxapine is selected from one of the following:

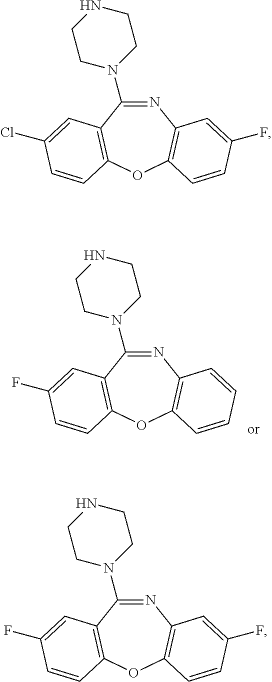

or a pharmaceutically acceptable salt thereof.

In one aspect, the invention features a method of making a fluorinated amoxapine, for example, a fluorinated amoxapine shown above, using a method described herein.

In one aspect, the invention features a fluorinated clomipramine, for example, a derivative of clomipramine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated clomipramine has the following formula:

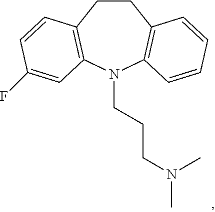

or a pharmaceutically acceptable salt thereof.

In one aspect, the invention features a method of making a fluorinated clomipramine, for example, the fluorinated clomipramine shown above, using a method described herein.

In one aspect, the invention features a fluorinated imipramine, for example, a derivative of imipramine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated imipramine has the following formula:

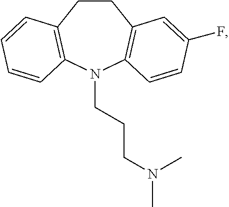

or a pharmaceutically acceptable salt thereof.

In one aspect, the invention features a method of making a fluorinated imipramine, for example, the fluorinated imipramine shown above, using a method described herein.

In one aspect, the invention features a fluorinated nortriptyline, for example, a derivative of nortriptyline wherein an aryl group has been substituted with one or more fluorine atoms, e.g., wherein a hydrogen substituent of an aryl group has been replaced with a fluorine. In some embodiments, the fluorine substituent is ¹⁹F. In some embodiments, the fluorine substituent is ¹⁸F. In one embodiment, the fluorinated nortriptyline has the following formula:

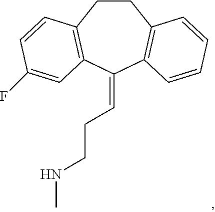

or a pharmaceutically acceptable salt thereof.

In one aspect, the invention features a method of making a fluorinated nortriptyline, for example, the fluorinated nortriptyline shown above, using a method described herein.

In one aspect, the invention features a fluorinated trimipramine, for example, a derivative of trimipramine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated trimipramine has the following formula:

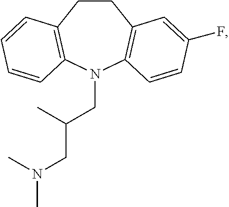

or a pharmaceutically acceptable salt thereof.

In one aspect, the invention features a method of making a fluorinated trimipramine, for example, the fluorinated trimipramine shown above, using a method described herein.

In one aspect, the invention features a fluorinated maprotiline, for example, a derivative of maprotiline wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated maprotiline has the following formula:

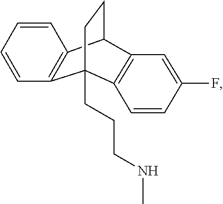

or a pharmaceutically acceptable salt thereof.

In one aspect, the invention features a method of making a fluorinated maprotiline, for example, the fluorinated maprotiline shown above, using a method described herein.

In one aspect, the invention features a fluorinated nefazodone, for example, a derivative of nefazodone wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated nefazodone is selected from one of the following:

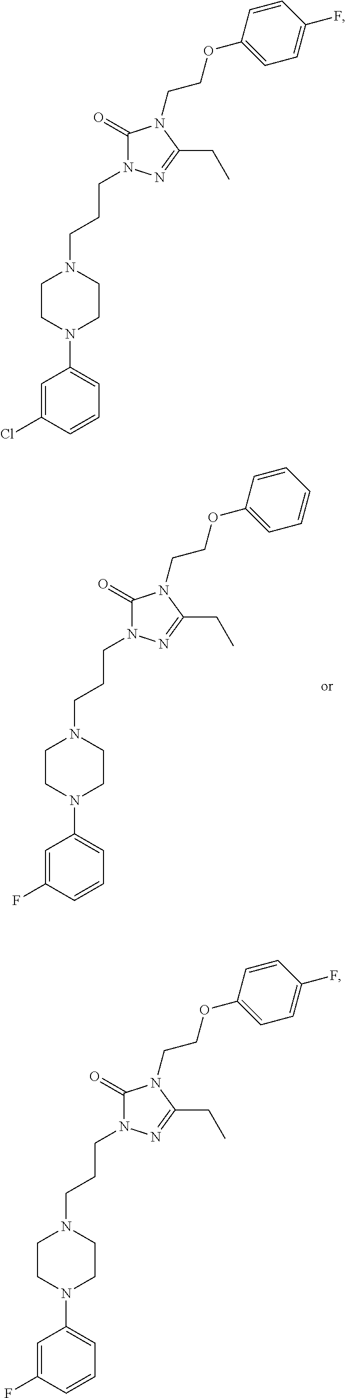

or a pharmaceutically acceptable salt thereof.

In one aspect, the invention features a method of making a fluorinated nefazodone, for example, a fluorinated nefazodone shown above, using a method described herein.

In one aspect, the invention features a fluorinated sibutramine, for example, a derivative of sibutramine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated sibutramine has the following formula:

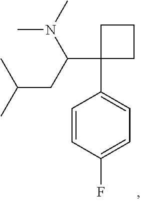

or a pharmaceutically acceptable salt thereof.

In one aspect, the invention features a method of making a fluorinated sibutramine, for example, the fluorinated sibutramine shown above, using a method described herein.

In one aspect, the invention features an ¹⁸F-substituted bupropion, for example, a derivative of bupropion wherein an aryl group has been substituted with one or more ¹⁸F atoms, e.g., wherein a hydrogen of an aryl group has been replaced with ¹⁸F. In one embodiment, the ¹⁸F-substituted bupropion has the following formula:

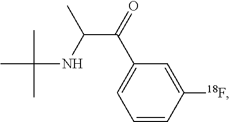

or a pharmaceutically acceptable salt thereof.

In one aspect, the invention features a method of making a fluorinated bupropion, for example, a fluorinated bupropion with the following formula, using a method described herein:

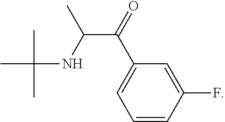

In one aspect, the invention features a fluorinated fluoxetine, for example, a derivative of fluoxetine wherein an aryl group has been substituted with one or more fluorine atoms, e.g., wherein a hydrogen or alkyl substituent of an aryl group has been replaced with a fluorine. In some embodiments, the fluorinated fluoxetine does not have either of the following formulae:

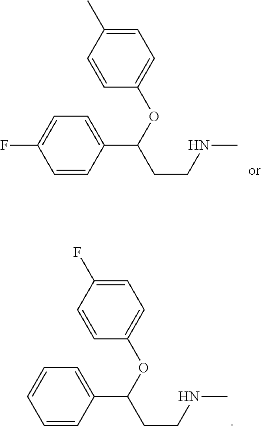

In some embodiments, the fluorine substituent is ¹⁹F. In some embodiments, the fluorine substituent is ¹⁸F. In some embodiments, the fluorinated fluoxetine has the following formula:

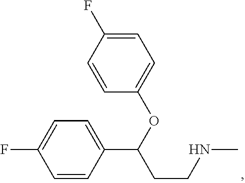

or a pharmaceutically acceptable salt thereof.

In one aspect, the invention features a method of making a fluorinated fluoxetine, including any of the three fluorinated fluoxetine structures shown above, using a method described herein.

In one aspect, the invention features an ¹⁸F-substituted fluoxetine, for example, a derivative of fluoxetine wherein an aryl group has been substituted with one or more ¹⁸F atoms, e.g., wherein a hydrogen or alkyl substituent of an aryl group has been replaced with ¹⁸F. In one embodiment, the ¹⁸F-substituted fluoxetine has the following formula:

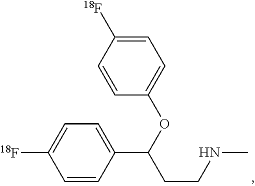

or a pharmaceutically acceptable salt thereof.

In one aspect, the invention features an ¹⁸F-substituted citalopram, for example, a derivative of citalopram wherein an aryl group has been substituted with one or more ¹⁸F atoms, e.g., wherein a hydrogen or cyano substituent of an aryl group has been replaced with ¹⁸F. In one embodiment, the ¹⁸F-substituted citalopram has the following formula:

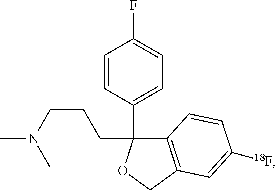

or a pharmaceutically acceptable salt thereof.

In one aspect, the invention features a method of making a fluorinated citalopram, for example, a fluorinated citalopram with the following formula, using a method described herein:

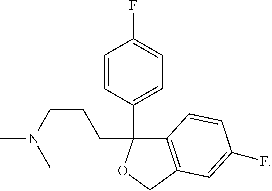

In one aspect, the invention features an ¹⁸F-substituted dosulepin, for example, a derivative of dosulepin wherein an aryl group has been substituted with one or more ¹⁸F atoms, e.g., wherein a hydrogen of an aryl group has been replaced with ¹⁸F. In one embodiment, the ¹⁸F-substituted dosulepin has the following formula:

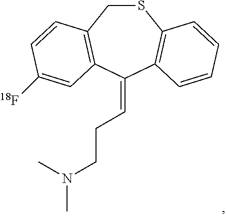

or a pharmaceutically acceptable salt thereof.

In one aspect, the invention features a method of making a fluorinated dosulepin, for example, a fluorinated dosulepin with the following formula, using a method described herein:

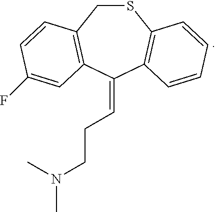

In one aspect, the invention features a fluorinated doxepin, for example, a derivative of doxepin wherein an aryl group has been substituted with one or more fluorine atoms, e.g., wherein a hydrogen of an aryl group has been replaced with a fluorine. In some embodiments, the fluorinated doxepin does not have the following formula:

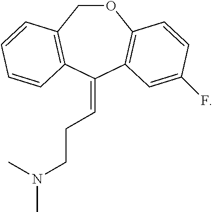

In some embodiments, the fluorine substituent is ¹⁹F. In some embodiments, the fluorine substituent is ¹⁸F. In some embodiments, the fluorinated doxepin is selected from one of the following:

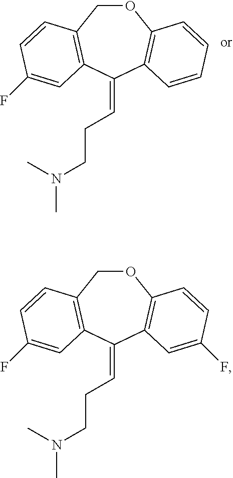

or a pharmaceutically acceptable salt thereof.

In one aspect, the invention features a method of making a fluorinated doxepin, including any of the three fluorinated doxepin structures shown above, using a method described herein.

In one aspect, the invention features an ¹⁸F-substituted doxepin, for example, a derivative of doxepin wherein an aryl group has been substituted with one or more ¹⁸F atoms, e.g., wherein a hydrogen of an aryl group has been replaced with ¹⁸F. In one embodiment, the ¹⁸F-substituted doxepin has the following formula:

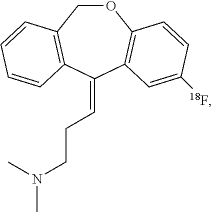

or a pharmaceutically acceptable salt thereof.

In one aspect, the invention features a fluorinated lofepramine, for example, a derivative of lofepramine wherein an aryl group has been substituted with one or more fluorine atoms, e.g., wherein a hydrogen or halogen substituent of an aryl group has been replaced with a fluorine. In some embodiments, the fluorinated lofepramine does not have the following formula:

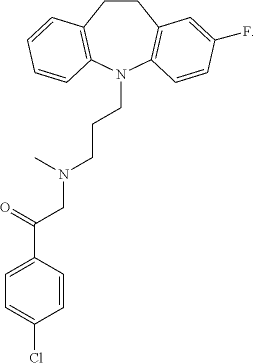

In some embodiments, the fluorine substituent is ¹⁹F. In some embodiments, the fluorine substituent is ¹⁸F. In some embodiments, the fluorinated lofepramine is selected from one of the following:

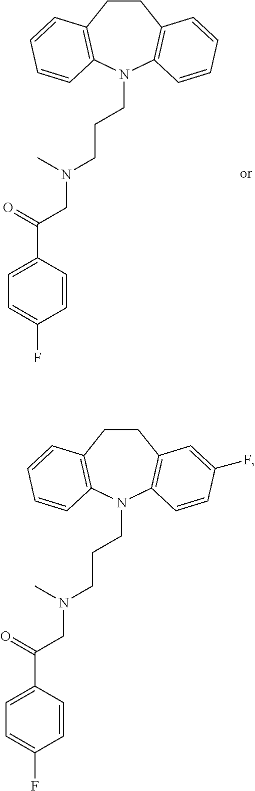

or a pharmaceutically acceptable salt thereof.

In one aspect, the invention features a method of making a fluorinated lofepramine, including any of the three fluorinated lofepramine structures shown above, using a method described herein.

In one aspect, the invention features an ¹⁸F-substituted lofepramine, for example, a derivative of lofepramine wherein an aryl group has been substituted with one or more ¹⁸F atoms, e.g., wherein a hydrogen or halogen substituent of an aryl group has been replaced with ¹⁸F. In one embodiment, the ¹⁸F-substituted lofepramine has the following formula:

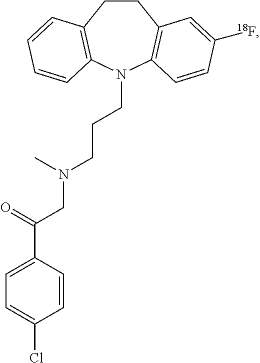

or a pharmaceutically acceptable salt thereof.

In one aspect, the invention features a fluorinated mianserin, for example, a derivative of mianserin wherein an aryl group has been substituted with one or more fluorine atoms, e.g., wherein a hydrogen of an aryl group has been replaced with a fluorine. In some embodiments, the fluorinated mianserin does not have the following formula:

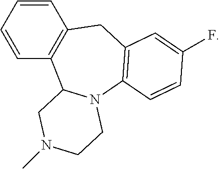

In some embodiments, the fluorine substituent is ¹⁹F. In some embodiments, the fluorine substituent is ¹⁸F. In some embodiments, the fluorinated mianserin is selected from one of the following:

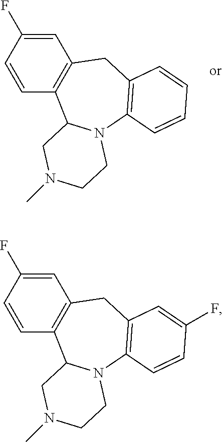

or a pharmaceutically acceptable salt thereof.

In one aspect, the invention features a method of making a fluorinated mianserin, including any of the three fluorinated mianserin structures shown above, using a method described herein.

In one aspect, the invention features an ¹⁸F-substituted mianserin, for example, a derivative of mianserin wherein an aryl group has been substituted with one or more ¹⁸F atoms, e.g., wherein a hydrogen of an aryl group has been replaced with ¹⁸F. In one embodiment, the ¹⁸F-substituted mianserin has the following formula:

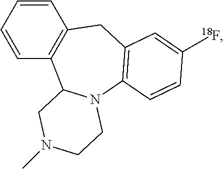

or a pharmaceutically acceptable salt thereof.

In one aspect, the invention features an ¹⁸F-substituted reboxetine, for example, a derivative of reboxetine wherein an aryl group has been substituted with one or more ¹⁸F atoms, e.g., wherein a hydrogen or an alkoxy substituent of an aryl group has been replaced with ¹⁸F. In one embodiment, the ¹⁸F-substituted reboxetine is selected from one of the following:

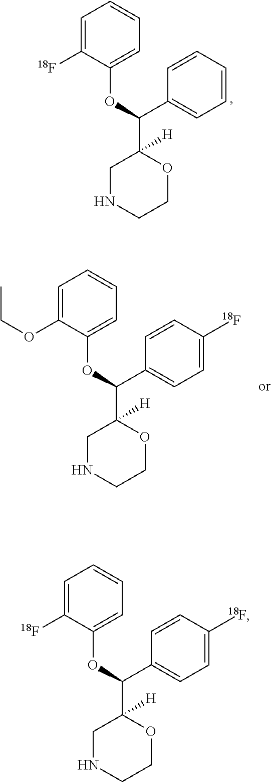

or a pharmaceutically acceptable salt thereof.

In one aspect, the invention features a method of making a fluorinated reboxetine, for example, a fluorinated reboxetine with the following formula, using a method described herein:

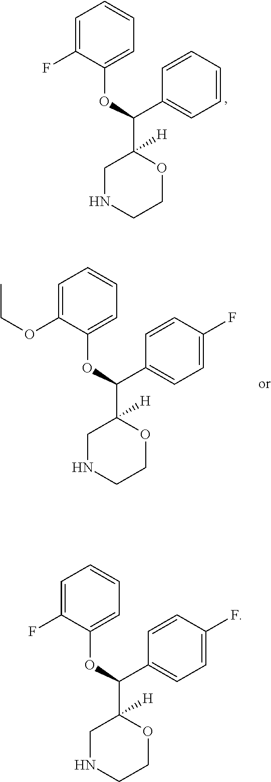

In one aspect, the invention features a method of making a fluorinated tryptophan, for example, a fluorinated tryptophan with the following formula, using a method described herein:

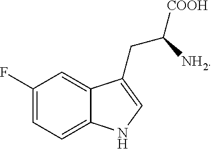

In one aspect, the invention features a fluorinated isocarboxazid, for example, a derivative of isocarboxazid wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated isocarboxazid has the following formula:

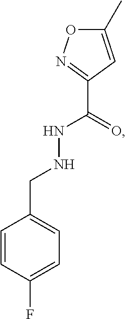

or a pharmaceutically acceptable salt thereof.

In one aspect, the invention features a method of making a fluorinated isocarboxazid, for example, the fluorinated isocarboxazid shown above, using a method described herein.

In one aspect, the invention features an ¹⁸F-substituted phenelzine, for example, a derivative of phenelzine wherein an aryl group has been substituted with one or more ¹⁸F atoms, e.g., wherein a hydrogen of an aryl group has been replaced with ¹⁸F. In one embodiment, the ¹⁸F-substituted phenelzine has the following formula:

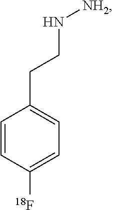

or a pharmaceutically acceptable salt thereof.

In one aspect, the invention features a method of making a fluorinated phenelzine, for example, a fluorinated phenelzine with the following formula, using a method described herein:

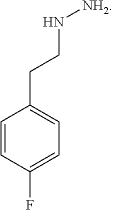

In one aspect, the invention features an ¹⁸F-substituted tranylcypromine, for example, a derivative of tranylcypromine wherein an aryl group has been substituted with one or more ¹⁸F atoms, e.g., wherein a hydrogen of an aryl group has been replaced with F. In one embodiment, the ¹⁸F-substituted tranylcypromine has the following formula:

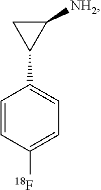

or a pharmaceutically acceptable salt thereof.

In one aspect, the invention features a method of making a fluorinated tranylcypromine, for example, a fluorinated tranylcypromine with the following formula, using a method described herein:

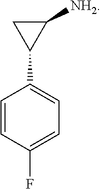

In one aspect, the invention features an ¹⁸F-substituted moclobemide, for example, a derivative of moclobemide wherein an aryl group has been substituted with one or more ¹⁸F atoms, e.g., wherein a hydrogen or halogen substituent of an aryl group has been replaced with ¹⁸F. In one embodiment, the ¹⁸F-substituted moclobemide has the following formula:

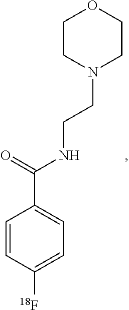

or a pharmaceutically acceptable salt thereof.

In one aspect, the invention features a method of making a fluorinated moclobemide, for example, a fluorinated moclobemide with the following formula, using a method described herein:

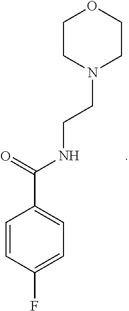

In one aspect, the invention features a fluorinated fosphenyloin, for example, a derivative of fosphenyloin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated fosphenyloin is selected from one of the following:

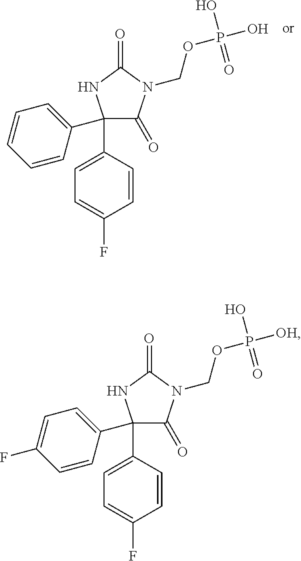

or a pharmaceutically acceptable salt thereof.

In one aspect, the invention features a method of making a fluorinated fosphenyloin, for example, a fluorinated fosphenyloin shown above, using a method described herein.

In one aspect, the invention features a fluorinated tolterodine, for example, a derivative of tolterodine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated tolterodine has the following formula:

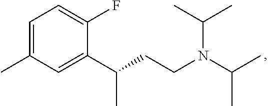

or a pharmaceutically acceptable salt thereof.

In one aspect, the invention features a method of making a fluorinated tolterodine, for example, the fluorinated tolterodine shown above, using a method described herein.

In one aspect, the invention features a fluorinated darifenacin, for example, a derivative of darifenacin wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated darifenacin is selected from one of the following:

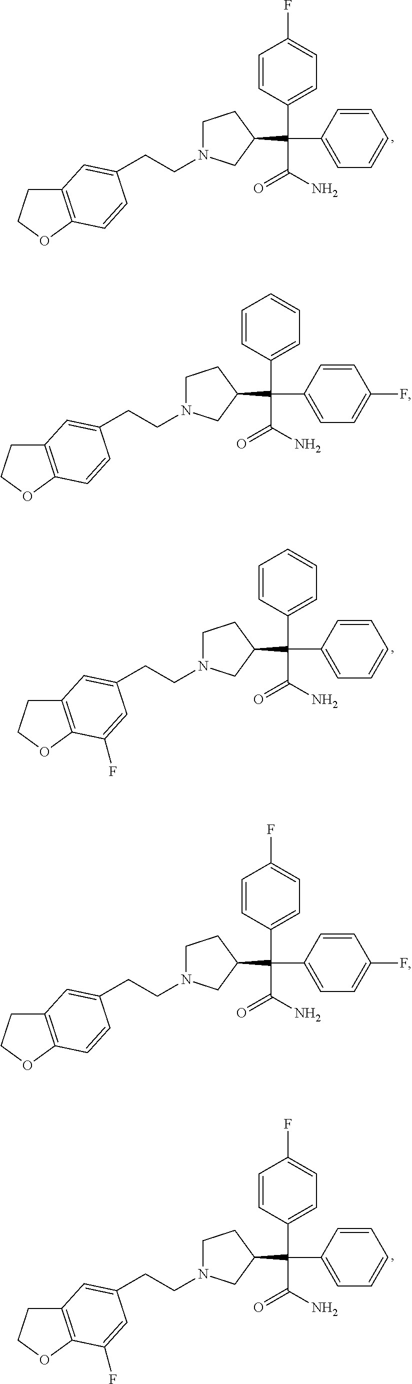

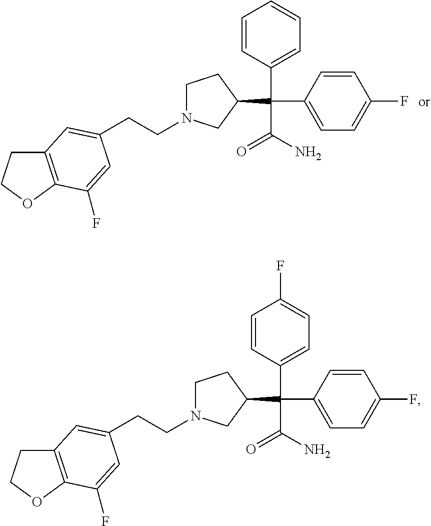

or a pharmaceutically acceptable salt thereof.

In one aspect, the invention features a method of making a fluorinated darifenacin, for example, a fluorinated darifenacin shown above, using a method described herein.

In one aspect, the invention features a fluorinated oxcarbazepine, for example, a derivative of oxcarbazepine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated oxcarbazepine has the following formula:

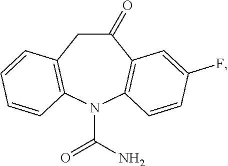

or a pharmaceutically acceptable salt thereof.

In one aspect, the invention features a method of making a fluorinated oxcarbazepine, for example, the fluorinated oxcarbazepine shown above, using a method described herein.

In one aspect, the invention features a fluorinated cabergoline, for example, a derivative of cabergoline wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated cabergoline has the following formula:

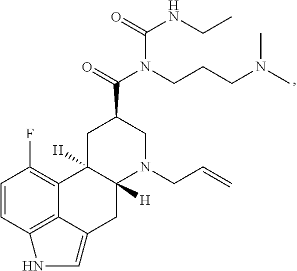

or a pharmaceutically acceptable salt thereof.

In one aspect, the invention features a method of making a fluorinated cabergoline, for example, the fluorinated cabergoline shown above, using a method described herein.

In one aspect, the invention features a fluorinated benserazide, for example, a derivative of benserazide wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated benserazide is selected from one of the following:

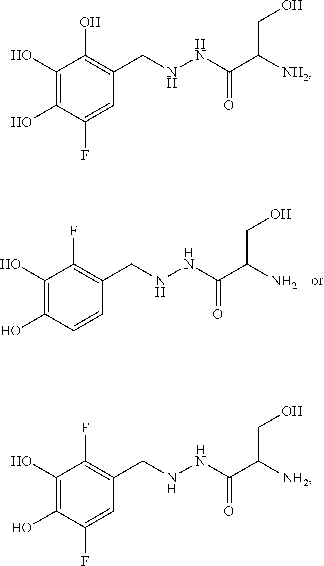

or a pharmaceutically acceptable salt thereof.

In one aspect, the invention features a method of making a fluorinated benserazide, for example, a fluorinated benserazide shown above, using a method described herein.

In one aspect, the invention features a fluorinated bromocriptine, for example, a derivative of bromocriptine wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated bromocriptine has the following formula:

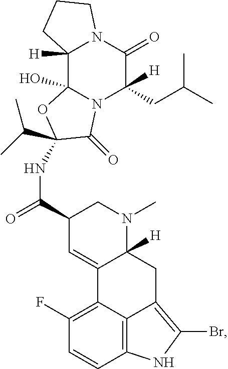

or a pharmaceutically acceptable salt thereof.

In one aspect, the invention features a method of making a fluorinated bromocriptine, for example, the fluorinated bromocriptine shown above, using a method described herein.

In one aspect, the invention features a fluorinated entacapone, for example, a derivative of entacapone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated entacapone has the following formula:

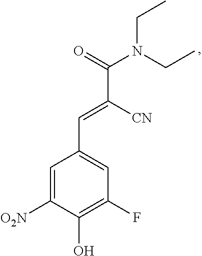

or a pharmaceutically acceptable salt thereof.

In one aspect, the invention features a method of making a fluorinated entacapone, for example, the fluorinated entacapone shown above, using a method described herein.

In one aspect, the invention features a fluorinated lisuride, for example, a derivative of lisuride wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In one embodiment, the fluorinated lisuride has the following formula:

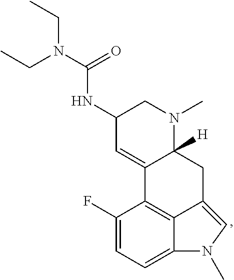

or a pharmaceutically acceptable salt thereof.

In one aspect, the invention features a method of making a fluorinated lisuride, for example, the fluorinated lisuride shown above, using a method described herein.

In one aspect, the invention features a fluorinated pergolide, for example, a derivative of pergolide wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated pergolide has the following formula:

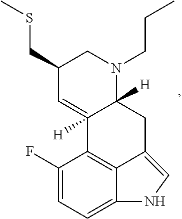

or a pharmaceutically acceptable salt thereof.

In one aspect, the invention features a method of making a fluorinated pergolide, for example, the fluorinated pergolide shown above, using a method described herein.

In one aspect, the invention features a fluorinated biperiden, for example, a derivative of biperiden wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated biperiden has the following formula:

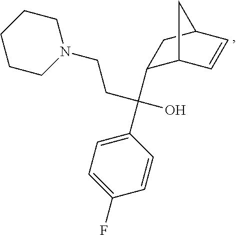

or a pharmaceutically acceptable salt thereof.

In one aspect, the invention features a method of making a fluorinated biperiden, for example, the fluorinated biperiden shown above, using a method described herein.

In one aspect, the invention features a fluorinated orphenadrine, for example, a derivative of orphenadrine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated orphenadrine is selected from one of the following:

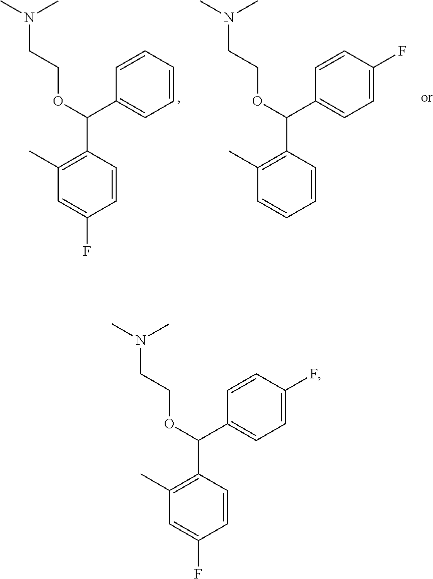

or a pharmaceutically acceptable salt thereof.

In one aspect, the invention features a method of making a fluorinated orphenadrine, for example, a fluorinated orphenadrine shown above, using a method described herein.

In one aspect, the invention features a fluorinated procyclidine, for example, a derivative of procyclidine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated procyclidine has the following formula:

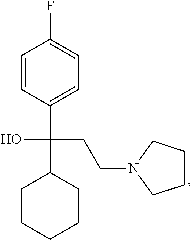

or a pharmaceutically acceptable salt thereof.

In one aspect, the invention features a method of making a fluorinated procyclidine, for example, the fluorinated procyclidine shown above, using a method described herein.

In one aspect, the invention features a fluorinated tetrabenazine, for example, a derivative of tetrabenazine wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated tetrabenazine has the following formula:

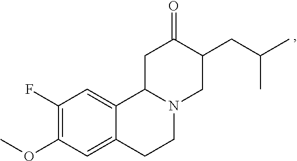

or a pharmaceutically acceptable salt thereof.

In one aspect, the invention features a method of making a fluorinated tetrabenazine, for example, the fluorinated tetrabenazine shown above, using a method described herein.

In one aspect, the invention features an ¹⁸F-substituted lamotrigine, for example, a derivative of lamotrigine wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted lamotrigine has the following formula:

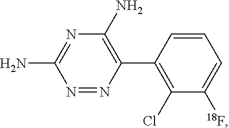

or a pharmaceutically acceptable salt thereof.

In one aspect, the invention features a method of making a fluorinated lamotrigine, for example, a fluorinated lamotrigine with the following formula, using a method described herein:

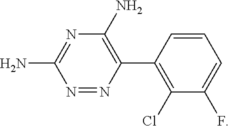

In one aspect, the invention features an ¹⁸F-substituted solifenacin, for example, a derivative of solifenacin wherein an aryl group has been substituted with one or more ¹⁸F atoms, e.g., wherein a hydrogen of an aryl group has been replaced with ¹⁸F. In one embodiment, the ¹⁸F-substituted solifenacin has the following formula:

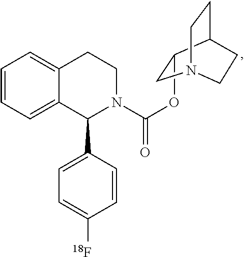

or a pharmaceutically acceptable salt thereof.

In one aspect, the invention features a method of making a fluorinated solifenacin, for example, a fluorinated solifenacin with the following formula, using a method described herein:

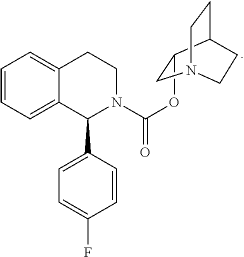

In one aspect, the invention features an ¹⁸F-substituted clonazepam, for example, a derivative of clonazepam wherein an aryl group has been substituted with one or more ¹⁸F atoms, e.g., wherein a hydrogen or halogen substituent of an aryl group has been replaced with ¹⁸F. In one embodiment, the ¹⁸F-substituted clonazepam has the following formula:

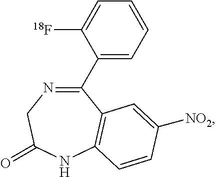

or a pharmaceutically acceptable salt thereof.

In one aspect, the invention features a method of making a fluorinated clonazepam, for example, a fluorinated clonazepam with the following formula, using a method described herein:

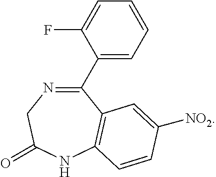

In one aspect, the invention features an ¹⁸F-substituted phenyloin, for example, a derivative of phenyloin wherein an aryl group has been substituted with one or more ¹⁸F atoms, e.g., wherein a hydrogen of an aryl group has been replaced with ¹⁸F. In one embodiment, the ¹⁸F-substituted phenyloin is selected from one of the following:

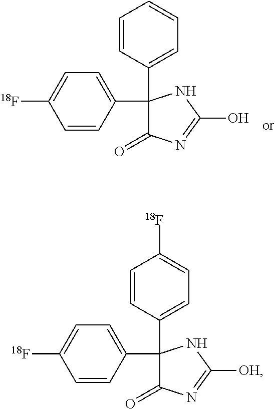

or a pharmaceutically acceptable salt thereof.

In one aspect, the invention features a method of making a fluorinated phenyloin, for example, a fluorinated phenyloin with either of the following formulae, using a method described herein:

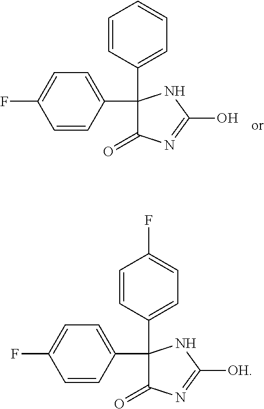

In one aspect, the invention features a fluorinated carbidopa, for example, a derivative of carbidopa wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In some embodiments, the fluorinated carbidopa has the following formula:

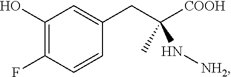

or a pharmaceutically acceptable salt thereof.

In one aspect, the invention features a method of making a fluorinated carbidopa, for example, the fluorinated carbidopa shown above.

In one aspect, the invention features an ¹⁸F-substituted levodopa, for example, a derivative of levodopa wherein an aryl group has been substituted with one or more ¹⁸F atoms, e.g., wherein a hydrogen or hydroxy substituent of an aryl group has been replaced with ¹⁸F. In one embodiment, the ¹⁸F-substituted levodopa has the following formula:

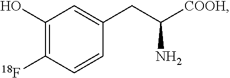

or a pharmaceutically acceptable salt thereof.

In one aspect, the invention features a method of making a fluorinated levodopa, for example, a fluorinated levodopa with the following formula, using a method described herein:

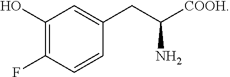

In one aspect, the invention features an ¹⁸F-substituted baclofen, for example, a derivative of baclofen wherein an aryl group has been substituted with one or more ¹⁸F atoms, e.g., wherein a hydrogen or halogen substituent of an aryl group has been replaced with ¹⁸F.

In one embodiment, the ¹⁸F-substituted baclofen has the following formula:

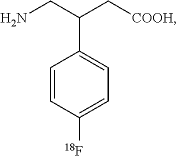

or a pharmaceutically acceptable salt thereof.

In one aspect, the invention features a method of making a fluorinated baclofen, for example, a fluorinated baclofen with the following formula, using a method described herein:

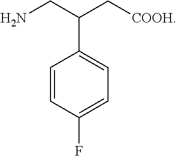

In one aspect, the invention features an ¹⁸F-substituted zonisamide, for example, a derivative of zonisamide wherein an aryl group has been substituted with one or more ¹⁸F atoms, e.g., wherein a hydrogen of an aryl group has been replaced with ¹⁸F. In one embodiment, the ¹⁸F-substituted zonisamide has the following formula:

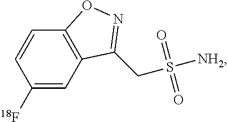

or a pharmaceutically acceptable salt thereof.

In one aspect, the invention features a method of making a fluorinated zonisamide, for example, a fluorinated zonisamide with the following formula, using a method described herein:

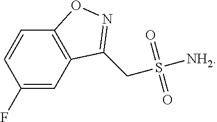

In one aspect, the invention features an ¹⁸F-substituted primidone, for example, a derivative of primidone wherein an aryl group has been substituted with one or more ¹⁸F atoms, e.g., wherein a hydrogen of an aryl group has been replaced with ¹⁸F. In one embodiment, the ¹⁸F-substituted primidone has the following formula:

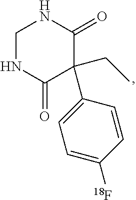

or a pharmaceutically acceptable salt thereof.

In one aspect, the invention features a fluorinated domperidone, for example, a derivative of domperidone wherein an aryl or heteroaryl group has been substituted with one or more fluorine atoms, e.g., wherein a hydrogen or halogen substituent of an aryl or heteroaryl group has been replaced with a fluorine. In some embodiments, the fluorinated domperidone does not have the following formula:

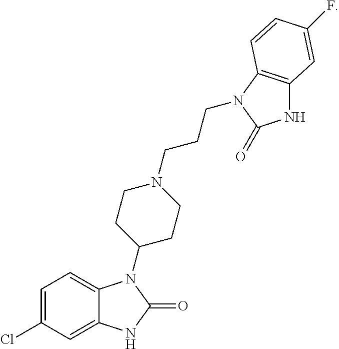

In some embodiments, the fluorine substituent is ¹⁹F. In some embodiments, the fluorine substituent is ¹⁸F. In some embodiments, the fluorinated domperidone is selected from one of the following:

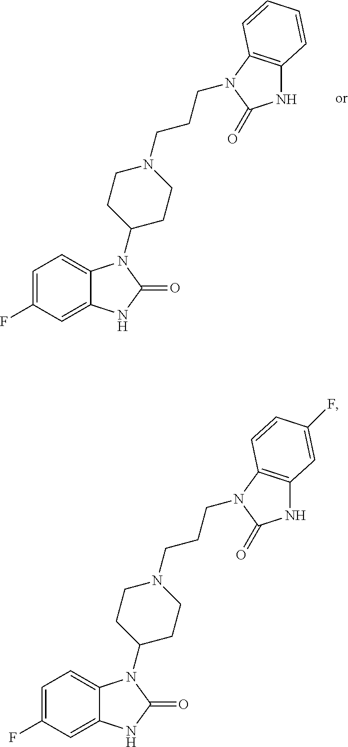

or a pharmaceutically acceptable salt thereof.

In one aspect, the invention features a method of making a fluorinated domperidone, including any of the three fluorinated domperidone structures shown above, using a method described herein.

In one aspect, the invention features an ¹⁸F-substituted domperidone, for example, a derivative of domperidone wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted domperidone has the following formula:

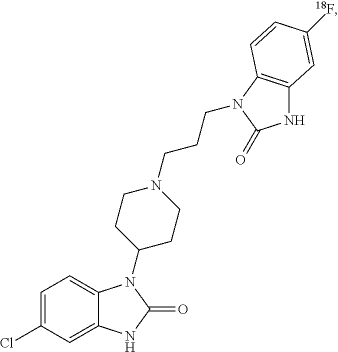

or a pharmaceutically acceptable salt thereof.

In one aspect, the invention features an ¹⁸F-substituted phenobarbital, for example, a derivative of phenobarbital wherein an aryl group has been substituted with one or more ¹⁸F atoms, e.g., wherein a hydrogen of an aryl group has been replaced with ¹⁸F. In one embodiment, the ¹⁸F-substituted phenobarbital has the following formula:

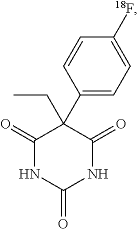

or a pharmaceutically acceptable salt thereof.

In one aspect, the invention features a method of making a fluorinated phenobarbital, for example, a fluorinated phenobarbital with the following formula, using a method described herein:

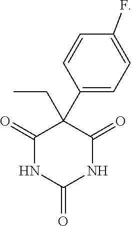

In one aspect, the invention features a fluorinated clobazam, for example, a derivative of clobazam wherein an aryl group has been substituted with one or more fluorine atoms, e.g., wherein a hydrogen or halogen substituent of an aryl group has been replaced with a fluorine. In some embodiments, the fluorinated clobazam does not have the following formula:

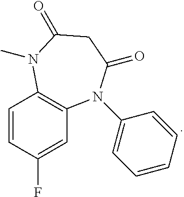

In some embodiments, the fluorine substituent is ¹⁹F. In some embodiments, the fluorine substituent is ¹⁸F. In some embodiments, the fluorinated clobazam is selected from one of the following:

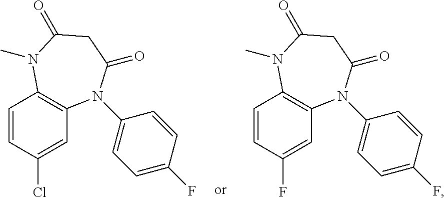

or a pharmaceutically acceptable salt thereof.

In one aspect, the invention features a method of making a fluorinated clobazam, including any of the three fluorinated clobazam structures shown above, using a method described herein.

In one aspect, the invention features an ¹⁸F-substituted clobazam, for example, a derivative of clobazam wherein an aryl group has been substituted with one or more ¹⁸F atoms, e.g., wherein a hydrogen or halogen substituent of an aryl group has been replaced with ¹⁸F. In one embodiment, the ¹⁸F-substituted clobazam has the following formula:

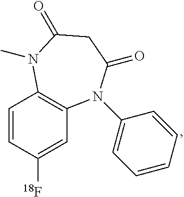

or a pharmaceutically acceptable salt thereof.

In one aspect, the invention features a method of making a fluorinated selegiline, for example, a fluorinated selegiline with the following formula, using a method described herein:

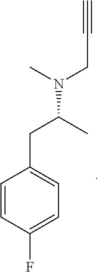

In one aspect, the invention features an ¹⁸F-substituted benzatropine, for example, a derivative of benzatropine wherein an aryl group has been substituted with one or more ¹⁸F atoms, e.g., wherein a hydrogen of an aryl group has been replaced with ¹⁸F. In one embodiment, the ¹⁸F-substituted benzatropine is selected from one of the following:

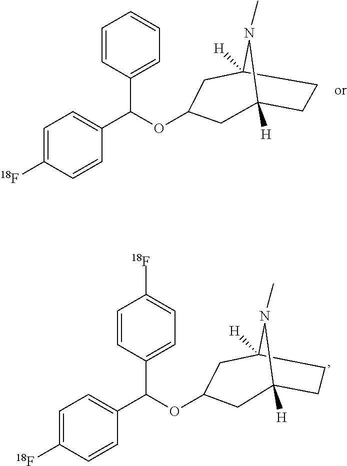

or a pharmaceutically acceptable salt thereof.

In one aspect, the invention features a method of making a fluorinated benzatropine, for example, a fluorinated benzatropine with one of the following formulae, using a method described herein:

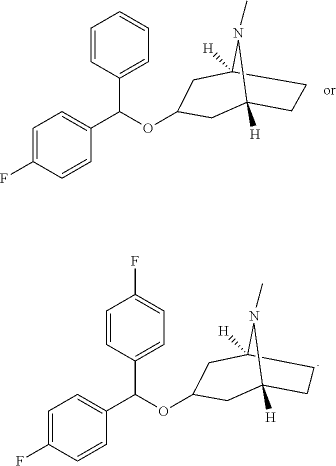

In one aspect, the invention features an ¹⁸F-substituted trihexyphenidyl, for example, a derivative of trihexyphenidyl wherein an aryl group has been substituted with one or more ¹⁸F atoms, e.g., wherein a hydrogen of an aryl group has been replaced with ¹⁸F. In one embodiment, the ¹⁸F-substituted trihexyphenidyl has the following formula:

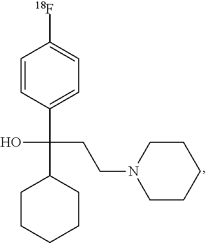

or a pharmaceutically acceptable salt thereof.

In one aspect, the invention features a method of making a fluorinated trihexyphenidyl, for example, a fluorinated trihexyphenidyl with the following formula, using a method described herein:

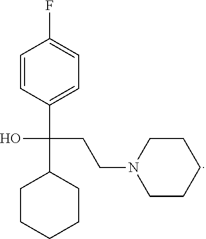

In one aspect, the invention features an ¹⁸F-substituted riluzole, for example, a derivative of riluzole wherein an aryl group has been substituted with one or more ¹⁸F atoms, e.g., wherein a hydrogen or haloalkoxy of an aryl group has been replaced with ¹⁸F. In one embodiment, the ¹⁸F-substituted riluzole has the following formula:

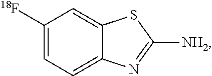

or a pharmaceutically acceptable salt thereof.

In one aspect, the invention features a method of making a fluorinated riluzole, for example, a fluorinated riluzole with the following formula, using a method described herein:

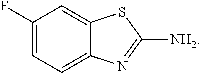

In one aspect, the invention features a fluorinated aripiprazole, for example, a derivative of aripiprazole wherein an aryl or heteroaryl group has been substituted with one or more fluorine atoms, e.g., wherein a hydrogen or halogen substituent of an aryl or heteroaryl group has been replaced with a fluorine. In some embodiments, the fluorine substituent is ¹⁹F. In some embodiments, the fluorine substituent is ¹⁸F. In some embodiments, the fluorinated aripiprazole has the following formula:

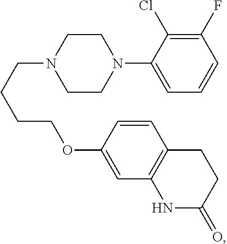

or a pharmaceutically acceptable salt thereof.

In one aspect, the invention features a method of making a fluorinated aripiprazole, for example, the fluorinated aripiprazole shown above, using a method described herein.

In one aspect, the invention features a fluorinated olanzapine, for example, a derivative of olanzapine wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated olanzapine has the following formula:

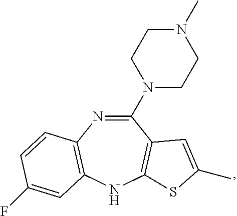

or a pharmaceutically acceptable salt thereof.

In one aspect, the invention features a method of making a fluorinated olanzapine, for example, the fluorinated olanzapine shown above, using a method described herein.

In one aspect, the invention features a fluorinated eszopiclone, for example, a derivative of eszopiclone wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In some embodiments, the fluorinated eszopiclone has the following formula:

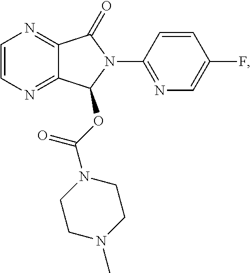

or a pharmaceutically acceptable salt thereof.

In one aspect, the invention features a method of making a fluorinated eszopiclone, for example, the fluorinated eszopiclone shown above, using a method described herein.

In one aspect, the invention features a fluorinated alprazolam, for example, a derivative of alprazolam wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated alprazolam is selected from one of the following:

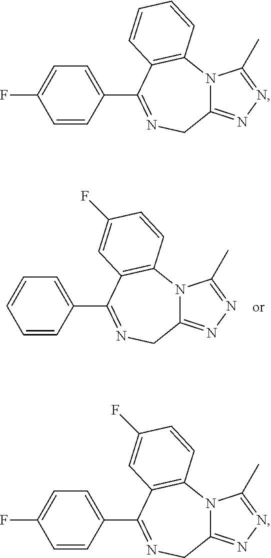

or a pharmaceutically acceptable salt thereof.

In one aspect, the invention features a method of making a fluorinated alprazolam, for example, a fluorinated alprazolam shown above, using a method described herein.

In one aspect, the invention features a fluorinated flunitrazepam, for example, a derivative of flunitrazepam wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated flunitrazepam has the following formula:

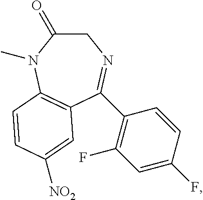

or a pharmaceutically acceptable salt thereof.

In one aspect, the invention features a method of making a fluorinated flunitrazepam, for example, the fluorinated flunitrazepam shown above, using a method described herein.

In one aspect, the invention features a fluorinated flurazepam, for example, a derivative of flurazepam wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated flurazepam is selected from one of the following:

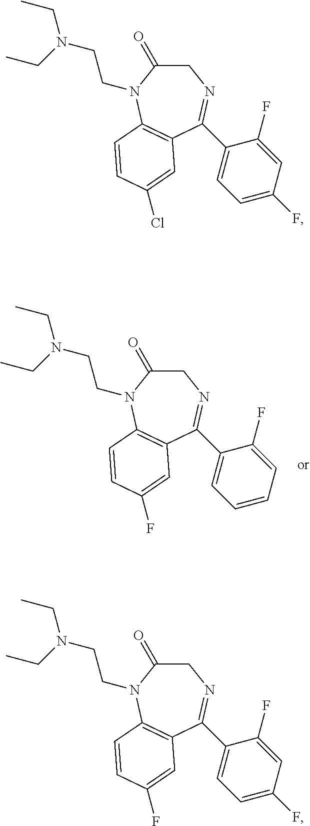

or a pharmaceutically acceptable salt thereof.

In one aspect, the invention features a method of making a fluorinated flurazepam, for example, a fluorinated flurazepam shown above, using a method described herein.

In one aspect, the invention features a fluorinated zaleplon, for example, a derivative of zaleplon wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated zaleplon has the following formula:

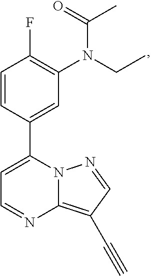

or a pharmaceutically acceptable salt thereof.

In one aspect, the invention features a method of making a fluorinated zaleplon, for example, the fluorinated zaleplon shown above, using a method described herein.

In one aspect, the invention features a fluorinated clomethiazole, for example, a derivative of clomethiazole wherein a heteroaryl group has been substituted with one or more fluorine atoms, e.g., wherein a hydrogen or alkyl substituent of a heteroaryl group has been replaced with a fluorine. In some embodiments, the fluorine substituent is ¹⁹F. In some embodiments, the fluorine substituent is ¹⁸F. In one embodiment, the fluorinated clomethiazole has the following formula:

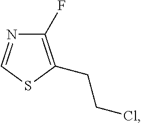

or a pharmaceutically acceptable salt thereof.

In one aspect, the invention features a method of making a fluorinated clomethiazole, for example, the fluorinated clomethiazole shown above, using a method described herein.

In one aspect, the invention features a fluorinated chlordiazepoxide, for example, a derivative of chlordiazepoxide wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated chlordiazepoxide is selected from one of the following:

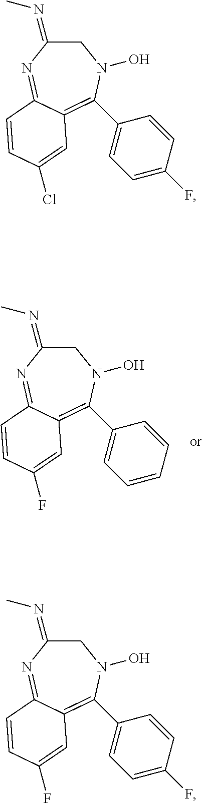

or a pharmaceutically acceptable salt thereof.

In one aspect, the invention features a method of making a fluorinated chlordiazepoxide, for example, a fluorinated chlordiazepoxide shown above, using a method described herein.

In one aspect, the invention features a fluorinated clorazepate, for example, a derivative of clorazepate wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated clorazepate is selected from one of the following:

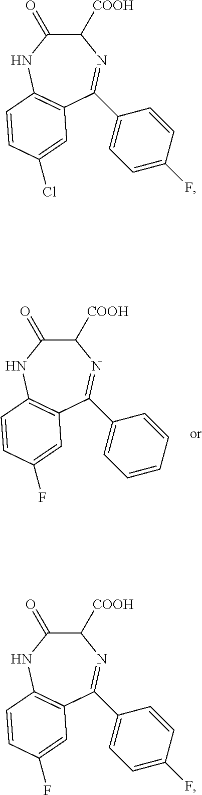

or a pharmaceutically acceptable salt thereof.

In one aspect, the invention features a method of making a fluorinated clorazepate, for example, a fluorinated clorazepate shown above, using a method described herein.

In one aspect, the invention features a fluorinated oxazepam, for example, a derivative of oxazepam wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated oxazepam is selected from one of the following:

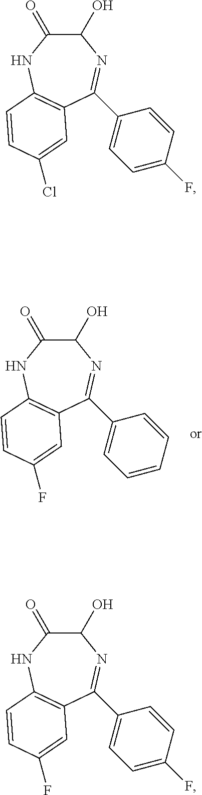

or a pharmaceutically acceptable salt thereof.

In one aspect, the invention features a method of making a fluorinated oxazepam, for example, a fluorinated oxazepam shown above, using a method described herein.

In one aspect, the invention features a fluorinated pericyazine, for example, a derivative of pericyazine wherein an aryl group has been substituted with one or more fluorine atoms, e.g., wherein a hydrogen or cyano substituent of an aryl group has been replaced with a fluorine. In some embodiments, the fluorine substituent is ¹⁹F. In some embodiments, the fluorine substituent is ¹⁸F. In one embodiment, the fluorinated pericyazine has the following formula:

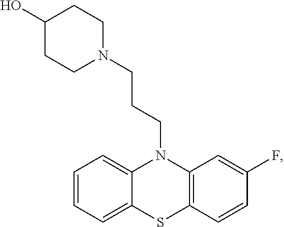

or a pharmaceutically acceptable salt thereof.

In one aspect, the invention features a method of making a fluorinated pericyazine, for example, the fluorinated pericyazine shown above, using a method described herein.

In one aspect, the invention features a fluorinated sulpiride, for example, a derivative of sulpiride wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated sulpiride has the following formula:

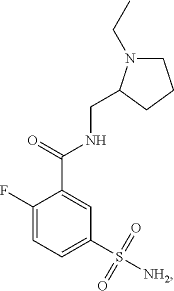

or a pharmaceutically acceptable salt thereof.

In one aspect, the invention features a method of making a fluorinated sulpiride, for example, the fluorinated sulpiride shown above, using a method described herein.

In one aspect, the invention features a fluorinated thioridazine, for example, a derivative of thioridazine wherein an aryl group has been substituted with one or more fluorine atoms, e.g., wherein a hydrogen or thioether substituent of an aryl group has been replaced with a fluorine. In some embodiments, the fluorine substituent is ¹⁹F. In some embodiments, the fluorine substituent is ¹⁸F. In one embodiment, the fluorinated thioridazine has the following formula:

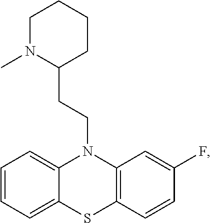

or a pharmaceutically acceptable salt thereof.

In one aspect, the invention features a method of making a fluorinated thioridazine, for example, the fluorinated thioridazine shown above, using a method described herein.

In one aspect, the invention features a fluorinated zuclopenthixol, for example, a derivative of zuclopenthixol wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated zuclopenthixol has the following formula:

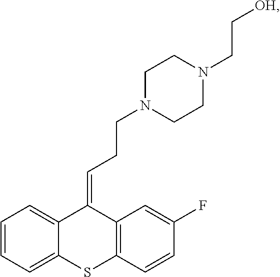

or a pharmaceutically acceptable salt thereof.

In one aspect, the invention features a method of making a fluorinated zuclopenthixol, for example, the fluorinated zuclopenthixol shown above, using a method described herein.

In one aspect, the invention features a fluorinated amisulpride, for example, a derivative of amisulpride wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated amisulpride has the following formula:

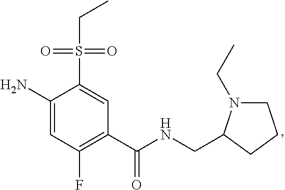

or a pharmaceutically acceptable salt thereof.

In one aspect, the invention features a method of making a fluorinated amisulpride, for example, the fluorinated amisulpride shown above, using a method described herein.

In one aspect, the invention features a fluorinated zotepine, for example, a derivative of zotepine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated zotepine has the following formula:

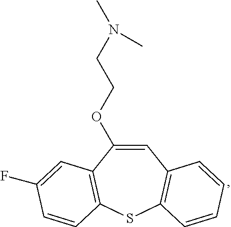

or a pharmaceutically acceptable salt thereof.

In one aspect, the invention features a method of making a fluorinated zotepine, for example, the fluorinated zotepine shown above, using a method described herein.

In one aspect, the invention features a fluorinated flupentixol, for example, a derivative of flupentixol wherein an aryl group has been substituted with one or more fluorine atoms, e.g., wherein a hydrogen or haloalkyl substituent of an aryl group has been replaced with a fluorine. In some embodiments, the fluorine substituent is ¹⁹F. In some embodiments, the fluorine substituent is ¹⁸F. In one embodiment, the fluorinated flupentixol has the following formula:

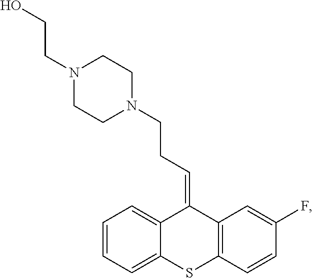

or a pharmaceutically acceptable salt thereof.

In one aspect, the invention features a method of making a fluorinated flupentixol, for example, the fluorinated flupentixol shown above, using a method described herein.

In one aspect, the invention features a fluorinated pipotiazine palmitate, for example, a derivative of pipotiazine palmitate wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated pipotiazine palmitate has the following formula:

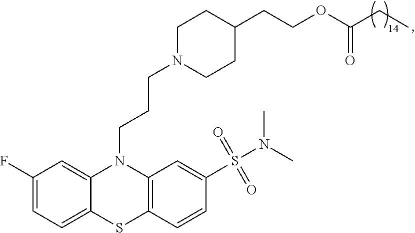

or a pharmaceutically acceptable salt thereof.

In one aspect, the invention features a method of making a fluorinated pipotiazine palmitate, for example, the fluorinated pipotiazine palmitate shown above, using a method described herein.

In one aspect, the invention features a fluorinated carbamazepine, for example, a derivative of carbamazepine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated carbamazepine is selected from one of the following:

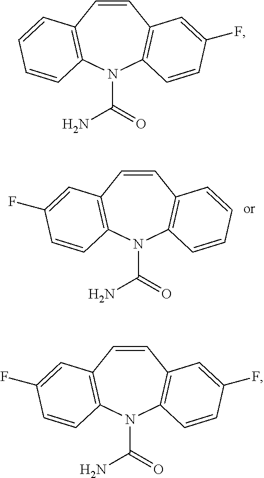

or a pharmaceutically acceptable salt thereof.

In one aspect, the invention features a method of making a fluorinated carbamazepine, for example, a fluorinated carbamazepine shown above, using a method described herein.

In one aspect, the invention features a fluorinated galantamine, for example, a derivative of galantamine wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated galantamine has the following formula:

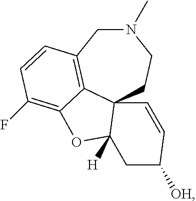

or a pharmaceutically acceptable salt thereof.

In one aspect, the invention features a method of making a fluorinated galantamine, for example, the fluorinated galantamine shown above, using a method described herein.

In one aspect, the invention features a fluorinated rivastigmine, for example, a derivative of rivastigmine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated rivastigmine is selected from one of the following:

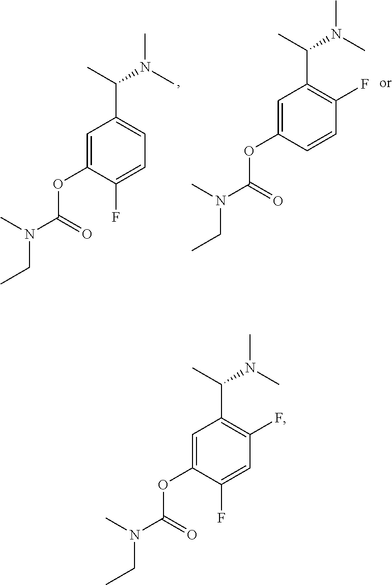

or a pharmaceutically acceptable salt thereof.

In one aspect, the invention features a method of making a fluorinated rivastigmine, for example, a fluorinated rivastigmine shown above, using a method described herein.

In one aspect, the invention features a fluorinated quetiapine, for example, a derivative of quetiapine wherein an aryl or heteroaryl group has been substituted with one or more fluorine atoms, e.g., wherein a hydrogen of an aryl or heteroaryl group has been replaced with a fluorine. In some embodiments, the fluorinated quetiapine does not have the following formula:

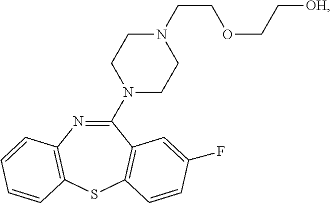

In some embodiments, the fluorine substituent is ¹⁹F. In some embodiments, the fluorine substituent is ¹⁸F. In some embodiments, the fluorinated quetiapine is selected from one of the following:

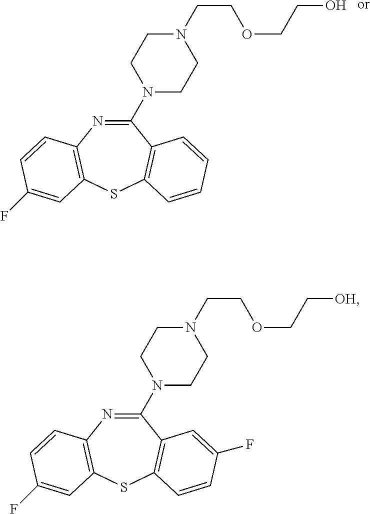

or a pharmaceutically acceptable salt thereof.

In one aspect, the invention features a method of making a fluorinated quetiapine, including any of the three fluorinated quetiapine structures shown above, using a method described herein.

In one aspect, the invention features an ¹⁸F-substituted quetiapine, for example, a derivative of quetiapine wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted quetiapine has the following formula:

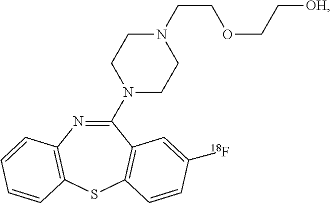

or a pharmaceutically acceptable salt thereof.

In one aspect, the invention features an ¹⁸F-substituted lamotrigine, for example, a derivative of lamotrigine wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted lamotrigine has the following formula:

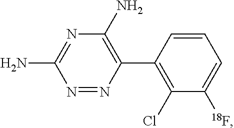

or a pharmaceutically acceptable salt thereof.

In one aspect, the invention features a method of making a fluorinated lamotrigine, for example, a fluorinated lamotrigine with the following formula, using a method described herein:

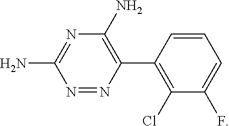

In one aspect, the invention features a method of making a fluorinated amphetamine, for example, a fluorinated amphetamine with the following formula, using a method described herein:

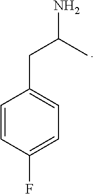

In one aspect, the invention features an ¹⁸F-substituted methylphenidate, for example, a derivative of methylphenidate wherein an aryl group has been substituted with one or more ¹⁸F atoms, e.g., wherein a hydrogen of an aryl group has been replaced with ¹⁸F. In one embodiment, the ¹⁸F-substituted methylphenidate has the following formula:

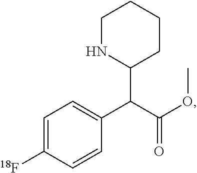

or a pharmaceutically acceptable salt thereof.

In one aspect, the invention features a method of making a fluorinated methylphenidate, for example, a fluorinated methylphenidate with the following formula, using a method described herein:

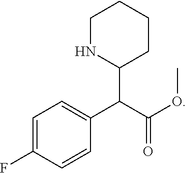

In one aspect, the invention features a method of making a fluorinated donepezil, for example, a fluorinated donepezil with the following formula, using a method described herein:

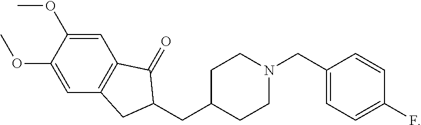

In one aspect, the invention features an ¹⁸F-substituted zolpidem, for example, a derivative of zolpidem wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted zolpidem has the following formula:

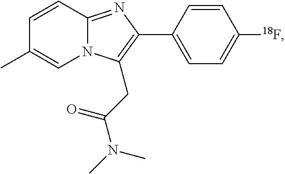

or a pharmaceutically acceptable salt thereof.

In one aspect, the invention features a method of making a fluorinated zolpidem, for example, a fluorinated zolpidem with the following formula, using a method described herein:

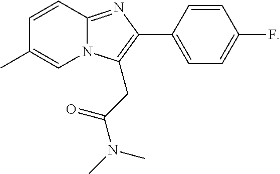

In one aspect, the invention features an ¹⁸F-substituted modafinil, for example, a derivative of modafinil wherein an aryl group has been substituted with one or more ¹⁸F atoms, e.g., wherein a hydrogen of an aryl group has been replaced with ¹⁸F. In one embodiment, the ¹⁸F-substituted modafinil has the following formula:

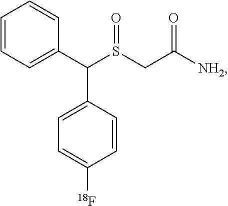

or a pharmaceutically acceptable salt thereof.

In one aspect, the invention features a method of making a fluorinated modafinil, for example, a fluorinated modafinil with the following formula, using a method described herein:

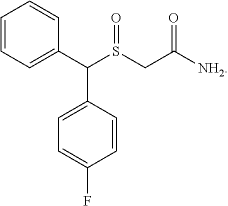

In one aspect, the invention features an ¹⁸F-substituted ziprasidone, for example, a derivative of ziprasidone wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted ziprasidone has the following formula:

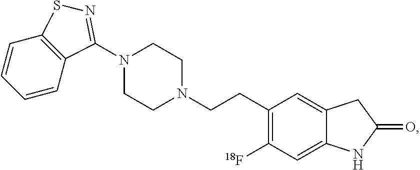

or a pharmaceutically acceptable salt thereof.

In one aspect, the invention features a method of making a fluorinated ziprasidone, for example, a fluorinated ziprasidone with the following formula, using a method described herein:

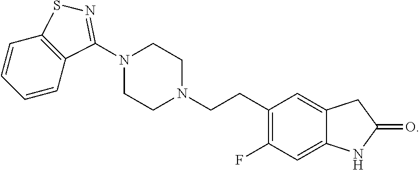

In one aspect, the invention features a fluorinated lorazepam, for example, a derivative of lorazepam wherein an aryl group has been substituted with one or more fluorine atoms, e.g., wherein a hydrogen or halogen substituent of an aryl group has been replaced with a fluorine. In some embodiments, the fluorinated lorazepam does not have the following formula:

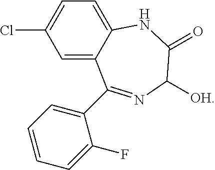

In some embodiments, the fluorine substituent is ¹⁹F. In some embodiments, the fluorine substituent is ¹⁸F. In some embodiments, the fluorinated lorazepam is selected from one of the following:

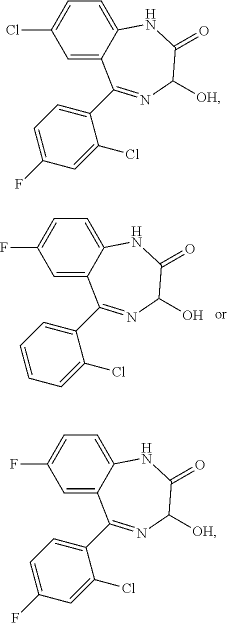

or a pharmaceutically acceptable salt thereof.

In one aspect, the invention features a method of making a fluorinated lorazepam, including any of the four fluorinated lorazepam structures shown above, using a method described herein.

In one aspect, the invention features an ¹⁸F-substituted lorazepam, for example, a derivative of lorazepam wherein an aryl group has been substituted with one or more ¹⁸F atoms, e.g., wherein a hydrogen or halogen substituent of an aryl group has been replaced with ¹⁸F. In one embodiment, the ¹⁸F-substituted lorazepam has the following formula:

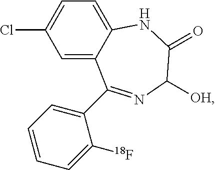

or a pharmaceutically acceptable salt thereof.

In one aspect, the invention features an ¹⁸F-substituted clonazepam, for example, a derivative of clonazepam wherein an aryl group has been substituted with one or more ¹⁸F atoms, e.g., wherein a hydrogen or halogen substituent of an aryl group has been replaced with ¹⁸F. In one embodiment, the ¹⁸F-substituted clonazepam has the following formula:

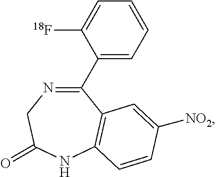

or a pharmaceutically acceptable salt thereof.

In one aspect, the invention features a method of making a fluorinated clonazepam, for example, a fluorinated clonazepam with the following formula, using a method described herein:

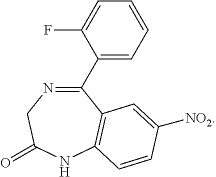

In one aspect, the invention features an ¹⁸F-substituted diazepam, for example, a derivative of diazepam wherein an aryl group has been substituted with one or more ¹⁸F atoms, e.g., wherein a hydrogen or halogen substituent of an aryl group has been replaced with ¹⁸F. In one embodiment, the ¹⁸F-substituted diazepam has the following formula:

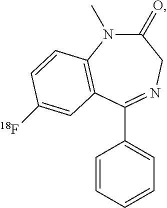

or a pharmaceutically acceptable salt thereof.

In one aspect, the invention features a method of making a fluorinated diazepam, for example, a fluorinated diazepam with the following formula, using a method described herein:

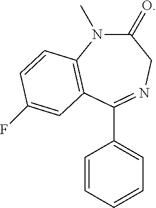

In one aspect, the invention features an ¹⁸F-substituted clozapine, for example, a derivative of clozapine wherein an aryl group has been substituted with one or more ¹⁸F atoms, e.g., wherein a hydrogen or halogen substituent of an aryl group has been replaced with ¹⁸F. In one embodiment, the ¹⁸F-substituted clozapine has the following formula:

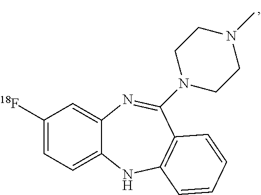

or a pharmaceutically acceptable salt thereof.

In one aspect, the invention features a method of making a fluorinated clozapine, for example, a fluorinated clozapine with the following formula, using a method described herein:

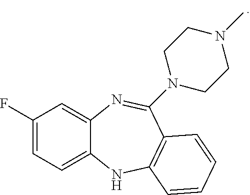

In one aspect, the invention features a fluorinated temazepam, for example, a derivative of temazepam wherein an aryl group has been substituted with one or more fluorine atoms, e.g., wherein a hydrogen or halogen substituent of an aryl group has been replaced with a fluorine. In some embodiments, the fluorinated temazepam does not have the following formula:

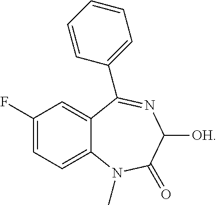

In some embodiments, the fluorine substituent is ¹⁹F. In some embodiments, the fluorine substituent is ¹⁸F. In some embodiments, the fluorinated temazepam is selected from one of the following:

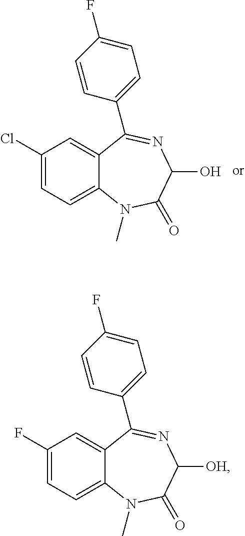

or a pharmaceutically acceptable salt thereof.

In one aspect, the invention features a method of making a fluorinated temazepam, including any of the three fluorinated temazepam structures shown above, using a method described herein.

In one aspect, the invention features an ¹⁸F-substituted temazepam, for example, a derivative of temazepam wherein an aryl group has been substituted with one or more ¹⁸F atoms, e.g., wherein a hydrogen or halogen substituent of an aryl group has been replaced with ¹⁸F. In one embodiment, the ¹⁸F-substituted temazepam has the following formula:

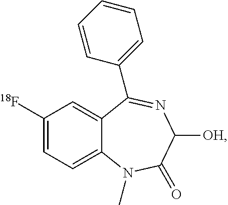

or a pharmaceutically acceptable salt thereof.

In one aspect, the invention features a method of making a fluorinated dextroamphetamine, for example, a fluorinated dextroamphetamine with the following formula, using a method described herein:

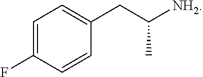

In one aspect, the invention features an ¹⁸F-substituted nitrazepam, for example, a derivative of nitrazepam wherein an aryl group has been substituted with one or more ¹⁸F atoms, e.g., wherein a hydrogen of an aryl group has been replaced with ¹⁸F. In one embodiment, the ¹⁸F-substituted nitrazepam has the following formula:

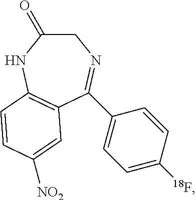

or a pharmaceutically acceptable salt thereof.

In one aspect, the invention features a method of making a fluorinated nitrazepam, for example, a fluorinated nitrazepam with the following formula, using a method described herein:

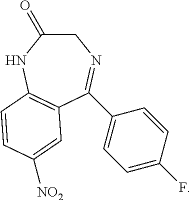

In one aspect, the invention features a fluorinated loprazolam, for example, a derivative of loprazolam wherein an aryl group has been substituted with one or more fluorine atoms, e.g., wherein a hydrogen or halogen substituent of an aryl group has been replaced with a fluorine. In some embodiments, the fluorinated loprazolam does not have the following formula:

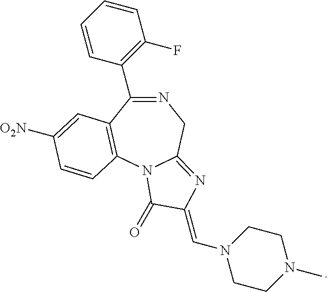

In some embodiments, the fluorine substituent is ¹⁹F. In some embodiments, the fluorine substituent is ¹⁸F. In some embodiments, the fluorinated loprazolam is selected from one of the following:

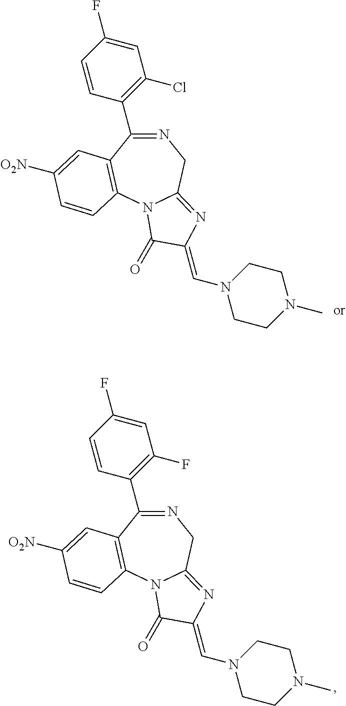

or a pharmaceutically acceptable salt thereof.

In one aspect, the invention features a method of making a fluorinated loprazolam, including any of the three fluorinated loprazolam structures shown above, using a method described herein.

In one aspect, the invention features an ¹⁸F-substituted loprazolam, for example, a derivative of loprazolam wherein an aryl group has been substituted with one or more ¹⁸F atoms, e.g., wherein a hydrogen or halogen substituent of an aryl group has been replaced with ¹⁸F. In one embodiment, the ¹⁸F-substituted loprazolam has the following formula:

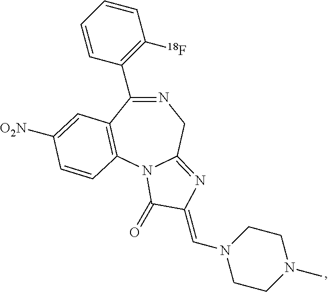

or a pharmaceutically acceptable salt thereof.

In one aspect, the invention features an ¹⁸F-substituted buspirone, for example, a derivative of buspirone wherein a heteroaryl group has been substituted with one or more ¹⁸F atoms, e.g., wherein a hydrogen of a heteroaryl group has been replaced with ¹⁸F. In one embodiment, the ¹⁸F-substituted buspirone has the following formula:

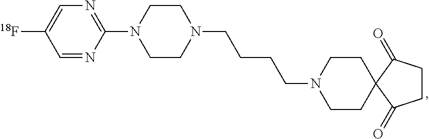

or a pharmaceutically acceptable salt thereof.

In one aspect, the invention features a method of making a fluorinated buspirone, for example, a fluorinated buspirone with the following formula, using a method described herein:

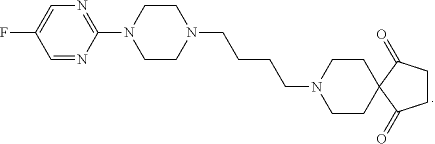

In one aspect, the invention features a fluorinated benperidol, for example, a derivative of benperidol wherein an aryl group has been substituted with one or more fluorine atoms, e.g., wherein a hydrogen of an aryl group has been replaced with a fluorine. In some embodiments, the fluorinated benperidol does not have the following formula:

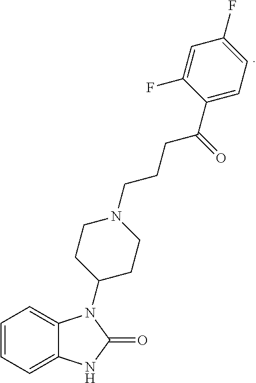

In some embodiments, the fluorine substituent is ¹⁹F. In some embodiments, the fluorine substituent is ¹⁸F. In some embodiments, the fluorinated benperidol is selected from one of the following:

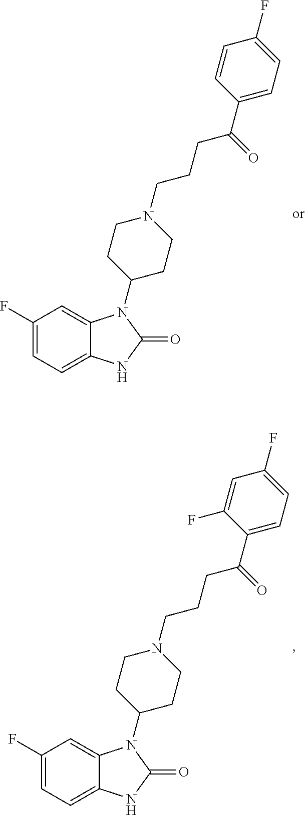

or a pharmaceutically acceptable salt thereof.

In one aspect, the invention features a method of making a fluorinated benperidol, including any of the three fluorinated benperidol structures shown above, using a method described herein.

In one aspect, the invention features an ¹⁸F-substituted benperidol, for example, a derivative of benperidol wherein an aryl group has been substituted with one or more ¹⁸F atoms, e.g., wherein a hydrogen of an aryl group has been replaced with ¹⁸F. In one embodiment, the ¹⁸F-substituted benperidol is selected from one of the following:

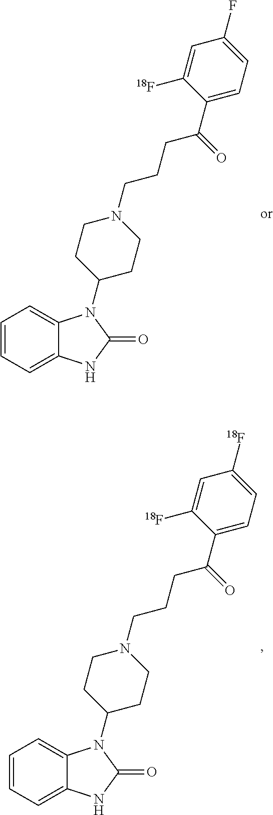

or a pharmaceutically acceptable salt thereof.

In one aspect, the invention features an ¹⁸F-substituted chlorpromazine or an ¹⁸F-substituted promazine, for example, a derivative of chlorpromazine or promazine wherein an aryl group has been substituted with one or more ¹⁸F atoms, e.g., wherein a hydrogen or a halogen substituent of an aryl group has been replaced with ¹⁸F. In one embodiment, the ¹⁸F-substituted chlorpromazine or promazine has the following formula:

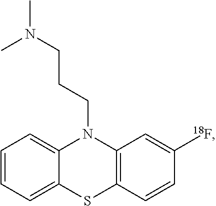

or a pharmaceutically acceptable salt thereof.

In one aspect, the invention features a method of making a fluorinated chlorpromazine or a fluorinated promazine, for example, a fluorinated chlorpromazine or fluorinated promazine with the following formula, using a method described herein:

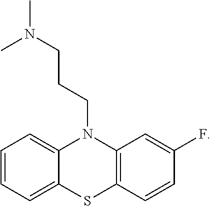

In one aspect, the invention features an ¹⁸F-substituted fluphenazine or an ¹⁸F-substituted perphenazine, for example, a derivative of fluphenazine or perphenazine wherein an aryl group has been substituted with one or more ¹⁸F atoms, e.g., wherein a hydrogen, a halogen substituent or a haloalkyl substituent of an aryl group has been replaced with ¹⁸F. In one embodiment, the ¹⁸F-substituted fluphenazine or ¹⁸F-substituted perphenazine has the following formula:

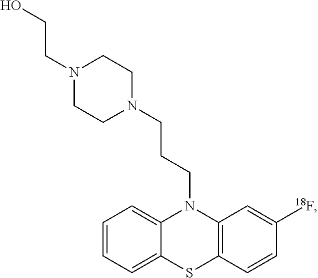

or a pharmaceutically acceptable salt thereof.

In one aspect, the invention features a method of making a fluorinated fluphenazine or a fluorinated perphenazine, for example, a fluorinated fluphenazine or fluorinated perphenazine with the following formula, using a method described herein:

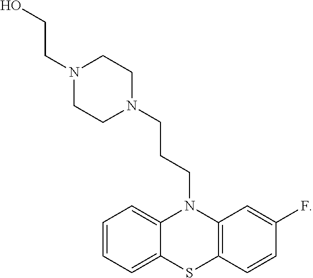

In one aspect, the invention features an ¹⁸F-substituted haloperidol, for example, a derivative of haloperidol wherein an aryl group has been substituted with one or more ¹⁸F atoms, e.g., wherein a hydrogen or a halogen substituent of an aryl group has been replaced with ¹⁸F. In one embodiment, the ¹⁸F-substituted haloperidol has the following formula:

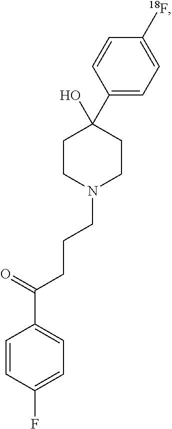

or a pharmaceutically acceptable salt thereof.

In one aspect, the invention features a method of making a fluorinated haloperidol, for example, a fluorinated haloperidol with one of the following formulae, using a method described herein:

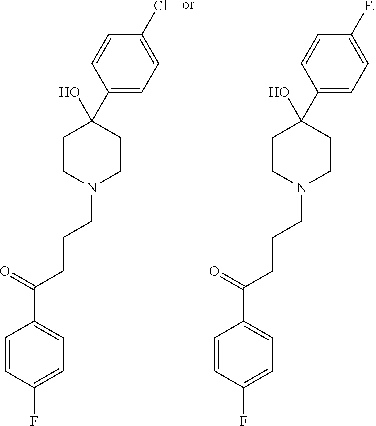

In one aspect, the invention features an ¹⁸F-substituted methotrimeprazine, for example, a derivative of methotrimeprazine wherein an aryl group has been substituted with one or more ¹⁸F atoms, e.g., wherein a hydrogen or an alkoxy substituent of an aryl group has been replaced with ¹⁸F. In one embodiment, the ¹⁸F-substituted methotrimeprazine has the following formula:

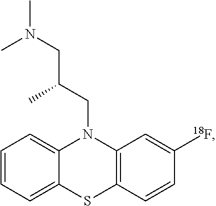

or a pharmaceutically acceptable salt thereof.

In one aspect, the invention features a method of making a fluorinated methotrimeprazine, for example, a fluorinated methotrimeprazine with the following formula, using a method described herein:

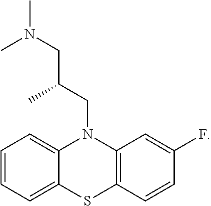

In one aspect, the invention features a fluorinated loxapine, for example, a derivative of loxapine wherein an aryl or heteroaryl group has been substituted with one or more fluorine atoms, e.g., wherein a hydrogen or halogen substituent of an aryl or heteroaryl group has been replaced with a fluorine. In some embodiments, the fluorinated loxapine does not have the following formula:

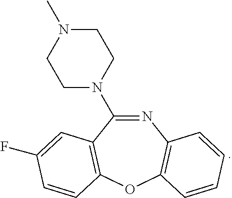

In some embodiments, the fluorine substituent is ¹⁹F. In some embodiments, the fluorine substituent is ¹⁸F. In some embodiments, the fluorinated loxapine is selected from one of the following:

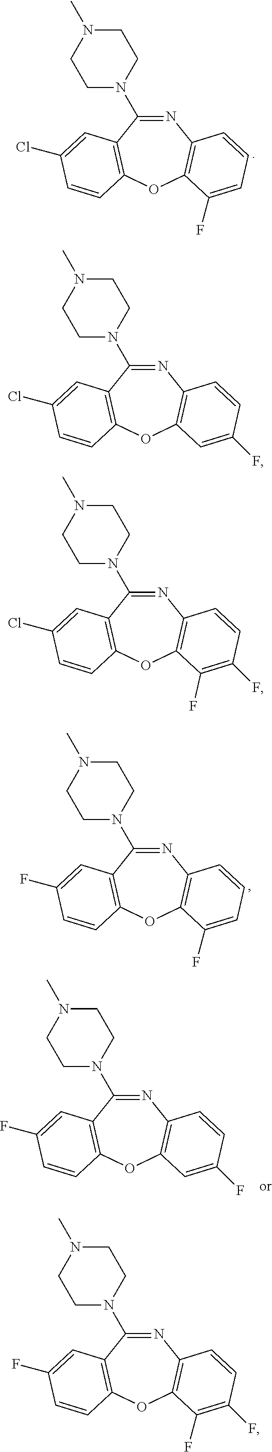

or a pharmaceutically acceptable salt thereof.

In one aspect, the invention features a method of making a fluorinated loxapine, including any of the seven fluorinated loxapine structures shown above, using a method described herein.

In one aspect, the invention features an ¹⁸F-substituted loxapine, for example, a derivative of loxapine wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted loxapine has the following formula:

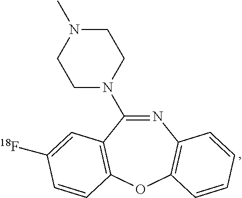

or a pharmaceutically acceptable salt thereof.

In one aspect, the invention features a fluorinated oxypertine, for example, a derivative of oxypertine wherein an aryl or heteroaryl group has been substituted with one or more fluorine atoms, e.g., wherein a hydrogen or alkoxy substituent of an aryl or heteroaryl group has been replaced with a fluorine. In some embodiments, the fluorinated oxypertine does not have the following formula:

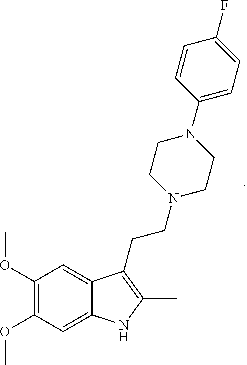

In some embodiments, the fluorine substituent is ¹⁹F. In some embodiments, the fluorine substituent is ¹⁸F. In some embodiments, the fluorinated oxypertine is selected from one of the following:

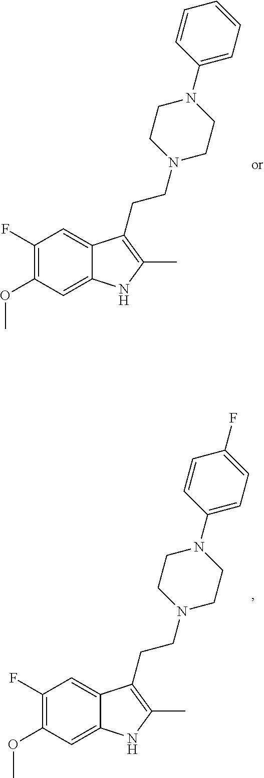

or a pharmaceutically acceptable salt thereof.

In one aspect, the invention features a method of making a fluorinated oxypertine, including any of the three fluorinated oxypertine structures shown above, using a method described herein.

In one aspect, the invention features an ¹⁸F-substituted oxypertine, for example, a derivative of oxypertine wherein an aryl or heteroaryl group has been substituted with one or more ¹⁸F atoms, e.g., wherein a hydrogen or alkoxy substituent of an aryl or heteroaryl group has been replaced with ¹⁸F. In one embodiment, the ¹⁸F-substituted oxypertine has the following formula:

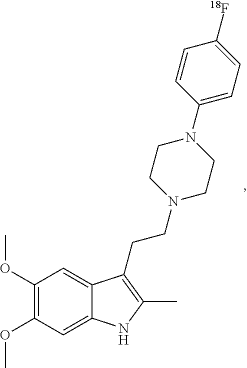

or a pharmaceutically acceptable salt thereof.

In one aspect, the invention features a fluorinated pimozide, for example, a derivative of pimozide wherein an aryl or heteroaryl group has been substituted with one or more fluorine atoms, e.g., wherein a hydrogen or halogen substituent of an aryl or heteroaryl group has been replaced with a fluorine. In some embodiments, the fluorinated pimozide does not have the following formula:

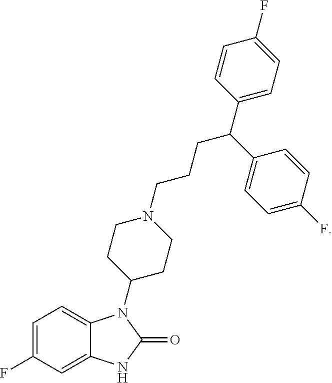

In some embodiments, the fluorine substituent is ¹⁹F. In some embodiments, the fluorine substituent is ¹⁸F. In some embodiments, the fluorinated pimozide is selected from one of the following:

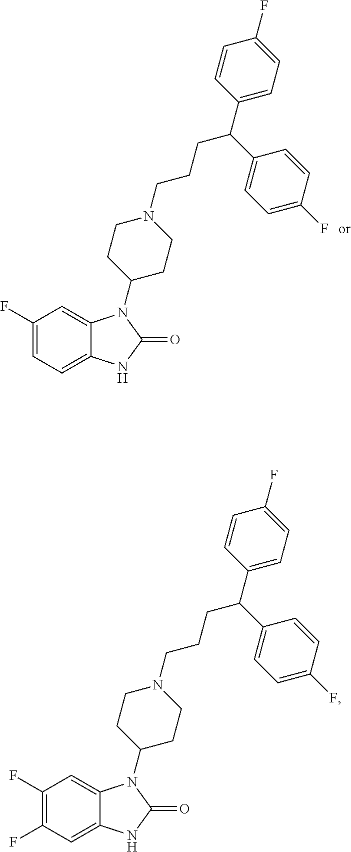

or a pharmaceutically acceptable salt thereof.

In one aspect, the invention features a method of making a fluorinated pimozide, including any of the three fluorinated pimozide structures shown above, using a method described herein.

In one aspect, the invention features an ¹⁸F-substituted pimozide, for example, a derivative of pimozide wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted pimozide has the following formula:

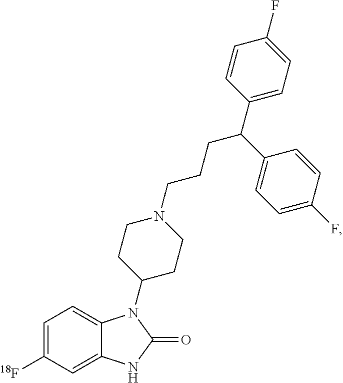

or a pharmaceutically acceptable salt thereof.

In one aspect, the invention features an ¹⁸F-substituted prochlorperazine or an ¹⁸F-substituted trifluoperazine, for example, a derivative of prochlorperazine or trifluoperazine wherein an aryl group has been substituted with one or more ¹⁸F atoms, e.g., wherein a hydrogen, a halogen substituent or a haloalkyl substituent of an aryl group has been replaced with ¹⁸F. In one embodiment, the ¹⁸F-substituted prochlorperazine or ¹⁸F-substituted trifluoperazine has the following formula:

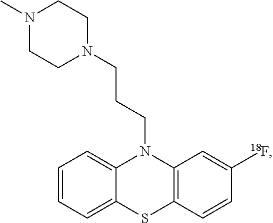

or a pharmaceutically acceptable salt thereof.

In one aspect, the invention features a method of making a fluorinated prochlorperazine or a fluorinated trifluoperazine, for example, a fluorinated prochlorperazine or a fluorinated trifluoperazine with the following formula, using a method described herein:

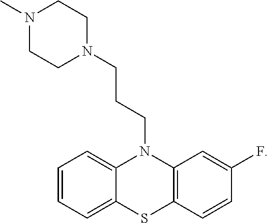

In one aspect, the invention features an ¹⁸F-substituted benzodiazepine, for example, a derivative of benzodiazepine wherein an aryl group has been substituted with one or more ¹⁸F atoms, e.g., wherein a hydrogen of an aryl group has been replaced with ¹⁸F. In one embodiment, the ¹⁸F-substituted benzodiazepine is selected from one of the following:

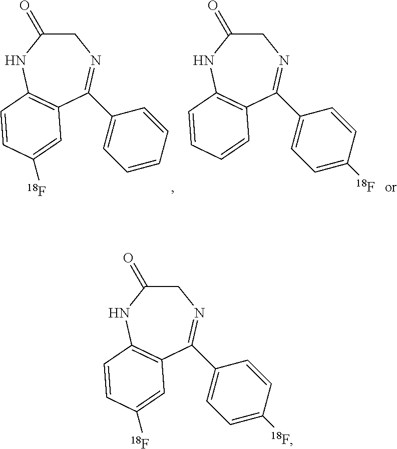

or a pharmaceutically acceptable salt thereof.

In one aspect, the invention features a method of making a fluorinated benzodiazepine, for example, a fluorinated benzodiazepine with one of the following formulae, using a method described herein:

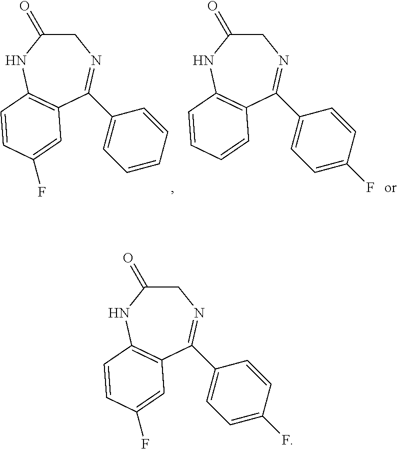

In one aspect, the invention features a fluorinated metaxalone, for example, a derivative of metaxalone wherein an aryl or heteroaryl group has been substituted with one or more fluorine atoms, e.g., wherein a hydrogen or alkyl substituent of an aryl or heteroaryl group has been replaced with a fluorine. In some embodiments, the fluorine substituent is ¹⁹F. In some embodiments, the fluorine substituent is ¹⁸F. In some embodiments, the fluorinated metaxalone is selected from one of the following:

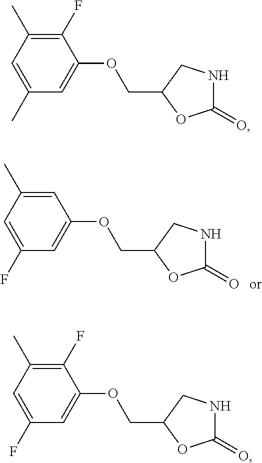

or a pharmaceutically acceptable salt thereof.

In one aspect, the invention features a method of making a fluorinated metaxalone, for example, a fluorinated metaxalone shown above, using a method described herein.

In one aspect, the invention features a fluorinated tizanidine, for example, a derivative of tizanidine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated tizanidine has the following formula:

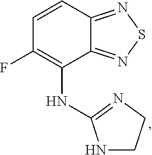

or a pharmaceutically acceptable salt thereof.

In one aspect, the invention features a method of making a fluorinated tizanidine, for example, the fluorinated tizanidine shown above, using a method described herein.

In one aspect, the invention features a fluorinated benzonatate, for example, a derivative of benzonatate wherein an aryl group has been substituted with one or more fluorine atoms, e.g., wherein a hydrogen or alkylamino substituent of an aryl group has been replaced with a fluorine. In some embodiments, the fluorine substituent is ¹⁹F. In some embodiments, the fluorine substituent is ¹⁸F. In one embodiment, the fluorinated benzonatate has the following formula:

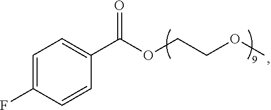

or a pharmaceutically acceptable salt thereof.

In one aspect, the invention features a method of making a fluorinated benzonatate, for example, the fluorinated benzonatate shown above, using a method described herein.

In one aspect, the invention features an ¹⁸F-substituted lidocaine, for example, a derivative of lidocaine wherein an aryl group has been substituted with one or more ¹⁸F atoms, e.g., wherein a hydrogen of an aryl group has been replaced with ¹⁸F. In one embodiment, the ¹⁸F-substituted lidocaine has the following formula:

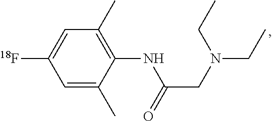

or a pharmaceutically acceptable salt thereof.

In one aspect, the invention features a method of making a fluorinated lidocaine, for example, a fluorinated lidocaine with the following formula, using a method described herein:

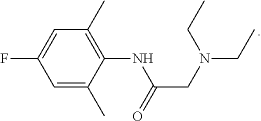

In one aspect, the invention features an ¹⁸F-substituted acetaminophen, for example, a derivative of acetaminophen wherein an aryl group has been substituted with one or more ¹⁸F atoms, e.g., wherein a hydrogen or hydroxy substituent of an aryl group has been replaced with ¹⁸F. In one embodiment, the ¹⁸F-substituted acetaminophen has the following formula:

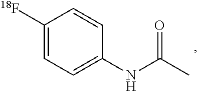

or a pharmaceutically acceptable salt thereof.

In one aspect, the invention features a method of making a fluorinated acetaminophen, for example, a fluorinated acetaminophen with the following formula, using a method described herein:

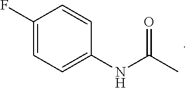

In one aspect, the invention features a fluorinated tramadol, for example, a derivative of tramadol wherein an aryl group has been substituted with one or more fluorine atoms, e.g., wherein a hydrogen or alkoxy substituent of an aryl group has been replaced with a fluorine. In some embodiments, the fluorinated tramadol does not have the following formula:

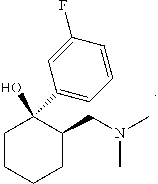

In some embodiments, the fluorine substituent is ¹⁹F. In some embodiments, the fluorine substituent is ¹⁸F. In some embodiments, the fluorinated tramadol has the following formula:

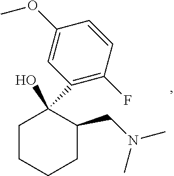

or a pharmaceutically acceptable salt thereof.

In one aspect, the invention features a method of making a fluorinated tramadol, including either of the two fluorinated tramadol structures shown above, using a method described herein.

In one aspect, the invention features an ¹⁸F-substituted tramadol, for example, a derivative of tramadol wherein an aryl group has been substituted with one or more ¹⁸F atoms, e.g., wherein a hydrogen or alkoxy substituent of an aryl group has been replaced with ¹⁸F. In one embodiment, the ¹⁸F-substituted tramadol has the following formula:

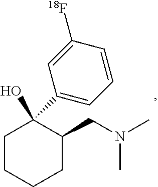

or a pharmaceutically acceptable salt thereof.

In one aspect, the invention features a fluorinated ketamine, for example, a derivative of ketamine wherein an aryl group has been substituted with one or more fluorine atom, e.g., wherein a hydrogen or halogen substituent of an aryl group has been replaced with a fluorine. In some embodiments, the fluorinated ketamine does not have the following formula:

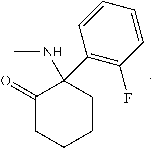

In some embodiments, the fluorine substituent is ¹⁹F. In some embodiments, the fluorine substituent is ¹⁸F. In some embodiments, the fluorinated ketamine is selected from one of the following:

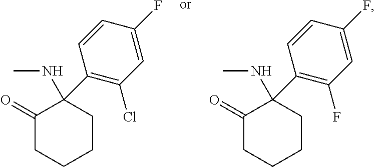

or a pharmaceutically acceptable salt thereof.

In one aspect, the invention features a method of making a fluorinated ketamine, including any of the three fluorinated ketamine structures shown above, using a method described herein.

In one aspect, the invention features an ¹⁸F-substituted ketamine, for example, a derivative of ketamine wherein an aryl group has been substituted with one or more ¹⁸F atoms, e.g., wherein a hydrogen or halogen substituent of an aryl group has been replaced with ¹⁸F. In one embodiment, the ¹⁸F-substituted ketamine has the following formula:

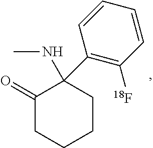

or a pharmaceutically acceptable salt thereof.

In one aspect, the invention features a fluorinated lansoprazole, for example, a derivative of lansoprazole wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated lansoprazole has the following formula:

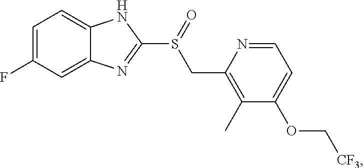

or a pharmaceutically acceptable salt thereof.

In one aspect, the invention features a method of making a fluorinated lansoprazole, for example, the fluorinated lansoprazole shown above, using a method described herein.

In one aspect, the invention features a fluorinated rabeprazole, for example, a derivative of rabeprazole wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated rabeprazole has the following formula:

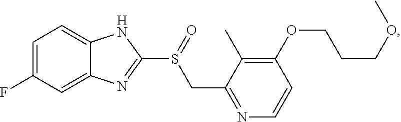

or a pharmaceutically acceptable salt thereof.

In one aspect, the invention features a method of making a fluorinated rabeprazole, for example, the fluorinated rabeprazole shown above, using a method described herein.

In one aspect, the invention features a fluorinated tamsulosin, for example, a derivative of tamsulosin wherein an aryl or heteroaryl group has been substituted with one or more fluorine atoms, e.g., wherein a hydrogen or alkoxy substituent of an aryl or heteroaryl group has been replaced with a fluorine. In some embodiments, the fluorine substituent is ¹⁹F. In some embodiments, the fluorine substituent is ¹⁸F. In some embodiments, the fluorinated tamsulosin has the following formula:

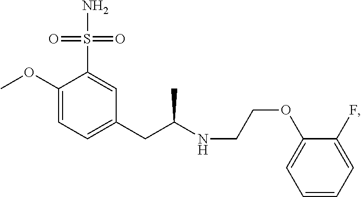

or a pharmaceutically acceptable salt thereof.

In one aspect, the invention features a method of making a fluorinated tamsulosin, for example, the fluorinated tamsulosin shown above, using a method described herein.

In one aspect, the invention features a fluorinated ethinyl estradiol, for example, a derivative of ethinyl estradiol wherein an aryl group has been substituted with one or more fluorine atoms, e.g., wherein a hydrogen or hydroxyl substituent of an aryl group has been replaced with a fluorine. In some embodiments, the fluorine substituent is ¹⁹F. In some embodiments, the fluorine substituent is ¹⁸F. In some embodiments, the fluorinated ethinyl estradiol has the following formula:

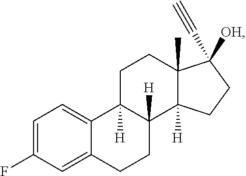

or a pharmaceutically acceptable salt thereof.

In one aspect, the invention features a method of making a fluorinated ethinyl estradiol, for example, the fluorinated ethinyl estradiol shown above, using a method described herein.

In one aspect, the invention features a fluorinated imiquimod, for example, a derivative of imiquimod wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated imiquimod has the following formula:

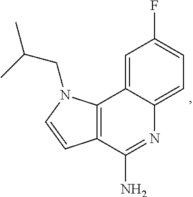

or a pharmaceutically acceptable salt thereof.

In one aspect, the invention features a method of making a fluorinated imiquimod, for example, the fluorinated imiquimod shown above, using a method described herein.

In one aspect, the invention features a fluorinated cinacalcet, for example, a derivative of cinacalcet wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated cinacalcet has the following formula:

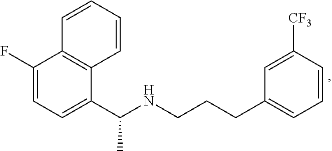

or a pharmaceutically acceptable salt thereof.

In one aspect, the invention features a method of making a fluorinated cinacalcet, for example, the fluorinated cinacalcet shown above, using a method described herein.

In one aspect, the invention features a fluorinated olopatadine, for example, a derivative of olopatadine wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated olopatadine has the following formula:

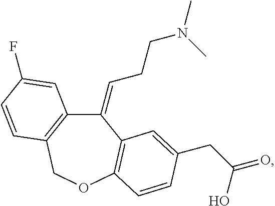

or a pharmaceutically acceptable salt thereof.

In one aspect, the invention features a method of making a fluorinated olopatadine, for example, the fluorinated olopatadine shown above, using a method described herein.

In one aspect, the invention features a fluorinated bimatoprost, for example, a derivative of bimatoprost wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated bimatoprost has the following formula:

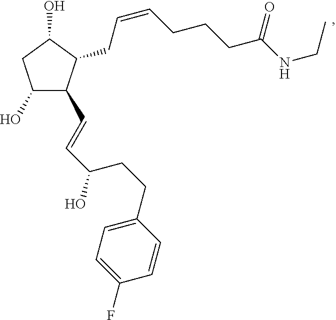

or a pharmaceutically acceptable salt thereof.

In one aspect, the invention features a method of making a fluorinated bimatoprost, for example, the fluorinated bimatoprost shown above, using a method described herein.

In one aspect, the invention features a fluorinated adapalene, for example, a derivative of adapalene wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated adapalene has the following formula:

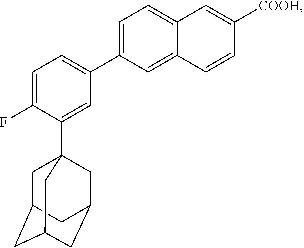

or a pharmaceutically acceptable salt thereof.

In one aspect, the invention features a method of making a fluorinated adapalene, for example, the fluorinated adapalene shown above, using a method described herein.

In one aspect, the invention features a fluorinated brimonidine, for example, a derivative of brimonidine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated brimonidine has the following formula:

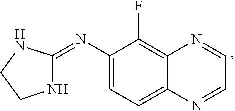

or a pharmaceutically acceptable salt thereof.

In one aspect, the invention features a method of making a fluorinated brimonidine, for example, the fluorinated brimonidine shown above, using a method described herein.

In one aspect, the invention features a fluorinated furosemide, for example, a derivative of furosemide wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated furosemide has the following formula:

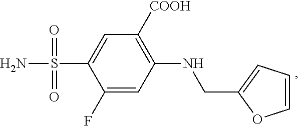

or a pharmaceutically acceptable salt thereof.

In one aspect, the invention features a method of making a fluorinated furosemide, for example, the fluorinated furosemide shown above, using a method described herein.

In one aspect, the invention features a fluorinated terazosin, for example, a derivative of terazosin wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated terazosin methadone is selected from one of the following:

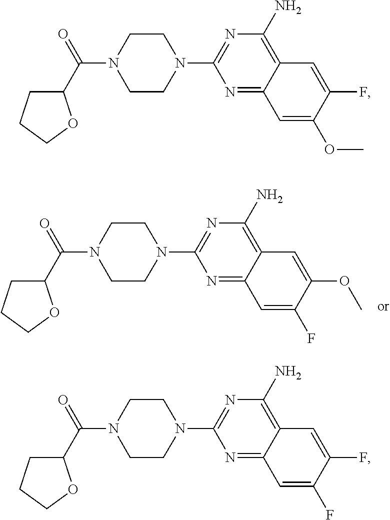

or a pharmaceutically acceptable salt thereof.

In one aspect, the invention features a method of making a fluorinated terazosin, for example, a fluorinated terazosin shown above, using a method described herein.

In one aspect, the invention features a fluorinated metolazone, for example, a derivative of metolazone wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated metolazone has the following formula:

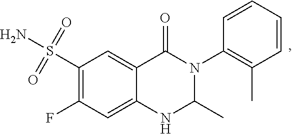

or a pharmaceutically acceptable salt thereof.

In one aspect, the invention features a method of making a fluorinated metolazone, for example, the fluorinated metolazone shown above, using a method described herein.

In one aspect, the invention features an ¹⁸F-substituted esomeprazole, for example, a derivative of esomeprazole wherein an aryl or heteroaryl group has been substituted with one or more ¹⁸F atoms, e.g., wherein a hydrogen or methoxy substituent of an aryl or heteroaryl group has been replaced with ¹⁸F. In one embodiment, the ¹⁸F-substituted esomeprazole has the following formula:

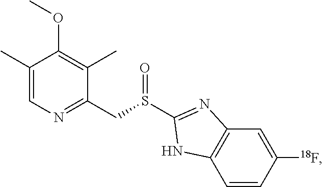

or a pharmaceutically acceptable salt thereof.

In one aspect, the invention features a method of making a fluorinated esomeprazole, for example, a fluorinated esomeprazole with the following formula, using a method described herein:

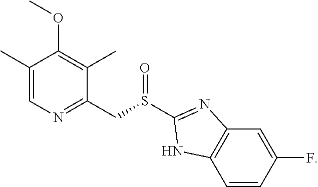

In one aspect, the invention features an ¹⁸F-substituted mycophenolic acid, for example, a derivative of mycophenolic acid wherein an aryl group has been substituted with one or more ¹⁸F atoms, e.g., wherein a hydrogen or hydroxy substituent of an aryl group has been replaced with ¹⁸F. In one embodiment, the ¹⁸F-substituted mycophenolic acid has the following formula:

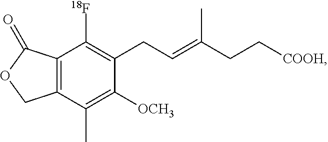

or a pharmaceutically acceptable salt thereof.

In one aspect, the invention features a method of making a fluorinated mycophenolic acid, for example, a fluorinated mycophenolic acid with the following formula, using a method described herein:

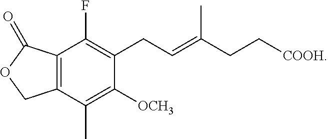

In one aspect, the invention features an ¹⁸F-substituted estrone (e.g., a component of Premarin), for example, a derivative of estrone wherein an aryl group has been substituted with one or more ¹⁸F atoms, e.g., wherein a hydrogen of an aryl group has been replaced with ¹⁸F. In one embodiment, the ¹⁸F-substituted estrone has the following formula:

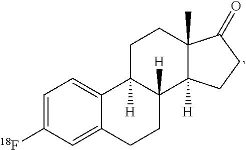

or a pharmaceutically acceptable salt thereof.

In one aspect, the invention features a method of making a fluorinated estrone, for example, a fluorinated estrone with the following formula, using a method described herein:

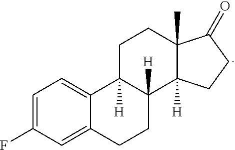

In one aspect, the invention features an ¹⁸F-substituted levothyroxine, for example, a derivative of levothyroxine wherein an aryl group has been substituted with one or more ¹⁸F atoms, e.g., wherein a hydrogen or halogen substituent of an aryl group has been replaced with ¹⁸F. In one embodiment, the ¹⁸F-substituted levothyroxine is selected from one of the following:

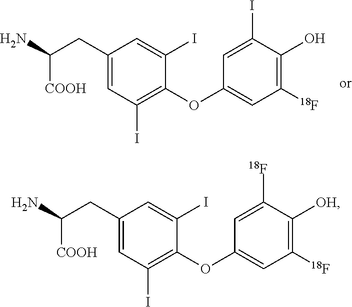

or a pharmaceutically acceptable salt thereof.

In one aspect, the invention features a method of making a fluorinated levothyroxine, for example, a fluorinated levothyroxine with one of the following formulae, using a method described herein:

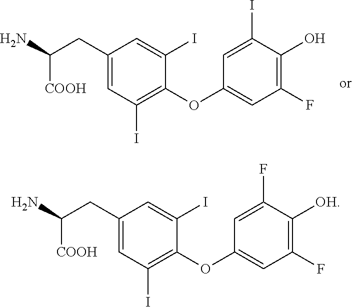

In one aspect, the invention features a fluorinated omeprazole, for example, a derivative of omeprazole wherein a heteroaryl has been substituted with one or more fluorine atoms, e.g., wherein a hydrogen or alkoxy substituent of a heteroaryl group has been replaced with a fluorine. In some embodiments, the fluorinated omeprazole does not have the following formula:

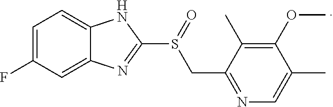

In some embodiments, the fluorine substituent is ¹⁹F. In some embodiments, the fluorine substituent is ¹⁸F. In some embodiments, the fluorinated omeprazole has the following formula:

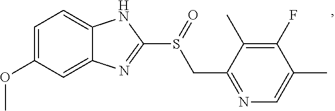

or a pharmaceutically acceptable salt thereof.

In one aspect, the invention features a method of making a fluorinated omeprazole, including either of the two fluorinated omeprazole structures shown above, using a method described herein.

In one aspect, the invention features an ¹⁸F-substituted omeprazole, for example, a derivative of omeprazole wherein an aryl or heteroaryl group has been substituted with one or more ¹⁸F atoms, e.g., wherein a hydrogen or alkoxy substituent of an aryl or heteroaryl group has been replaced with ¹⁸F. In one embodiment, the ¹⁸F-substituted omeprazole has the following formula:

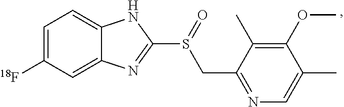

or a pharmaceutically acceptable salt thereof.

In one aspect, the invention features an ¹⁸F-substituted ondansetron, for example, a derivative of ondansetron wherein an aryl group has been substituted with one or more ¹⁸F atoms, e.g., wherein a hydrogen or halogen substituent of an aryl group has been replaced with ¹⁸F. In one embodiment, the ¹⁸F-substituted ondansetron has the following formula:

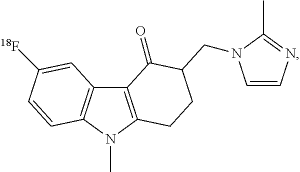

or a pharmaceutically acceptable salt thereof.

In one aspect, the invention features a method of making a fluorinated ondansetron, for example, a fluorinated ondansetron with the following formula, using a method described herein:

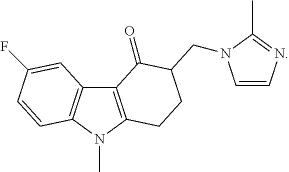

In one aspect, the invention features an ¹⁸F-substituted metoclopramide, for example, a derivative of metoclopramide wherein an aryl group has been substituted with one or more ¹⁸F atoms, e.g., wherein a hydrogen or halogen substituent of an aryl group has been replaced with ¹⁸F. In one embodiment, the ¹⁸F-substituted metoclopramide has the following formula:

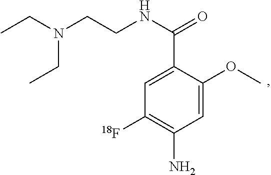

or a pharmaceutically acceptable salt thereof.

In one aspect, the invention features a method of making a fluorinated metoclopramide, for example, a fluorinated metoclopramide with the following formula, using a method described herein:

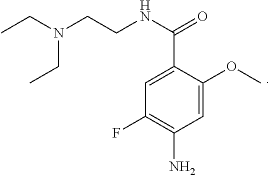

In one aspect, the invention features an ¹⁸F-substituted torsemide, for example, a derivative of torsemide wherein an aryl group has been substituted with one or more ¹⁸F atoms, e.g., wherein a hydrogen or alkyl substituent of an aryl group has been replaced with ¹⁸F. In one embodiment, the ¹⁸F-substituted torsemide has the following formula:

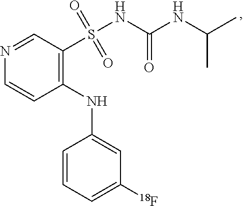

or a pharmaceutically acceptable salt thereof.

In one aspect, the invention features a method of making a torsemide, for example, a fluorinated torsemide with the following formula, using a method described herein:

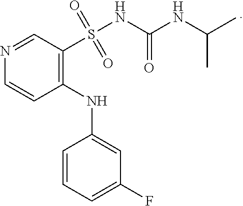

In one aspect, the invention features an ¹⁸F-substituted estradiol, for example, a derivative of estradiol wherein an aryl group has been substituted with one or more ¹⁸F atoms, e.g., wherein a hydrogen or hydroxy substituent of an aryl group has been replaced with ¹⁸F. In one embodiment, the ¹⁸F-substituted estradiol has the following formula:

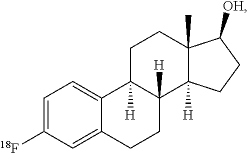

or a pharmaceutically acceptable salt thereof.

In one aspect, the invention features a method of making a fluorinated estradiol, for example, a fluorinated estradiol with the following formula, using a method described herein:

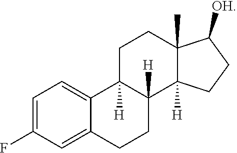

In one aspect, the invention features a method of making a fluorinated nicotine, for example, a fluorinated nicotine with the following formula, using a method described herein:

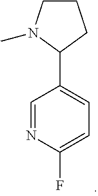

In one aspect, the invention features a fluorinated ezetimibe, for example, a derivative of ezetimibe wherein an aryl or heteroaryl group has been substituted with one or more fluorine atoms, e.g., wherein a hydrogen or hydroxyl substituent of an aryl or heteroaryl group has been replaced with a fluorine. In some embodiments, the fluorine substituent is ¹⁹F. In some embodiments, the fluorine substituent is ¹⁸F. In some embodiments, the fluorinated ezetimibe has the following formula:

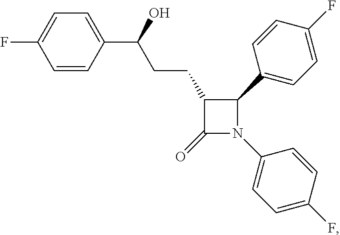

or a pharmaceutically acceptable salt thereof.

In one aspect, the invention features a method of making a fluorinated ezetimibe, for example, the fluorinated ezetimibe shown above, using a method described herein.

In one aspect, the invention features a fluorinated gemfibrozil, for example, a derivative of gemfibrozil wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated gemfibrozil has the following formula:

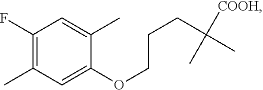

or a pharmaceutically acceptable salt thereof.

In one aspect, the invention features a method of making a fluorinated gemfibrozil, for example, the fluorinated gemfibrozil shown above, using a method described herein.

In one aspect, the invention features a fluorinated simfibrate, for example, a derivative of simfibrate wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated simfibrate is selected from one of the following:

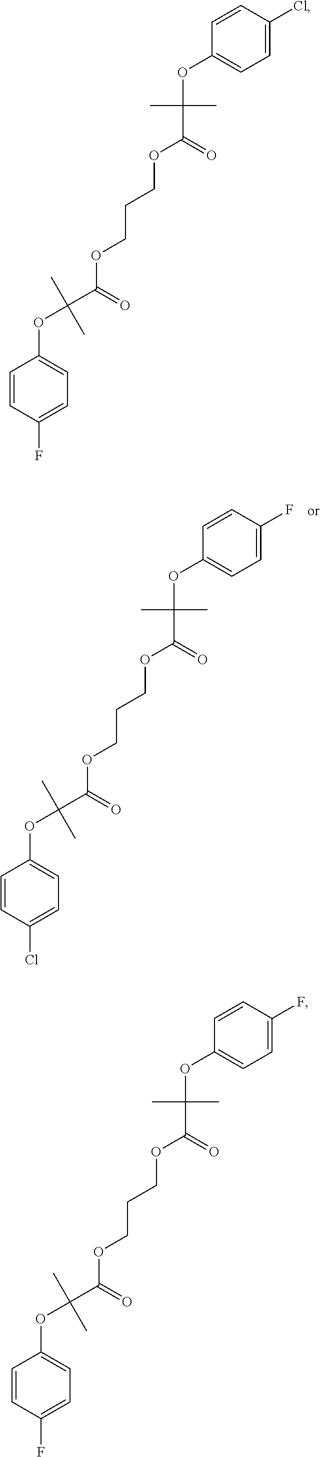

or a pharmaceutically acceptable salt thereof.

In one aspect, the invention features a method of making a fluorinated simfibrate, for example, a fluorinated simfibrate shown above, using a method described herein.

In one aspect, the invention features a fluorinated ronifibrate, for example, a derivative of ronifibrate wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated ronifibrate is selected from one of the following:

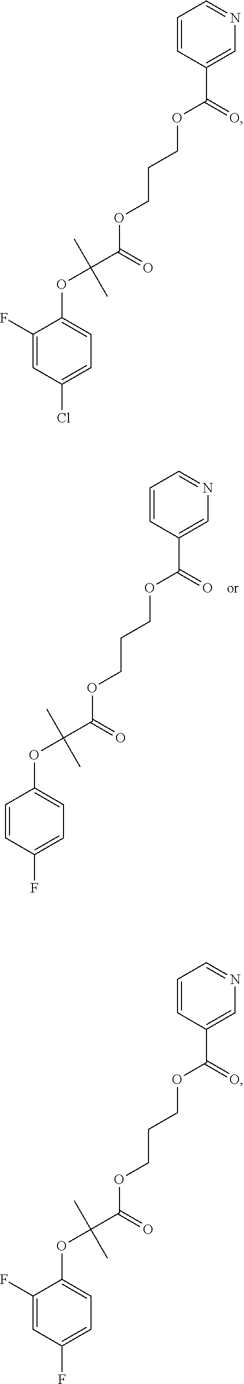

or a pharmaceutically acceptable salt thereof.

In one aspect, the invention features a method of making a fluorinated ronifibrate, for example, a fluorinated ronifibrate shown above, using a method described herein.

In one aspect, the invention features a fluorinated ciprofibrate, for example, a derivative of ciprofibrate wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated ciprofibrate has the following formula:

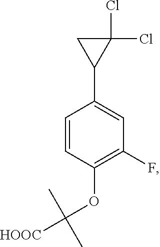

or a pharmaceutically acceptable salt thereof.

In one aspect, the invention features a method of making a fluorinated ciprofibrate, for example, the fluorinated ciprofibrate shown above, using a method described herein.

In one aspect, the invention features a fluorinated clofibride, for example, a derivative of clofibride wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated clofibride is selected from one of the following:

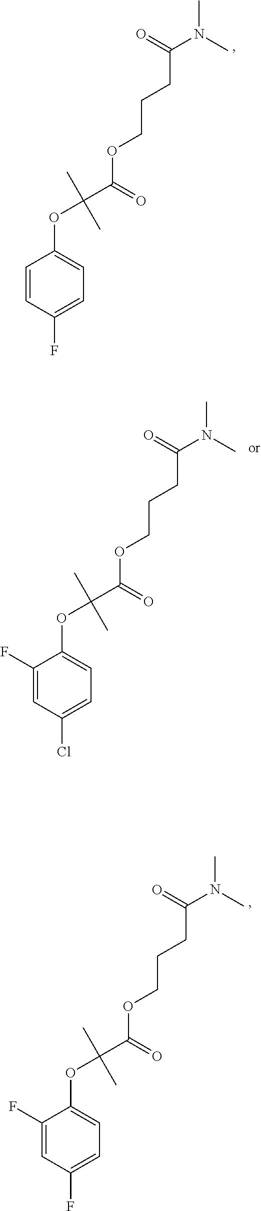

or a pharmaceutically acceptable salt thereof.

In one aspect, the invention features a method of making a fluorinated clofibride, for example, a fluorinated clofibride shown above, using a method described herein.

In one aspect, the invention features a fluorinated nicofuranose, for example, a derivative of nicofuranose wherein a heteroaryl group has been substituted with one or more fluorine atoms, e.g., wherein a hydrogen of a heteroaryl group has been replaced with a fluorine. In some embodiments, the fluorine substituent is ¹⁹F. In some embodiments, the fluorine substituent is ¹⁸F. In one embodiment, the fluorinated nicofuranose is selected from one of the following:

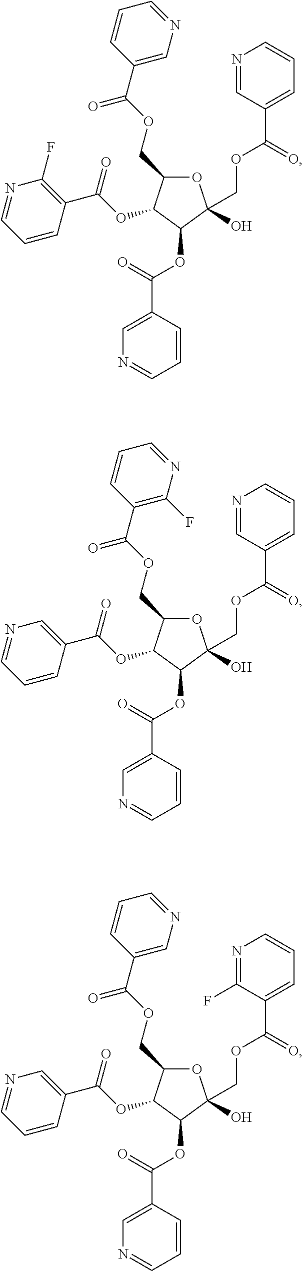

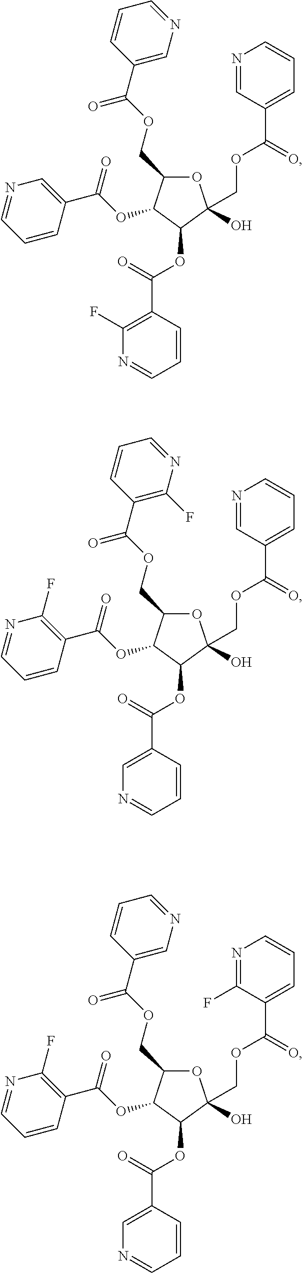

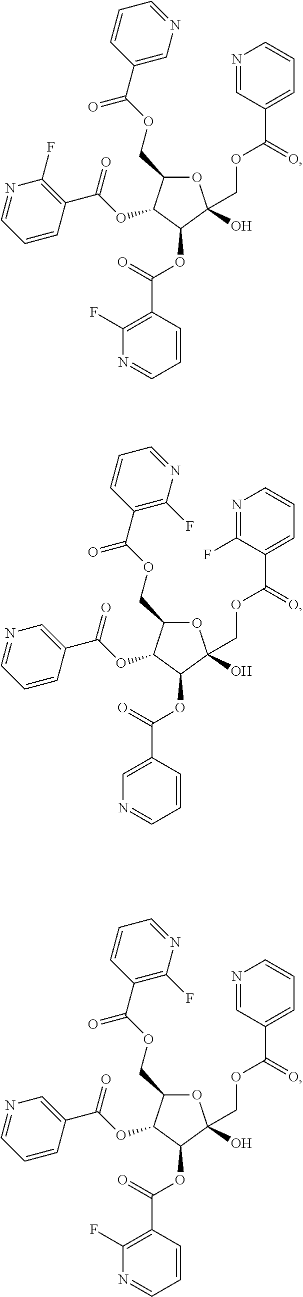

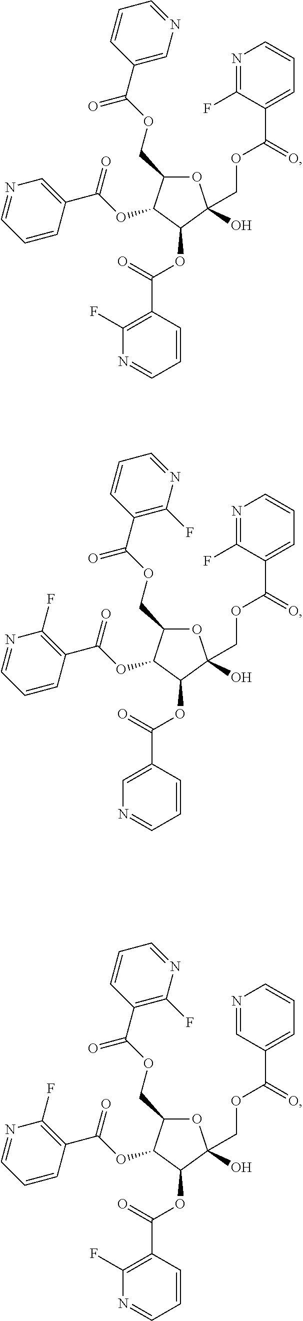

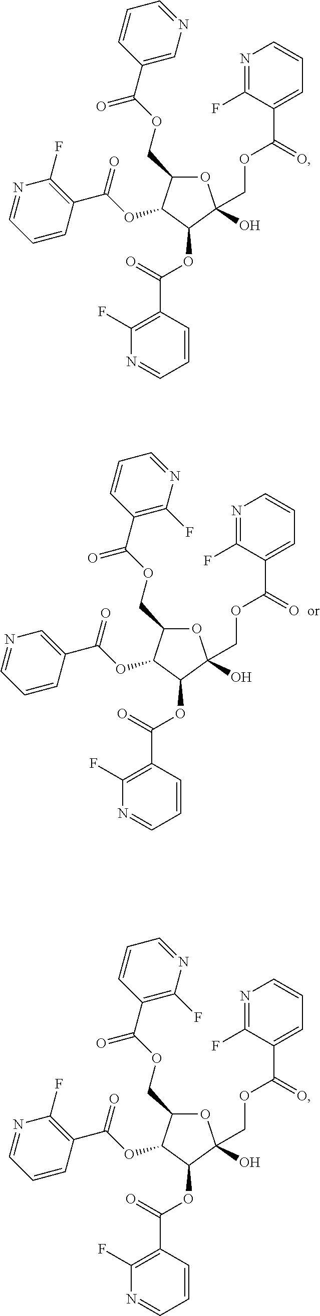

or a pharmaceutically acceptable salt thereof.

In one aspect, the invention features a method of making a fluorinated nicofuranose, for example, a fluorinated nicofuranose shown above, using a method described herein.

In one aspect, the invention features a fluorinated dextrothyroxine, for example, a derivative of dextrothyroxine wherein an aryl group has been substituted with one or more fluorine atoms, e.g., wherein a hydrogen, halogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated dextrothyroxine is selected from one of the following:

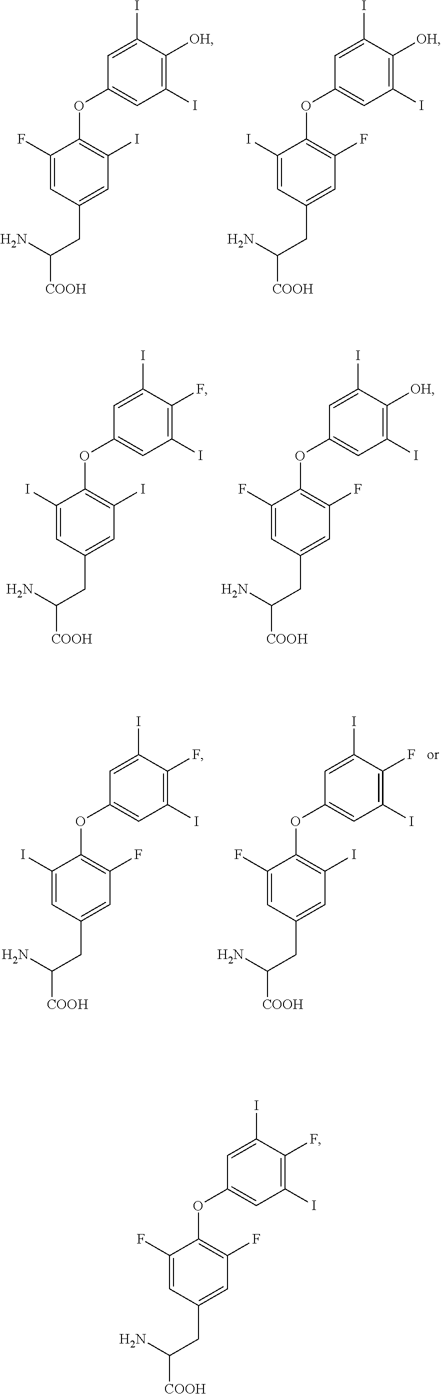

or a pharmaceutically acceptable salt thereof.

In one aspect, the invention features a method of making a fluorinated dextrothyroxine, for example, a fluorinated dextrothyroxine shown above, using a method described herein.

In one aspect, the invention features a fluorinated pyridoxal 5′-phosphate, for example, a derivative of pyridoxal 5′-phosphate wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated pyridoxal 5′-phosphate has the following formula:

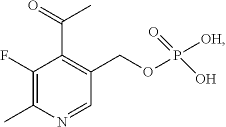

or a pharmaceutically acceptable salt thereof.

In one aspect, the invention features a method of making a fluorinated pyridoxal 5′-phosphate, for example, the fluorinated pyridoxal 5′-phosphate shown above, using a method described herein.

In one aspect, the invention features a fluorinated pioglitazone, for example, a derivative of pioglitazone wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated pioglitazone has the following formula:

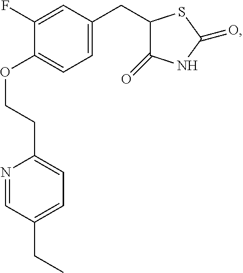

or a pharmaceutically acceptable salt thereof.

In one aspect, the invention features a method of making a fluorinated pioglitazone, for example, the fluorinated pioglitazone shown above, using a method described herein.

In one aspect, the invention features a fluorinated rosiglitazone, for example, a derivative of rosiglitazone wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated rosiglitazone has the following formula:

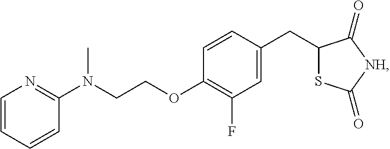

or a pharmaceutically acceptable salt thereof.

In one aspect, the invention features a method of making a fluorinated rosiglitazone, for example, the fluorinated rosiglitazone shown above, using a method described herein.

In one aspect, the invention features a fluorinated glipizide, for example, a derivative of glipizide wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated glipizide has the following formula:

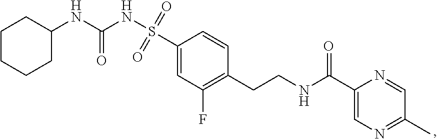

or a pharmaceutically acceptable salt thereof.

In one aspect, the invention features a method of making a fluorinated glipizide, for example, the fluorinated glipizide shown above, using a method described herein.

In one aspect, the invention features a fluorinated glimepiride, for example, a derivative of glimepiride wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated glimepiride has the following formula:

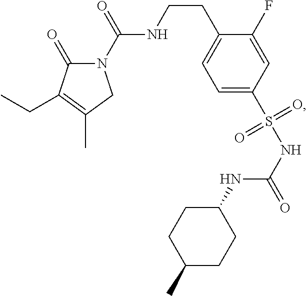

or a pharmaceutically acceptable salt thereof.

In one aspect, the invention features a method of making a fluorinated glimepiride, for example, the fluorinated glimepiride shown above, using a method described herein.

In one aspect, the invention features a fluorinated tetrahydrocannabinol, for example, a derivative of tetrahydrocannabinol wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated tetrahydrocannabinol has the following formula:

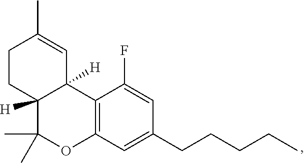

or a pharmaceutically acceptable salt thereof.

In one aspect, the invention features a method of making a fluorinated tetrahydrocannabinol, for example, the fluorinated tetrahydrocannabinol shown above, using a method described herein.

In one aspect, the invention features a fluorinated nabilone, for example, a derivative of nabilone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated nabilone has the following formula:

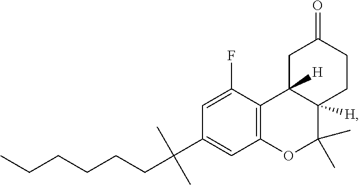

or a pharmaceutically acceptable salt thereof.

In one aspect, the invention features a method of making a fluorinated nabilone, for example, the fluorinated nabilone shown above, using a method described herein.

In one aspect, the invention features an ¹⁸F-substituted fenofibrate, for example, a derivative of fenofibrate wherein an aryl group has been substituted with one or more ¹⁸F atoms, e.g., wherein a hydrogen of an aryl group has been replaced with ¹⁸F. In one embodiment, the ¹⁸F-substituted fenofibrate has the following formula:

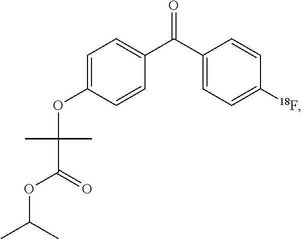

or a pharmaceutically acceptable salt thereof.

In one aspect, the invention features a method of making a fluorinated fenofibrate, for example, a fluorinated fenofibrate with the following formula, using a method described herein:

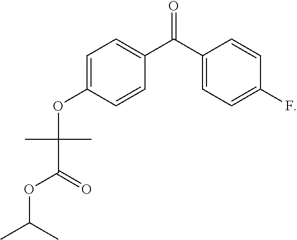

In one aspect, the invention features an ¹⁸F-substituted phentermine, for example, a derivative of phentermine wherein an aryl group has been substituted with one or more ¹⁸F atoms, e.g., wherein a hydrogen of an aryl group has been replaced with ¹⁸F. In one embodiment, the ¹⁸F-substituted phentermine has the following formula:

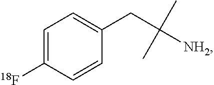

or a pharmaceutically acceptable salt thereof.

In one aspect, the invention features a method of making a fluorinated phentermine, for example, a fluorinated phentermine with the following formula, using a method described herein:

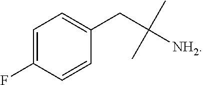

In one aspect, the invention features an ¹⁸F-substituted glyburide, for example, a derivative of glyburide wherein an aryl group has been substituted with one or more ¹⁸F atoms, e.g., wherein a hydrogen or halogen substituent of an aryl group has been replaced with ¹⁸F. In one embodiment, the ¹⁸F-substituted glyburide has the following formula:

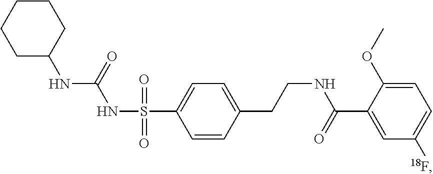

or a pharmaceutically acceptable salt thereof.

In one aspect, the invention features a method of making a fluorinated glyburide, for example, a fluorinated glyburide with the following formula, using a method described herein:

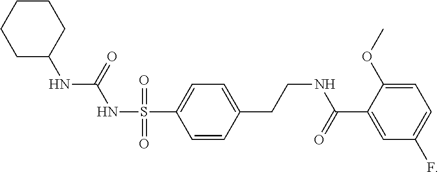

In one aspect, the invention features an ¹⁸F-substituted clofibrate, for example, a derivative of clofibrate wherein an aryl group has been substituted with one or more ¹⁸F atoms, e.g., wherein a hydrogen or halogen substituent of an aryl group has been replaced with ¹⁸F. In one embodiment, the ¹⁸F-substituted clofibrate has the following formula:

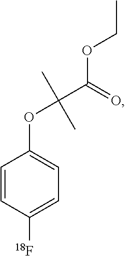

or a pharmaceutically acceptable salt thereof.

In one aspect, the invention features a method of making a fluorinated clofibrate, for example, a fluorinated clofibrate with the following formula, using a method described herein:

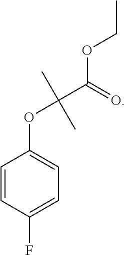

In one aspect, the invention features an ¹⁸F-substituted niacin, for example, a derivative of niacin wherein a heteroaryl group has been substituted with one or more ¹⁸F atoms, e.g., wherein a hydrogen of a heteroaryl group has been replaced with ¹⁸F. In one embodiment, the ¹⁸F-substituted niacin has the following formula:

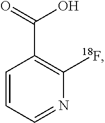

or a pharmaceutically acceptable salt thereof.

In one aspect, the invention features a method of making a fluorinated niacin, for example, a fluorinated niacin with the following formula, using a method described herein:

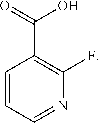

In one aspect, the invention features an ¹⁸F-substituted benfluorex, for example, a derivative of benfluorex wherein an aryl group has been substituted with one or more ¹⁸F atoms, e.g., wherein a hydrogen of an aryl group has been replaced with ¹⁸F. In one embodiment, the ¹⁸F-substituted benfluorex has the following formula:

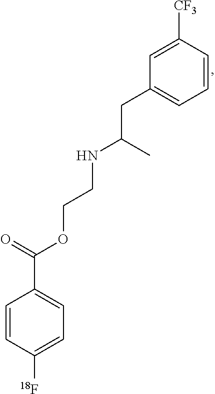

or a pharmaceutically acceptable salt thereof.

In one aspect, the invention features a method of making a fluorinated benfluorex, for example, a fluorinated benfluorex with the following formula, using a method described herein:

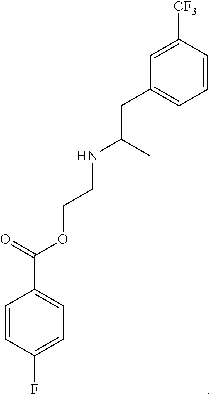

In one aspect, the invention features a fluorinated anastrozole, for example, a derivative of anastrozole wherein an aryl or heteroaryl group has been substituted with one or more fluorine atoms, e.g., wherein a hydrogen of an aryl or heteroaryl group has been replaced with a fluorine. In some embodiments, the fluorine substituent is ¹⁹F. In some embodiments, the fluorine substituent is ¹⁸F. In some embodiments, the fluorinated anastrozole has the following formula:

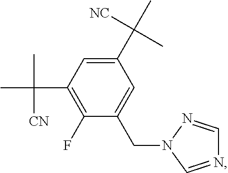

or a pharmaceutically acceptable salt thereof.

In one aspect, the invention features a method of making a fluorinated anastrozole, for example, the fluorinated anastrozole shown above, using a method described herein.

In one aspect, the invention features a fluorinated bicalutamide, for example, a derivative of bicalutamide wherein an aryl group has been substituted with one or more fluorine atoms, e.g., wherein a hydrogen or cyano substituent of an aryl group has been replaced with a fluorine. In some embodiments, the fluorine substituent is ¹⁹F. In some embodiments, the fluorine substituent is ¹⁸F. In one embodiment, the fluorinated bicalutamide has the following formula:

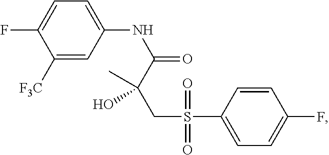

or a pharmaceutically acceptable salt thereof.

In one aspect, the invention features a method of making a fluorinated bicalutamide, for example, the fluorinated bicalutamide shown above, using a method described herein.

In one aspect, the invention features a fluorinated granisetron, for example, a derivative of granisetron wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated granisetron has the following formula:

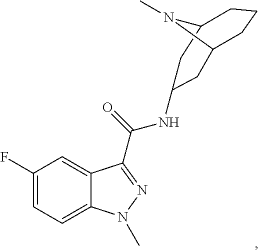

or a pharmaceutically acceptable salt thereof.

In one aspect, the invention features a method of making a fluorinated granisetron, for example, the fluorinated granisetron shown above, using a method described herein.

In one aspect, the invention features a fluorinated raloxifene, for example, a derivative of raloxifene wherein an aryl or heteroaryl group has been substituted with one or more fluorine atoms, e.g., wherein a hydrogen of an aryl or heteroaryl group has been replaced with a fluorine. In some embodiments, the fluorinated raloxifene does not have the following formula:

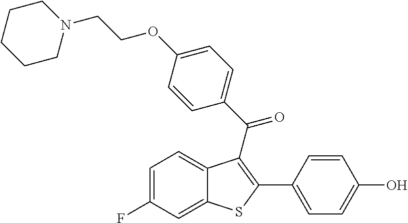

In some embodiments, the fluorine substituent is ¹⁹F. In some embodiments, the fluorine substituent is ¹⁸F. In some embodiments, the fluorinated raloxifene is selected from one of the following:

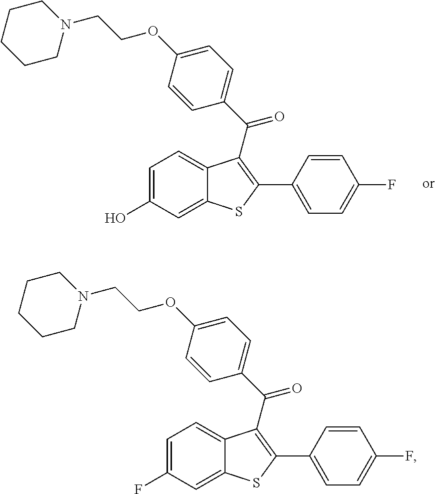

or a pharmaceutically acceptable salt thereof.

In one aspect, the invention features a method of making a fluorinated raloxifene, including any of the three fluorinated raloxifene structures shown above, using a method described herein.

In one aspect, the invention features an ¹⁸F-substituted raloxifene, for example, a derivative of raloxifene wherein an aryl or heteroaryl group has been substituted with one or more ¹⁸F atoms, e.g., wherein a hydrogen or hydroxy substituent of an aryl or heteroaryl group has been replaced with ¹⁸F. In one embodiment, the ¹⁸F-substituted raloxifene has the following formula:

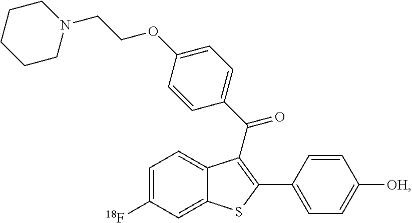

or a pharmaceutically acceptable salt thereof.

In one aspect, the invention features a fluorinated imatinib, for example, a derivative of imatinib wherein an aryl or heteroaryl group has been substituted with one or more fluorine atoms, e.g., wherein a hydrogen or alkyl substituent of an aryl or heteroaryl ring has been replaced with a fluorine. In some embodiments, the fluorinated imatinib does not have either of the following formulae:

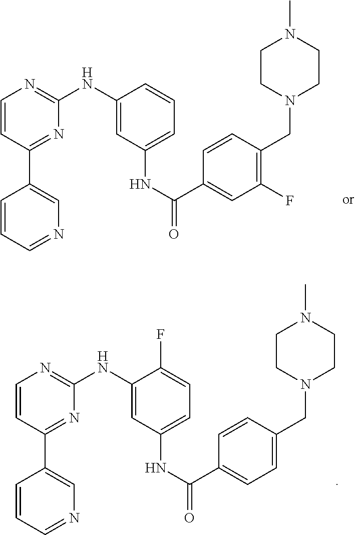

In some embodiments, each fluorine substituent is independently ¹⁹F. In some embodiments, each fluorine substituent is independently ¹⁸F. In some embodiments, the fluorinated imatinib has the following formula:

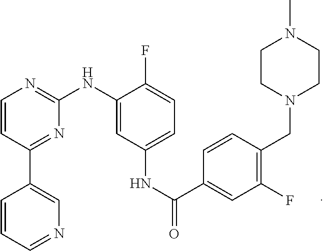

or a pharmaceutically acceptable salt thereof.

In one aspect, the invention features a method of making a fluorinated imatinib, including any of the three fluorinated imatinib structures shown above, using a method described herein.

In one aspect, the invention features an ¹⁸F-substituted imatinib, for example, a derivative of imatinib wherein an aryl group has been substituted with one or more ¹⁸F atoms, e.g., wherein a hydrogen or alkyl substituent of an aryl group has been replaced with ¹⁸F. In one embodiment, the ¹⁸F-substituted imatinib is selected from one of the following:

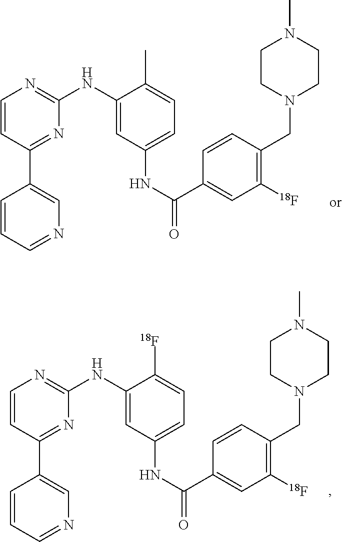

or a pharmaceutically acceptable salt thereof.

In one aspect, the invention features a fluorinated letrozole, for example, a derivative of letrozole wherein an aryl group has been substituted with one or more fluorine atoms, e.g., wherein a hydrogen or a cyano substituent of an aryl group has been replaced with a fluorine. In some embodiments, the fluorinated letrozole does not have the following formula:

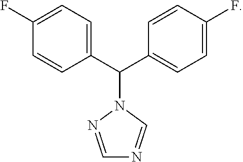

In some embodiments, the fluorine substituent is ¹⁹F. In some embodiments, the fluorine substituent is ¹⁸F. In some embodiments, the fluorinated letrozole has the following formula:

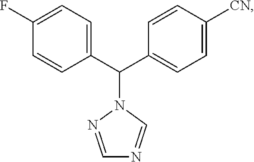

or a pharmaceutically acceptable salt thereof.

In one aspect, the invention features a method of making a fluorinated letrozole, including any of the two fluorinated letrozole structures shown above, using a method described herein.

In one aspect, the invention features an ¹⁸F-substituted letrozole, for example, a derivative of letrozole wherein an aryl group has been substituted with one or more ¹⁸F atoms, e.g., wherein a hydrogen or a cyano substituent of an aryl group has been replaced with ¹⁸F. In one embodiment, the ¹⁸F-substituted letrozole has the following formula:

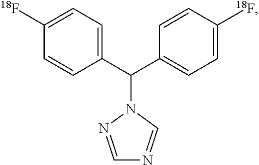

or a pharmaceutically acceptable salt thereof.

In one aspect, the invention features an ¹⁸F-substituted erlotinib, for example, a derivative of erlotinib wherein an aryl group has been substituted with one or more ¹⁸F atoms, e.g., wherein a hydrogen of an aryl group has been replaced with ¹⁸F. In one embodiment, the ¹⁸F-substituted erlotinib has the following formula:

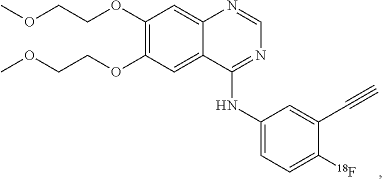

or a pharmaceutically acceptable salt thereof.

In one aspect, the invention features a method of making a fluorinated erlotinib, for example, a fluorinated erlotinib with the following formula, using a method described herein:

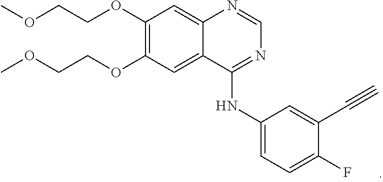

In one aspect, the invention features an ¹⁸F-substituted thalidomide, for example, a derivative of thalidomide wherein an aryl or heteroaryl group has been substituted with one or more ¹⁸F atoms, e.g., wherein a hydrogen of an aryl or heteroaryl group has been replaced with ¹⁸F. In one embodiment, the ¹⁸F-substituted thalidomide has the following formula:

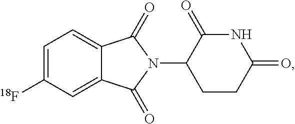

or a pharmaceutically acceptable salt thereof.

In one aspect, the invention features a method of making a fluorinated thalidomide, for example, a fluorinated thalidomide with the following formula, using a method described herein:

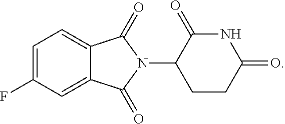

In one aspect, the invention features a fluorinated desmethyltamoxifen, for example, a derivative of desmethyltamoxifen wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In some embodiments, the fluorinated desmethyltamoxifen is selected from one of the following:

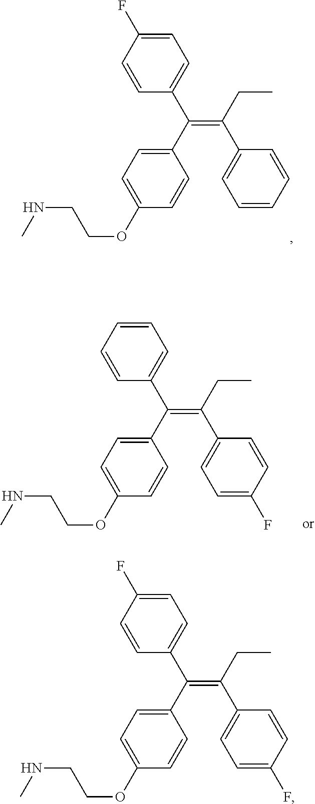

or a pharmaceutically acceptable salt thereof.

In one aspect, the invention features a method of making a fluorinated desmethyltamoxifen, for example, a fluorinated desmethyltamoxifen shown above, using a method described herein.

In one aspect, the invention features a fluorinated tamoxifen, for example, a derivative of tamoxifen wherein an aryl group has been substituted with one or more fluorine atoms, e.g., wherein a hydrogen of an aryl group has been replaced with a fluorine. In some embodiments, the fluorinated tamoxifen does not have either of the following formulae:

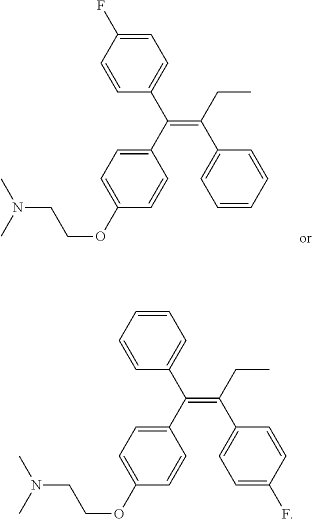

In some embodiments, the fluorine substituent is ¹⁹F. In some embodiments, the fluorine substituent is ¹⁸F. In some embodiments, the fluorinated tamoxifen has the following formula:

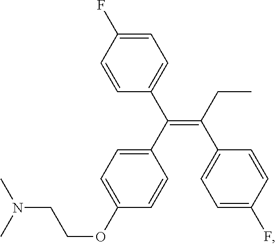

or a pharmaceutically acceptable salt thereof.

In one aspect, the invention features a method of making a fluorinated tamoxifen, including any of the three fluorinated tamoxifen structures shown above, using a method described herein.

In one aspect, the invention features an ¹⁸F-substituted tamoxifen, for example, a derivative of tamoxifen wherein an aryl group has been substituted with one or more ¹⁸F atoms, e.g., wherein a hydrogen of an aryl group has been replaced with ¹⁸F. In one embodiment, the ¹⁸F-substituted tamoxifen is selected from one of the following:

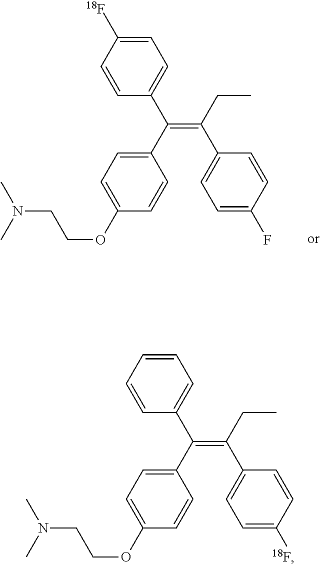

or a pharmaceutically acceptable salt thereof.

In one aspect, the invention features an ¹⁸F-substituted tropisetron, for example, a derivative of tropisetron wherein an aryl group has been substituted with one or more ¹⁸F atoms, e.g., wherein a hydrogen of an aryl group has been replaced with ¹⁸F. In one embodiment, the ¹⁸F-substituted tropisetron has the following formula:

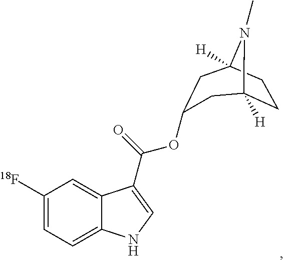

or a pharmaceutically acceptable salt thereof.

In one aspect, the invention features a method of making a fluorinated tropisetron, for example, a fluorinated tropisetron with the following formula, using a method described herein:

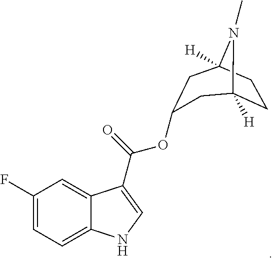

In one aspect, the invention features a composition comprising a compound described herein (e.g., a pharmaceutical composition comprising a compound described herein).

In one aspect, the invention features a kit comprising a compound or composition described herein.

In some embodiments, a compound described herein can be administered to a subject to treat a disorder described herein, e.g., a disorder that can be treated with an opioid analgesic, or an opioid dependence disorder.

In some embodiments, a compound described herein (e.g., a fluorinated derivative of a pharmaceutical agent) has one or more properties that are superior to a corresponding unfluorinated derivatives of that pharmaceutical agent (e.g., where the corresponding unfluorinated derivative is either without a fluorine in the structure or does not include the same fluorine substitution pattern as the fluorinated derivative described herein). In some embodiments, the improved property is improved metabolic stability, improved penetration across the blood brain barrier, reduced penetration across the blood brain barrier, or improved solubility.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl).

The term, “cyano” refers to a —CN radical.

The terms “alkylamino” and “dialkylamino” refer to —NH(alkyl) and —NH(alkyl)₂ radicals respectively. The term “hydroxy” refers to an OH radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

### DETAILED DESCRIPTION

### Compounds

Described herein are fluorinated compounds, e.g., fluorinated derivatives of a pharmaceutical agent. In some embodiments, the compound includes one or more fluorine moieties on an aryl or heteroaryl ring within the pharmaceutical agent.

In some embodiments the compound is a fluorinated derivative of an antibiotic, antiviral or antifungal agent described herein (e.g., a beta-lactam, a protease inhibitor or a triazole derivative). Exemplary compounds include the following:

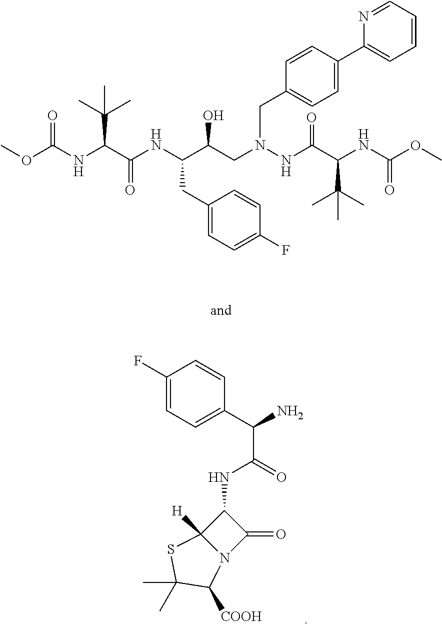

In some embodiments the compound is a fluorinated derivative of an antihypertensive agent, including those described herein (e.g., an angiotensin-converting enzyme (ACE) inhibitor, a beta blocker or a calcium channel blocker). Exemplary compounds include the following:

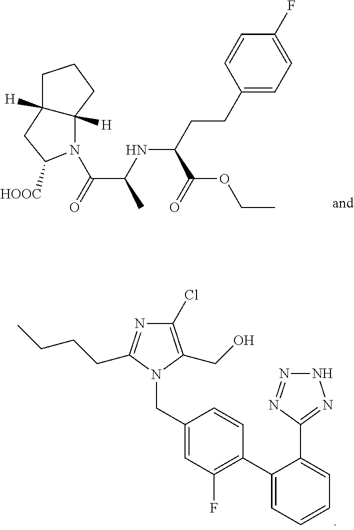

In some embodiments the compound is a fluorinated derivative of an anti-inflammatory, antihistamine or antimigraine agent (e.g., a non-steroidal anti-inflammatory drug (NSAID), an H₁-receptor antagonist or an ergot alkaloid). Exemplary compounds include the following:

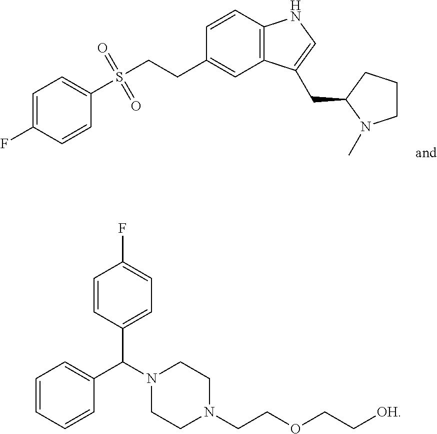

In some embodiments the compound is a fluorinated derivative of a cardiovascular agent described herein (e.g., a beta blocker, calcium channel blocker, antiplatelet agent, anticoagulant or vasodilator). Exemplary compounds include:

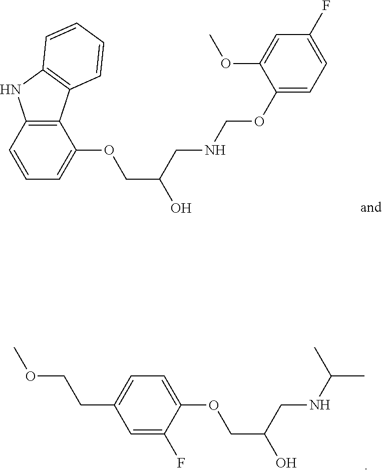

In some embodiments the compound is a fluorinated derivative of an antidepressant (e.g., a tricyclic antidepressant, a monoamine oxidase inhibitor (MAOI) or a selective serotonin reuptake inhibitor (SSRI)). Exemplary compounds include the following:

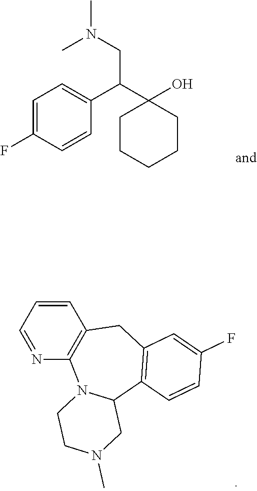

In some embodiments the compound is a fluorinated derivative of an agent for treating a movement disorder, such as a movement disorder described herein (e.g., an antiepileptic agent, an antimuscarinic agent or a dopaminergic agent). Exemplary compounds include the following:

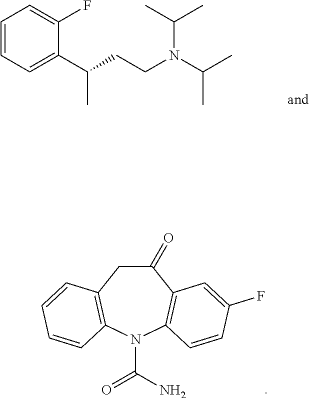

In some embodiments the compound is a fluorinated derivative of an antipsychotic agent (e.g., a phenazine, a thioxanthene, or an atypical antipsychotic agent). Exemplary compounds include the following:

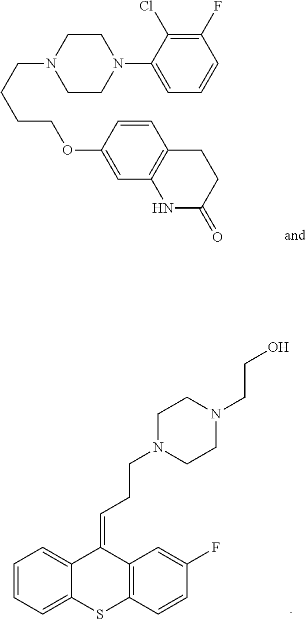

In some embodiments the compound is a fluorinated derivative of an agent used to treat pain as described herein (e.g., a muscle relaxant, an anesthetic or an analgesic). Exemplary compounds include the following:

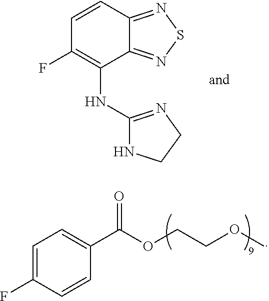

In some embodiments the compound is a fluorinated derivative of a pharmaceutical agent. Exemplary compounds include the following:

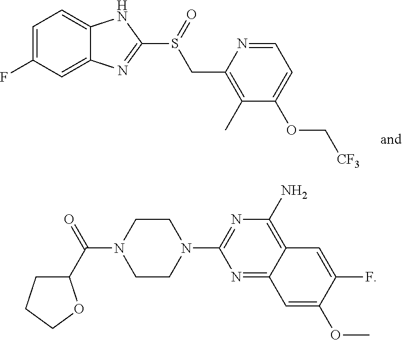

In some embodiments the compound is a fluorinated derivative of an agent for treating metabolic disorders described herein (e.g., hypolipidemic agents, cholesterol absorption inhibitors, thiazolidinediones and lipid modifying agents). Exemplary compounds include the following:

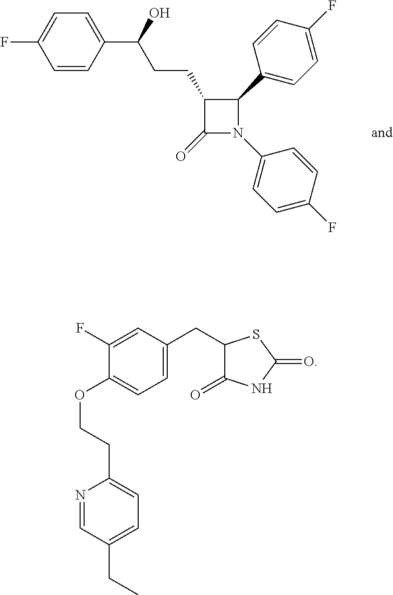

In some embodiments the compound is a fluorinated derivative of an agent for treating cancer or a side-effect of cancer (e.g., aromatase inhibitors, 5-HT₃ antagonists, and tyrosine kinase inhibitors). Exemplary compounds include the following:

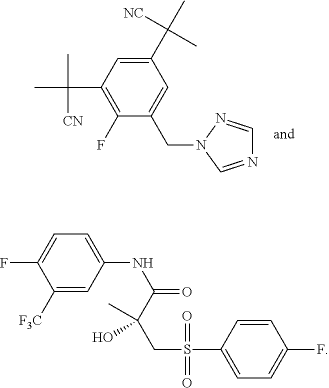

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention.

The compounds of this invention may also be represented in multiple tautomeric forms. In such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60). Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Synthetic Methods

Described herein are methods of making a fluorine-containing compound (e.g., a compound described herein). The compounds described herein can be synthesized via a variety of methods, included Ag or Pd mediated methods. In general, the methods include an organic compound to be fluorinated, a fluorinating agent, and either a silver salt or a palladium complex.

Compounds to be Fluorinated

Exemplary compounds such as a pharmaceutical agent or a precursor thereof or a derivative thereof, include those described herein. The compound may be a small organic molecule or a large organic molecule. A small organic molecule includes any molecule having a molecular weight of less than 1000 g/mol, of less than 900 g/mol, of less than 800 g/mol, of less than 700 g/mol, of less than 600 g/mol, of less than 500 g/mol, of less than 400 g/mol, of less than 300 g/mol, of less than 200 g/mol or of less than 100 g/mol. A large organic molecule include any molecule of between 1000 g/mol to 5000 g/mol, of between 1000 g/mol to 4000 g/mol, of between 1000 g/mol to 3000 g/mol, of between 1000 g/mol to 2000 g/mol, or of between 1000 g/mol to 1500 g/mol. Organic compounds include aryl compounds, heteroaryl compounds, carbocyclic compounds, heterocyclic compounds, aliphatic compounds, heteroaliphatic compounds. In a preferred embodiment, the organic compound is an aryl compound (e.g., a phenyl compound), or a heteroaryl compound (e.g. a quinolyl or indolyl compound).

In some embodiments, the compound contains a chiral center. In some embodiments, the compound is further substituted with one or more functional groups (e.g., alcohols, aldehydes, ketones, alkenes, alkoxy groups, cyano groups, amides and N-oxides). In some embodiments, the functional groups are unprotected. In some embodiments, the compound is a precursor of a pharmaceutically acceptable compound.

Fluorinating Agents

As generally described above, the process utilizes a fluorinating agent. In some embodiments, the fluorinating agent is an electrophilic fluorinating agent. In some embodiments, the fluorinating agent is commercially available. In some embodiments, the electrophilic fluorinating agent is also an inorganic fluorinating agent. Exemplary electrophilic fluorinating agents include, but are not limited to, N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, N-fluoropyridinium triflate, an N-fluoroarylsulfonimide (e.g., N-fluorobenzenesulfonimide), N-chloromethyl-N′-fluorotriethylenediammonium bis(tetrafluoroborate))(Selectfluor®, N-chloromethyl-N′-fluorotriethylenediammonium bis(hexafluorophosphate), N-chloromethyl-N′-fluorotriethylenediammonium bis(triflate) and XeF₂. In some embodiments, the fluorinating agent is Selectfluor®. In some embodiments, the fluorinating agent is N-fluoropyridinium triflate. In some embodiments, the fluorinating agent is N-fluoro-2,4,6-trimethylpyridinium triflate. In some embodiments, the fluorinating agent is N-fluoro-2,4,6-trimethylpyridinium tetrafluoroborate. In some embodiments, the fluorinating agent is N-fluoro-benzenesulfonimide. In some embodiments, the fluorinating agent is xenon difluoride.

The fluorinating agent may be enriched with a particular isotope of fluorine. In some embodiments, the fluorinating agent is labeled with ¹⁹F (i.e., transfers an ¹⁹F fluorine substituent to the organic compound). In some embodiments, reaction of the ¹⁹F fluorinating agent in the inventive process provides a fluorinated ¹⁹F-labeled organic compound.

In some embodiments, the fluorinating agent is labeled with ¹⁸F (i.e., transfers an ¹⁸F fluorine substituent to the organic compound). In some embodiments, reaction of the ¹⁸F fluorinating agent in the inventive process provides a fluorinated ¹⁸F-labeled organic compound.

However, in some embodiments, the fluorinating agent is labeled with a mixture of ¹⁸F and ¹⁹F. In some embodiments, reaction of the mixture of ¹⁹F and ¹⁸F fluorinating agent in the inventive process provides a mixture of fluorinated ¹⁹F-labeled organic compound and fluorinated ¹⁸F-labeled organic compound.

Any of the above fluorinated agents may be labeled as ¹⁹F or ¹⁸F.

For example, in some embodiments, the fluorinating agent is ¹⁹F-labeled N-(chloromethyl)-N′-fluorotriethylenediamine bis(tetrafluoroborate) (Selectfluor®) or ¹⁹F-labeled XeF₂. In some embodiments, the fluorinating agent is ¹⁹F-labeled N-(chloromethyl)-N′-fluorotriethylenediamine bis(tetrafluoroborate))(Selectfluor®. In some embodiments, the fluorinating agent is ¹⁹F-labeled XeF₂.

In some embodiments, the fluorinating agent is ¹⁸F-labeled N-(chloromethyl)-N′-fluorotriethylenediamine bis(tetrafluoroborate) (Selectfluor®) or ¹⁸F-labeled XeF₂. In some embodiments, the fluorinating agent is ¹⁸F-labeled N-(chloromethyl)-N′-fluorotriethylenediamine bis(tetrafluoroborate))(Selectfluor®. In some embodiments, the fluorinating agent is ¹⁸F-labeled XeF₂.

Exemplary methods include the following.

### Ag(I)-Mediated Fluorination

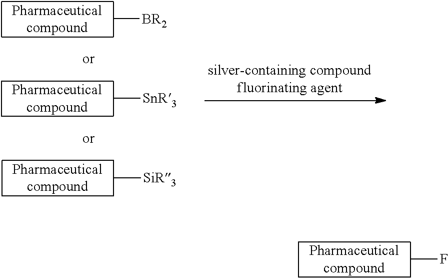

Upon reaction of an organic compound comprising an organostannane, a boron substituent or a silane substituent, with a silver-containing compound and a fluorinating agent, the method provides a fluorinated organic compound in which the organostannane, boron substituent or silane substituent is replaced with a fluorine substituent. In some embodiments, the organostannane, boron substituent or silane substituent is attached to an aryl or heteroaryl moiety of the organic compound. For examples, see Schemes 1-6.

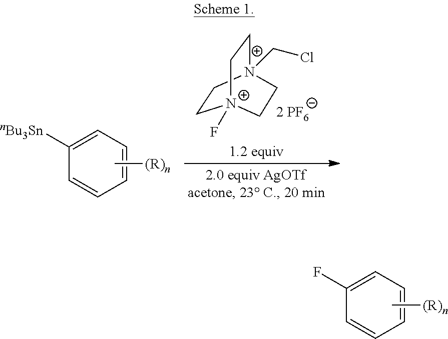

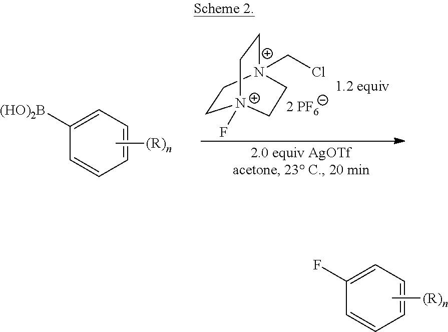

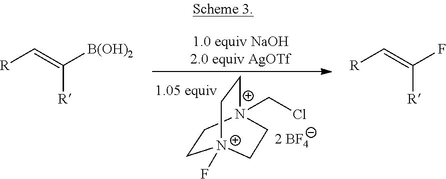

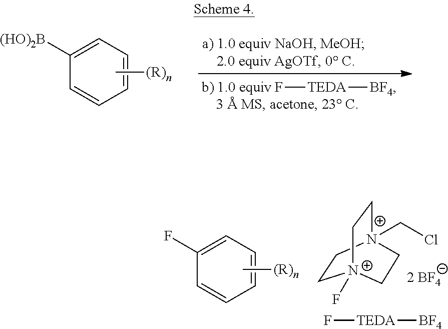

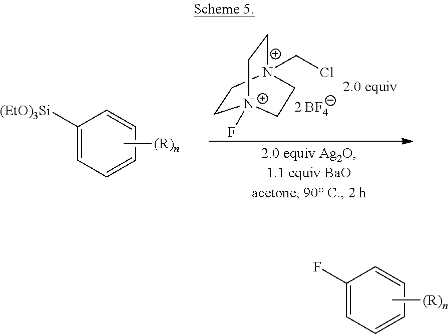

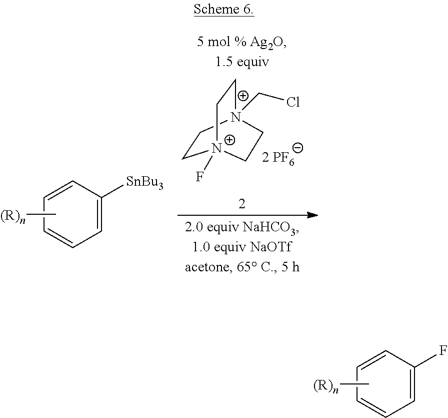

In the above Schemes 1-6, R and R′ are substituents and n may be 0, 1, 2, 3, 4 or 5. Exemplary substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkylamino, dialkylamino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙaryl (where n is 0-2), S(O)ₙheteroaryl (where n is 0-2), S(O)ₙheterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). The substituents are independently any one single, or any subset of the aforementioned substituents. A substituent may itself be substituted with any one of the above substituents. In some embodiments, two R groups may be taken together to form a ring, e.g., an aryl, heteroaryl, cyclyl or heterocyclyl ring, which may itself be further substituted with any one of the above substituents.

In some embodiments, the method uses a catalytic amount of silver. Exemplary methods of fluorinating a compound using Ag are described in P.C.T. Application No. PCT/US2009/065339, filed Nov. 20, 2009, which is incorporated herein by reference in its entirety.

Boron Substituents

Methods of fluorinating an organic compound are described herein. In some embodiments, the organic compound comprises a boron substituent. The boron substituent may be of the formula:

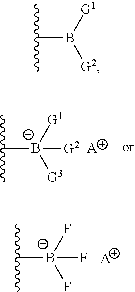

wherein G¹, G² and G³ are, independently, —OH, —OR, or —R, wherein each R is, independently, optionally substituted aliphatic, optionally substituted heteroaliphatic, optionally substituted aryl, or optionally substituted heteroaryl, or G¹ and G² are joined to form an optionally substituted 5- to 8-membered ring having at least one O atom directly attached to B, wherein the ring is comprised of carbon atoms and optionally one or more additional heteroatoms independently selected from the group consisting of N, S, and O. A⁺ may be a metal cation or ammonium.

As used herein, a boron substituent is intended to encompass free boronic acid substituents (i.e., wherein G¹ and G² are both —OH) and oligomeric anhydrides thereof (including dimers, trimers, and tetramers, and mixtures thereof), boronic ester substituents (i.e., wherein G¹ is —OH or —OR and G² is —OR), borinic acid substituents (i.e., wherein G¹ is —OH and G² is —R), borinic ester substituents (i.e., wherein G¹ is —OR and G² is —R), trihydroxoborates (i.e., wherein G¹, G² and G³ are all —OH), and trialkoxyborates (i.e., wherein G¹, G² and G³ are all —OR, e.g., —OCH₃).

In some embodiments, G¹ and G² are joined to form a 5-membered ring. Exemplary 5-membered rings include:

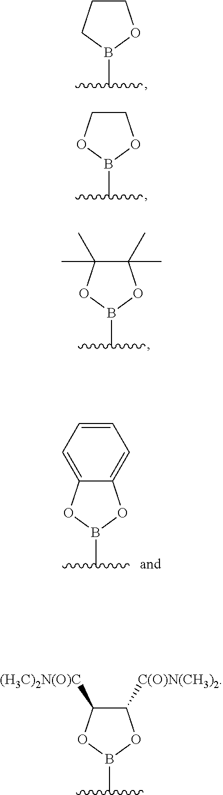

In some embodiments, G¹ and G² are joined to form a 6-membered ring. Exemplary 6-membered rings include:

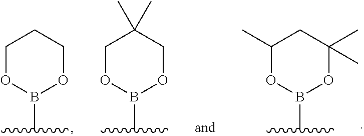

In some embodiments, G¹ and G² are joined to form an 8-membered ring. Exemplary 8-membered rings include:

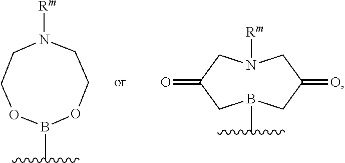

wherein R^{m} is hydrogen, a suitable amino protecting group, or an optionally substituted aliphatic, optionally substituted heteroaliphatic, optionally substituted aryl, or optionally substituted heteroaryl group.

Furthermore, as used herein, a boron substituent is also intended to encompass a trifluoroborate substituent. For example, in some embodiments, a boron substituent is a group of the formula:

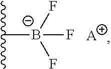

wherein A^{α} is a metal cation or ammonium.

Furthermore, as used herein, a boron substituent is also intended to encompass trihydroxy- and trialkoxy borates. For example, in some embodiments, a boron substituent is a group of the formulae:

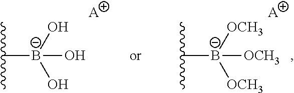

wherein A^{α} is a metal cation or ammonium.

Exemplary metal cations include lithium, sodium, potassium, magnesium, and calcium cations. In some embodiments, the metal cation is a potassium cation.

An organic compound comprising a boron substituent may be obtained via a variety of known methods. For example, a halogen-containing precursor may be reacted with a boron-containing compound to generate the organic compound comprising a boron substituent. An unactivated C—H bond may also be borylated, for example, using a suitable catalyst.

Silane Substituents

Methods of fluorinating an organic compound are described herein. In some embodiments, the organic compound comprises a silane substituent. The silane substituent may be a trialkoxysilane, e.g., trimethoxysilane or triethoxysilane. The silane substituent may be a trihydroxysilane.

An organic compound comprising a silane substituent may be obtained via a variety of known methods. For example, a Grignard-containing precursor may be reacted with a silicon-containing compound (e.g., a tetraalkoxysilane) to generate the organic compound comprising a silane substituent. In another example, a halogen-containing precursor or a triflyl-containing precursor may be reacted with a silicon-containing compound (e.g., a tetraalkoxysilane) in the presence of a suitable catalyst (e.g., a Pd⁰ or Rh¹ catalyst) to generate the organic compound comprising a silane substituent.

Organostannanes

Methods of fluorinating an organic compound are described herein. In some embodiments, the organic compound comprises an organostannane. The organostannane may be a trialkylstannane, e.g., trimethylstannane or tributylstannane.

Silver-Containing Compounds

The Ag methods described herein generally include a silver-containing compound. The silver-containing compound may be a silver complex or a silver salt, e.g., a silver(I) salt. Exemplary silver salts include silver(I) salts such as silver(I) fluoride, silver(I) acetate, silver(I) tetrafluoroborate, silver(I) perchlorate, silver(I) nitrate, silver(I) carbonate, silver(I) cyanide, silver(I) benzoate, silver(I) triflate, silver(I) hexafluorophosphate, silver(I) hexafluoroantimonate, silver(I) oxide, silver(I) nitrite and silver(I) phosphate. In preferred embodiments, the silver salt is silver(I) triflate or silver(I) oxide.

### Pd(II)-Mediated Fluorination

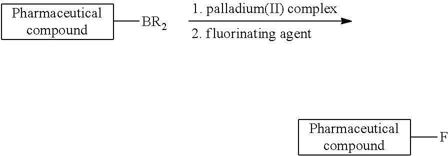

Upon reaction of an organic compound comprising a boron substituent with a palladium(II) complex and a fluorinating agent, the method provides a fluorinated organic compound in which the boron substituent is replaced with a fluorine substituent. In some embodiments, the boron substituent is attached to an aryl or heteroaryl moiety of the organic compound. For example, see Scheme 7.

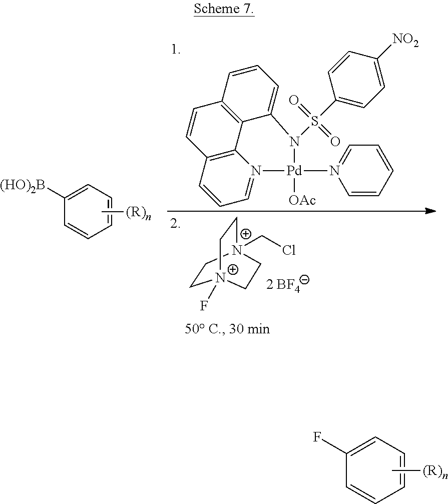

Exemplary methods of fluorinating a compound using a Pd(II) complex are described in WO2009/100014, which is incorporated herein by reference in its entirety.

### Palladium (II) Complexes

In some embodiments, a stoichiometric amount of the palladium (II) complex is used.

In some embodiments, the palladium (II) complex comprises a bidentate ligand. In some embodiments, the palladium (II) complex comprises a tridentate ligand.

In some embodiments, the palladium (II) complex is crystalline. Alternatively, in some embodiments, the palladium (II) complex is amorphous.

In certain embodiments, the palladium(II) complex is not a salt. Alternatively, in certain embodiments, the palladium(II) complex is a salt. For example, in certain embodiments, the palladium(II) complex is a salt of tetrafluoroborate (BF₄⁻), tetraphenylborate (BPh4⁻), hexafluorophosphate (PF6⁻), tetrakis[3,5-bis(trifluoromethyl)phenyl]borate ([BArF₄]⁻), tetrakis(pentafluorophenyl)borate (B(C₆F₅)₄⁻), antimohexafluoride (SbF₆⁻), or trifluoromethansulfonate (triflate, CF₃SO₃⁻). In certain embodiments, the palladium(II) complex is a salt of tetrafluoroborate (BF₄⁻).

In some embodiments, the palladium (II) complex is a palladium (II) dimer complex.

In some embodiments, the palladium (II) complex is generated in situ from a complex in the 0 oxidation state (i.e., a “palladium (0) complex”) and one or more ligands.

Exemplary ligands include, but are not limited to, halogens (e.g., iodide, bromide, chloride, fluoride), solvents (e.g., hydroxide, water, ammonia, acetonitrile, dimethylsulfoxide, dimethylformamide, dimethylacetamide), sulfide, cyanide, carbon monoxide, thiocyanate, isothiocyanate, nitrate, nitrite, azide, oxalate, olefins (e.g., dibenzylidineacetone (dba)), optionally substituted pyridines (py) (e.g., 2,2′,5′,2-terpyridine (terpy), bipyridine (bipy) and other pyridine ligands as described herein), optionally substituted aryl (e.g., phenyl (Ph), phenanthroline (phen), biphenyl), phosphines (e.g., triphenylphosphine (PPh₃), 1,2-bis(diphenylphosphino)ethane (dppe), tricyclohexylphosphine (PCy₃), tri(o-tolyl)phosphine (P(o-tol)₃), tris(2-diphenylphosphineethyl)amine (np3)), amino ligands (e.g., ethylenediamine (en), diethylenetriamine (dien), tris(2-aminoethyl)amine (tren), triethylenetetramine (trien), ethylenediaminetetraacetate (EDTA)), acyloxy ligands (e.g., acetylaceonate (acac), O-acetate (—OAc)), and alkyloxy ligands (e.g., —OMe, OiPr, OtBu).

As one of ordinary skill in the art would understand, the ligands are chosen to satisfy the valency of palladium. Thus, in some embodiments, the ligands are chosen to satisfy the valency of a palladium complex as +2.

Exemplary palladium (II) complexes include, but are not limited to, palladium (II) bromide, palladium (II) chloride, palladium (II) iodide, palladium (II) fluoride, palladium (II) acetate, palladium (II) acetylacetonate, palladium (II) oxide, palladium (II) cyanide, palladium (II) sulfide, palladium (II) sulfate, palladium (II) 2,4-pentanedionate, allyl palladium (II) chloride dimer, bis(acetonitrile)dichloropalladium (II), trans-bis(benzonitrile)dichloropalladium (II), and trichloro-bis(triphenylphosphine)palladium (II).

Exemplary palladium (0) complexes include, but are not limited to, Pd₂ dba₃, Pd₂ dba₃-CHCl₃, and tetrakis(triphenylphosphine)palladium (0).

Other exemplary ligands are provided as groups R^{L1} and R^{L2}, described below and herein. Furthermore, other exemplary bidentate and tridentate palladium (II) complexes are provided in the following formulae, described below and herein.

For example, in some embodiments, the palladium (II) complex comprises a bidentate or tridentate ligand to provide a complex of the formula (I):

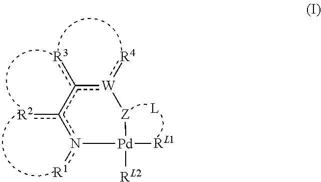

wherein:

Pd represents palladium of valency of +2;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group;

when W is —C— or —C(R^{d})— then:
(i) Z is a bond, —O—, —S—, C(R^{d})₂—, —C(R^{d})═C(R^{d})—, C(R^{d})═N—, or —N(R^{e})—;

or
(ii) Z is —N— joined via a linker group -L- to the group R^{L1} to form a 5- to 7-membered palladacycle, wherein -L- is selected from absent, —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring;

or
(iii) Z is —N—S(O)₂—R^{e3} and the linker group -L- is absent;

or

when W is —N— or —N(R^{e})—, then Z is a bond, —C(R^{d})₂—, —C(R^{d})═C(R^{d})—, or —C(R^{d})═N—;

or

when W is —SO₂— or ═N—, then R₄ is absent;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein the each of curved dotted lines [symbol] independently represents optional joining of an optionally substituted 5- to 7-membered ring, and

wherein [symbol] represents a single or double bond.

In some embodiments, R¹ and R² are joined to form an optionally substituted 5- to 6-membered heteroaryl, aryl, heterocyclic or carbocyclic ring. In some embodiments, R¹ and R² are joined to form an optionally substituted 5-membered heteroaryl, aryl, heterocyclic or carbocyclic ring. In some embodiments, R¹ and R² are joined to form an optionally substituted 6-membered heteroaryl, aryl, heterocyclic or carbocyclic ring.

In some embodiments, R² and R³ are joined to form an optionally substituted 5- to 6-membered heteroaryl, aryl, heterocyclic or carbocyclic ring. In some embodiments, R² and R³ are joined to form an optionally substituted 5-membered heteroaryl, aryl, heterocyclic or carbocyclic ring. In some embodiments, R² and R³ are joined to form an optionally substituted 6-membered heteroaryl, aryl, heterocyclic or carbocyclic ring.

In some embodiments, R³ and R⁴ are joined to form an optionally substituted 5- to 6-membered heteroaryl, aryl, heterocyclic or carbocyclic ring. In some embodiments, R³ and R⁴ are joined to form an optionally substituted 5-membered heteroaryl, aryl, heterocyclic or carbocyclic ring. In some embodiments, R³ and R⁴ are joined to form an optionally substituted 6-membered heteroaryl, aryl, heterocyclic or carbocyclic ring.

Any of the optionally substituted 5- to 6-membered heteroaryl, aryl, heterocyclic or carbocyclic rings formed by joining R¹ and R², R² and R³ and/or R³ and R⁴ can be, for example, an optionally substituted 5- to 6-membered heteroaryl, an optionally substituted 6-membered aryl, an optionally substituted 5- to 6-membered heterocyclic or an optionally substituted 5- to 6-membered carbocyclic ring.

Exemplary 5-membered heteroaryl rings include, but are not limited to, optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

Exemplary 5-membered heterocyclic rings include, but are not limited to, optionally substituted pyrrolidinyl, optionally substituted tetrahydrofuranyl, optionally substituted tetrahydrothiophenyl, and optionally substituted 1,3 dithiolanyl.

Exemplary 6-membered heterocyclic rings include, but are not limited to, optionally substituted piperidinyl, optionally substituted piperazinyl, optionally substituted morpholinyl, optionally substituted tetrahydropyranyl and optionally substituted dioxanyl.

Exemplary 5-membered carbocyclic rings include, but are not limited to, optionally substituted cyclopentyl and optionally substituted cyclopentenyl.

Exemplary 6-membered carbocyclic rings include, but are not limited to, optionally substituted cyclohexyl and optionally substituted cyclohexenyl.

In some embodiments, R² and R³ are not joined together to form a cyclic structure.

In some embodiments, R³ and R⁴ are not joined together to form a cyclic structure.

In some embodiments, both R¹ and R² and R² and R³ are joined to form rings, but R³ and R⁴ are not joined together to form a cyclic structure.

In some embodiments, both R¹ and R² and R³ and R⁴ are joined to form rings, but R² and R³ are not joined together to form a cyclic structure.

In some embodiments, both R² and R³ and R³ and R⁴ are joined to form rings, but R¹ and R² are not joined together to form a cyclic structure.

Palladium (II) Complexes with Bidentate Ligand

In some embodiments, Z is not joined via a linker group -L- to the group R^{L1} to form a 5- to 7-membered palladacycle.

For example, in some embodiments, the palladium (II) complex comprises a bidentate ligand. In some embodiments, the palladium (II) complex is of the formula (I-a):

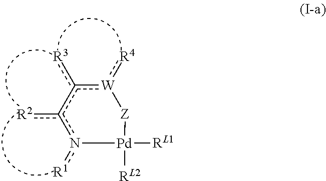

wherein Pd, [symbol] , [symbol] , W, R^{L1}, R^{L2}, Z, R¹, R², R³ and R⁴ are as defined above and herein.

In some embodiments, R¹ and R² are joined to form an optionally substituted 6-membered pyridinyl ring to provide a palladium (II) complex of the formula (I-b):

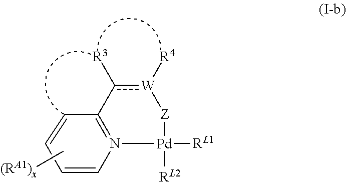

wherein

Pd, [symbol] , [symbol] , W, R^{L1}, R^{L2}, Z, R³, and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{A1a}, —C(═O)N(R^{A1c})², —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In some embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{Ala}. In some embodiments, each instance of R^{A1} is, independently, hydrogen, halogen, optionally substituted C₁₋₆ alkyl, —NO₂, —CF₃, or —OR^{A1a}. In some embodiments, each instance of R^{A1} is, independently, hydrogen, —CH₃, -tBu, —CN, —NO₂, —CF₃, or —OCH₃. In some embodiments, each instance of R^{A1} is hydrogen.

In some embodiments, R³ and R⁴ are joined to form an optionally substituted aryl ring to provide a palladium (II) complex of the formula (I-c):

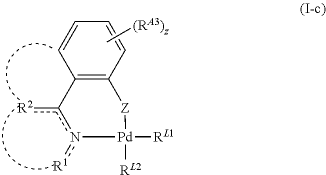

wherein

Pd, [symbol] , [symbol] , R¹, R², R^{L1}, R^{L2}, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In some embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In some embodiments, each instance of R^{A3} is, independently, hydrogen, halogen, optionally substituted C₁₋₆ alkyl, —NO₂, —CF₃, or —OR^{A3a}. In some embodiments, each instance of R^{A3} is, independently, hydrogen, —CH₃, -tBu, —CN, —NO₂, —CF₃, or —OCH₃. In some embodiments, each instance of R^{A3} is hydrogen.

In some embodiments, R¹ and R² are joined to form an optionally substituted 6-membered pyridinyl ring and R³ and R⁴ are joined to form an optionally substituted aryl ring to provide a palladium (II) complex of the formula (I-d):

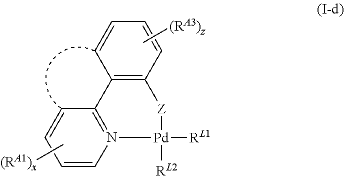

Pd, [symbol] , [symbol] , R^{A1}, R^{A3}, R^{L1}, R^{L2}, x, z, and Z are as defined above and herein.

In some embodiments, R¹ and R² are joined to form an optionally substituted 6-membered pyridinyl ring and R² and R³ are joined to form an optionally substituted 6-membered aryl ring, to provide a palladium (II) catalyst of the formula (I-e):

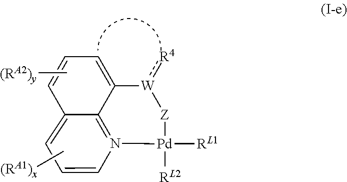

wherein

Pd, [symbol] , [symbol] , W, R^{A1}, R^{L1}, R^{L2}, R⁴, x, and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2a})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In some embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In some embodiments, each instance of R^{A2} is, independently, hydrogen, halogen, optionally substituted C₁₋₆ alkyl, —NO₂, —CF₃, or —OR^{A2a}. In some embodiments, each instance of R^{A2} is, independently, hydrogen, —CH₃, -tBu, —CN, —NO₂, —CF₃, or —OCH₃. In some embodiments, each instance of R^{A2} is hydrogen.

In some embodiments, R² and R³ are joined to form an optionally substituted 6-membered aryl ring to provide a palladium (II) catalyst of the formula (I-f):

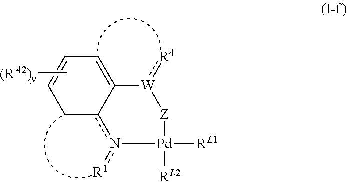

wherein Pd, [symbol] , [symbol] , W, R^{A2}, R¹, R⁴, R^{L1}, R^{L2}, y and Z are as defined above and herein.

In some embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the bidentate palladium (II) complex of the formula (I-g):

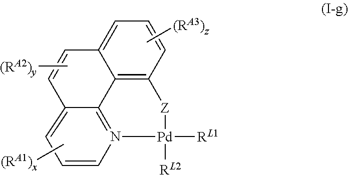

wherein Pd, R^{L1}, R^{L2}, Z, R^{A1}, R^{A2}, R^{A3}, x, y and z are as defined above and herein.

In some embodiments, wherein R² and R³ are not joined to form an optionally substituted 5- to 6-membered ring, the palladium (II) complex is of the formula (I-h):

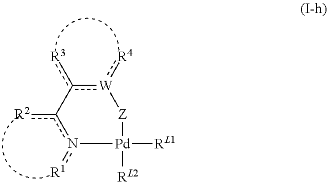

wherein Pd, [symbol] , [symbol] , W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring; and

R³ and R⁴ are optionally joined to form an optionally substituted 5- to 7-membered heteroaryl, aryl, heterocyclic or carbocyclic ring.

In some embodiments, wherein R² and R³ are not joined to form a cyclic structure, the palladium (II) complex is of the formula (I-i):

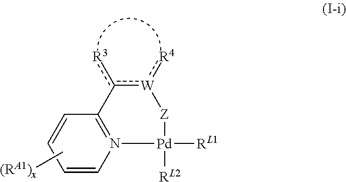

wherein Pd, [symbol] , [symbol] , W, R³, R⁴, R^{L1}, R^{L2}, R^{A1} and x are as defined above and herein.

In some embodiments, wherein R² and R³ are not joined to form a cyclic structure, the palladium (II) complex is of the formula (I-j):

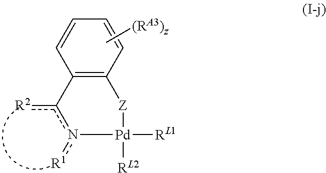

wherein Pd, [symbol] , [symbol] , R¹, R², R^{L1}, R^{L2}, R^{A3}, Z and z are as defined above and herein.

In some embodiments, wherein R² and R³ are not joined to form a cyclic structure, the palladium (II) complex is of the formula (I-k):

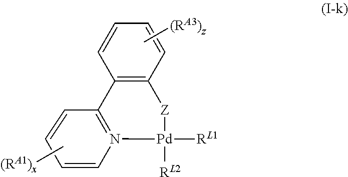

wherein Pd, R^{L1}, R^{L2}, R^{A1}, R^{A3}, Z, z and x are as defined above and herein.

In some embodiments, in any of the above formulae Z is a bond. In other embodiments, Z is

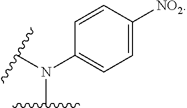

In other embodiments, Z is

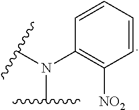

In some embodiments, wherein R² and R³ are not joined to form a cyclic structure and Z is a bond, the palladium (II) complex is of the formula (I-l):

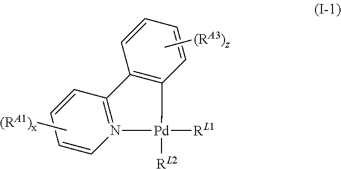

wherein R^{L1}, R^{L2}, R^{A1}, R^{A3}, z and x are as defined above and herein.

In some embodiments, the palladium (II) complex is of the formula (I-k):

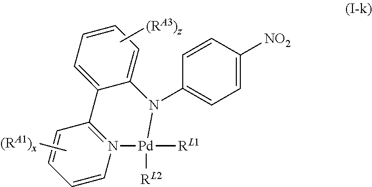

wherein R^{L1}, R^{L2}, R^{A1}, R^{A3}, z, and x are as defined above and herein.

In some embodiments, the palladium (II) complex is of the formula (I-l′):

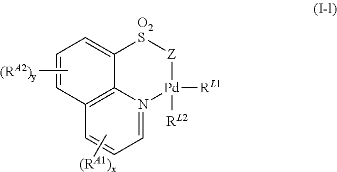

wherein Pd, R^{L1}, R^{L2}, R^{Al}, R^{A2}, x, y, and Z are as defined above and herein.

In some embodiments, the palladium (II) complex is of the formula (I-m′):

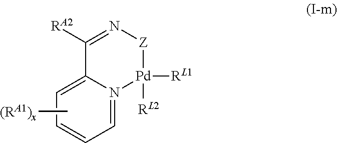

wherein Pd, R^{L1}, R^{L2}, R^{A1}, R^{A2}, x, and Z are as defined above and herein.

In some embodiments, the palladium (II) complex is of the formula (I-n′):

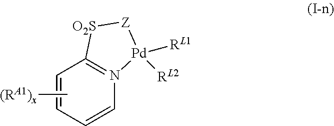

wherein Pd, R^{L1}, R^{L2}, R^{A1}, x, and Z are as defined above and herein.

Palladium (II) Complexes with Tridentate Ligand

In some embodiments, Z is joined via a linker group -L- to the group R^{L1} to form a 5- to 7-membered palladacycle.

In some embodiments, the palladium (II) catalyst comprises a tridentate ligand. In some embodiments, the palladium (II) catalyst of the formula (I-a′):

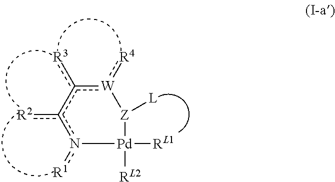

wherein

Pd, [symbol] , [symbol] , W, R^{L1}, R^{L2}, R¹, R², R³, and R⁴ are as defined above and herein;

Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring; and

the curved solid line [symbol] represents joining of the 5- to 7-membered palladacycle.

In some embodiments, R¹ and R² are joined to form an optionally substituted 6-membered pyridinyl ring to provide a palladium (II) comlex of the formula (Ib′):

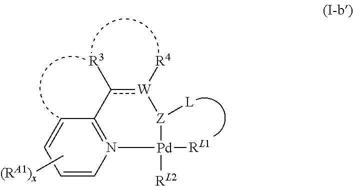

wherein

Pd, [symbol] , [symbol] , [symbol] , W, L, R^{L1}, R^{L2}, Z, R³ and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{A1a}, —C(═O)N(R^{A1c})₂, —C(═NR^{A1c})R^{A1d}, —C(═NR^{A1c})OR^{A1a}, —C(═NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In some embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In some embodiments, each instance of R^{A1} is, independently, hydrogen, halogen, optionally substituted C₁₋₆ alkyl, —NO₂, —CF₃, or —OR^{A1a}. In some embodiments, each instance of R^{A1} is, independently, hydrogen, —CH₃, -tBu, —CN, —NO₂, —CF₃, or —OCH₃. In some embodiments, each instance of R^{A1} is hydrogen.

In some embodiments, R³ and R⁴ are joined to form an optionally substituted aryl ring to provide a palladium (II) complex of the formula (I-c′):

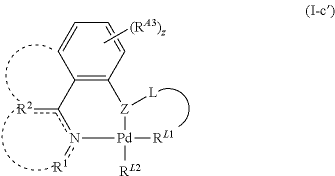

wherein

Pd, [symbol] , [symbol] , [symbol] , L, R¹, R², R^{L1}, R^{L2}, z, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In some embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In some embodiments, each instance of R^{A3} is, independently, hydrogen, halogen, optionally substituted C₁₋₆ alkyl, —NO₂, —CF₃, or —OR^{A3a}. In some embodiments, each instance of R^{A3} is, independently, hydrogen, —CH₃, -tBu, —CN, —NO₂, —CF₃, or —OCH₃. In some embodiments, each instance of R^{A3} is hydrogen.

In some embodiments, R¹ and R² are joined to form an optionally substituted 6-membered pyridinyl ring and R³ and R⁴ are joined to form an optionally substituted aryl ring to provide a palladium (II) complex of the formula (I-d′):

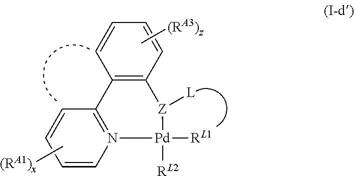

wherein Pd, [symbol] , [symbol] , [symbol] , L, R^{A1}, R^{A3}, R^{L1}, R^{L2}, x, z, and Z are as defined above and herein.

In some embodiments, R¹ and R² are joined to form an optionally substituted 6-membered pyridinyl ring and R² and R³ are joined to form an optionally substituted 6-membered aryl ring, to provide a palladium (II) catalyst of the formula (I-e′):

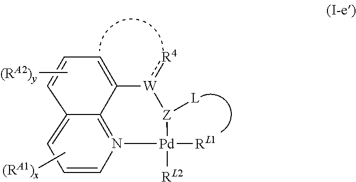

wherein Pd, [symbol] , [symbol] , [symbol] , L, W, R^{A1}, R^{L1}, R^{L2}, R⁴, x and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2C} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In some embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In some embodiments, each instance of R^{A2} is, independently, hydrogen, halogen, optionally substituted C₁₋₆ alkyl, —NO₂, —CF₃, or —OR^{A2a}. In some embodiments, each instance of R^{A2} is, independently, hydrogen, —CH₃, -tBu, —CN, —NO₂, —CF₃, or —OCH₃. In some embodiments, each instance of R^{A2} is hydrogen.

In some embodiments, R² and R³ are joined to form an optionally substituted 6-membered aryl ring to provide a palladium (II) catalyst of the formula (I-f′):

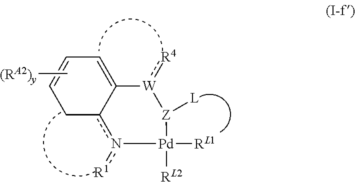

wherein Pd, [symbol] , [symbol] , [symbol] , L, W, R^{A2}, R¹, R⁴, R^{L1}, R^{L2}, y and Z are as defined above and herein.

In some embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the palladium (II) complex of the formula (I-g′):

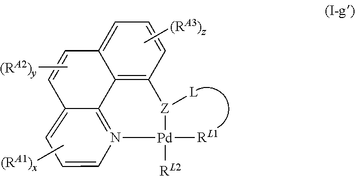

wherein [symbol] , L, R^{L1}, R^{L2}, Z, R^{A1}, R^{A2}, R^{A3}, x, y and z are as defined above and herein.

In some embodiments, wherein R² and R³ are not joined to form an optionally substituted 5- to 6-membered ring, the palladium (II) complex is of the formula (I-h′):

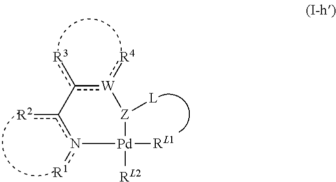

wherein Pd, [symbol] , [symbol] , [symbol] , L, W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring; and

R³ and R⁴ are optionally joined to form an optionally substituted 5- to 7-membered heteroaryl, aryl, heterocyclic or carbocyclic ring.

In some embodiments, wherein R² and R³ are not joined to form a cyclic structure, the palladium (II) complex is of the formula (I-i′):

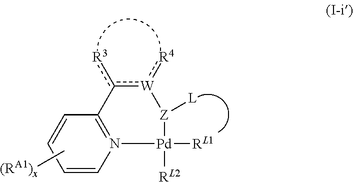

wherein Pd, [symbol] , [symbol] , [symbol] , L, W, R³, R⁴, R^{L1}, R^{L2}, A^{A1} and x are as defined above and herein.

In some embodiments, wherein R² and R³ are not joined to form a cyclic structure, the palladium (II) complex is of the formula (I-j′):

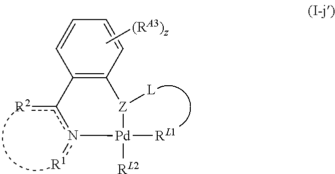

wherein Pd, [symbol] , [symbol] , [symbol] , L, R¹, R², R^{L1}, R^{L2}, R^{A3} and z are as defined above and herein.

In some embodiments, wherein R² and R³ are not joined to form a cyclic structure, the palladium (II) complex is of the formula (I-k′):

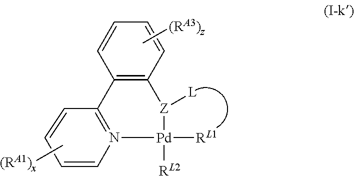

wherein Pd, [symbol] , L, R^{L1}, R^{L2}, R^{A1}, R^{A3}, Z, z and x are as defined above and herein.

Groups R^{L1} and R^{L2}

As defined generally above, R^{L1} and R^{L2} are, independently, halogen, optionally substituted aliphatic, optionally substituted heteroaliphatic, optionally substituted aryl, optionally substituted heteroaryl, —OR^{a}, —SR^{b}, —N(R^{c})₃, —N(R^{c})₂, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, heteroaliphatic, aryl, heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, heteroaliphatic, aryl, heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted 5- to 6-membered heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring; and

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group.

In some embodiments, at least one of R^{L1} and R^{L2} is selected from halogen, —OR^{a}, —SR^{b}, —N(R^{c})₃, —N(R^{c})₂, or —P(R^{x})₃. In some embodiments, both R^{L1} and R^{L2} are, independently, selected from halogen, —OR^{a}, —SR^{b}, —N(R^{c})₃, —N(R^{c})₂, or —P(R^{x})₃.

In some embodiments, R^{L1} is halogen, OR^{a}, —SR^{b}, or —N(R^{c})₂ and R^{L2} is —N(R^{c})₂. In some embodiments, R^{L1} is halogen, —OR^{a} or —N(R^{c})₂, and R^{L2} is —N(R^{c})₂. In some embodiments, R^{L1} is halogen or —OR^{a}, and R^{L2} is —N(R^{c})₂. In some embodiments, R^{L1} is and R^{L2} is —N(R^{c})₂. In some embodiments, R^{L1} is halogen and R^{L2} is —N(R^{c})₂. In some embodiments, R^{L1} is —OR^{a} and R^{L2} is —N(R^{c})₂. In some embodiments, both R^{L1} and R^{L2} are independently —N(R^{c})₂.

In some embodiments, R^{L1} is halogen. In some embodiments, R^{L1} is —Cl. In some embodiments, R^{L1} is —Br. In some embodiments, R^{L1} is —I. In some embodiments, R^{L1} is —F.

In some embodiments, R^{L1} is —OR^{a}.

In some embodiments, R^{L1} is —OC(═O)R^{a1} wherein R^{a1} is an optionally substituted aliphatic, optionally substituted heteroaliphatic, optionally substituted aryl or optionally substituted heteroaryl group. In some embodiments, R^{L1} is —OC(═O)R^{a1} wherein R^{a1} is an optionally substituted aliphatic group. In some embodiments, R^{L1} is —OC(═O)R^{a1} wherein R^{a1} is an optionally substituted C₁₋₆ alkyl group. In some embodiments, R^{L1} is —OC(═O)R^{a1} wherein R^{a1} is an optionally substituted C₁₋₄ alkyl group. In some embodiments, R^{L1} is —OC(═O)R^{a1} wherein R^{a1} is an optionally substituted C₁₋₂ alkyl group. In some embodiments, R^{L1} is —OC(═O)CH₃.

In some embodiments, R^{L1} is —P(R^{X})₃.

In some embodiments, R^{L2} is —N(R^{c})₂.

In some embodiments, R^{L2} is —N(R^{c})₂ wherein two R^{c} groups are joined to form the group C(R^{c1}), wherein R^{c1} is an optionally substituted aliphatic, optionally substituted heteroaliphatic, optionally substituted aryl or optionally substituted heteroaryl group. In some embodiments, R^{L2} is —N(R^{c})₂ wherein two R^{c} groups are joined to form the group C(R^{c1}), wherein R^{c1} is an optionally substituted aliphatic group. In some embodiments, R^{L2} is —N(R^{c})₂ wherein two R^{c} groups are joined to form the group C(R^{c1}), wherein R^{c1} is an optionally substituted C₁₋₆ alkyl group. In some embodiments, R^{L2} is —N(R^{c})₂ wherein two R^{c} groups are joined to form the group C(CH₃) or C(CH₂Ph).

In some embodiments, R^{L2} is —N(R^{c})₂ wherein two R^{c} groups are joined to form an optionally substituted heterocyclic or heteroaryl ring.

In some embodiments, R^{L2} is —N(R^{c})₂ wherein two R^{c} groups are joined to form an optionally substituted 5- to 6-membered heterocyclic or heteroaryl ring.

In some embodiments, R^{L2} is —N(R^{c})₂ wherein two R^{c} groups are joined to form an optionally substituted 5-membered heterocyclic ring. Exemplary 5-membered heterocyclic rings include, but are not limited to, an optionally substituted pyrrolidinyl ring.

In some embodiments, R^{L2} is —N(R^{c})₂ wherein two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

In some embodiments, R^{L2} is —N(R^{c})₂ wherein two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In some embodiments, R^{L2} is —N(R^{c})₂ wherein two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In some embodiments, R^{L2} is an optionally substituted pyridinyl ring.

In some embodiments, R^{L1} is —N(R^{c})₂.

In some embodiments, R^{L1} is —N(R^{c})₂ wherein two R^{c} groups are joined to form the group C(R^{c1}), wherein R^{c1} is an optionally substituted aliphatic, optionally substituted heteroaliphatic, optionally substituted aryl or optionally substituted heteroaryl group. In some embodiments, R^{c1} is —N(R^{c})₂ wherein two R^{c} groups are joined to form the group ≡C(R^{c1}), wherein R^{c1} is an optionally substituted aliphatic group. In some embodiments, R^{L1} is —N(R^{c})₂ wherein two R^{c} groups are joined to form the group C(R^{c1}), wherein R^{c1} is an optionally substituted C₁₋₆ alkyl group. In some embodiments, R^{L1} is —N(R^{c})₂ wherein two R^{c} groups are joined to form the group C(CH₃) or C(CH₂Ph).

In some embodiments, R^{L1} is —N(R^{c})₂ wherein two R^{c} groups are joined to form an optionally substituted 5- to 6-membered heterocyclic or heteroaryl ring.

In some embodiments, R^{L1} is —N(R^{c})₂ wherein two R^{c} groups are joined to form an optionally substituted 5-membered heterocyclic ring. Exemplary 5-membered heterocyclic rings are provided above and herein.

In some embodiments, R^{L1} is —N(R^{c})₂ wherein two R^{c} groups are joined to form an optionally substituted 5-membered heteroaryl ring. Exemplary 5-membered heteroaryl rings are provided above and herein.

In some embodiments, R^{L1} is —N(R^{c})₂ wherein two R^{c} groups are joined to form an optionally substituted 6-membered heterocyclic ring. Exemplary 6-membered heterocyclic rings are provided above and herein.

In some embodiments, R^{L1} is —N(R^{c})₂ wherein two R^{c} groups are joined to form an optionally substituted 6-membered heteroaryl ring. Exemplary 6-membered heteroaryl rings are provided above and herein.

In some embodiments, R^{L1} is an optionally substituted pyridinyl ring. Optionally substituted pyridinyl rings include, but are not limited to, rings of the formula:

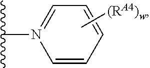

wherein each instance of R^{A4} is, independently, hydrogen, halogen, optionally substituted aliphatic, optionally substituted heteroaliphatic, optionally substituted aryl, optionally substituted heteroaryl, —CN, —NO₂, —NC, —OR^{A4a}, —SR^{A4b}, —N(R^{A4c})₂, —C(═O)R^{A4d}, —C(═O)OR^{A4a}, —C(═O)N(R^{A4c})₂, —C(═NR^{A4d})R^{A4d}, —(═NR^{A4c})OR^{A4a}, —C(═NR^{A4c})N(R^{A4c})₂, —S(O)₂R^{A4d}, —S(O)R^{A4d}, or two R^{A4} groups adjacent to each other are joined to form a 5- to 6-membered aryl, heteroaryl, heterocyclic or carbocyclic ring, wherein R^{A4a} is hydrogen, an optionally substituted aliphatic, optionally substituted heteroaliphatic, optionally substituted aryl, optionally substituted heteroaryl or a suitable hydroxyl protecting group; wherein R^{A4b} is hydrogen, an optionally substituted aliphatic, optionally substituted heteroaliphatic, optionally substituted aryl, optionally substituted heteroaryl or a suitable thiol protecting group; wherein each R^{A4c} is, independently, hydrogen, an optionally substituted aliphatic, optionally substituted heteroaliphatic, optionally substituted aryl, optionally substituted heteroaryl or a suitable amino protecting group, or two R^{A4c} groups are joined together to form a heterocyclic or heteroaryl group; and wherein each R^{A4d} independently, an optionally substituted aliphatic, optionally substituted heteroaliphatic, optionally substituted aryl, or an optionally substituted heteroaryl group, and

w is an integer between 0 to 5, inclusive.

In some embodiments, the optionally substituted pyridinyl ring is of the formulae:

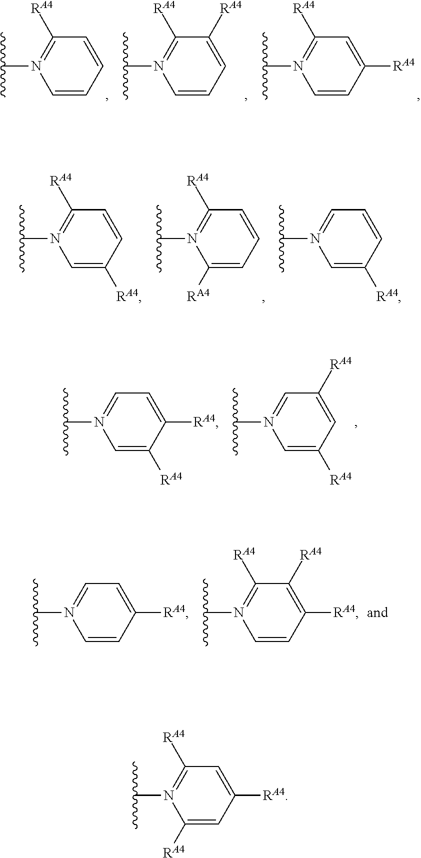

In some embodiments, the optionally substituted pyridinyl ring is:

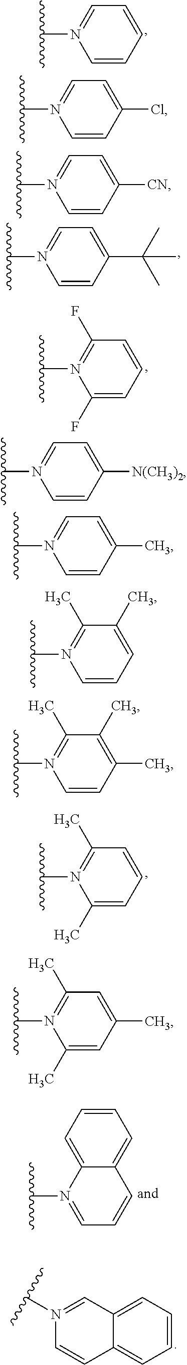

In some embodiments, R^{L2} is —P(R^{X})₃. In some embodiments, R^{X} is optionally substituted aliphatic. In some embodiments, R^{X} is optionally substituted aryl. In some embodiments, R^{X} is optionally substituted alkoxy. In some embodiments, R^{X} is optionally substituted aryloxy. In some embodiments, R^{L2} is —P(Me)₃. In some embodiments, R^{L2} is —P(Et)₃. In some embodiments, R^{L2} is —P(tert-Bu)₃. In some embodiments, R^{L2} is —P(Cy)₃. In some embodiments, R^{L2} is —P(Ph)₃. In some embodiments, R^{L2} is —PMe(Ph)₂. In some embodiments, R^{L2} is —PF₃. In some embodiments, R^{L2} is —P(OMe)₃. In some embodiments, R^{L2} is —P(OEt)₃. In some embodiments, R^{L2} is —P(OPh)₃.

Z, L, and R^{L1}

As generally defined above, in some embodiments, Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted membered heterocyclic or heteroaryl ring.

In some embodiments, R^{L1} is —N(R^{c})₂ optionally joined to Z via a linker group -L- to form a 5- to 7-membered palladacycle, wherein two R^{c} groups are joined to form an optionally substituted membered heterocyclic or heteroaryl ring.

In some embodiments, two R^{c} groups are joined to form an optionally substituted 5-membered heterocyclic ring. Exemplary 5-membered heterocyclic rings include, but are not limited to, an optionally substituted pyrrolidinyl ring.

In some embodiments, two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

In some embodiments, two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In some embodiments, two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In some embodiments, two R^{c} groups are joined to form an optionally substituted bicyclic heteroaryl ring. Exemplary bicyclic heteroaryl rings include, but are not limited to, optionally substituted quinolinyl and optionally substituted isoquinolinyl.

In some embodiments, two R^{c} groups are joined to form an optionally substituted pyridinyl ring. In some embodiments, two R^{c} groups are joined to form an optionally substituted quinolinyl ring.

For example, in some embodiments, wherein two R^{c} groups are joined to form an optionally substituted pyridinyl ring, the group provided by Z, L and R^{L1} is of the formulae:

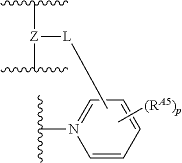

wherein:

Z is —N—;
L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is and integer between 0 to 5, inclusive.

In some embodiments, wherein two R^{c} groups are joined to form an optionally substituted quinolinyl ring, the group provided by Z, L and R^{L1} is of the formulae:

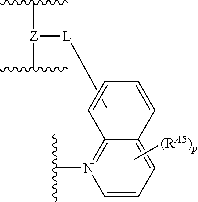

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is and integer between 0 to 5, inclusive.

In some embodiments, -L- is —C(═O)—.

In some embodiments, -L- is —C(═O)O—.

In some embodiments, -L- is —C(═O)N(R^{e3})—.

In some embodiments, -L- is —C(═NR^{e3})—.

In some embodiments, -L- is —C(═NR^{e3})O—.

In some embodiments, -L- is —C(═NR^{e3})N(R^{e3})—.

In some embodiments, -L- is —S(O)₂—.

In some embodiments, -L- is—S(O)—.

In some embodiments, the group provided by Z, L and R^{L1} is of the formulae:

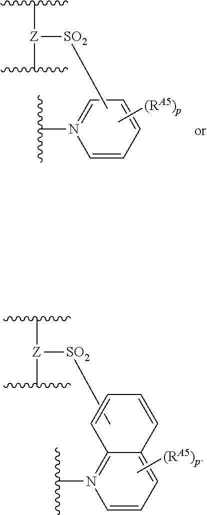

In some embodiments, the group provided by Z, L and R^{L1} is of the formulae:

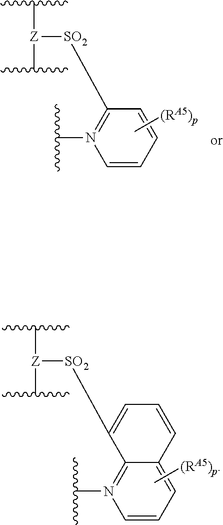

In some embodiments, the group provided by Z, L and R^{L1} is:

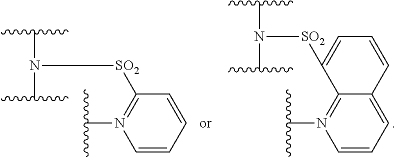

Group Z

In some embodiments, Z is not linked to the ligand R^{L1} as in the case of a palladium (II) complex with a bidentate ligand. As defined generally above, in some embodiments, Z is a bond, —O—, —S—, —C(R^{d})₂—, —C(R^{d})═C(R^{d})—, —C(R^{d})═N—, or —N(R^{e})—;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted membered heterocyclic or heteroaryl ring.

In some embodiments, Z is a bond.

In some embodiments, Z is —C(R^{d})₂—. In some embodiments, Z is —CH₂—.

In some embodiments, Z is —C(R^{d})═C(R^{d})—. In some embodiments, Z is —CH═CH—.

In some embodiments, Z is —C(R^{d})═N—. In some embodiments, Z is —CH═N—

In some embodiments, Z is —O—.

In some embodiments, Z is —S—.

In some embodiments, Z is —NR^{e}—.

In some embodiments, wherein Z is —NR^{e}—, the R^{e} group is of the formula —S(O)₂R^{e1}, wherein R^{e1} is an optionally substituted aliphatic, optionally substituted heteroaliphatic, optionally substituted aryl or optionally substituted heteroaryl group. In some embodiments, the R^{e} group is of the formula —S(O)₂R^{e1}, wherein R^{e1} is an optionally substituted aryl or optionally substituted heteroaryl group. In some embodiments, the R^{e} group is of the formula —S(O)₂R^{e1}, wherein R^{e1} is an optionally substituted heteroaryl group. In some embodiments, the R^{e} group is of the formula —S(O)₂R^{e1}, wherein R^{e1} is an optionally substituted aryl group.

Exemplary —S(O)₂R^{e1} groups include, but are not limited to:

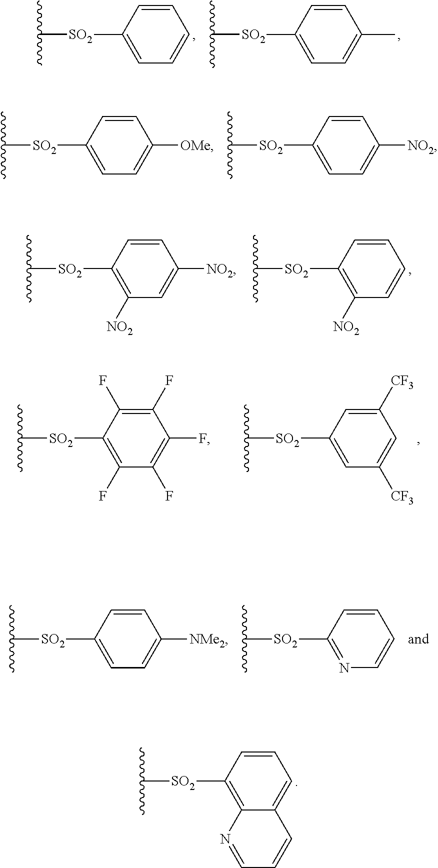

In some embodiments, Z is of the formula:

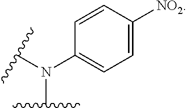

In some embodiments, Z is of the formula:

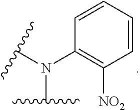

In some embodiments, Z is of the formula:

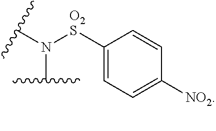

In some embodiments, Z is of the formula:

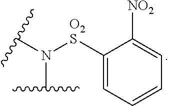

Exemplary Palladium(II) complexes

In some embodiments, the palladium(II) complex is selected from any of the following complexes:

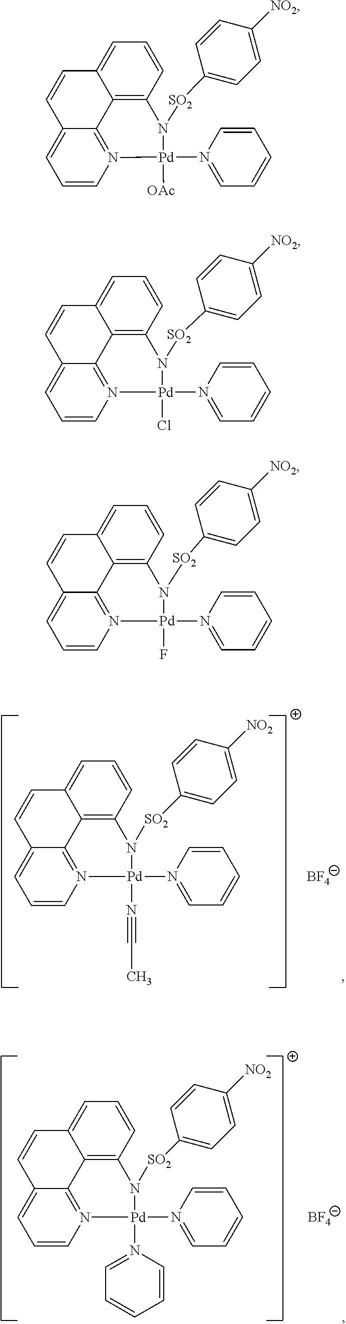

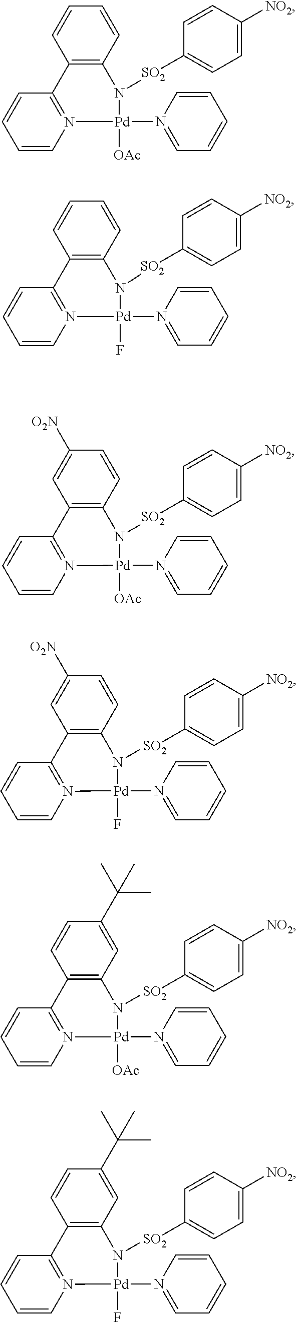

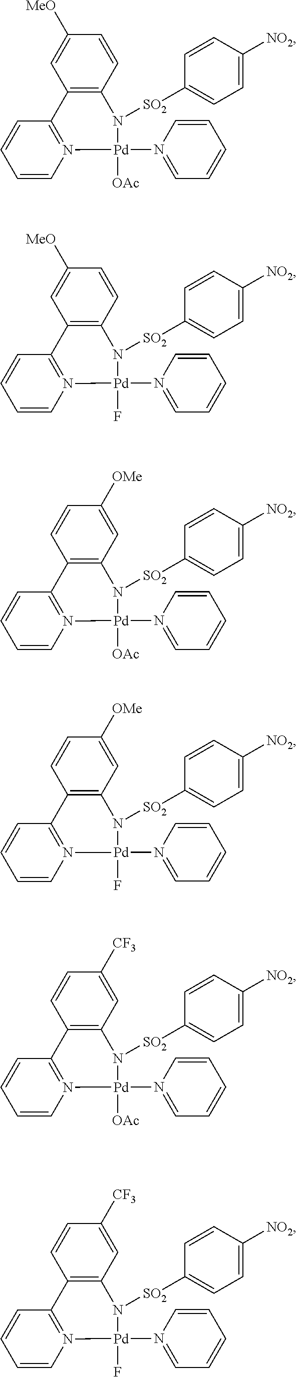

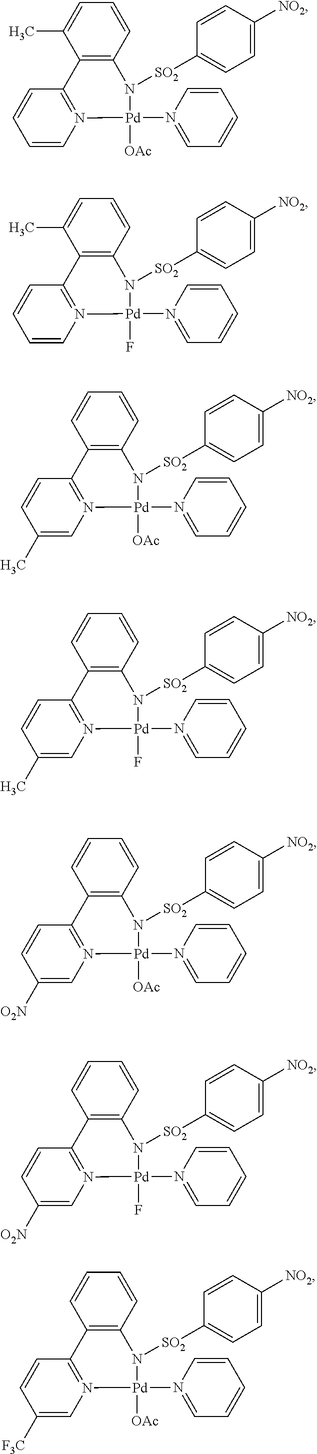

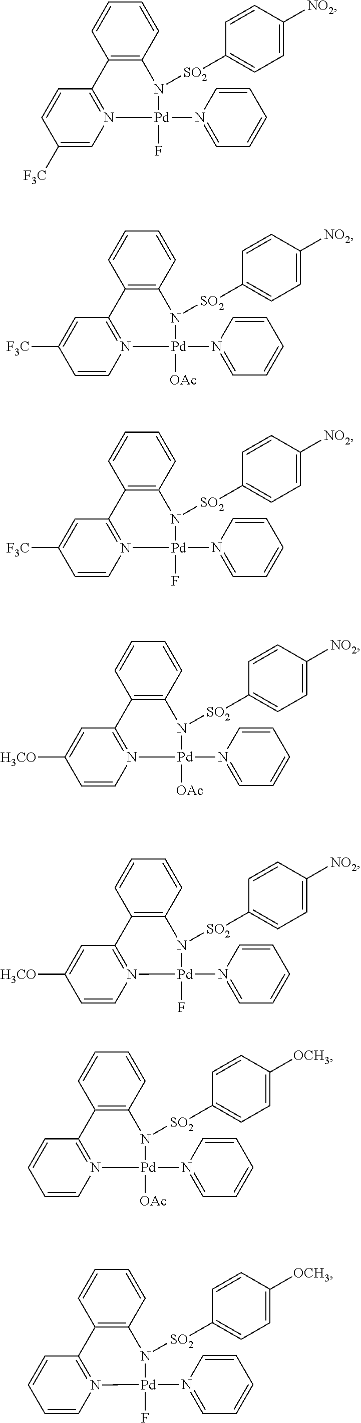

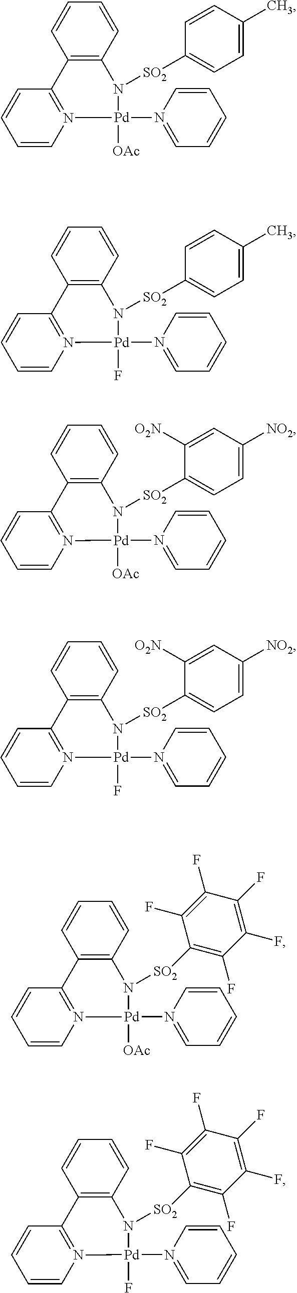

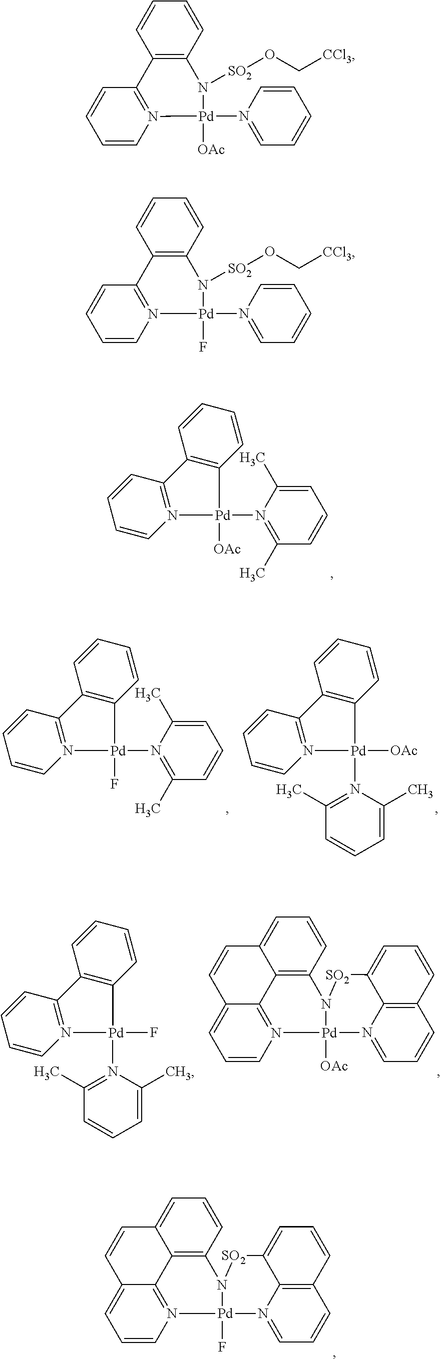

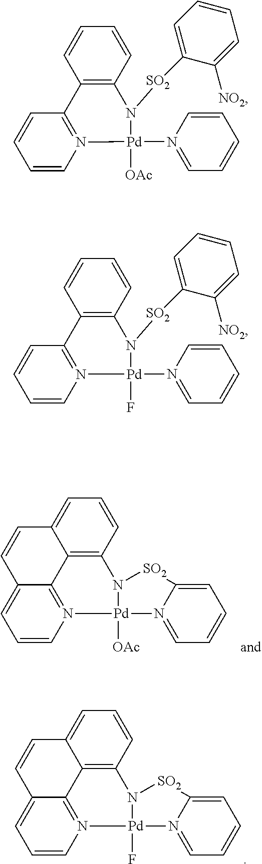

In some embodiments, the palladium (II) complex is of the formula:

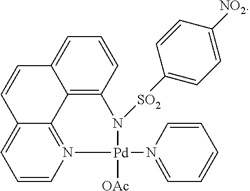

In some embodiments, the palladium(II) complex is of the formula:

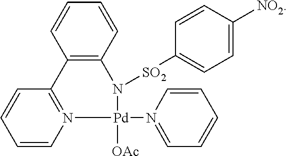

In some embodiments, the palladium(II) complex is of the formula:

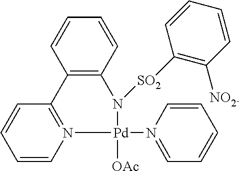

In some embodiments, the palladium(II) complex is of the formula:

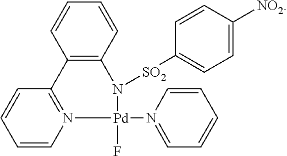

Fluorination with High-Valent Pd(IV)-Fluoride Complexes

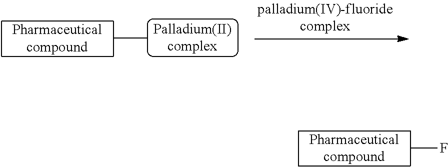

Upon reaction of an organopalladium(II) complex with a high-valent Pd(IV)-fluoride complex, the method provides a fluorinated organic compound in which the organic compound is fluorinated at the position at which it was bound to the palladium(II) center. In some embodiments, the organic compound is attached to the palladium(II) center (and subsequently fluorinated) via an aryl or heteroaryl moiety. For example, see Scheme 8.

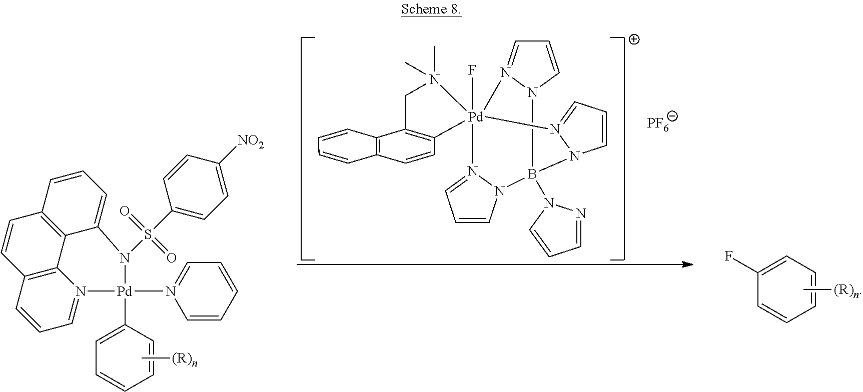

Exemplary methods of fluorinating a compound using a Pd(IV) complex are described in WO2009/149347, which is incorporated herein by reference in its entirety.

Palladium (IV) Complexes

In some embodiments, the complex is a Pd (IV) complex. Typically, the complex comprises one or more bidentate or tridentate ligands. Such ligands, particularly “scorpionate ligands,” are thought to stabilize the octahedral coordination sphere of the palladium (IV) center and thus prevent reductive elimination or other reductive pathways from an octahedral d⁶ palladium (IV) to a square planar d⁸ palladium (II).

In some embodiments, the inventive high-valent palladium fluoride complex is of the formula:

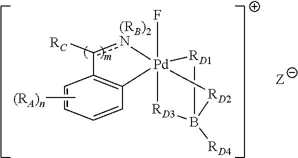

wherein:

the dashed line represents the presence or absence of a bond;

Pd has a valency of +4;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring;

R_{D1}, R_{D2}, R_{D}3, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

Z⁻ is an anion such as halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate; and

F comprises ¹⁸F or ¹⁹F.

In some embodiments, the inventive high-valent palladium fluoride complex is of the formula:

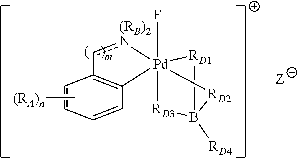

wherein

the dashed line represents the presence or absence of a bond;

Pd has a valency of +4;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R_{D1}, R_{D2}, R_{D3}, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

Z⁻ is an anion such as halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate; and

F comprises ¹⁸F or ¹⁹F.

The counteranion Z⁻ may be any suitable anion. In some embodiments, the counteranion has a charge of −1. In some embodiments, the counteranion has a charge of −2. In some embodiments, the counteranion has a charge of −3. The counteranion may be an organic or inorganic anion. In some embodiments, the counteranion is an inorganic anion such as phosphate, hexafluorophosphate, hexafluoroantimonate, sulfate, perchlorate, azide, a halide such as fluoride, chloride, bromide or iodide, etc. In other embodiments, the counteranion is an organic anion such as a carboxylate (e.g., acetate), sulfonate, phosphonate, borate, etc. In some embodiments, the counteranion is trifluoromethanesulfonate (triflate). In some embodiments, the counteranion is tosylate. In some embodiments, the counteranion is mesylate. In some embodiments, the counteranion is hexafluorophosphate. In some embodiments, the counteranion is tetraphenylborate. In some embodiments, the counteranion is tetrafluoroborate. In some embodiments, the counteranion tetrakis(pentafluorophenyl)borate. In some embodiments, the counteranion is hexafluoroanimonate. In some embodiments, the counterion is [B[3,5-(CF₃)₂C₆H₃]₄]⁻, commonly abbreviated as [BAr^{F}₄]⁻.

In some embodiments, n is 0, in which case the phenyl ring is unsubstituted. In some embodiments, n is 1. In some embodiments, n is 2. In some embodiments, n is 3. In some embodiments, n is 4. For the case where n is 1 or more, the substituents on the phenyl ring may have any substitution pattern.

In some embodiments, m is 0. In some embodiments, m is 1. In some embodiments, m is 2. In some embodiments, m is 3.

In some embodiments, the dashed line represents a bond, thus forming an imine moiety. In other embodiments, the dashed line represents the absence of a bond resulting in only a single bond between the carbon atom and nitrogen atom.

In some embodiments, at least one R_{A} is halogen. In some embodiments, at least one occurrence of R_{A} is aliphatic. In some embodiments, at least one occurrence of R_{A} is C₁-C₆ alkyl. In some embodiments, at least one occurrence of R_{A} is methyl. In some embodiments, at least one occurrence of R_{A} is ethyl. In some embodiments, at least one occurrence of R_{A} is propyl. In some embodiments, at least one occurrence of R_{A} is butyl. In some embodiments, at least one occurrence of R_{A} is heteroaliphatic. In some embodiments, at least one occurrence of R_{A} is acyl. In some embodiments, at least one occurrence of R_{A} is aryl. In some embodiments, at least one occurrence of R_{A} is heteroaryl. In some embodiments, at least one occurrence of R_{A} is —OR′. In some embodiments, at least one occurrence of R_{A} is —N(R′)₂. In some embodiments, at least one occurrence of R_{A} is —SR′. In some embodiments, at least one occurrence of R_{A} is —NO₂. In some embodiments, at least one occurrence of R_{A} is —CN. In some embodiments, at least one occurrence of R_{A} is —SCN.

In some embodiments, two occurrences of R_{A} taken together form a cyclic moiety. Such a cyclic moeity may be carbocyclic or heterocyclic. In some embodiments, the cyclic moiety is a substituted or unsubstituted phenyl moiety. In some embodiments, the cyclic moiety is an unsubstituted phenyl moiety. In some embodiments, the cyclic moiety is a substituted or unsubstituted heteroaryl moiety.

In some embodiments, at least one occurrence of R_{B} is hydrogen. In some embodiments, both R_{B} are hydrogen. In some embodiments, at least one occurrence of R_{B} is aliphatic. In some embodiments, both occurrences of R_{B} are aliphatic. In some embodiments, both occurrences of R_{B} are C₁-C₆ alkyl. In some embodiments, both occurrences of R_{B} are methyl. In some embodiments, both occurrences of R_{B} are ethyl. In some embodiments, both occurrences of R_{B} are propyl. In some embodiments, both occurrences of R_{B} are butyl. In some embodiments, at least one occurrence of R_{B} is heteroaliphatic. In some embodiments, both occurrences of R_{B} are heteroaliphatic. In some embodiments, at least one occurrence of R_{B} is acyl. In some embodiments, at least one occurrence of R_{B} is aryl. In some embodiments, at least one occurrence of R_{B} is heteroaryl.

In some embodiments, both R_{B} are the same. In some embodiments, the two R_{B} are different.

In some embodiments, both R_{B} are taken together to form a heterocyclic moiety. In some embodiments, both R_{B} are taken together to form a 5-membered heterocyclic moiety. In some embodiments, both R_{B} are taken together to form a 6-membered heterocyclic moiety. In some embodiments, both R_{B} are taken together to form an optionally substituted heteroaryl moiety.

In some embodiments, one R_{B} moiety is covalently attached to a methylene group connecting the phenyl ring to the N atom, thus forming a heterocyclic moiety. Such a heterocyclic moiety may be a heteroaryl moiety. For example, in some embodiments, the heterocyclic moiety is a pyridinyl moiety.

In some embodiments, R_{C} is hydrogen. In some embodiments, R_{C} is aliphatic. In some embodiments, R_{C} is C₁-C₆ alkyl. In some embodiments, R_{C} is methyl. In some embodiments, R_{C} is ethyl. In some embodiments, R_{C} is propyl. In some embodiments, R_{C} is butyl. In some embodiments, R_{C} is heteroaliphatic. In some embodiments, R_{C} is heteroaliphatic. In some embodiments, R_{C} is acyl. In some embodiments, R_{C} is aryl. In some embodiments, R_{C} is heteroaryl. In some embodiments, one R_{B} and R_{C} are taken together to form a heterocyclic moiety. In some embodiments, one R_{B} and R_{C} are taken together to form a 5-membered heterocyclic moiety. In some embodiments, one R_{B} and R_{C} are taken together to form a 6-membered heterocyclic moiety. In some embodiments, one R_{B} and R_{C} are taken together to form an optionally substituted heteroaryl moiety.

In some embodiments, R_{D1}, R_{D2}, R_{D3} and R_{D4} all represent optionally substituted heteroaryl moieties. In some embodiments, at least one of R_{D1}, R_{D2}, R_{D3} and R_{D4} is an unsubstituted heteroaryl moiety. In some embodiments, R_{D1}, R_{D2}, R_{D3} and R_{D4} are all unsubstituted heteroaryl moieties. In some embodiments, R_{D1}, R_{D2}, R_{D3} and R_{D4} are all optionally substituted 5-membered heteroaryl moieties. In some embodiments, R_{D1}, R_{D2}, R_{D3} and R_{D4} are all nitrogen-containing 5-membered heteroaryl moieties, which are optionally substituted. In some embodiments, R_{D1}, R_{D2}, R_{D3} and R_{D4} are all optionally substituted pyrazolyl moieties. In some embodiments, R_{D1}, R_{D2}, R_{D3} and R_{D4} are all optionally substituted imidazolyl moieties. In some embodiments, R_{D1}, R_{D2}, R_{D3} and R_{D4} are all optionally substituted pyrrolyl moieties. In some embodiments, R_{D1}, R_{D2}, R_{D3} and R_{D4} are all are optionally substituted thiazolyl moieties. In some embodiments, R_{D1}, R_{D2}, R_{D3} and R_{D4} are all optionally substituted oxazolyl moieties. In some embodiments, R_{D1}, R_{D2}, R_{D3} and R_{D4} are all optionally substituted 6-membered heteroaryl moieties. In some embodiments, R_{D1}, R_{D2}, R_{D3} and R_{D4} are all nitrogen-containing 6-membered heteroaryl moieties, which are optionally substituted. In some embodiments, R_{D1}, R_{D2}, R_{D3} and R_{D4} are all optionally substituted pyridinyl moieties. In some embodiments, R_{D1}, R_{D2}, R_{D3} and R_{D4} are all optionally substituted pyrazinyl moieties. In some embodiments, R_{D1}, R_{D2}, R_{D3} and R_{D4} are all optionally substituted pyrimidinyl moieties. In some embodiments, R_{D1}, R_{D2}, R_{D3} and R_{D4} are all optionally substituted pyridazinyl moieties. In some embodiments, all of R_{D1}, R_{D2}, R_{D3} and R_{D4} of the borate ligand are the same. In other embodiments, all of R_{D1}, R_{D2}, R_{D3} and R_{D4} of the borate ligand are not the same. For example, a combination of heterocycle may constitute the borate ligand. In some embodiments, a combination of heteroaryl moieties may constitute the borate ligand.

In some embodiments, the palladium complex comprises a bidentate ligand of one of the formulae:

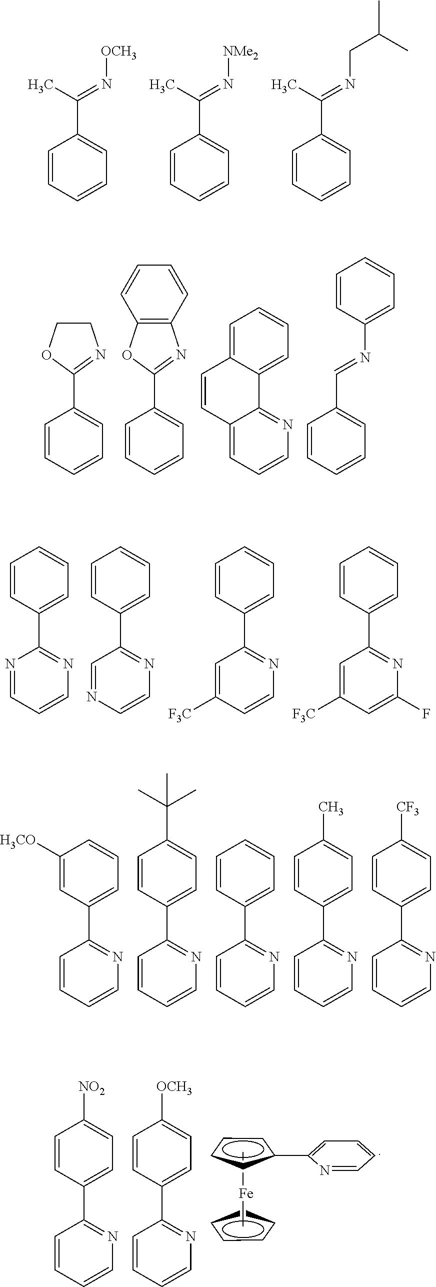

These ligands make a five-membered ring with the palladium atom with the nitrogen and a carbon coordinated to the central palladium.

In some embodiments, the palladium complex is of the formula:

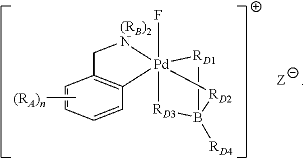

In some embodiments, the palladium complex is of the formula:

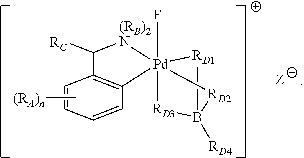

In some embodiments, the palladium complex is of the formula:

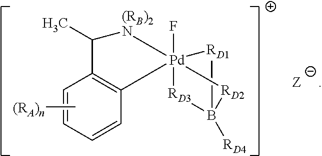

In some embodiments, the palladium complex is of the formula:

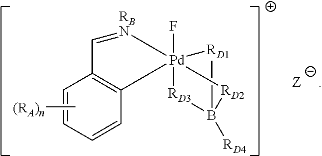

In some embodiments, the palladium complex is of the formula:

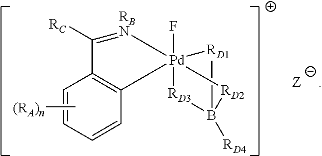

In some embodiments, the palladium complex is of the formula:

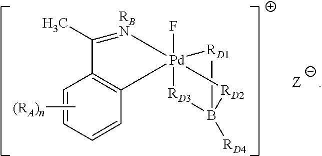

In some embodiments, the palladium complex is of the formula:

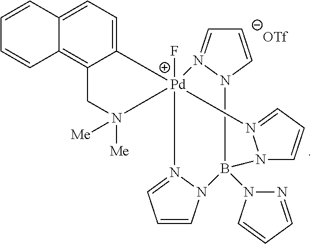

In some embodiments, the palladium complex is of the formula:

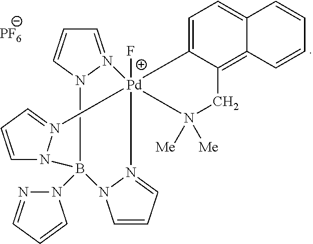

Preparation of High-Valent Palladium Fluoride Complexes

The inventive palladium complexes are typically prepared starting from disodium tetrachloropalladate. As would be appreciated by one of skill in the art, other palladium salts may also be used to prepare the inventive complexes. The starting material is subjected to cyclometallation to yield a palladium(II) chloride dimer. The chloride ligands are then substituted using the desired borate ligand to yield a palladium(II) borate, which is then oxidized with a fluorine-containing oxidizing reagent (e.g., 1-fluoro-pyridinium triflate, 2,4,6-trimethylpyridinium hexafluorophosphate, etc.) to yield the inventive palladium(IV) complex. An exemplary synthesis of a palladium(IV) fluoride complex is shown in FIG. 1.

In some embodiments, the method of preparing an inventive palladium(IV) fluoride complex comprises (1) cyclometallating a palladium(II) salt with a bidentate ligand comprising a carbon-based with a carbon donor and a nitrogen donor to yield a palladium(II) chloride dimer; (2) reacting the palladium(II) dimer with a tridentate borate ligand under suitable conditions to yield a palladium(II) borate; and oxidizing the palladium(II) borate with a fluorinating reagent under suitable conditions to yield a palladium(IV) fluoride complex.

In some embodiments, the bidentate ligand is of the formula:

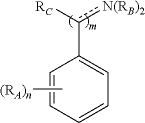

wherein

the dashed line represents the presence or absence of a bond;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two RA may be taken together to form a substituted or unsubstituted carbocyclic, heterocyclic, aryl or heteroaryl ring; and

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring.

In some embodiments, the borate ligand is tetrapyrazolylborate. In some embodiments, the borate ligand is phenyltris(methimazolyl)borate.

In some embodiments, an intermediate in the synthesis of a palladium(IV) fluoride complex is of the formula:

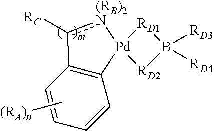

wherein

the dashed line represents the presence or absence of a bond;

Pd has a valency of +2;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring; and

R_{D1}, R_{D2}, R_{D3}, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl.

In some embodiments, n is 0, in which case the phenyl ring is unsubstituted. In some embodiments, n is 1. In some embodiments, n is 2. In some embodiments, n is 3. In some embodiments, n is 4. For the case where n is 1 or more, the substituents on the phenyl ring may have any substitution pattern.

In some embodiments, m is 0. In some embodiments, m is 1. In some embodiments, m is 2. In some embodiments, m is 3.

In some embodiments, the dashed line represents a bond, thus forming an imine moiety. In other embodiments, the dashed line represents the absence of a bond resulting in only a single bond between the carbon atom and nitrogen atom.

In some embodiments, at least one R_{A} is halogen. In some embodiments, at least one occurrence of R_{A} is aliphatic. In some embodiments, at least one occurrence of R_{A} is C₁-C₆ alkyl. In some embodiments, at least one occurrence of R_{A} is methyl. In some embodiments, at least one occurrence of R_{A} is ethyl. In some embodiments, at least one occurrence of R_{A} is propyl. In some embodiments, at least one occurrence of R_{A} is butyl. In some embodiments, at least one occurrence of R_{A} is heteroaliphatic. In some embodiments, at least one occurrence of R_{A} is acyl. In some embodiments, at least one occurrence of R_{A} is aryl. In some embodiments, at least one occurrence of R_{A} is heteroaryl. In some embodiments, at least one occurrence of R_{A} is —OR′. In some embodiments, at least one occurrence of R_{A} is —N(R′)₂. In some embodiments, at least one occurrence of R_{A} is —SR′. In some embodiments, at least one occurrence of R_{A} is —NO₂. In some embodiments, at least one occurrence of R_{A} is —CN. In some embodiments, at least one occurrence of R_{A} is —SCN.

In some embodiments, two occurrences of R_{A} taken together form a cyclic moiety. Such a cyclic moeity may be carbocyclic or heterocyclic. In some embodiments, the cyclic moiety is a substituted or unsubstituted phenyl moiety. In some embodiments, the cyclic moiety is an unsubstituted phenyl moiety. In some embodiments, the cyclic moiety is a substituted or unsubstituted heteroaryl moiety.

In some embodiments, at least one occurrence of R_{B} is hydrogen. In some embodiments, both R_{B} are hydrogen. In some embodiments, at least one occurrence of R_{B} is aliphatic. In some embodiments, both occurrences of R_{B} are aliphatic. In some embodiments, both occurrences of R_{B} are C₁-C₆ alkyl. In some embodiments, both occurrences of R_{B} are methyl. In some embodiments, both occurrences of R_{B} are ethyl. In some embodiments, both occurrences of R_{B} are propyl. In some embodiments, both occurrences of R_{B} are butyl. In some embodiments, at least one occurrence of R_{B} is heteroaliphatic. In some embodiments, both occurrences of R_{B} are heteroaliphatic. In some embodiments, at least one occurrence of R_{B} is acyl. In some embodiments, at least one occurrence of R_{B} is aryl. In some embodiments, at least one occurrence of R_{B} is heteroaryl.

In some embodiments, both R_{B} are the same. In some embodiments, the two R_{B} are different.

In some embodiments, both R_{B} are taken together to form a heterocyclic moiety. In some embodiments, both R_{B} are taken together to form a 5-membered heterocyclic moiety. In some embodiments, both R_{B} are taken together to form a 6-membered heterocyclic moiety. In some embodiments, both R_{B} are taken together to form an optionally substituted heteroaryl moiety.

In some embodiments, one R_{B} moity is covalently attached to a methylene group connecting the phenyl ring to the N atom, thus forming a heterocyclic moiety. Such a heterocyclic moiety may be a heteroaryl moiety. For example, in some embodiments, the heterocyclic moiety is a pyridinyl moiety.

In some embodiments, R_{D1}, R_{D2}, R_{D3} and R_{D4} all represent optionally substituted heteroaryl moieties. In some embodiments, at least one of R_{D1}, R_{D2}, R_{D3} and R_{D4} is an unsubstituted heteroaryl moiety. In some embodiments, R_{D1}, R_{D2}, R_{D3} and R_{D4} are all unsubstituted heteroaryl moieties. In some embodiments, R_{D1}, R_{D2}, R_{D3} and R_{D4} are all optionally substituted 5-membered heteroaryl moieties. In some embodiments, R_{D1}, R_{D2}, R_{D3} and R_{D4} are all nitrogen-containing 5-membered heteroaryl moieties, which are optionally substituted. In some embodiments, R_{D1}, R_{D2}, R_{D3} and R_{D4} are all optionally substituted pyrazolyl moieties. In some embodiments, R_{D1}, R_{D2}, R_{D3} and R_{D4} are all optionally substituted imidazolyl moieties. In some embodiments, R_{D1}, R_{D2}, R_{D3} and R_{D4} are all optionally substituted pyrrolyl moieties. In some embodiments, R_{D1}, R_{D2}, R_{D3} and R_{D4} are all are optionally substituted thiazolyl moieties. In some embodiments, R_{D1}, R_{D2}, R_{D3} and R_{D4} are all optionally substituted oxazolyl moieties. In some embodiments, R_{D1}, R_{D2}, R_{D3} and R_{D4} are all optionally substituted 6-membered heteroaryl moieties. In some embodiments, R_{D1}, R_{D2}, R_{D3} and R_{D4} are all nitrogen-containing 6-membered heteroaryl moieties, which are optionally substituted. In some embodiments, R_{D1}, R_{D2}, R_{D3} and R_{D4} are all optionally substituted pyridinyl moieties. In some embodiments, R_{D1}, R_{D2}, R_{D3} and R_{D4} are all optionally substituted pyrazinyl moieties. In some embodiments, R_{D1}, R_{D2}, R_{D3} and R_{D4} are all optionally substituted pyrimidinyl moieties. In some embodiments, R_{D1}, R_{D2}, R_{D3} and R_{D4} are all optionally substituted pyridazinyl moieties. In some embodiments, all of R_{D1}, R_{D2}, R_{D3} and R_{D4} of the borate ligand are the same. In other embodiments, all of R_{D1}, R_{D2}, R_{D3} and R_{D4} of the borate ligand are not the same. For example, a combination of heterocycle may constitute the borate ligand. In some embodiments, a combination of heteroaryl moieties may constitute the borate ligand.

In some embodiments, the intermediate comprises a bidentate ligand of one of the formulae:

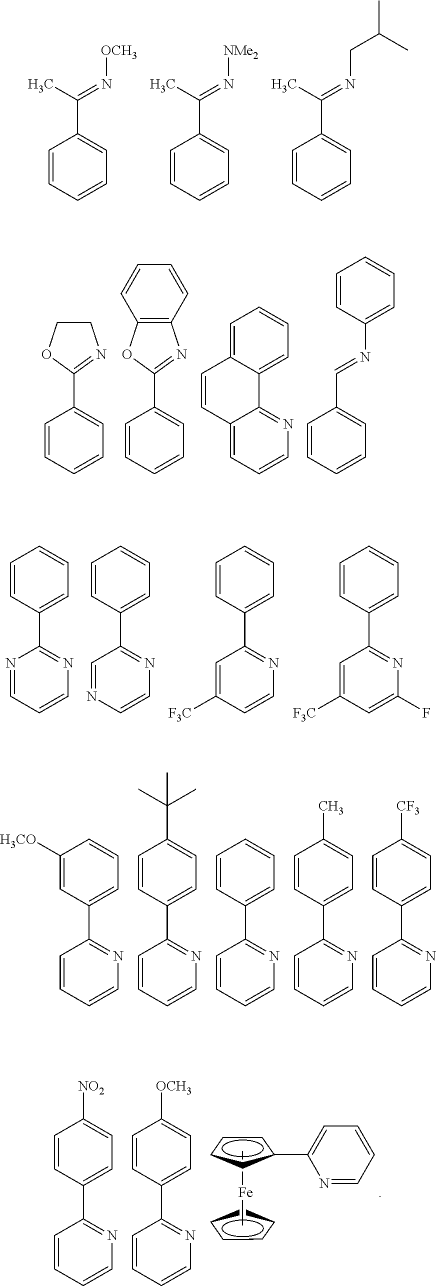

These ligands make a five-membered ring with the palladium atom with the nitrogen and a carbon coordinated to the central palladium.

In some embodiments, the intermediate is of the formula:

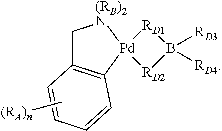

In some embodiments, the intermediate is of the formula:

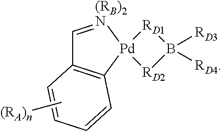

In some embodiments, the intermediate is of the formula:

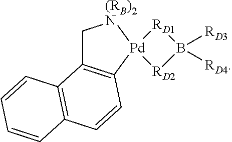

In some embodiments, the intermediate is of the formula:

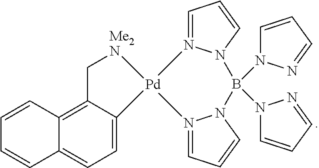

As can be appreciated by the skilled artisan, alternative methods of synthesizing the compounds of the formulae herein will be evident to those of ordinary skill in the art.

Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Methods of Treatment

The compounds and compositions described herein can be administered to cells in culture, e.g. in vitro or ex vivo, or to a subject, e.g., in vivo, to treat, prevent, and/or diagnose a variety of disorders, including those described herein below.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

Described herein are compounds and compositions useful as pharmaceutical agents. In general, the compounds described herein are fluorinated derivatives of a pharmaceutical agent. Also envisioned herein are other pharmaceutical agents and agents for treating a disorder described herein, wherein one or more fluorine moieties have been added to the pharmaceutical agent, e.g., replacing a hydrogen or functional group such as an —OH with a fluorine.

Antibiotics

Antibiotics are chemotherapeutic agents with activity against microorganisms such as bacteria. Antibacterial action generally falls within one of four mechanisms, three involve the inhibition or regulation of enzymes involved in cell wall biosynthesis, nucleic acid metabolism and repair, or protein synthesis. The fourth mechanism involves the disruption of membrane structure, like a pore-former, a common one being polymixin B.

Exemplary diseases caused by bacteria include, e.g., Anthrax, Bacterial Meningitis, Botulism, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme disease, Melioidosis, Rheumatic Fever, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus, and Urinary

Tract Infections.

Exemplary bacterial pathogens include, e.g., Acinetobacter baumannii, Bacillus anthracis, Bacillus subtilis, Chlamydia pneumoniae, Clostridium perfringens, Coagulase Negative Staphylococcus, E. coli, Enterococcus faecalis, Enterococcus faecium, Enterobacter sps., Haemophilus influenzae, Helicobacter pylori, Klebsiella pneumoniae, Legionella pneumophila, Moraxella catarralis, Mycoplasma pneumoniae, Mycobacterium tuberculosis, Preteus mirabilis, Proteus sps., Pseudomonas aeruginosa, Salmonella typhi, Serratia marcesens, Shigella flexneri, Staphylococcus aureus, Staphylococcus epidermidis, mutans, Streptococcus pneumoniae, and Streptococcus pyogenes. Bacterial pathogens may also include bacteria that cause resistant bacterial infections.

General Classes of Antibiotics and Mechanism

Aminoglycosides: Inhibit protein synthesis by binding to a portion of the bacterial ribosome. Most of them are bacteriocidal (i.e., cause bacterial cell death).

Bacitracin: Inhibits cell wall production by blocking the step in the process (recycling of the membrane lipid carrier) which is needed to add on new cell wall subunits.

Beta-lactam antibiotics: Antibiotics in this group contain a specific chemical structure (i.e., a beta-lactam ring). This includes penicillins, cephalosporins, carbapenems and monobactams. They inhibit the synthesis of the peptidoglycan layer of bacterial cell walls, Gram positive, by binding to PBP, penicillin binding protein, that is the last step in cell wall synthesis. Although, some Gram negative organisms seem to be susceptible.

Cephalosporins: These are similar to penicillins in their mode of action but they treat a broader range of bacterial infections. They have structural similarities to penicillins and many subjects with allergies to penicillins also have allergic reactions to cephalosporins.

Chloramphenicol: Inhibits protein synthesis by binding to a subunit of bacterial ribosomes (50S).

Glycopeptides (e.g., vancomycin): Interfere with cell wall development by blocking the attachment of new cell wall subunits (muramyl pentapeptides).

Macrolides (e.g., erythromycin) and Lincosamides (e.g., clindamycin): Inhibit protein synthesis by binding to a subunit of the bacterial ribosome (50S).

Penicillins: Inhibits formation of the bacterial cell wall by blocking cross-linking of the cell wall structure. The cell wall is a needed protective casing for the bacterial cell.

Quinolones: Blocks DNA synthesis by inhibiting one of the enzymes (DNA gyrase) needed in this process. (ciprofloxacin is a fluoroquinolone)

Rifampin: Inhibits RNA synthesis by inhibiting one of the enzymes (DNA-dependent RNA polymerase) needed in this process. RNA is needed to make proteins.

Glycopeptide: Like vancoymcin, inhibits cell wall synthesis.

Tetracyclines: Inhibit protein synthesis by binding to the subunit of the bacterial ribosome (30S subunit).

Trimethoprim and Sulfonamides: Blocks cell metabolism by inhibiting enzymes which are needed in the biosynthesis of folic acid which is a necessary cell compound.

Antivirals

Antiviral drugs are a class of medication used specifically for treating viral infections. Antiviral action generally falls into one of three mechanisms: to interfere with the ability of a virus to infiltrate a target cell (e.g., amantadine, rimantadine and pleconaril), to inhibit the synthesis of virus (e.g., nucleoside analogues, e.g., acyclovir and zidovudine (AZT)), and to inhibit the release of virus (e.g., zanamivir and oseltamivir).

Exemplary viral diseases include acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, and herpes zoster.

Exemplary viral pathogens include Adenovirus, Coxsackievirus, Dengue virus, Encephalitis Virus, Epstein-Barr virus, Hepatitis A virus, Hepatitis B virus, Hepatitis C virus, Herpes simplex virus type 1, Herpes simplex virus type 2, cytomegalovirus, Human herpesvirus type 8, Human immunodeficiency virus, Influenza virus, measles virus, Mumps virus, Human papillomavirus, Parainfluenza virus, Poliovirus, Rabies virus, Respiratory syncytial virus, Rubella virus, Varicella-zoster virus, West Nile virus, and Yellow fever virus. Viral pathogens may also include viruses that cause resistant viral infections.

Antifungals

Fungi are the causal agents of a variety of diseases in humans including, but not limited to Aspergilloses, Blastomycosis, Candidasis, Coccidioidomycosis, Cryptococcosis, Histoplasmosis, Mycetomas, Paracoccidioidomycosis, and Tinea pedis.

Furthermore, persons with immuno-deficiencies are particularly susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

An antifungal drug is a medication used to treat fungal infections such as athlete's foot, ringworm, candidiasis (thrush), serious systemic infections such as cryptococcal meningitis, and others.

General Classes of Antifungals and Mechanism

Polyene antifungals: bind with sterols in the fungal cell membrane, principally ergosterol, and changes the transition temperature (Tg) of the cell membrane. Imidazole and Triazole antifungals: inhibit the enzyme cytochrome P450 14α-demethylase, which is required in fungal cell membrane synthesis.

Allylamines: inhibit the enzyme squalene epoxidase, which is required for ergosterol synthesis.

Echinocandins: inhibit the synthesis of glucan in the cell wall, probably via the enzyme 1,3-β glucan synthase.

Antihypertensives

Antihypertensives are a class of drugs that are used in medicine and pharmacology to treat hypertension.

Hypertension, also referred to as high blood pressure, HTN or HPN, is a medical condition in which the blood pressure is chronically elevated. Hypertension can be classified either essential (primary) or secondary. Essential hypertension indicates that no specific medical cause can be found to explain a patient's condition. Secondary hypertension indicates that the high blood pressure is a result of (i.e., secondary to) another condition, such as kidney disease or tumors (pheochromocytoma and paraganglioma). Hypertension can cause symptoms such as headache, dizziness, blurred vision, or nausea. Persistent hypertension is one of the risk factors for strokes, heart attacks, heart failure, arterial aneurysm, and chronic renal failure. Causes of hypertension include obesity, sodium sensitivity, rennin homeostasis, insulin resistance, sleep apnea, genetics, aging, liquorice, renal disease, adrenal cortical abnormalities, Cushing's syndrome, aortic coarctation, certain medications (e.g., NSAIDs and steroids), sudden withdrawal of various antihypertensive medications, and pregnancy.

General Classes of Antihypertensives and Mechanism

Diuretics: help the kidneys eliminate excess salt and water from the body's tissues and blood.

Adrenergic receptor antagonists, e.g., Beta blockers, Alpha blockers, and Mixed Alpha+Beta blockers: antagonize adrenergic receptor function

Adrenergic receptor agonists: agonize adrenergic receptor function.

Calcium channel blockers: block the entry of calcium into muscle cells in artery walls.

ACE inhibitors: inhibit the activity of Angiotensin-converting enzyme (ACE), an enzyme responsible for the conversion of angiotensin I into angiotensin II, a potent vasoconstrictor.

Angiotensin II receptor antagonists: antagonize the activation of angiotensin receptors.

Aldosterone antagonists: antagonise the action of aldosterone at mineralocorticoid receptors: often used as adjunctive therapy, in combination with other drugs, for the management of chronic heart failure.

Vasodilators: act directly on arteries to relax their walls so blood can move more easily through them.

Centrally acting adrenergic drugs: stimulate alpha-receptors in the brain which open peripheral arteries easing blood flow. These drugs are often administered in combination with a diuretic.

Adrenergic neuron blockers: block the function of adrenergic neuron.

Anti-Inflammatory

Inflammation is the complex biological response of vascular tissues to harmful stimuli, such as pathogens, damaged cells, or irritants. It is a protective attempt by the organism to remove the injurious stimuli as well as initiate the healing process for the tissue. Inflammation which runs unchecked can lead to a host of diseases, such as hay fever, atherosclerosis, and rheumatoid arthritis.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

The causes of inflammation include burns, chemical irritants, frostbite, toxins, infection by pathogens, necrosis, physical injury (e.g., blunt and penetration), immune reactions due to hypersensitivity, ionizing radiation, and foreign bodies (e.g., splinters and dirt).

Exemplary abnormalities associated with inflammation include Allergies, Asthma, Autoimmune diseases, cancer, Chronic inflammation, Chronic prostatitis, Glomerulonephritis, Hypersensitivities, Inflammatory bowel diseases, Leukocyte defects, Myopathies, Pelvic inflammatory disease, Reperfusion injury, Rheumatoid arthritis, Transplant rejection, Vasculitis.

Anti-inflammatory refers to the property of a substance or treatment that reduces inflammation. There are two types of anti-inflammatory drugs: Steroidal anti-inflammatory drugs and Non-steroidal anti-inflammatory drugs.

Many steroids, specifically glucocorticoids, reduce inflammation or swelling by binding to cortisol receptors. These drugs are often referred to as corticosteroids.

Non-steroidal anti-inflammatory drugs (NSAIDs), alleviate pain by counteracting the cyclooxygenase (COX) enzyme.

Antihistamines

Histamine is a biogenic amine involved in local immune responses as well as regulating physiological function in the gut and acting as a neurotransmitter. Most histamine in the body is generated in granules in mast cells or in white blood cells, e.g., basophils. Mast cells are especially numerous at sites of potential injury—the nose, mouth, and feet, internal body surfaces, and blood vessels. Non-mast cell histamine is found in several tissues, including the brain, where it functions as a neurotransmitter. Another important site of histamine storage and release is the enterochromaffin-like (ECL) cell of the stomach. The most important pathophysiologic mechanism of mast cell and basophil histamine release is immunologic. These cells, if sensitized by IgE antibodies attached to their membranes, degranulate when exposed to the appropriate antigen. Certain amines and alkaloids, including such drugs as morphine, and curare alkaloids, can displace histamine in granules and cause its release. Antibiotics like polymyxin are also found to be stimulating histamine release.

Histamine exerts its actions by combining with specific cellular histamine receptors. The four histamine receptors that have been discovered are designated H1 through H4. Histamine plays an important role in chemotaxis of white blood cells. Histamine is involved in immune system disorders and allergies.

Antihistamines or histamine antagonists are agents which serve to inhibit the release or action of histamine. Antihistamine can be used to describe any histamine antagonist, but it is usually reserved for the classical antihistamines that act upon the H1 histamine receptor.

Antihistamines are used as treatment for allergies, which indicate excessive release of histamines by the body.

General Classes of Antihistamines and Mechanism

H1-receptor antagonists: inverse agonists at the histamine H1-receptor. Clinically, H1 antagonists are used to treat allergic reactions.

H2-receptor antagonists: inverse agonists. H2 histamine receptors are found principally in the parietal cells of the gastric mucosa. H2 antagonists are used to reduce the secretion of gastric acid, treating gastrointestinal conditions including peptic ulcers and gastroesophageal reflux disease.

H3-receptor antagonists. H3 histamine receptors are expressed in the central nervous system and to a lesser extent the peripheral nervous system, where they act as autoreceptors in presynaptic histaminergic neurons, and also control histamine turnover by feedback inhibition of histamine synthesis and release. H3-receptor antagonists have a stimulant and nootropic effect.

H4-receptor antagonists. H4 histamine receptors are highly expressed in bone marrow and white blood cells and regulate zymosan-induced neutrophil release from bone marrow and subsequent infiltration in the pleurisy model along with L-selectin. H4-receptor antagonists have an immunomodulatory role.

Inhibitors of histamine release: stabilize the mast cells to prevent degranulation and mediator release.

Tricyclic antidepressants and antipsychotics: some (e.g., promethazine) show antihistamine effect.

Vitamin C: alleviate shock by inhibiting deaminizing proteins which release histamine.

Migraine

Migraine is a neurological syndrome characterized by altered bodily perceptions, headaches, and nausea. The typical migraine headache is unilateral and pulsating, lasting from 4 to 72 hours; symptoms include nausea, vomiting, photophobia (increased sensitivity to bright light), and hyperacusis (increased sensitivity to noise).

The types of migraine include migraine without aura, migraine with aura, Basilar type migraine, familiar hemiplegic migraine, abdominal migraine, acephalgic migraine, and menstrual migraine. Four phases for migraine include prodrome phase (occurs hours and days before headache), aura phase (immediately precedes headache), pain phase (also known as headache phase), and postdrome phase. Causes of migraine might include cortical spreading depression, inappropriate contraction and expansion of brain blood vessels, abnormal serotonin level, or irritated nerves in the brain stem.

Treatments for migraine include paracetamol or non-steroidal anti-inflammatory drug (NSAIDs).

Cardiovascular Disease

Cardiovascular disease or cardiovascular diseases refers to the class of diseases that involve the heart or blood vessels (arteries and veins). It is often used to refer to those related to atherosclerosis (arterial disease), which have similar causes, mechanisms, and treatments. Types of cardiovascular diseases include, e.g., Aneurysm, Angina, Atherosclerosis, Cerebrovascular Accident (Stroke), Cerebrovascular disease, Congestive Heart Failure, Coronary Artery Disease, and Myocardial infarction (Heart Attack). Exemplary biomarkers which may reflect a higher risk of cardiovascular disease include, e.g., higher fibrinogen and PAI-1 blood concentrations, elevated homocysteine, elevated blood levels of asymmetric dimethylarginine, high inflammation as measured by C-reactive protein, and elevated blood levels of brain natriuretic peptide (BNP).

An aneurysm is a localized, blood-filled dilation of a blood vessel caused by disease or weakening of the vessel wall. Aneurysms often occur in arteries at the base of the brain (the circle of Willis) and in the aorta (the main artery coming out of the heart, a so-called aortic aneurysm). As the size of an aneurysm increases, there is an increased risk of rupture, which can result in severe hemorrhage or other complications including death. Treatment of arterial aneurysms includes, e.g., surgical intervention, and minimally invasive endovascular techniques.

Angina pectoris, commonly known as angina, is severe chest pain due to ischemia (a lack of blood and hence oxygen supply) of the heart muscle, generally due to obstruction or spasm of the coronary arteries. Coronary artery disease (due to atherosclerosis of the cardiac arteries) is a main cause of angina. Treatments of angina pectoris include, e.g., relief of symptoms, slowing progression of the disease, and reduction of future events, especially heart attacks and death. Drugs used to treat angina include, e.g., aspirin, Beta blockers (e.g., carvedilol, propranolol and atenolol), nitroglycerin, Calcium channel blockers (e.g., nifedipine and amlodipine), isosorbide mononitrate, nicorandil, If inhibitor (e.g., ivabradine), ACE inhibitors, and statins.

Atherosclerosis is a disease affecting arterial blood vessels. It is a chronic inflammatory response in the walls of arteries, in large part due to the accumulation of macrophage white blood cells and promoted by low density lipoproteins (LDL) without adequate removal of fats and cholesterol from the macrophages by functional high density lipoproteins (HDL). It is commonly referred to as a “hardening” or “furring” of the arteries. It is caused by the formation of multiple plaques within the arteries. Treatments for atherosclerosis include, e.g., statins, diet and diet supplements, surgical intervention, and prophylaxis.

A Cerebrovascular Accident (stroke) is the rapidly developing loss of brain functions due to a disturbance in the blood vessels supplying blood to the brain. This can be due to ischemia (lack of blood supply) caused by thrombosis or embolism (in ischemic stroke) or due to a hemorrhage (in hemorrhagic stroke). Risk factors for stroke include advanced age, hypertension, previous stroke or transient ischemic attack (TIA), diabetes, high cholesterol, cigarette smoking and atrial fibrillation. Treatments of ischemic stroke include, e.g., pharmacologic thrombolysis with drugs (e.g., tissue plasminogen activator (tPA)), mechanical thrombectomy, and therapeutic hypothermia. Treatments of hemorrhagic stroke include, e.g., neurosurgical evaluation to detect and treat the cause of the bleeding, and keeping blood pressure, blood sugar, and oxygenation at optimum levels.

Cerebrovascular disease is a group of brain dysfunctions related to disease of blood vessels supplying the brain. Hypertension is an important cause that damages the blood vessel lining endothelium exposing the underlying collagen where platelets aggregate to initiate a repairing process which is not always complete and perfect. Risk factors include, e.g., aging, diabetes, smoking, and ischemic heart disease. Treatments include, e.g. surgical interventions, endovascular procedures, and antiplatelet agents.

Congestive Heart Failure is a condition in which a problem with the structure or function of the heart impairs its ability to supply sufficient blood flow to meet the body's needs. Common causes of heart failure include myocardial infarction and other forms of ischemic heart disease, hypertension, valvular heart disease and cardiomyopathy. Heart failure can cause a large variety of symptoms such as shortness of breath (e.g., orthopnea), coughing, ankle swelling and reduced exercise capacity. Treatments commonly include, e.g., lifestyle measures (such as decreased salt intake) and medications, and sometimes devices or surgery.

Coronary disease (or coronary heart disease) refers to the failure of coronary circulation to supply adequate circulation to cardiac muscle and surrounding tissue. The typical cause of coronary heart disease is a condition known as atherosclerosis, which takes place with plaque and fatty build up on the artery walls, narrowing the vessels. The risk factors include, e.g., aging, diabetes, genetics, high blood pressure, high level of LDL, increased levels of C-reactive protein, fibrinogen, or homocysteine, lack of sufficient physical activity, low level of cholesterol (HDL), menopause, obesity, and smoking. Symptoms include, e.g., angina, heart attack, and shortness of breath. Treatments include, e.g., angioplasty with stenting, coronary artery bypass surgery, medication, minimally invasive heart surgery, proper diet and exercise, and quitting smoking.

Myocardial infarction (MI or AMI for acute myocardial infarction), commonly known as a heart attack, occurs when the blood supply to part of the heart is interrupted. This is often due to occlusion (blockage) of a coronary artery following the rupture of a vulnerable atherosclerotic plaque, which is an unstable collection of lipids (e.g., cholesterol) and white blood cells (e.g., macrophages) in the wall of an artery. The resulting ischemia (restriction in blood supply) and oxygen shortage can cause damage and/or death (infarction) of heart muscle tissue (myocardium). Symptoms of acute myocardial infarction include sudden chest pain, shortness of breath, nausea, vomiting, palpitations, sweating, and anxiety. Treatments of myocardial infarction include, e.g., first line agents (e.g., oxygen, aspirin, glyceryl trinitrate (nitroglycerin) and analgesia (e.g., morphine), beta blockers, anticoagulation (e.g., heparin), and antiplatelet agents (e.g., clopidogrel)), Reperfusion (e.g., Thrombolytic therapy, Percutaneous coronary intervention, Coronary artery bypass surgery), Monitoring for arrhythmias, Cardiac rehabilitation, and Secondary prevention.

Depressive Disorders

Depressive disorders include Major depressive disorder, Dysthymia, Depressive Disorder Not Otherwise Specified (DD-NOS), Recurrent brief depression (RBD), and Minor depression.

Major depressive disorder (also known as clinical depression, major depression, unipolar depression, or unipolar disorder) is a mental disorder characterized by a pervasive low mood, low self-esteem, and loss of interest or pleasure in normally enjoyable activities. Types of Major depressive disorder include, e.g., Atypical depression, Melancholic depression, Psychotic depression, Catatonic depression, Postpartum depression, and Seasonal affective disorder.

Dysthymia is a mood disorder that falls within the depression spectrum. It is considered a chronic depression, but with less severity than major depressive disorder. This disorder tends to be a chronic, long-lasting illness.

Depressive Disorder Not Otherwise Specified (DD-NOS) is a depressive disorder that is impairing but do not fit any the officially specified diagnoses. Examples of disorders in this category include those sometimes described as Minor Depressive Disorder and Recurrent Brief Depressive Disorder.

Recurrent Brief Depression (RBD) is a mental disorder characterized by intermittent depressive episodes, in women not related to menstrual cycles, occurring at least once a month over at least one year or more fulfilling the diagnostic criteria for major depressive episodes except for duration which in RBD is less than 14 days, typically 2-4 days. Despite the short duration of the depressive episodes, such episodes are severe and suicidal ideation and impaired function is rather common.

General Classes of Antidepressant and Mechanism

Selective serotonin reuptake inhibitors (SSRIs): prevent the reuptake of serotonin (also known as 5-hydroxytryptamine, or 5-HT) by the presynaptic neuron, thus maintaining higher levels of 5-HT in the synapse.

Serotonin-norepinephrine reuptake inhibitors (SNRIs): work on both norepinephrine and 5-HT.

Noradrenergic and specific serotonergic antidepressants (NASSAs): increase norepinephrine (noradrenaline) and serotonin neurotransmission by blocking presynaptic alpha-2 adrenergic receptors while at the same time minimizing serotonin related side-effects by blocking certain serotonin receptors.

Norepinephrine (noradrenaline) reuptake inhibitors (NRIs): have a positive effect on concentration and motivation via e.g., norepinephrine (noradrenaline).

Norepinephrine-dopamine reuptake inhibitors (NDRIs): inhibit the neuronal reuptake of dopamine and norepinephrine (noradrenaline).

Tricyclic antidepressants (TCAs): block the reuptake of certain neurotransmitters such as norepinephrine (noradrenaline) and serotonin.

Monoamine oxidase inhibitors (MAOIs): block the enzyme monoamine oxidase which breaks down the neurotransmitters dopamine, serotonin, and norepinephrine (noradrenaline).

### Movement Disorders

Exemplary movement disorders include, for example, Akathisia, Akinesia, Athetosis, Ataxia, Ballismus, Hemiballismus, Bradykinesia, Cerebral palsy, Chorea (e.g., Sydenham's chorea, Rheumatic chorea and Huntington's disease), Dystonia (e.g., Dystonia muscularum, Blepharospasm, Writer's cramp, and Spasmodic torticollis), Geniospasm Myoclonus, Parkinson's disease, Restless Legs Syndrome RLS (WittMaack-Ekboms disease), Spasms, Stereotypic movement disorder, Stereotypy, Tardive dyskinesia, Tic disorders, Tourette's syndrome, Tremor, and Wilson's disease. Treatment generally depends upon the underlying disorder.

Parkinson's Disease

Parkinson's disease (Parkinson disease or PD) is a degenerative disorder of the central nervous system that often impairs the sufferer's motor skills, speech, and other functions. It is characterized by muscle rigidity, tremor, a slowing of physical movement (bradykinesia) and, in extreme cases, a loss of physical movement (akinesia). The primary symptoms are the results of decreased stimulation of the motor cortex by the basal ganglia, normally caused by the insufficient formation and action of dopamine, which is produced in the dopaminergic neurons of the brain. Motor symptoms can include, e.g., tremor, rigidity, Akinesia/bradykinesia, postural instability, speech and swallowing disturbances. Secondary symptoms may include high level cognitive dysfunction and subtle language problems, such as slowed reaction time, executive dysfunction, dementia, and short-term memory loss. PD is both chronic and progressive. Causes for PD include, e.g., genetic mutations, toxins, and head trauma.

There are other disorders that are called Parkinson-plus diseases. These include: e.g., multiple system atrophy (MSA), progressive supranuclear palsy (PSP) and corticobasal degeneration (CBD). These Parkinson-plus diseases may progress more quickly than typical idiopathic Parkinson disease.

Treatments for PD include, e.g., Levodapa, COMT inhibitors, Dopamine agonists, MAO-B inhibitors, surgery and deep brain stimulation, and neurorehabilitation.

Epilepsy

Epilepsy is a common chronic neurological disorder that is characterized by recurrent unprovoked seizures. These seizures are transient signs and/or symptoms due to abnormal, excessive or synchronous neuronal activity in the brain. There are many different epilepsy syndromes, each presenting with its own unique combination of seizure type, typical age of onset, EEG findings, treatment, and prognosis. Exemplary epilepsy syndromes include, e.g., Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, West syndrome, Dravet's syndrome, Progressive myoclonic epilepsies, and Lennox-Gastaut syndrome (LGS).

Genetic, congenital, and developmental conditions are often associated with epilepsy among younger patients. Tumors might be a cause for patients over age 40. Head trauma and central nervous system infections may cause epilepsy at any age.

Treatments include, e.g., surgical treatment, pharmacologic treatment, Ketogenic diet, electrical stimulation, Vagus Nerve Stimulation (VNS), Responsive Neurostimulator System (RNS), Deep brain stimulation (DBS), Noninvasive surgery, Avodance therapy, Warning systems, and alternative or complementary medicine.

Antipsychotic Compounds

Antipsychotics are a group of psychoactive drugs commonly but not exclusively used to treat psychosis, e.g., schizophrenia, mania and dementia. Psychosis means abnormal condition of the mind, and is a generic psychiatric term for a mental state often described as involving a “loss of contact with reality”. Symptoms of psychosis may have one or more of the following: hallucinations, delusions, thought disorder, or lack of insight. The symptoms are similar in nature to mental confusion and delirium.

Causes of psychosis are generally classified as “organic” or “functional”. Organic conditions were primarily medical or pathophysiological, whereas, functional conditions are primarily psychiatric or psychological. Functional causes of psychosis include, e.g., brain tumors, drug abuse, schizophrenia, bipolar disorder, severe clinical depression, severe psychosocial stress, sleep deprivation, some focal epileptic disorders, and exposure to some traumatic event. Pathophysiological causes include, e.g., neurological disorders, electrolyte disorders, hypoglycemia, lupus, AIDS, leprosy, malaria, Adult-onset vanishing white matter leukoencephalopathy, Late-onset metachromatic leukodystrophy, Cerebral involvement of scleroderma, and Hashimoto's encephalopathy.

Many antipsychotic drugs tend to block D2 receptors in the dopamine pathways of the brain. Typical antipsychotics are not particularly selective and also block Dopamine receptors in the mesocortical pathway, tuberoinfundibular pathway, and the nigrostriatal pathway. Atypical antipsychotic drugs have a similar blocking effect on D2 receptors. Some also block or partially block serotonin receptors (particularly 5HT2A, C and 5HT1A receptors): ranging from risperidone, which acts overwhelmingly on serotonin receptors, to amisulpride, which has no serotonergic activity.

Exemplary antipsychotics include, e.g., Butyrophenones, Phenothiazines, Thioxanthenes, Clozapine, Olanzapine, Risperidone, Quetiapine, Ziprasidone, Amisulpride, Asenapine and Paliperidone, Aripiprazole, Dopamine partial agonists, Bifeprunox, norclozapine, Tetrabenazine, Cannabidiol, and Metabotropic glutamate receptor 2 agonists.

Mania

Mania is a severe medical condition characterized by extremely elevated mood, energy, unusual thought patterns and sometimes psychosis.

There are several possible causes for mania including drug abuse and brain tumors, but it is often associated with bipolar disorder, where episodes of mania may cyclically alternate with episodes of major depression. These cycles may relate to diurnal rhythms and environmental stressors. Mania varies in intensity, from mild mania (known as hypomania) to full-blown mania with psychotic features (hallucinations and delusions).

Symptoms of mania include rapid speech, racing thoughts, decreased need for sleep, hypersexuality, hypersensitivity, hyper-religiosity, hyperactivity, euphoria, impulsiveness, grandiosity, irritability, anger or rage, delusions, and increased interest in goal-directed activities. Mild forms of mania, known as hypomania, cause little or no impairment.

Acute mania in bipolar disorder can be treated with mood stabilizers and/or antipsychotic medication. They work by blocking the receptor for the neurotransmitter dopamine and allowing serotonin to still work, but in diminished capacity. Long-term treatment can focus on prophylactic treatment to try to stabilize the patient's mood, e.g., through a combination of pharmacotherapy and psychotherapy. Lithium is a mood stabilizer that can be used to prevent further manic and depressive episodes. Anticonvulsants such as valproic acid and carbamazepine can also be used for prophylaxis. Other exemplary drug solutions include, e.g., lamotrigine and Clonazepam. The calcium-channel blocker, verapamil is useful in the treatment of hypomania.

Dementia

Dementia is the progressive decline in cognitive function due to damage or disease in the body beyond what might be expected from normal aging. Dementia is a non-specific illness syndrome in which affected areas of cognition may be memory, attention, language, and problem solving. Symptoms of dementia can be classified as either reversible or irreversible, depending upon the etiology of the disease.

Types of dementia include, e.g., Cortical dementias, e.g., Alzheimer's disease, Vascular dementia including Binswanger's disease, Dementia with Lewy bodies (DLB), Alcohol-Induced Persisting Dementia (e.g., Korsakoffs syndrome and Wernicke's encephalopathy), Frontotemporal lobar degenerations (FTLD), (e.g., Pick's disease, Frontotemporal dementia, Semantic dementia, and Progressive non-fluent aphasia), Creutzfeldt-Jakob disease, Dementia pugilistica, Moyamoya disease, Thebestia, Posterior cortical atrophy (Benson's syndrome); Subcortical dementias, e.g., Dementias due to Huntington's disease, Hypothyroidism, Parkinson's disease, Vitamin B1 deficiency, Vitamin B12 deficiency, Folate deficiency, Syphilis, Subdural hematoma, Hypercalcaemia, Hypoglycemia, AIDS, multiple etiologies, other general medical conditions, and dementia not otherwise specified.

Treatment for dementia include, e.g., Cholinesterase inhibitors, Acetylcholinesterase inhibitors, N-methyl-D-aspartate Blockers, Amyloid deposit inhibitors, Antipsychotic drugs, Antidepressant drugs, Anxiolytic drugs, and selegiline.

Pain

Pain is unpleasant sensation induced by noxious stimuli and generally received by specialized nerve endings.

Pain or sensitivity to pain and touch may be indicated in a variety of diseases, disorders or conditions, including, but not limited to, diabetic neuropathy, cardiovascular disease, breast pain, psoriasis, eczema, dermatitis, burn, post-herpetic neuralgia (shingles), nociceptive pain, peripheral neuropathic and central neuropathic pain, chronic pain, cancer and tumor pain, spinal cord injury, crush injury and trauma induced pain, migraine, cerebrovascular and vascular pain, sickle cell disease pain, rheumatoid arthritis pain, musculoskeletal pain including signs and symptoms of osteoarthritis and rheumatoid arthritis, orofacial and facial pain including dental, temporomandibular disorder and cancer related, lower back or pelvic pain, surgical incision related pain, inflammatory and non-inflammatory pain, visceral pain, psychogenic pain, soft tissue inflammatory pain, fibromyalgia-related pain, reflex sympathetic dystrophy, and pain resulting from kidney stones or urinary tract infection. The compounds and methods of the invention may be used in the treatment of chronic, as well as acute pain. Chronic or acute pain may be the result of injury, age, or disease.

There are at least three major types of pain: nociceptive, neuropathic and psychogenic. Nociceptive pain results from tissue damage. Types of nociceptive pain include superficial somatic pain (or cutaneous pain), deep somatic pain, and visceral pain. Neuropathic pain may occur when there is damage to or dysfunction of nerves in the peripheral or central nervous system. Psychogenic pain is pain associated with psychological factors.

Brain areas in relation with pain include the somatosensory cortex which mostly accounts for the sensory discriminative dimension of pain, and the limbic system, of which the thalamus and the anterior cingulate cortex are involved in the affective dimension. Ion channels have been implicated in reception or transmission of pain. For example, the involvement of N-type calcium channels in the synaptic transmissions that convey pain signals from sensory afferent nerve cells to the central nervous system has been recognized. Certain naturally occurring peptide neurotoxins that specifically block N-type calcium channel have been shown to act as extremely potent and efficient analgesics in a wide range of animal pain models, including models of inflammatory and neuropathic pain. The available evidence suggests that N-type calcium channel blockers are at least as efficacious as opiates, are devoid of a number of the typical opiate side effects (e.g. respiratory depression) and that the analgesic effect is not subject to tolerance development.

Pain can be generally managed or treated by a variety of approaches, including a multidisciplinary approach that includes pharmacologic measures (analgesics such as narcotics or NSAIDs and pain modifiers such as tricyclic antidepressants or anticonvulsants), non-pharmacologic measures (such as interventional procedures, physical therapy and physical exercise, application of ice and/or heat), and psychological measures (such as biofeedback and cognitive therapy).

Exemplary animal models of pain include, but are not limited to, the Chung model (Chung et al. (2004) Methods Mol Med 99: 35-45; Kim and Chung (1992) Pain 50: 355-363), the carageenan induced hyperalgesia model and the Freund's complete adjuvant induced hyperalgesia model (Walker et al. (2003) Journal of Pharmacol Exp Ther 304: 56-62; McGaraughty et al. (2003) Br J Pharmacol 140: 1381-1388; Honore et al. (2005) J Pharmacol Exp Ther.), the thermal injury model (Jones and Sorkin, 1998, Brain Res 810: 93-99; Nozaki-Taguchi and Yaksh, 1998, Neuroscience Lett 254: 25-28; Jun and Yaksh, 1998, Anesth Analg 86: 348-354), the formalin model (Yaksh et al., 2001, J Appl Physiol 90: 2386-2402), the Bennett Model (Xanthos et al. (2004) J Pain 5: S1), the rat neurogenic inflammation model, (see Buzzi et al (1990) Br J Pharmacol; 99:202-206), the Burstein Model (see Strassman et al., (1996) Nature 384: 560-564), CFA model (Nagakura et al., 2003, J Pharmacol Exp Ther 306: 490-497), and the acute pain model (Valenzano et al. (2005) Neuropharmacology 48: 658-672).

Disorders for which Diuretic Treatment is Indicated

A diuretic is any drug that elevates the rate of urination and thus provides a means of forced diuresis. Diuretics are used to treat a variety of disorders, including, for example, heart failure, liver cirrhosis, hypertension and certain kidney diseases. Some diuretics help to make the urine more alkaline and are helpful in increasing excretion of substances such as aspirin in cases of overdose or poisoning.

Enlarged Prostate

Benign prostatic hyperplasia (BPH) also known as nodular hyperplasia, benign prostatic hypertrophy (technically a misnomer) or benign enlargement of the prostate (BEP) refers to the increase in size of the prostate in middle-aged and elderly men. It is characterized by hyperplasia of prostatic stromal and epithelial cells, resulting in the formation of large, fairly discrete nodules in the periurethral region of the prostate. When sufficiently large, the nodules compress the urethral canal to cause partial, or sometimes virtually complete, obstruction of the urethra which interferes the normal flow of urine.

Benign prostatic hyperplasia symptoms are classified as storage or voiding. Storage symptoms include urinary frequency, urgency (compelling need to void that can not be deferred), urgency incontinence and voiding at night (nocturia). Voiding symptoms include weak urinary stream, hesitency (needing to wait for the stream to begin), intermittency (when the stream starts and stops intermittently), straining to void, dysuria (burning sensation in the urethra), and dribbling.

BPH can be treated with medication (e.g., Alpha blockers and 5α-reductase inhibitors), a minimally invasive procedure or, in extreme cases, surgery that removes the prostate. Minimally invasive procedures include Transurethral needle ablation of the prostate (TUNA) and Transurethral microwave thermotherapy (TUMT). These outpatient procedures may be followed by the insertion of a temporary Prostatic stent, to allow normal voluntary urination, without exacerbating irritative symptoms.

Gastrointestinal Disorders

There are a number of diseases and conditions affecting the gastrointestinal system, including, e.g., Colorectal cancer, Diverticulitis, Gastroenteritis, Giardiasis, Inflammatory bowel disease (Crohn's disease and ulcerative colitis), Irritable bowel syndrome, Pancreatitis, Cholera and Peptic ulcer disease.

Colorectal cancer, also called colon cancer or large bowel cancer, includes cancerous growths in the colon, rectum and appendix. The first symptoms of colon cancer are usually vague, like bleeding, weight loss, and fatigue (tiredness). Local (bowel) symptoms are rare until the tumor has grown to a large size. Treatments include, e.g., surgery, chemotherapy, radiation therapy and immunotherapy.

Diverticulitis develops from diverticulosis, which involves the formation of pouches (diverticula) on the outside of the colon. Diverticulitis results if one of these diverticula becomes inflamed or infected. The most common symptom of diverticulitis is abdominal pain. Treatments include, e.g., conservative medical management, including bowel rest (i.e., no food taken by mouth), IV fluid resuscitation, and broad-spectrum antibiotics. Recurring acute attacks or complications, such as peritonitis, abscess, or fistula may require surgery.

Gastroenteritis (also known as gastro, gastric flu, and stomach flu, although unrelated to influenza) is inflammation of the gastrointestinal tract, involving both the stomach and the small intestine (see also gastritis and enteritis) and resulting in acute diarrhea. Treatments include, e.g., rehydration, antibiotics, antidiarrheal agent, and antiemetic drugs.

Giardiasis, popularly known as beaver fever or backpacker's diarrhea, is a disease caused by the flagellate protozoan Giardia lamblia (also sometimes called Giardia intestinalis and Giardia duodenalis). Symptoms include loss of appetite, fever, explosive diarrhea, hematuria (blood in urine), loose or watery stool, stomach cramps, upset stomach, projectile vomiting (uncommon), bloating, flatulence, and burping (often sulphurous). Treatments include, e.g., metronidazole, albendazole, quinacrine and timidazole.

Inflammatory bowel disease (IBD) is a group of inflammatory conditions of the large intestine and small intestine. The major types of IBD are Crohn's disease and ulcerative colitis. Treatment of IBD may require immunosuppression to control the symptom. Severe cases may require surgery.

Irritable bowel syndrome (IBS), also called spastic colon, is a functional bowel disorder characterized by mild to severe abdominal pain, discomfort, bloating and alteration of bowel habits. Treatments include, e.g., diet and medication.

Pancreatitis is the inflammation of the pancreas. There are two types of pancreatitis, acute and chronic. Treatments include, e.g., pain relief, adequate replacement fluids and salts, limitation of oral intake, monitoring and assessment for, and treatment of, the various complications.

Cholera, sometimes known as Asiatic or epidemic cholera, is an infectious gastroenteritis caused by enterotoxin-producing strains of the bacterium Vibrio cholerae. Cholera can be treated with oral rehydration therapy and antibiotics.

A peptic ulcer, also known as ulcus pepticum, PUD or peptic ulcer disease, is an ulcer (defined as mucosal erosions equal to or greater than 0.5 cm) of an area of the gastrointestinal tract that is usually acidic and thus extremely painful. Patients with ulcer-like symptoms are often treated with antacids or H2 antagonists before EGD is undertaken.

Hormone Therapy

Hormone therapy can be used to treat a condition or a hormone related disorder. Hormone replacement therapy (HRT) is a system of medical treatment for surgically menopausal, perimenopausal and to a lesser extent postmenopausal women, based on the assumption that the treatment may prevent discomfort caused by diminished circulating estrogen and progesterone hormones. It involves the use of one or more of a group of medications designed to artificially boost hormone levels. The main types of hormones involved are estrogens, progesterone or progestins, and sometimes testosterone. Types of hormone replacement therapy include, e.g., Conjugated equine estrogens (CEE) and Bioidentical hormone replacement therapy (BHRT).

Hyperparathyroidism

Hyperparathyroidism is overactivity of the parathyroid glands resulting in excess production of parathyroid hormone (PTH). The parathyroid hormone regulates calcium and phosphate levels and helps to maintain these levels. Overactivity of one or more of the parathyroid glands causes high calcium levels (hypercalcemia) and low levels of phosphate in the blood.

There are three types of hyperparathyroidism. Primary hyperparathyroidism results from a hyperfunction of the parathyroid glands themselves. Secondary hyperparathyroidism is the reaction of the parathyroid glands to a hypocalcemia caused by something other than a parathyroid pathology, e.g. chronic renal failure. Tertiary hyperparathyroidism is a state of autonomous hypersecretion of parathyroid hormone (PTH) due to chronic stimulation by hypocalcemia and resulting in hypercalcemia.

Common manifestations of hyperparathyroidism include weakness and fatigue, depression, aches and pains, decreased appetite, feelings of nausea and vomiting, constipation, polyuria, polydipsia, cognitive impairment, kidney stones and osteoporosis.

Treatments of primary hyperparathyroidism include surgical removal of the gland(s) containing adenomas and medication, e.g., estrogen replacement therapy in postmenopausal women and bisphosphonates. Treatments of secondary hyperparathyroidism include, e.g., dietary restriction of phosphorus, supplements with the active form of vitamin D (calcitriol), phosphate binders, and calcimimetics cinacalcet.

Glaucoma

Glaucoma is a group of diseases of the optic nerve involving loss of retinal ganglion cells in a characteristic pattern of optic neuropathy. Raised intraocular pressure is a significant risk factor for developing glaucoma. Untreated glaucoma leads to permanent damage of the optic nerve and resultant visual field loss, which can progress to blindness.

Glaucoma can be divided roughly into two main categories, “open angle” or chronic glaucoma and “closed angle” or acute glaucoma. Open angle, chronic glaucoma tends to progress more slowly and so the patient may not notice it until the disease has progressed quite significantly. Angle closure, acute glaucoma appears suddenly and often with painful side effects and so is usually diagnosed quickly, although damage and loss of vision can also occur very suddenly.

Symptoms include, e.g., patchy peripheral vision, visual loss, loss of contrast sensitivity.

Treatments include, e.g., medications and surgery (e.g., canaloplasty, laser surgery, trabeculectomy, glaucoma drainage implants, and veterinary implant).

Vomiting and Nausea

Vomiting and nausea (the sensation of unease and discomfort in the stomach with an urge to vomit) can be treated using an antiemetic agentAnti-emetics can also be used to treat motion sickness and the side effects of opioid analgesics, general anaesthetics and chemotherapy directed against cancer.

Nicotine Dependence

Nicotine acts on the brain to produce a number of effects. Specifically, its addictive nature has been found to show that nicotine activates reward pathways—the circuitry within the brain that regulates feelings of pleasure and euphoria.

Dopamine is one of the key neurotransmitters actively involved in the brain. By increasing the levels of dopamine within the reward circuits in the brain, nicotine acts as a chemical with intense addictive qualities. Nicotine causes down-regulation of the production of dopamine and other stimulatory neurotransmitters as the brain attempts to compensate for artificial stimulation. In addition, the sensitivity of nicotinic acetylcholine receptors decreases. To compensate for this compensatory mechanism, the brain in turn upregulates the number of receptors, convoluting its regulatory effects with compensatory mechanisms meant to counteract other compensatory mechanisms. The net effect is an increase in reward pathway sensitivity, opposite of other drugs of abuse such as cocaine and heroin, which reduce reward pathway sensitivity. This neuronal brain alteration persists for months after administration ceases.

Metabolic Disorders

A “metabolic disorder” is a disease or disorder characterized by an abnormality or malfunction of metabolism. One category of metabolic disorders is disorders of glucose, insulin, or lipid metabolism. For example, the subject can be insulin resistant, e.g., have insulin-resistance diabetes. In one embodiment, a compound described herein can be used to modify (e.g., decrease) insulin, glucose, or lipid levels in a subject. Treatment with a compound may be in an amount effective to improve one or more symptoms of the metabolic disorder.

Metabolic Syndrome

In some instances, the invention provides a method of treating metabolic syndrome, including administering to a subject an effective amount of a compound described herein.

The metabolic syndrome (e.g., Syndrome X) is characterized by a group of metabolic risk factors in one person. They include: central obesity (excessive fat tissue in and around the abdomen), atherogenic dyslipidemia (blood fat disorders—mainly high triglycerides and low HDL cholesterol—that foster plaque buildups in artery walls); insulin resistance or glucose intolerance (the body can't properly use insulin or blood sugar); prothrombotic state (e.g., high fibrinogen or plasminogen activator inhibitor [−1] in the blood); raised blood pressure (i.e., hypertension) (130/85 mmHg or higher); and proinflammatory state (e.g., elevated high-sensitivity C-reactive protein in the blood).

The underlying causes of this syndrome are overweight/obesity, physical inactivity and genetic factors. People with metabolic syndrome are at increased risk of coronary heart disease, other diseases related to plaque buildups in artery walls (e.g., stroke and peripheral vascular disease) and type 2 diabetes. Metabolic syndrome is closely associated with a generalized metabolic disorder called insulin resistance, in which the body can't use insulin efficiently.

Obesity

Obesity is a condition in which excess body fat has accumulated to such an extent that health may be negatively affected. Excessive body weight is associated with various diseases, including cardiovascular diseases, diabetes mellitus type 2, obstructive sleep apnea, certain types of cancer, and osteoarthritis. Obesity can be clinically assessed by, e.g., body mass index (BMI), waist circumference and waist-hip ratio, and body fat percentage. Causes for obesity include diet, sedentary lifestyle, genetics, medical and psychiatric illness, socioeconomic, and Gut Flora.

Obesity management include, e.g., behavioral therapy, dieting, exercise, medication, surgery, and clinical protocols.

Anti-obesity drugs operate through one or more of the following mechanisms:

Suppression of the appetite (e.g., Catecholamines and their derivatives (such as amphetamine-based drugs) and drugs blocking the cannabinoid receptors); increase of the body's metabolism; or interference with the body's ability to absorb specific nutrients in food (e.g., Orlistat, OTC fiber supplements glucomannan and guar gum).

Exemplary anti-obesity drugs include Orlistat, Sibutramine, Metformin, Byetta, Symlin and Rimonabant.

Diabetes

Diabetes mellitus, often referred to as diabetes, is a syndrome of disordered metabolism, usually due to a combination of hereditary and environmental causes, resulting in abnormally high blood sugar levels (hyperglycemia).

Type 1 diabetes mellitus is characterized by loss of the insulin-producing beta cells of the islets of Langerhans in the pancreas, leading to a deficiency of insulin. This type of diabetes can be further classified as immune-mediated or idiopathic. Type 2 diabetes mellitus is characterized differently due to insulin resistance or reduced insulin sensitivity, combined with relatively reduced, and sometimes absolute, insulin secretion. The defective responsiveness of body tissues to insulin often involves the insulin receptor in cell membranes. Gestational diabetes mellitus (GDM) resembles type 2 diabetes in several respects, involving a combination of relatively inadequate insulin secretion and responsiveness.

The classical triad of diabetes symptoms is polyuria, polydipsia and polyphagia, which are, respectively, frequent urination, increased thirst and consequent increased fluid intake, and increased appetite. Diabetes can cause many complications. Acute complications (hypoglycemia, ketoacidosis, or nonketotic hyperosmolar coma) may occur if the disease is not adequately controlled. Serious long-term complications include cardiovascular disease, chronic renal failure, retinal damage, nerve damage, and microvascular damage, which may cause erectile dysfunction and poor wound healing.

Both type 1 and type 2 diabetes are at least partly inherited. Type 1 diabetes appears to be triggered by some (mainly viral) infections, or less commonly, by stress or environmental exposure (such as exposure to certain chemicals or drugs). There is a genetic element in individual susceptibility to some of these triggers which has been traced to particular HLA genotypes. There is a stronger inheritance pattern for type 2 diabetes. Genes significantly associated with developing type 2 diabetes, include TCF7L2, PPARG, FTO, KCNJ11, NOTCH2, WFS1, CDKAL1, IGF2BP2, SLC30A8, JAZF1, and HHEX. Moreover, obesity (which is an independent risk factor for type 2 diabetes) is strongly inherited.

Treatment for Diabetes mellitus type 1 usually requires insulin. Treatments for Diabetes mellitus type 2 often include (1) agents which increase the amount of insulin secreted by the pancreas, (2) agents which increase the sensitivity of target organs to insulin, and (3) agents which decrease the rate at which glucose is absorbed from the gastrointestinal tract.

Exemplary anti-diabetic drugs include, e.g., Insulin, Secretagogues, Alpha-glucosidase inhibitors, Peptide analogs, DPP-4 inhibitors, Amylin analogues, PPARalpha/gamma ligands, SGLT inhibitors, and FBPase inhibitors.

Lipid-Modifying

Lipid-modifying drugs are a diverse group of pharmaceuticals that are used in the treatment of hyperlipidemias.

Hyperlipidemia is the presence of raised or abnormal levels of lipids and/or lipoproteins in the blood. Lipids (fatty molecules) are transported in a protein capsule, and the density of the lipids and type of protein determines the fate of the particle and its influence on metabolism. There are at least five types of Hyperlipidemia.

Hyperlipidemia type I is due to a deficiency of lipoprotein lipase (LPL) or altered apolipoprotein C2, resulting in elevated chylomicrons, the particles that transfer fatty acids from the digestive tract to the liver.

Hyperlipidemia type II is further classified into type Ha and type IIb, depending mainly on whether there is elevation in the triglyceride level in addition to LDL cholesterol. Type IIa may be sporadic, polygenic, or truly familial as a result of a mutation either in the LDL receptor gene on chromosome 19 or the ApoB gene. In Type IIb, the high VLDL levels are due to overproduction of substrates, including triglycerides, acetyl CoA, and an increase in B-100 synthesis. While dietary modification is the initial approach, many patients require treatment with statins (HMG-CoA reductase inhibitors) to reduce cardiovascular risk. If the triglyceride level is markedly raised, fibrates may be preferable due to their beneficial effects. Combination treatment of statins and fibrates, while highly effective, causes a markedly increased risk of myopathy and rhabdomyolysis and is therefore only done under close supervision. Other agents commonly added to statins are ezetimibe, niacin and bile acid sequestrants.

Hyperlipidemia type III is generally due to high chylomicrons and IDL (intermediate density lipoprotein).

Hyperlipidemia type IV is generally due to high triglycerides.

Hyperlipidemia type V is similar to type I, but with high VLDL in addition to chylomicrons. It is also associated with glucose intolerance and hyperuricemia

There are several classes of lipid-modifying drugs. They may differ in both their impact on the cholesterol profile and adverse effects. For example, some may lower the low density lipoprotein (LDL), e.g., LDL-C more so than others, while others may preferentially increase high density lipoprotein (HDL). Clinically, the choice of an agent will depend on the patient's cholesterol profile, cardiovascular risk, and the liver and kidney functions of the patient, evaluated against the balancing of risks and benefits of the medications.

Exemplary lipid-modifying drugs include, e.g., statins, fibrates, niacin, bile acid sequestrants (resins), ezetimibe, phytosterols, Orlistat, CETP Inhibitors, squalene synthase inhibitor, ApoA-1 Milano, and AGI-1067.

Cancer

Cancer (malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). Most cancers form a tumor but some, like leukemia, do not.

Cancers are often caused by abnormalities in the genetic material of the transformed cells. These abnormalities may be due to the effects of carcinogens, such as tobacco smoke, radiation, chemicals, or infectious agents. Other cancer-promoting genetic abnormalities may be randomly acquired through errors in DNA replication, or are inherited, and thus present in all cells from birth. The heritability of cancers is usually affected by complex interactions between carcinogens and the host's genome. Other aspects of the genetics of cancer pathogenesis, such as DNA methylation, and microRNAs are also recognized as important. Genetic abnormalities found in cancer typically affect two general classes of genes. Cancer-promoting oncogenes are typically activated in cancer cells, giving those cells new properties, such as hyperactive growth and division, protection against programmed cell death, loss of respect for normal tissue boundaries, and the ability to become established in diverse tissue environments. Tumor suppressor genes are then inactivated in cancer cells, resulting in the loss of normal functions in those cells, such as accurate DNA replication, control over the cell cycle, orientation and adhesion within tissues, and interaction with protective cells of the immune system.

Cancer symptoms can be divided into three groups, e.g., Local symptoms: unusual lumps or swelling (tumor), hemorrhage (bleeding), pain and/or ulceration. Compression of surrounding tissues may cause symptoms such as jaundice (yellowing the eyes and skin); Symptoms of metastasis (spreading): enlarged lymph nodes, cough and hemoptysis, hepatomegaly (enlarged liver), bone pain, fracture of affected bones and neurological symptoms; and Systemic symptoms: weight loss, poor appetite, fatigue and cachexia (wasting), excessive sweating (night sweats), anemia and specific paraneoplastic phenomena, i.e. specific conditions that are due to an active cancer, such as thrombosis or hormonal changes.

Exemplary cancers include, e.g., solid tumors and hematological cancers. Solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Additional exemplary solid tumors include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematological cancers include, e.g., leukemia or lymphomas. Metastases of the aforementioned cancers can also be treated or prevented in accordance with the methods described herein.

Cancer can be treated by, e.g., surgery, chemotherapy, radiation therapy, immunotherapy, monoclonal antibody therapy or other methods.

Examples of surgical procedures for cancer include mastectomy for breast cancer and prostatectomy for prostate cancer. Occasionally, surgery is necessary to control symptoms, such as spinal cord compression or bowel obstruction. This is referred to as palliative treatment.

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy.

Chemotherapy is the treatment of cancer with drugs (“anticancer drugs”) that can destroy cancer cells. Chemotherapy usually refers to cytotoxic drugs which affect rapidly dividing cells in general. Chemotherapy drugs interfere with cell division in various possible ways, e.g. with the duplication of DNA or the separation of newly formed chromosomes.

Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors imatinib and gefitinib. Monoclonal antibody therapy is another strategy in which the therapeutic agent is an antibody which specifically binds to a protein on the surface of the cancer cells. Examples include the anti-HER 2/neu antibody trastuzumab used in breast cancer, and the anti-CD20 antibody rituximab, used in a variety of B-cell malignancies.

Cancer immunotherapy refers to a diverse set of therapeutic strategies designed to induce the patient's own immune system to fight the tumor.

Hormonal therapy inhibits the growth of some cancers by providing or blocking certain hormones.

Angiogenesis inhibitors, such as bevacizumab, prevent the extensive growth of blood vessels (angiogenesis) that tumors require to survive.

Compositions and Routes of Administration

The compositions delineated herein include the compounds delineated herein (e.g., a compound described herein), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Kits

A compound described herein described herein can be provided in a kit. The kit includes (a) a compound described herein, e.g., a composition that includes a compound described herein, and, optionally (b) informational material. The informational material can be descriptive, instructional, marketing or other material that relates to the methods described herein and/or the use of a compound described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

What is claimed is:

1. A fluorinated compound selected from the following: a fluorinated venlafaxine, a fluorinated duloxetine, a fluorinated varenicline, a fluorinated atomoxetine, a fluorinated sertraline, a fluorinated trazodone, a fluorinated mirtazapine, a fluorinated amitriptyline, a fluorinated amoxapine, a fluorinated clomipramine, a fluorinated imipramine, a fluorinated nortriptyline, a fluorinated trimipramine, a fluorinated maprotiline, a fluorinated nefazodone, a fluorinated sibutramine, a fluorinated fluoxetine, a fluorinated doxepin and a fluorinated lofepramine.

2. The compound of claim 1, wherein the compound is a fluorinated duloxetine.

3. The compound of claim 1, wherein the compound is a fluorinated varenicline.

4. The compound of claim 1, wherein the compound is a fluorinated atomoxetine.

5. The compound of claim 1, wherein the compound is a fluorinated sertraline of one of the following formulae:

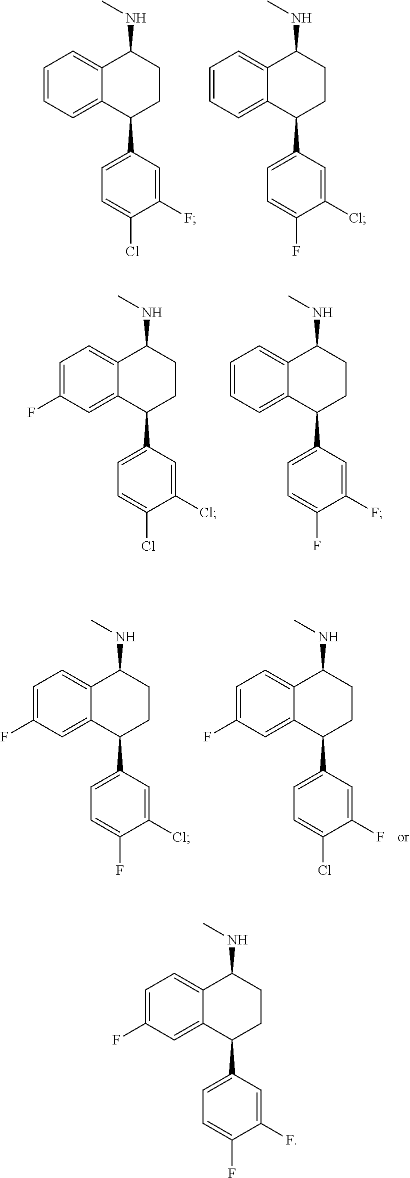

6. The compound of claim 1, wherein the compound is a fluorinated trazodone of the following formula:

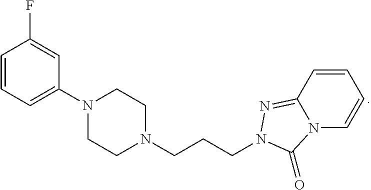

7. The compound of claim 1, wherein the compound is a fluorinated mirtazapine of the following formula:

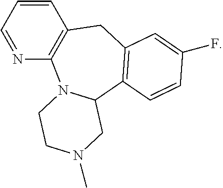

8. The compound of claim 1, wherein the compound is a fluorinated amitriptyline of the following formula:

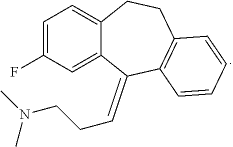

9. The compound of claim 1, wherein the compound is a fluorinated amoxapine of one of the following formulae:

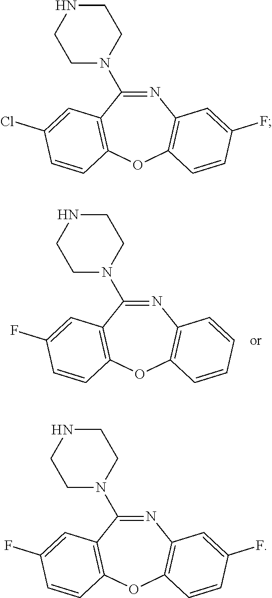

10. The compound of claim 1, wherein the compound is a fluorinated clomipramine of the following formula:

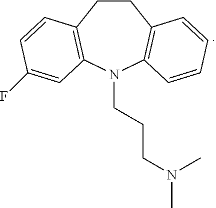

11. The compound of claim 1, wherein the compound is a fluorinated imipramine of the following formula:

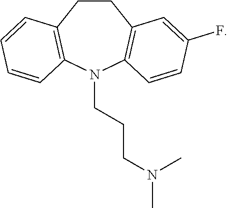

12. The compound of claim 1, wherein the compound is a fluorinated nortriptyline of the following formula:

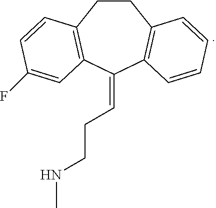

13. The compound of claim 1, wherein the compound is a fluorinated trimipramine of the following formula:

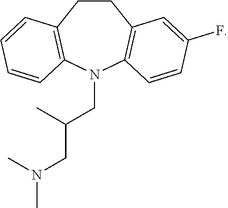

14. The compound of claim 1, wherein the compound is a fluorinated maprotiline of the following formula:

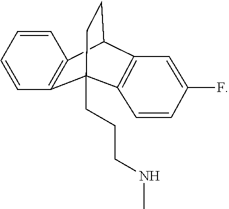

15. The compound of claim 1, wherein the compound is a fluorinated nefazodone of one of the following formulae:

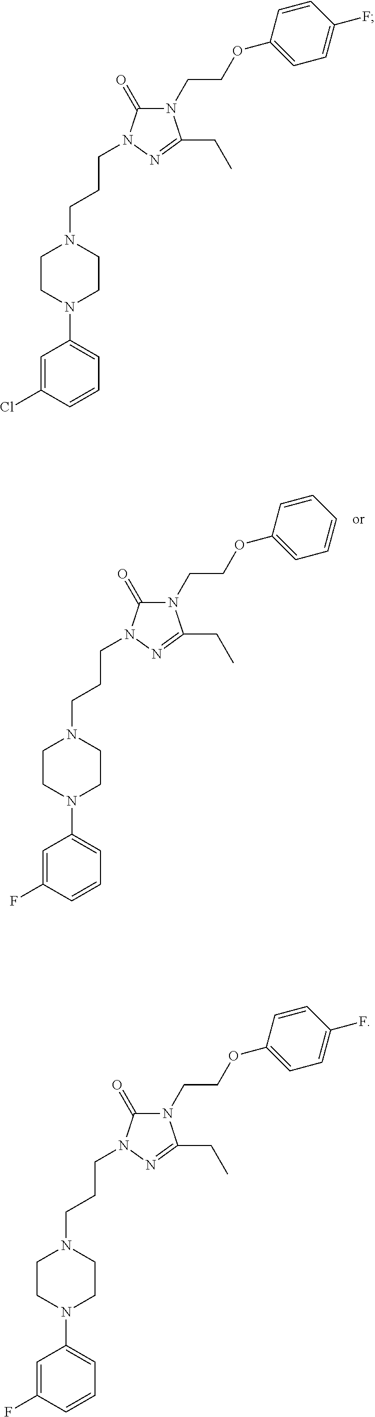

16. The compound of claim 1, wherein the compound is a fluorinated sibutramine.

17. (canceled)

18. The compound of claim 1, wherein the compound is a fluorinated fluoxetine of the following formula:

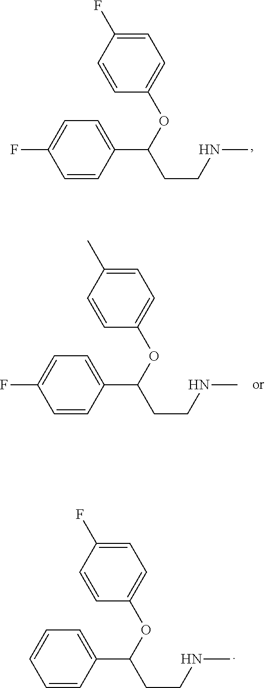

19.-21. (canceled)

22. The compound of claim 1, wherein the compound is a fluorinated doxepin of one of the following formulae:

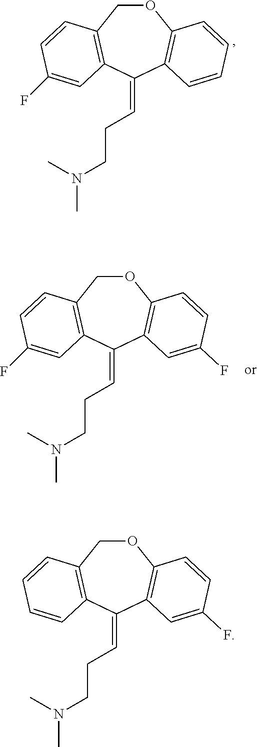

23. (canceled)

24. The compound of claim 1, wherein the compound is a fluorinated lofepramine of one of the following formulae:

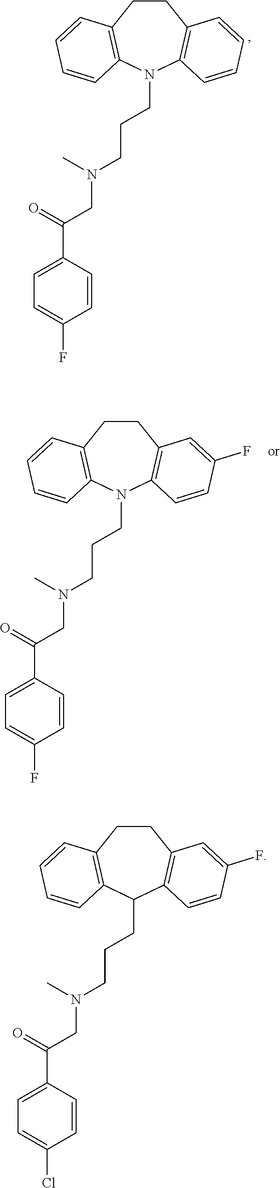

25.-141. (canceled)

142. The compound of claim 1, wherein the compound is a fluorinated venlafaxine of the following formula: [chemical structure]
